(19) United States
(12) Patent Application Publication
(10) Pub. No.: US 2013/0115683 A2
(43) Pub. Date: May 9, 2013

(54) METHOD FOR THE PREPARATION OF A PHARMACEUTICAL COMPOSITION

(71) Applicants: Nikolaus MACHUY, Berlin (DE); Alexander KARLAS, Berlin (DE); Thomas MEYER, Berlin (DE)

(72) Inventors: Nikolaus MACHUY, Berlin (DE); Alexander KARLAS, Berlin (DE); Thomas MEYER, Berlin (DE)

(73) Assignee: Max-Planck-Gesellschaft zur Foerderung der Wissenschaften e.V., Muenchen (DE)

(21) Appl. No.: 13/395,143

(22) PCT Filed: Sep. 10, 2010
(86) PCT No.: PCT/EP2010/063333

§ 371 Date: Jun. 26, 2012

(30) Foreign Application Priority Data
Sep. 11, 2010 (EP) 09170077.3

(51) Int. Cl. C12N 7/02; A01K 67/027; C12N 5/10

(52) U.S. Cl. 435/239; 435/325; 435/349; 435/366; 435/371; 435/369; 800/13

(57) ABSTRACT

The present invention relates to a method for the preparation of a pharmaceutical composition for the prevention or/and treatment of an influenza virus infection.

The present invention relates to a method for the production of a pharmaceutical composition for the prevention or/and treatment of an influenza virus infection.

Furthermore, the present invention relates to a pharmaceutical composition for the prevention or/and treatment of an influenza virus infection.

In view of the threatening influenza pandemic, there is an acute need to develop and make available lastingly effective drugs. In Germany alone the annual occurrence of influenza causes between 5,000 and 20,000 deaths a year (source: Robert-Koch Institute). The recurring big influenza pandemics are especially feared. The first big pandemic, the so-called “Spanish Flu”, cost about 40 million lives in the years 1918-1919 including a high percentage of healthy, middle-aged people. A similar pandemic could be caused by the H5N1 influenza virus (2, 3), which at the moment replicates mainly in birds, if acquired mutations enable the virus to be transmitted from person to person. More recently, a novel influenza virus variant has emerged, i.e. the influenza A (H1N1) ‘swine flu’ strain (4), posing an unpredictable pandemic threat. The probability of a human pandemic has recently grown more acute with the spreading of bird flu (H5N1) worldwide and the infection of domestic animals. It is only a question of time until a highly pathogenic human influenza-recombinant emerges. The methods available at the moment for prophylaxis or therapy of an influenza infection, such as vaccination with viral surface proteins or the use of antiviral drugs (neuraminidase inhibitors or ion channel blockers), have various disadvantages. Already at this early stage resistance is appearing against one of our most effective preparations (Tamiflu), which may make it unsuitable to contain a pandemic. A central problem in the use of vaccines and drugs against influenza is the variability of the pathogen. Up to now the development of effective vaccines has required accurate prediction of the pathogen variant. Drugs directed against viral components can rapidly lose their effectiveness because of mutations of the pathogen.

An area of research which has received little attention up to now is the identification of critical target structures in the host cell. Viruses are dependent on certain cellular proteins to be able to replicate within the host. The knowledge of such cellular factors that are essential for viral replication but dispensable (at least temporarily) for humans could lead to the development of novel drugs. Rough estimates predict about 500 genes in the human genome which are essential for viral multiplication. Of these, 10% at least are probably dispensable temporarily or even permanently for the human organism. Inhibition of these genes and their products, which in contrast to the viral targets are constant in their structure, would enable the development of a new generation of antiviral drugs in the shortest time. Inhibition of such gene products could overcome the development of viral escape mutants that are not longer sensitive to antiviral drugs. Amongst other gene families kinases that are important regulatory proteins within the cell are often hijacked by viruses to manipulate the constitution of the host cell.

Influenza A is a negative-stranded RNA virus that exhibits an array of strategies to facilitate successful survival within mammalian host cells (5). Upon infection, binding of innate immune receptors, such as the cellular protein retinoic acid-inducible gene I (RIG-I), with their cognate ligands triggers the transient expression of dozens of immune and inflammation related genes (6, 7). In particular, subsequent induction of type I interferon stimulates the up-regulation of GTPases with intrinsic antiviral activity, such as the myxovirus resistance (Mx) proteins. The antiviral activity of Mx proteins against members of the orthomyxovirus family was first observed in mice (8). The nucleus-located Mx1 protein confers protection against otherwise lethal infections with influenza virus, including strains of the pandemic 1918 and the highly lethal H5N1 influenza viruses (9, 10). The human ortholog, MxA, localizes to the cytoplasm and is thought to act by binding and inactivating incoming viral nucleocapsids (11). Interestingly, human MxA reportedly exhibits a protective function in transgenic mice against various RNA viruses (12). To counteract these innate response strategies, influenza viruses employ their NS1 protein; for example, by reducing interferon-β (IFN-β) production or by blocking expression of the antiviral proteins 2′-5′ oligoadenylate synthetase (OAS) and protein kinase R (PKR) (13). However, the active suppression of MxA previously observed during influenza A infections ((1). T. D. Carroll et al., J. Immunol. 180, 2385 (2008) in vitro and in vivo is currently not completely understood.

An intriguing strategy employed by viral agents to regulate their infectious potential is the use of microRNAs (miRNAs); a class of ˜22 nt long non-protein-coding short interfering RNA molecules, known as key post-transcriptional regulators of gene expression (14). Viruses with large genomes can encode their own miRNAs to alter host physiology and enhance replication (15). Conversely, the small RNA genome hepatitis C virus can manipulate expression of host cell miR-122 to foster its replication (16).

Common strategies for the production of influenza virus vaccines are based upon influenza virus replication in embryonated hens' eggs or in cell culture. Virus replication in cell culture or embryonated eggs is a time-consuming and expensive procedure. Therefore, it is the problem of the present invention to improve the methods for the influenza vaccine production.

An object of the present invention is a method for the preparation of an influenza virus comprising the steps:
a providing a modified cell, a modified embryonated egg or/and a modified non-human organism capable of replicating an influenza virus, wherein the capability of influenza virus replication is increased compared with influenza virus replication in the absence of the modification,
b contacting the cell, the embryonated egg or/and the organism of (a) with an influenza virus,
c cultivating the cell, the embryonated egg or/and the non-human organism under conditions allowing the replication of the influenza virus, and
d isolating the influenza virus or/and at least on component thereof produced in step (c).

From the influenza virus of step (d), a pharmaceutical composition for the prevention or/and treatment of an influenza virus infection may be prepared, optionally together with a pharmaceutically acceptable carrier, adjuvant, diluent or/and additive.

Another object of the present invention is a method for the preparation of a pharmaceutical composition for the prevention or/and treatment of an influenza virus infection, comprising the steps:
a providing a modified cell, a modified embryonated egg or/and a modified non-human organism capable of replicating an influenza virus, wherein the capability of influenza virus replication is increased compared with influenza virus replication in the absence of the modification,
b contacting the cell, the embryonated egg or/and the organism of (a) with an influenza virus,
c cultivating the cell, the embryonated egg or/and the non-human organism under conditions allowing the replication of the influenza virus,
d isolating the influenza virus or/and at least one component thereof produced in step (c), and,
e preparing the pharmaceutical composition from the influenza virus or/and the components thereof isolated in step (d), optionally together with a pharmaceutically acceptable carrier, adjuvant, diluent or/and additive.

A reference herein to the “method” or “method of the present invention” is a reference to the method for the preparation of an influenza virus and to the method for the preparation of a pharmaceutical composition for the prevention or/and treatment of an influenza virus infection.

The cell employed in step (a) may be any cell capable of being infected with an influenza virus. Cell lines suitable for the production of an influenza virus are known. Preferably the cell is a mammalian cell or an avian cell. Also preferred is a human cell. Also preferred is an epithelial cell, such as a lung epithelial cell. The cell may be a cell line. A suitable lung epithelial cell line is A594. Another suitable cell is the human embryonic kidney cell line 293T. In one embodiment of the present invention, the method of the present invention employs a cell as described herein.

The non-human organism employed in step (a) may be any organism capable of being infected with an influenza virus. Preferably the organism is an organism employed in the production of an influenza vaccine. More preferable, the organism is an embryonated egg, such as an embryonated hen's egg. The person skilled in the art know methods of obtaining such organism. The methods for obtained an embryonated egg by fertilization are known. Inducing influenza virus replication by inoculation with an influenza virus is known. In one embodiment of the present invention, the method of the present invention employs a non-human organism or/and an embryonated egg, as described herein.

Step (a) of the present invention may include the provision of a cell, an embryonated egg or/and a non-human organism modified as described herein, or may include the step of modification.

It is preferred that a modified cell or/and a modified embryonated egg is provided in step (a) and employed in steps (b), (c) and (d), or in steps (b), (c) (d) and (e), as described herein.

“Modification of the cell, the embryonated egg or/and non-human organism”, as used herein, includes downregulation or/and upregulation of the expression or/and activity of at least one gene or/and gene product in the cell, the egg or/and the organism.

“Modification of the cell, the embryonated egg or/and the non-human organism”, as described herein, may include contacting the cell, the embryonated egg or/and the non-human organism with at least one modulator capable of increasing the influenza virus replication in the cell or/and the organism, compared with influenza virus replication in the absence of the modulator, wherein contacting may be performed before or after step (b), or simultaneously with step (b).

“Modification of the cell, the embryonated egg or/and non-human organism”, as described herein, may include the production or/and provision of a recombinant cell, a recombinant embryonated egg or/and recombinant non-human organism, wherein the expression or/and activity of at least one gene or/and gene product is modified so that the capability of the cell, the embryonated egg or/and the non-human organism of replicating an influenza virus is increased compared with influenza virus replication in the absence of the modification.

Preparation of a recombinant cell, a recombinant embryonated egg or/and recombinant non-human organism may include introduction of a nucleic acid molecule into the cell, the embryonated egg or/and the non-human organism, or/and deletion of a nucleic acid sequence in the cell, the egg or/and the organism. The nucleic acid molecule may be incorporated into the genome of the cell, of the embryonated egg or/and of the non-human organism. Thereby, sequences of the cell, the egg or/and the organism may be modified, replaced or/and deleted. The nucleic acid molecule may comprise a sequence heterologous to the cell or/and the organism. Incorporation of the nucleic acid molecule may be performed permanently or transiently. A recombinant embryonated egg or/and recombinant non-human organism may be prepared by manipulation of the germ line. In the context of the present invention, “embryonated egg” in particular refers to the embryo. For instance, “modification of the embryonated egg” is in particular a modification of the embryo.

The person skilled in the art knows methods of introducing a nucleic acid molecule into a cell, an embryonated egg or/and an organism, or/and methods of deletion of a nucleic acid sequence in the cell, the embryonated egg or/and the organism (“recombinant technology”, as employed herein). These methods may include transfection employing a suitable vector, such as a plasmid. These methods may also include homologous recombination of the nucleic acid molecule in the genome of the cell or/and the organism. The nucleic acid molecule may also be randomly inserted into the genome of the cell, the embryonated egg or/and the organism.

Tables 1a, 1b, 4 and 5 describe targets for modulation of influenza virus replication, wherein the targets may be suitable for the modification of the cell, the embryonated egg or/and non-human organism, either by contacting with a modulator, or by recombinant technology, as described herein.

“Modulation” in the context of the present invention may be “activation” or “inhibition”.

Examples of genes which upon downregulation increase the influenza virus replication are described in Tables 1a and 5. Thus, by increasing expression or/and activity of these genes or/and gene products thereof, the influenza virus replication can be reduced. A decreased expression or/and activity of these genes or/and gene products can be exploited in the method of the present invention by improvement of virus production.

The cell, the embryonated egg or/and non-human organism provided in step (a) may thus be a recombinant cell, a recombinant embryonated egg or/and recombinant non-human organism, wherein the gene expression or/and the activity of a gene selected from Tables 1a and 5 is downregulated.

Examples of genes which upon downregulation decrease the influenza virus replication are described in Table 1b and 4. Thus, by decreasing expression or/and activity of these genes or/and gene products, the influenza virus replication can be reduced. An increased expression or/and activity of these genes or/and gene products can be exploited in the method of the present invention by improvement of virus production.

The cell, embryonated egg or/and non-human organism provided in step (a) may thus be a recombinant cell, a recombinant embryonated egg or/and recombinant non-human organism, wherein the gene expression or/and the activity of a gene selected from Table 1b and Table 4 is upregulated. In particular upregulation of a gene selected from Table 1b and Table 4 is over-expression of said gene.

In the context of the present invention, a “target” includes
a a nucleotide sequence within a gene or/and a genome, in particular the within a human gene or/and the human genome,
b a nucleic acid, or/and a polypeptide encoded by the nucleotide sequence of (a).

The sequence of (a) or/and (b) may be involved in regulation of influenza virus replication in a host cell. The target may be directly or indirectly involved in the regulation of influenza virus replication. In particular, a target is suitable for increasing of influenza virus replication, either by activation of the target or by inhibition of the target.

Examples of targets are genes and partial sequences of genes, such as regulatory sequences. A target according to the present invention also includes a gene product such as RNA, in particular mRNA, tRNA, rRNA, miRNA, piRNA. A target may also include a polypeptide or/and a protein encoded by the target gene. Preferred gene products of a target gene are selected from mRNA, miRNA, polypeptide(s) or/and protein(s) encoded by the target gene. The most preferred gene product is a polypeptide or protein encoded by the target gene. A target protein or a target polypeptide may be posttranslationally modified or not.

A “Gene product” as used herein may be selected from RNA, in particular mRNA, tRNA, rRNA, miRNA, and piRNA. A “Gene product” may also be a polypeptide or/and a protein encoded by said gene.

In the context of the present invention, “activity” of the gene or/and gene product includes transcription, translation, post translational modification, post transcriptional regulation, modulation of the activity of the gene or/and gene product. The activity may be modulated by ligand binding, which ligand may be an activator or inhibitor. The activity may also be modulated by an miRNA molecule, an shRNA molecule, an siRNA molecule, an antisense nucleic acid, a decoy nucleic acid or/and any other nucleic acid, as described herein. The activity of the gene may also be modulated by recombinant technology, as described herein. Modulation may also be performed by a small molecule, an antibody, an aptamer, or/and a spiegelmer (mirror image aptamer).

The method of the present invention may be suitable for the production of a pharmaceutical composition for the prevention or/and treatment of an infection with any influenza virus.

The influenza virus may be any influenza virus suitable for vaccine production. The influenza virus may be an influenza A virus. The influenza A virus may be selected from influenza A viruses isolated so far from avian and mammalian organisms. In particular, the influenza A virus may be selected from H1N1, H1N2, H1N3, H1N4, H1N5, H1N6, H1N7, H1N9, H2N1, H2N2, H2N3, H2N4, H2N5, H2N7, H2N8, H2N9, H3N1, H3N2, H3N3, H3N4, H3N5, H3N6, H3N8, H4N1, H4N2, H4N3, H4N4, H4N5, H4N6, H4N8, H4N9, H5N1, H5N2, H5N3, H5N6, H5N7, H5N8, H5N9, H6N1, H6N2, H6N3, H6N4, H6N5, H6N6, H6N7, H6N8, H6N9, H7N1, H7N2, H7N3, H7N4, H7N5, H7N7, H7N8, H7N9, H9N1, H9N2, H9N3, H9N5, H9N6, H9N7, H9N8, H10N1, H10N3, H10N4, H10N6, H10N7, H10N8, H10N9, H11N2, H11N3, H11N6, H11N9, H12N1, H12N4, H12N5, H12N9, H13N2, H13N6, H13N8, H13N9, H14N5, H15N2, H15N8, H15N9 and H16N3. More particularly, the influenza A virus is selected from H1N1, H3N2, H7N7, H5N1. Even more particularly, the influenza A virus is strain Puerto Rico/8/34, the avian influenza virus isolate H5N1, the avian influenza strain A/FPV/Bratislava/79 (H7N7), strain A/WSN/33 (H1N1), strain A/Panama/99 (H3N2), or a swine flu strain H1N1.

The influenza virus may be an influenza B virus. In particular, the influenza B virus may be selected from representatives of the Victoria line and representatives of the Yamagata line.

In the method of the present invention, modification of the cell or/and organism according to step (a) to increase the influenza virus replication includes modulating the expression of a gene selected from Table 1A, Table 1B, Table 4 and Table 5, or/and a gene product thereof. In particular, modification of the cell or/and organism may activate the expression of a gene selected from Table 1B and Table 4 or/and a gene product thereof, or modification of the cell or/and organism may inhibit the expression of a gene selected from Tables 1A and 5 or/and a gene product thereof. Modulating the expression may be performed by contacting the cell, the embryonated egg or/and the organism with a modulator as described herein, or may be performed in a recombinant cell, a recombinant embryonated egg or/and recombinant organism, the production of which is described herein.

On the RNA level, inhibition may be performed by antisense nucleic acid, siRNA, shRNA, a decoy nucleic acid or/and a derivative thereof. On the level of the MxA polypeptide, inhibition may be performed by a small molecule, an antibody, an aptamer, a spiegelmer (mirror image aptamer).

Modification of the cell, of the embryonated egg or/and of the non-human organism may include the inhibition of the expression or/and gene product activity of a gene, wherein the gene comprises
a a nucleotide sequence selected from the sequences of Tables 1A and 5,
b a fragment of the sequence of (a) having a length of at least 70%, at least 80%, at least 90%, at least 95%, at least 98%, at least 99% of the sequence of (a),
c a sequence which is at least 70%, preferably at least 80%, more preferably at least 90% identical to the sequence of (a) or/and (b), or/and
d a sequence complementary to a sequence of (a), (b) or/and (c).

Modification of the cell, the embryonated egg or/and the non-human organism may include the activation of the expression or/and gene product activity of a gene, wherein the gene comprises
i a nucleotide sequence selected from the sequences of Table 1B and Table 4,
ii a fragment of the sequence of (i) having a length of at least 70%, at least 80%, at least 90%, at least 95%, at least 98%, at least 99% of the sequence of (i),
iii a sequence which is at least 70%, preferably at least 80%, more preferably at least 90% identical to the sequence of (i) or/and (ii), or/and
iv a sequence complementary to a sequence of (i), (ii) or/and (iii).

The at least one modulator capable of increasing the influenza virus replication may be capable of inhibiting expression or/and gene product activity of a gene, wherein the gene comprises
a a nucleotide sequence selected from the sequences of Tables 1A and 5,
b a fragment of the sequence of (a) having a length of at least 70%, at least 80%, at least 90%, at least 95%, at least 98%, at least 99% of the sequence of (a),
c a sequence which is at least 70%, preferably at least 80%, more preferably at least 90% identical to the sequence of (a) or/and (b), or/and
d a sequence complementary to a sequence of (a), (b) or/and (c).

The at least one modulator capable of increasing the influenza virus replication may be capable of activating the expression or/and gene product activity of a gene, wherein the gene comprises
i a nucleotide sequence selected from the sequences of Table 1B and Table 4,
ii a fragment of the sequence of (i) having a length of at least 70%, at least 80%, at least 90%, at least 95%, at least 98%, at least 99% of the sequence of (i),
iii a sequence which is at least 70%, preferably at least 80%, more preferably at least 90% identical to the sequence of (i) or/and (ii), or/and
iv a sequence complementary to a sequence of (i), (ii) or/and (iii).

The cell, the embryonated egg or/and non-human organism may be recombinantly modified, as described herein, so that expression or/and gene product activity of a gene is inhibited, wherein the gene comprises
a a nucleotide sequence selected from the sequences of Tables 1A and 5,
b a fragment of the sequence of (a) having a length of at least 70%, at least 80%, at least 90%, at least 95%, at least 98%, at least 99% of the sequence of (a),
c a sequence which is at least 70%, preferably at least 80%, more preferably at least 90% identical to the sequence of (a) or/and (b), or/and
d a sequence complementary to a sequence of (a), (b) or/and (c).

The cell, the embryonated egg or/and non-human organism may be recombinantly modified, as described herein, so that expression or/and gene product activity of a gene is activated, wherein the gene comprises
i a nucleotide sequence selected from the sequences of Table 1B and Table 4,
ii a fragment of the sequence of (i) having a length of at least 70%, at least 80%, at least 90%, at least 95%, at least 98%, at least 99% of the sequence of (i),
iii a sequence which is at least 70%, preferably at least 80%, more preferably at least 90% identical to the sequence of (i) or/and (ii), or/and
iv a sequence complementary to a sequence of (i), (ii) or/and (iii).

As used herein, a reference to a nucleotide sequence or/and a gene disclosed in one or more Tables of the present invention is understood to be a reference to a specific sequence disclosed in said Table(s), and a reference to a sequence characterized by an Accession Number, a Gene name, a Locus Link, a Symbol, a GeneID, a GeneSymbol, or/and a GenbankID disclosed in said Table(s). By reference to an Accession Number, a Gene name, a Locus Link, a Symbol, a GeneID, a GeneSymbol, or/and a GenbankID, the skilled person is able to identify the corresponding nucleotide sequence or/and amino acid sequence. A particular sequence may be characterized by one or more of an Accession Number, a Gene name, a Locus Link, a Symbol, a GeneID, a GeneSymbol, and a GenbankID, as indicated in the Tables. A reference to a gene disclosed in one or more Tables of the present invention is understood to be in particular a reference to a sequence, such as a gene sequence, characterized by an Accession Number, a Gene name, a Locus Link, a Symbol, a GeneID, a GeneSymbol, or/and a GenbankID disclosed in said Table(s).

Modification (including modulation and recombinant modification) may be a modification of a kinase or/and a modulator of a kinase binding polypeptide, wherein the at least one kinase or/and kinase binding polypeptide is encoded by a nucleic acid or/and gene selected from Table 1A and Table 1B.

In the method of the present invention, the at least one modulator capable of increasing the influenza virus replication may be an activator comprising:
i a nucleotide sequence selected from Table 1B and Table 4,
ii a fragment of the sequence of (a) having a length of at least 70%, at least 80%, at least 90%, at least 95%, at least 98%, at least 99% of the sequence of (i),
iii a sequence which is at least 70%, preferably at least 80%, more preferably at least 90% identical to the sequence of (i) or/and (ii), or/and
iv a sequence complementary to a sequence of (i), (ii) or/and (iii).

The at least one activator may be capable of activating expression or/and gene product activity of a gene comprising sequence (i), (ii) (iii) or/and (iv).

In the method of the present invention, the at least one modulator capable of increasing the influenza virus replication may be an inhibitor comprising:
a a nucleotide sequence selected from Tables 1A and 5,
b a fragment of the sequence of (a) having a length of at least 70%, at least 80%, at least 90%, at least 95%, at least 98%, at least 99% of the sequence of (a),
c a sequence which is at least 70%, preferably at least 80%, more preferably at least 90% identical to the sequence of (a) or/and (b), or/and
d a sequence complementary to a sequence of (a), (b) or/and (c).

The at least one inhibitor may be capable of inhibiting expression or/and gene product activity of a gene comprising sequence (a), (b) (c) or/and (d).

The at least modulator of influenza virus replication employed in the method of the present invention of the present invention may be selected from the group consisting of nucleic acids, nucleic acid analogues such as ribozymes, peptides, polypeptides, antibodies, aptamers, spiegelmers, small molecules and decoy nucleic acids.

The modulator of influenza virus replication may be a compound having a molecular weight smaller than 1000 Dalton or smaller than 500 Dalton. In the context of the present invention, “small molecule” refers to a compound having a molecular weight smaller than 1000 Dalton or smaller than 500 Dalton. In the method of the present invention, the small molecule may be directed against a polypeptide comprising
a an amino acid sequence encoded by a nucleic acid or/and gene selected from Table 1A, Table 1B, Table 4, and Table 5,
b a fragment of the sequence of (a) having a length of at least 70%, at least 80%, at least 90%, at least 95%, at least 98%, at least 99% of the sequence of (a), or/and
c an amino acid sequence which is at least 70%, preferably at least 80%, more preferably at least 90% identical to the sequence of (a).

The modulator of the present invention preferably comprises a nucleic acid, wherein the nucleic acid comprises a nucleotide sequence selected from the sequences of Table 2 and Table 4 and fragments thereof.

Preferably, the nucleic acid is selected from
1 (a) RNA, analogues and derivatives thereof,
2 (b) DNA, analogues and derivatives thereof, and
3 (c) combinations of (a) and (b).

Suitable inhibitors are RNA molecules capable of RNA interference. The modulator of the present invention, in particular the inhibitor of the present invention may comprise
i an RNA molecule capable of RNA interference, such as siRNA or/and shRNA,
ii a miRNA,
iii a precursor of the RNA molecule (i) or/and (ii),
iv a fragment of the RNA molecule (i), (ii) or/and (iii),
v a derivative of the RNA molecule of (i), (ii) (iii) or/and (iv), or/and
vi a DNA molecule encoding the RNA molecule of (i), (ii) (iii) or/and (iv).

A preferred modulator is
i a miRNA,
ii a precursor of the RNA molecule (i), or/and
iii a DNA molecule encoding the RNA molecule (i) or/and the precursor (ii).

Yet another preferred modulator is
i an RNA molecule capable of RNA interference, such as siRNA or/and shRNA,
ii a precursor of the RNA molecule (i), or/and
iii a DNA molecule encoding the RNA molecule (i) or/and the precursor (ii).

RNA molecules capable of RNA interference are described in WO 02/44321 the disclosure of which is included herein by reference. MicroRNAs are described in Bartel D (Cell 136:215-233, 2009), the disclosure of which is included herein by reference.

The RNA molecule of the present invention may be a double-stranded RNA molecule, preferably a double-stranded siRNA molecule with or without a single-stranded overhang alone at one end or at both ends. The siRNA molecule may comprise at least one nucleotide analogue or/and deoxyribonucleotide.

The RNA molecule of the present invention may be an shRNA molecule. The shRNA molecule may comprise at least one nucleotide analogue or/and deoxyribonucleotide.

The DNA molecule as employed in the present invention may be a vector.

The nucleic acid employed in the present invention may be an antisense nucleic acid or a DNA encoding the antisense nucleic acid.

The nucleic acid or/and nucleic acid fragment employed in the present invention may have a length of at least 15, preferably at least 17, more preferably at least 19, most preferably at least 21 nucleotides. The nucleic acid or/and the nucleic acid fragment may have a length of at the maximum 29, preferably at the maximum 27, more preferably at the maximum 25, especially more preferably at the maximum 23, most preferably at the maximum 22 nucleotides.

The nucleic acid employed in the present invention may be a microRNA (miRNA), a precursor, a fragment, or a derivative thereof. The miRNA may have the length of the nucleic acid as described herein. The miRNA may in particular have a length of about 22 nucleotides, more preferably 22 nucleotides.

The modulator of the present invention may comprise an antibody, wherein the antibody may be directed against a kinase or/and kinase binding polypeptide.

Preferably the antibody is directed against a kinase or/and kinase binding polypeptide comprising
a an amino acid sequence encoded by a nucleic acid or/and gene selected from Table 1A, and Table 1B,
b a fragment of the sequence of (a) having a length of at least 70%, at least 80%, at least 90%, at least 95%, at least 98%, at least 99% of the sequence of (a), or/and
c an amino acid sequence which is at least 70%, preferably at least 80%, more preferably at least 90% identical to the sequence of (a) or/and (b).

In another preferred embodiment, the antibody is directed against a polypeptide comprising
a an amino acid sequence encoded by a nucleic acid or/and gene selected from Table 4,
b a fragment of the sequence of (a) having a length of at least 70%, at least 80%, at least 90%, at least 95%, at least 98%, at least 99% of the sequence of (a), or/and
c an amino acid sequence which is at least 70%, preferably at least 80%, more preferably at least 90% identical to the sequence of (a) or/and (b).

In yet another preferred embodiment, the antibody is directed against a polypeptide comprising
a an amino acid sequence encoded by a nucleic acid or/and gene selected from Table 5,
b a fragment of the sequence of (a) having a length of at least 70%, at least 80%, at least 90%, at least 95%, at least 98%, at least 99% of the sequence of (a), or/and
c an amino acid sequence which is at least 70%, preferably at least 80%, more preferably at least 90% identical to the sequence of (a) or/and (b).

The antibody of the present invention may be a monoclonal or polyclonal antibody, a chimeric antibody, a chimeric single chain antibody, a Fab fragment or a fragment produced by a Fab expression library.

Techniques of preparing antibodies of the present invention are known by a skilled person. Monoclonal antibodies may be prepared by the human B-cell hybridoma technique or by the EBV-hybridoma technique (Köhler et al., 1975, Nature 256:495-497, Kozbor et al., 1985, J. Immunol. Methods 81, 31-42, Cote et al., PNAS, 80:2026-2030, Cole et al., 1984, Mol. Cell Biol. 62:109-120). Chimeric antibodies (mouse/human) may be prepared by carrying out the methods of Morrison et al. (1984, PNAS, 81:6851-6855), Neuberger et al. (1984, 312:604-608) and Takeda et al. (1985, Nature 314:452-454). Single chain antibodies may be prepared by techniques known by a person skilled in the art.

Recombinant immunoglobulin libraries (Orlandi et al, 1989, PNAS 86:3833-3837, Winter et al., 1991, Nature 349:293-299) may be screened to obtain an antibody of the present invention. A random combinatory immunoglobulin library (Burton, 1991, PNAS, 88:11120-11123) may be used to generate an antibody with a related specifity having a different idiotypic composition.

Another strategy for antibody production is the in vivo stimulation of the lymphocyte population.

Furthermore, antibody fragments (containing F(ab′)₂ fragments) of the present invention can be prepared by protease digestion of an antibody, e.g. by pepsin. Reducing the disulfide bonding of such F(ab′)₂ fragments results in the Fab fragments. In another approach, the Fab fragment may be directly obtained from an Fab expression library (Huse et al., 1989, Science 254:1275-1281).

Polyclonal antibodies of the present invention may be prepared employing an amino acid sequence encoded by a nucleic acid or/and gene selected from Table 1A, Table 1B, Table 4 and Table 5 or immunogenic fragments thereof as antigen by standard immunization protocols of a host, e.g. a horse, a goat, a rabbit, a human, etc., which standard immunization protocols are known by a person skilled in the art.

The antibody may be an antibody specific for a gene product of a target gene, in particular an antibody specific for a polypeptide or protein encoded by a target gene.

Aptamers and spiegelmers share binding properties with antibodies. Aptamers and spiegelmers are designed for specifically binding a target molecule.

The nucleic acid or the present invention may be selected from (a) aptamers, (b) DNA molecules encoding an aptamer, and (c) spiegelmers.

The skilled person knows aptamers. In the present invention, an “aptamer” may be a nucleic acid that can bind to a target molecule. Aptamers can be identified in combinational nucleic acid libraries (e.g. comprising >10¹⁵ different nucleic acid sequences) by binding to the immobilized target molecule and subsequent identification of the nucleic acid sequence. This selection procedure may be repeated one or more times in order to improve the specificity. The person skilled in the art knows suitable methods for producing an aptamer specifically binding a predetermined molecule. The aptamer may have a length of a nucleic acid as described herein. The aptamer may have a length of up to 300, up to 200, up to 100, or up to 50 nucleotides. The aptamer may have a length of at least 10, at least 15, or at least 20 nucleotides. The aptamer may be encoded by a DNA molecule. The aptamer may comprise at least one nucleotide analogue or/and at least one nucleotide derivatives, as described herein.

The skilled person knows spiegelmers. In the present invention; a “spiegelmer” may be a nucleic acid that can bind to a target molecule. The person skilled in the art knows suitable methods for production of a spiegelmer specifically binding a predetermined molecule. The spiegelmer comprises nucleotides capable of forming bindings which are nuclease resistant. Preferably the spiegelmer comprises L nucleotides. More preferably, the spiegelmer is an L-oligonucleotide. The spiegelmer may have a length of a nucleic acid as described herein. The spiegelmer may have a length of up to 300, up to 200, up to 100, or up to 50 nucleotides. The spiegelmer may have a length of at least 10, at least 15, or at least 20 nucleotides. The spiegelmer may comprise at least one nucleotide analogue or/and at least one nucleotide derivatives, as described herein.

The skilled person knows decoy nucleic acids. In the present invention, a “decoy” or “decoy nucleic acid” may be a nucleic acid capable of specifically binding a nucleic acid binding protein, such as a DNA binding protein. The decoy nucleic acid may be a DNA molecule, preferably a double stranded DNA molecule. The decoy nucleic acid comprises a sequence termed “recognition sequence” which can be recognized by a nucleic acid binding protein. The recognition sequence preferably has a length of at least 3, at least 5, or at least 10 nucleotides. The recognition sequence preferably has a length of up to 15, up to 20, or up to 25 nucleotides. Examples of nucleic acid binding proteins are transcription factors, which preferably bind double stranded DNA molecules. Transfection of a cell, an embryonated egg, or/and a non-human animal, as described herein, with a decoy nucleic acid may result in reduction of the activity of the nucleic acid binding protein to which the decoy nucleic acid binds. The decoy nucleic acid as described herein may have a length of nucleic acid molecules as described herein. The decoy nucleic acid molecule may have a length of up to 300, up to 200, up to 100, up to 50, up to 40, or up to 30 nucleotides. The decoy nucleic may have a length of at least 3, at least 5, at least 10, at least 15, or at least 20 nucleotides. The decoy nucleic acid may be encoded by a DNA molecule. The decoy nucleic acid may comprise at least one nucleotide analogue or/and at least one nucleotide derivatives, as described herein.

An RNA or/and a DNA molecule as described herein may comprise at least one nucleotide analogue. As used herein, “nucleotide analogue” may refer to building blocks suitable for a modification in the backbone, at least one ribose, at least one base, the 3′ end or/and the 5′ end in the nucleic acid. Backbone modifications include phosphorothioate linkage (PTs); peptide nucleic acids (PNAs); morpholino nucleic acids; phosphoroamidate-linked DNAs (PAs), which contain backbone nitrogen. Ribose modifications include Locked nucleic acids (LNA) e.g. with methylene bridge joining the 2′ oxygen of ribose with the 4′ carbon; 2′-deoxy-2′-fluorouridine; 2′-fluoro (2′-F); 2′-O-alkyl-RNAs (2-O-RNAs), e.g. 2′-O-methyl (2′-O-Me), 2′-O-methoxyethyl (2′-O-MOE). A modified base may be 2′-fluoropyrimidine. 5′ modifications include 5′-TAMRA-hexyl linker, 5′-Phosphate, 5′-Amino, 5′-Amino-C6 linker, 5′-Biotin, 5′-Fluorescein, 5′-Tetrachloro-fluorescein, 5′-Pyrene, 5′-Thiol, 5′-Amino, (12 Carbon) linker, 5′-Dabcyl, 5′-Cholesterol, 5′-DY547 (Cy3™ alternate). 3′ end modifications include 3′-inverted deoxythymidine, 3′-puromycin, 3′-dideoxy-cytidine, 3′-cholesterol, 3′-amino modifier (6 atom), 3′-DY547 (Cy3™ alternate).

In particular, nucleotide analogues as described herein are suitable building blocks in siRNA, antisense RNA, and aptamers.

As used herein, “nucleic acid analogue” refers to nucleic acids comprising at least one nucleotide analogue as described herein. Further, a nucleic acid molecule as described herein may comprise at least one deoxyribonucleotide and at least one ribonucleotide.

An RNA molecule of the present invention may comprise at least one deoxyribonucleotide or/and at least one nucleotide analogue. A DNA molecule of the present invention may comprise at least one ribonucleotide or/and at least one nucleotide analogue.

Derivatives as described herein refers to chemically modified compounds. Derivatives of nucleic acid molecules as described herein refers to nucleic acid molecules which are chemically modified. A modification may be introduced into the nucleic acid molecule, or/and into at least one nucleic acid building block employed in the production of the nucleic acid.

In the present invention the term “fragment” refers to fragments of nucleic acids, polypeptides and proteins. “Fragment” also refers to partial sequences of nucleic acids, polypeptides and proteins.

Fragments of polypeptides or/and peptides as employed in the present invention, in particular fragments of an amino acid sequence encoded by a nucleic acid or/and gene selected from Table 1A, Table 1B, Table 4 and Table 5 may have a length of at least 5 amino acid residues, at least 10, or at least 20 amino acid residues. The length of said fragments may be 200 amino acid residues at the maximum, 100 amino acid residues at the maximum, 60 amino acid residues at the maximum, or 40 amino acid residues at the maximum.

A fragment of an amino acid sequence as described herein may have a length of at least 70%, at least 80%, at least 90%, at least 95%, at least 98%, at least 99% of the sequence.

A fragment of a nucleotide sequence as described herein may have a length of at least 70%, at least 80%, at least 90%, at least 95%, at least 98%, at least 99% of the sequence.

A fragment of a nucleic acid molecule given in Tables 1A, 1B, 4 and 5 may have a length of up to 1000, up to 2000, or up to 3000 nucleotides. A nucleic acid fragment may have a length of an siRNA molecule, an miRNA molecule, an aptamer, a spiegelmer, or/and a decoy as described herein. A nucleic acid fragment may also have a length of up to 300, up to 200, up to 100, or up to 50 nucleotides. A nucleic acid fragment may also have a length of at least 3, at least 5, at least 10, at least 15, or at least 20 nucleotides.

In the method of the present invention, modulating the expression of a gene may be downregulation or upregulation, in particular of transcription or/and translation.

It can easily be determined by a skilled person if a gene is upregulated or downregulated. In the context of the present invention, upregulation (activation) of gene expression may be an upregulation by a factor of at least 2, preferably at least 4. Downregulation (inhibition) in the context of the present invention may be a reduction of gene expression by a factor of at least 2, preferably at least 4. Most preferred is essentially complete inhibition of gene expression, e.g. by RNA interference.

Modulation of the activity of a gene may be decreasing or increasing of the activity. “Inhibition of the activity” may be a decrease of activity of a gene or gene product by a factor of at least 2, preferably at least 4. “Inhibition of the activity” includes essentially complete inhibition of activity. “Activation of the activity” may be an increase of activity of a gene or gene product by a factor of at least 2, preferably at least 4.

In the present invention, specific embodiments of the methods, cells, organisms, and pharmaceutical compositions described herein refer to any individual gene, nucleic acid sequence or/and gene product described in the present application. In a specific embodiment, an individual gene is selected from the genes described in Tables 1, 4, and 5. Other specific embodiments refer to individual genes described in Tables 1, 4, and 5. In another specific embodiment, an individual gene product is selected from the gene products produced by the genes described in Tables 1, 4, and 5. Other specific embodiments refer to the individual gene products produced by the genes described in Tables 1, 4, and 5. In yet another specific embodiment, an individual nucleic acid sequence or nucleic acid molecule is selected from the nucleic acid molecules or nucleic acid sequences described in Tables 1, 2, 4 and 5. Other specific embodiments refer to the individual nucleic acid molecules or nucleic acid sequences described in Tables 1, 2, 4, and 5. Further specific embodiment refer to any combination of genes, gene products and nucleic acid molecules described in the Tables 1, 2, 3, 4, and 5. Combinations may comprise 2, 3, 4, 5, 6 ,7, 8, 9, 10 or even more different species. Table 3 refers to specific combinations of nucleic acid molecules.

Further specific embodiments of the present invention refer to sequences disclosed in Table 5. Specific embodiments of the present invention refer to any individual gene, nucleic acid molecule or/and gene product described in Table 5. In a specific embodiment, an individual gene is selected from the genes described in Table 5. Other specific embodiments refer to the individual genes described in Table 5. In another specific embodiment, an individual gene product is selected from the gene products produced by the genes described in Table 5. Other specific embodiments refer to the individual gene products produced by the genes described in Table 5. In yet another specific embodiment, an individual nucleic acid molecule or nucleic acid sequence is selected from the nucleic acid molecules or nucleic acid sequences described in Table 5. Other specific embodiments refer to the individual nucleic acid molecules or nucleic acid sequences described in Table 5. Further specific embodiments refer to any combination of genes, gene products and nucleic acid molecules described in the Tables 5, Combinations may comprise 2, 3, 4, 5, 6 ,7, 8, 9, 10 or even more different species.

Modification may be performed by a single nucleic acid species or by a combination of nucleic acids comprising 2, 3 4, 5, 6 or even more different nucleic acid species, which may be selected from Tables 1a, 1b, 2, 4 or/and 5 and fragments thereof. Preferred combinations are described in Table 3 (also referred herein as “pools”). Table 3 includes combinations of at least two kinase or/and kinase binding polypeptide genes. It is also preferred that the combination modifies the expression of a single gene, for instance selected from Table 1a, 1b, 4 and 5. A combination of two nucleic acid species is preferred. More preferred is a combination of two nucleic acids selected from Table 2. Even more preferred is a combination of two nucleic acids selected from the specific combinations disclosed in Table 2, wherein the two nucleic acids modify the expression of a single gene.

Modification, in particular modulation, may be a knock-down performed by RNA interference. The nucleic acid or the combination of nucleic acid species may be an siRNA, which may comprise a sequence selected from the sequences of Table 2, Table 4 and Table 5 and fragments thereof. It is preferred that the combination knocks down a single gene, for instance selected from Table 1b and Table 4. A combination of two siRNA species is preferred, which may be selected from those sequences of Table 2, which are derived from genes of Table 1b, and the sequences of Table 4 and Table 5, wherein the combination preferably knocks down a single gene.

“Activation of a gene or/and gene product” or “inhibition of a gene or/and gene product” by recombinant technology, which may be employed in step (a) of the present invention, may include any suitable method the person skilled in the art knows.

Preferred methods of activation of a gene of interest or/and the gene product thereof may be selected from
introducing at least one further copy of the gene to be activated into the cell or/and organism, either permanently or transiently,
increasing the transcription,
over-expression,
introducing a strong promoter into the gene, e.g. a CMV promoter,
introducing a suitable enhancer,
inhibition of trancriptionally active microRNA, wherein the microRNA inhibits the activity of the gene to be activated, wherein inhibition may be performed by a suitable nucleic acid molecule,
deletion of a miRNA binding site,
improvement of RNA processing including exportation from the nucleus, e.g. by 3′ terminally introducing post-transcriptional regulatory elements, e.g. from hepadna viruses, or by 3′ terminally introducing of one or more constitutive transport elements, e.g. from type D retroviruses, or/and by employing an intron which can be spliced,
improvement of translation by improvement of ribosomal binding and optimisation of the coding sequence or/and the 3′ UTR, e.g. by deletion of cryptic splicing sites, optimisation of GC content, deletion of killer motives and repeats, optimisation of the structure.

Preferred methods of inhibition of a gene of interest or/and the gene product thereof may be selected from
deleting at least one further copy of the gene to be inhibited in the cell or/and organism, wherein the gene is deleted completely or partially. For instance, the regulatory sequences or/and the coding sequences are deleted, completely or partially,
decreasing the transcription,
deleting an enhancer, if present,
introduction or/and activation of a trancriptionally active microRNA, wherein the microRNA inhibits the activity of the gene to be inhibited, wherein activation may be an activation of an endogeneous microRNA coding sequence, and introduction may be introduction of an exogeneous microRNA molecule,
introducing of an miRNA binding site,
reducing RNA processing including exportation from the nucleus, by deletion or/and modification of 3′ terminally introducing post-transcriptional regulatory elements or 3′ terminally introducing of one or more constitutive transport elements, if present, or by altering the intron-exon structure,
reducing translation by modification of ribosomal binding and the coding sequence or/and the 3′ UTR, e.g. by introducing of cryptic splicing sites, altering the GC content, introducing of killer motives and repeats.

The gene employed in the various embodiments of the present invention may be selected from any of the Tables 1A, 1B, 2, 4 and 5, or any combination thereof.

Contacting the cell or/and the organism according to step (b) with an influenza virus is known. In the case the non-human organism is an embryonated egg, the skilled person knows suitable methods of inoculating the egg with an influenza virus, for instance at a defined interval after fertilization. Known inoculation techniques may also be applied for administration of the modulator to the embryonated egg or/and for recombinant modification of the embryonated egg.

The skilled person knows methods according to step (c) of cultivating the cell, the embryonated egg or/and the non-human organism under conditions allowing the replication of the influenza virus. Suitable cell culture methods may be applied. In the case the non-human organism is an embryonated egg, the skilled person knows suitable methods, including incubation at elevated temperature, to allow influenza virus replication.

Isolating the influenza virus or/and the components thereof according to step (d) refers to any isolation procedure for viruses or/and components thereof known by a person skilled in the art. “Isolation” includes production of essentially pure or crude preparations or formulations of the virus or/and components thereof. Components of the virus include viral proteins, polypeptids, and nucleic acids encoding viral proteins or/and polypeptides. The life virus may also be isolated.

The person skilled in the art knows methods of preparation of the pharmaceutical composition according to step (e), optionally together with a pharmaceutically acceptable carrier, adjuvant, diluent or/and additive. The pharmaceutical composition produced by the method of the present invention may be an immunogenic composition. The pharmaceutical composition produced by the method of the present invention may also be a vaccine.

The pharmaceutical composition as described herein (produced by the method of the present invention,) is preferably for use in human or veterinary medicine. The pharmaceutical composition is preferably for use for the prevention, alleviation or/and treatment of an influenza virus infection.

The carrier in the pharmaceutical composition may comprise a delivery system. The person skilled in the art knows delivery systems suitable for the pharmaceutical composition of the present invention. The pharmaceutical composition may be delivered in the form of a naked nucleic acid, in combination with viral vectors, non viral vectors including liposomes, nanoparticles or/and polymers. The pharmaceutical composition or/and the nucleic acid may be delivered by electroporation.

Naked nucleic acids include RNA, modified RNA, DNA, modified DNA, RNA-DNA-hybrids, aptamer fusions, plasmid DNA, minicircles, transposons.

Viral vectors include poxviruses, adenoviruses, adeno-associated viruses, vesicular stomatitis viruses, alphaviruses, measles viruses, polioviruses, hepatitis B viruses, retroviruses, and lentiviruses.

Liposomes include stable nucleic acid-lipid particles (SNALP), cationic liposomes, cationic cardiolipin analogue-based liposomes, neutral liposomes, liposome-polycation-DNA, cationic immunoliposomes, immunoliposomes, liposomes containing lipophilic derivatives of cholesterol, lauric acid and lithocholic acid. Examples of compounds suitable for liposome formation are 1,2-dilauroyl-sn-glycero-3-phosphoethanolamine (DLPE); 1,2-dioleoyl-sn-glycero-3-phospho-L-serine (DOPS); cholesterol (CHOL); 1,2-dioleoyl-sn-glycero-3-phosphocholine (DOPC).

Nanoparticles include CaCO₃ nanoparticles, chitosan-coated nanoparticle, folated lipid nanoparticle, nanosized nucleic acid carriers.

Polymers include polyethylenimines (PEI), polyester amines (PEA), polyethyleneglycol(PEG)-oligoconjugates, PEG liposomes, polymeric nanospheres.

The pharmaceutical composition may be delivered in combination with atelocollagen, carbon nanotubes, cyclodextrin-containing polycations, fusion proteins (e.g. protamine-antibody conjugates).

Yet another subject of the present invention is a recombinant cell produced according to step (a) of the method of the present invention, as described herein.

Yet another subject of the present invention is a recombinant non-human organism produced according to step (a) of the method of the present invention, as described herein.

Yet another subject of the present invention is a recombinant embryonated egg produced according to step (a) of the method of the present invention, as described herein. The recombinant embryonated egg is preferably a recombinant embryonated hen's egg.

The invention is further illustrated by the following figures, tables and examples.

### FIGURE AND TABLE LEGENDS

FIG. 1: The experimental setting of the siRNA kinase screen of the example.

FIG. 2: The effect of transfected (control)-siRNAs in regard to luminescence data. This diagram shows a typical screening result from one 96 well plate. During all experiments several controls were included in triplets, like uninfected, transfected with a siRNA against luciferase, mock treated and siRNAs against the viral nucleoprotein gene (NP) from influenza A viruses. The difference of the luminescence between cells treated with luciferase siRNAs and anti-NP siRNAs was set to 100% inhibition per definition.

FIG. 3: The inhibition of influenza virus replication shown for all siRNAs tested in the example.

FIG. 4: The values “% inhibition” from all analyzed siRNAs were used to calculate the z-scores. Highly efficient siRNAs are labelled in pink showing more than 50% inhibition compared to the luciferase siRNA transfected control cells.

FIG. 5: The experimental setup of the genome wide siRNA screen (see Example 4).

Table 1: Results of the siRNa kinase screen: a: activation (“negative” inhibition) of virus replication in %, normalized against the cell number, and the standard deviation calculated using four independent experiments. b: inhibition of virus replication in %, normalized against the cell number, and the standard deviation calculated using four independent experiments. Pool X, wherein X denotes the number of the pool, refers to combinations described in Table 3.

Table 2: Oligonucleotide sequences employed in the siRNA kinase screen of example 1. Knock-down of a particular gene was performed (a) by a combination of two oligonucleotide sequences (“target 1” and “target 2”) specific for said gene, or (b) by pooled oligonucleotides specific for different genes (“Pool X”, wherein X denotes the number of the pool described in Table 3).

Table 3: Oligonucleotide pools employed in the siRNA kinase screen of the example.

Table 4: Oligonucleotide sequences employed in the siRNA screen of example 4. Up to four oligonucleotide sequences (“target sequence 1”, “target sequence 2”, “target sequence 3”, and “target sequence 4”) specific for a gene were employed (each in a separate test).

Table 5: Oligonucleotide sequences employed in the siRNA screen of example 4. Up to four oligonucleotide sequences (“target sequence 1”, “target sequence 2”, “target sequence 3”, and “target sequence 4”) specific for a gene were employed (each in a separate test). Knock-down of the genes described in this Table resulted in increase of virus replication.

### EXAMPLE 1

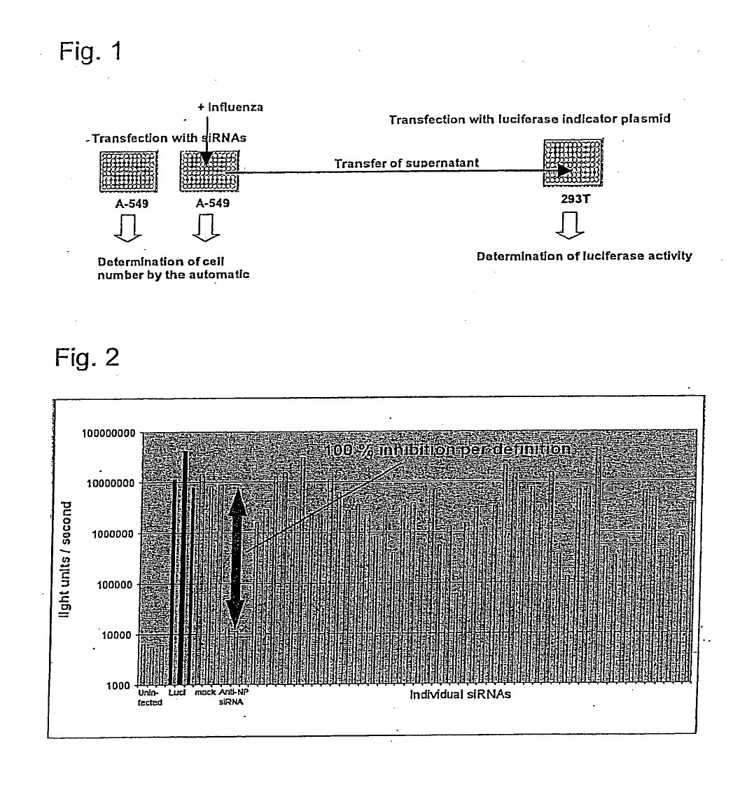

Since kinases are one of the most promising candidates that can influence virus progeny we used siRNAs against this group of genes to identify the individual role of each kinase or kinase binding polypeptide in respect of a modified replication of influenza viruses. All siRNAs were tested in four independent experiments. Since siRNAs against kinases can influence the replication of cells or are even cytotoxic, the effect of each individual siRNA transfection in regard to the cell number was analysed by using an automatic microscope. The amount of replication competent influenza viruses was quantified with an influenza reporter plasmid that was constructed using a RNA polymerase I promoter/terminator cassette to express RNA transcripts encoding the firefly luciferase flanked by the untranslated regions of the influenza A/WSN/33 nucleoprotein (NP) segment. Human embryonic kidney cells (293T) were transfected with this indicator plasmid one day before influenza infection. These cells were chosen, because they show a very strong amplification of the luciferase expression after influenza A virus infection. The cell based assay comprised the following steps (also FIG. 1 which describes the experimental setting of the siRNA kinase screen):

Day 1: Seeding of A549 cells (lung epithelial cells) in 96-well plates

Day 2: Transfection with siRNAs directed against kinases or kinase binding proteins

Day 3: Infection with influenza A/WSN/33+transfection of 293T cells with the influenza indicator plasmid

Day 4: Infection of 293T cells with the supernatant of A549 cells+determination of cell number by the automatic microscope

Day 5: Lysis of the indicator cells and performing the luciferase assay to quantify virus replication

For the identification of influenza relevant kinases the luminescence values were normalised against the cell number (measured after siRNA transfection and virus infection). Thereby unspecific effects due to the lower (or higher) cell numbers can be minimized.

Several controls were included to be able to demonstrate an accurate assay during the whole screening procedure (FIG. 2). The control siRNA against the viral nucleoprotein could nearly reduce the replication to levels of uninfected cells.

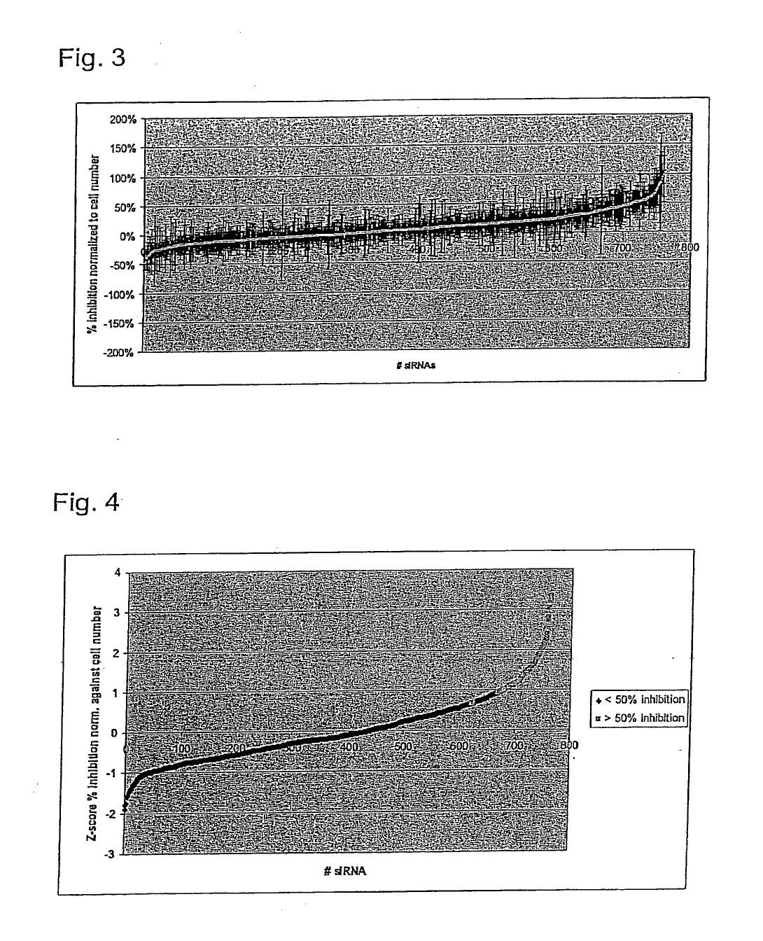

The illustration of the inhibition in percentage shows that some siRNAs can enhance the influenza virus replication, whereas others can inhibit the replication stronger (>113%) than the antiviral control siRNA against the influenza NP gene (FIG. 3). Thereby 47 siRNA decreased the replication more than 50%, 9 siRNAs showed more than 80% inhibition. The list of the results is provided in Table 1a and 1b, showing the activation (Table 1a, “negative” inhibition) and inhibition (Table 1b) of virus replication in %, normalized against the cell number, and the standard deviation calculated using four independent experiments.

Similar results were obtained using the calculation of z-scores. The z-score represents the distance between the raw score and the population mean in units of the standard deviation. The z-scores were calculated using the following equation:

where X is a raw score to be standardized, σ is the standard deviation of the population, and μ is the mean of the population.

### EXAMPLE 2

In a future experiment the antiviral effect will be validated in more detail by using individual siRNAs or shRNAs instead of pooled siRNAs. Furthermore new siRNAs (at least two additional siRNAs per identified gene) and shRNAs will be tested using the experimental setting of Example 1. Those confirmed genes that seem to be important for the replication of influenza viruses will then be knocked down in mice using intranasally administered siRNAs or shRNAs. For the evaluation of this antiviral therapy it is of highest importance to determine the efficiency of transportation of compounds to lung epithelial tissue. The success of a therapy depends on the combination of high efficient kinase inhibitors and adequate transport system. A potentially compatible and cost efficient agent is chitosan which we are applying for the delivery of siRNAs or shRNAs in in vivo studies successfully. We will apply the compounds either intranasally or administer them directly into the lung.

Efficient siRNAs or shRNAs should lead to a decreased viral titre within the lung tissue and due to this animals should be protected against an otherwise lethal influenza infection.

For testing the biological effect of the kinase inhibitors, we will divide the experiments in four parts:
1 Analysis of the kinase inhibitor distribution in the respiratory apparatus after intranasal application of compound/chitosan nano particles. Optimisation of the compound/chitosan concentration for best effectiveness. Further tests will only be performed in case of success.
2 In LD50 tests the absolute pathogenicity of the virus isolates Influenza A/Puerto Rico/8/34 and the Avian Influenza isolate (for test 4) will be estimated.
3 Test of antiviral effect of selected siRNAs or shRNAs after intranasal application and infection with Influenza A/Puerto Rico/8/34 by analyzing virus titre in lung tissue or survival rate (in certain cases).
4 Test of antiviral effect of selected siRNAs or shRNAs after intranasal application and infection with highly pathogenic Avian Influenza virus isolate (such as H5N1) by analyzing the virus titre in lung tissue or survival rate (in certain cases).

The used virus isolate is dependant on current development and spreading of the Avian Influenza. We aim at inhibiting the replication of the current prevalent strain in vivo efficiently

Kinase inhibitors against the confirmed genes will also be tested in mice regarding to an impaired virus replication.

The Max-Planck-Institut für Infektionsbiologie, Berlin, Germany, has genome-wide RNAi libraries that, in principle, enable the shutting-off of every single human gene in suitable cell cultures (A549 cells). So in the next level the screen will be expanded to a genome wide scale, because many additional cellular factors involved in the attachment, replication and budding of viruses are still unknown.

### EXAMPLE 3

Additional siRNAs (not only siRNAs against kinases or kinase binding proteins) and shRNAs will also be validated in regard to a decline of the replication of influenza A viruses. For the evaluation of these siRNAs and shRNAs the same experimental setting will be used as described in example 1, except that the cell number is quantified indirectly by using a commercial cell viability assay (instead of using an automated microscope) and that these siRNAs and shRNAs will be reverse transfected, i.e. cells will be added to the transfection mix already prepared in 384 well plates.

### EXAMPLE 4

Among the human genome hundreds of genes are presumably relevant for the replication of influenza viruses. Therefore the screening procedure of kinases and kinase binding factors (described in Example 1) was expanded to a genome wide scale analysing all known human genes by using about 59886 siRNAs.

The experimental setup was performed in a similar way as described in Example 1, except:
The screen was extended to genome wide level using 59886 siRNAs
Cells were seeded in 384 well plates.
Because of the huge number of transfected cells, not all cell numbers could be analysed by automated microscopy.
siRNAs were reversely transfected in freshly seeded A549 cells using the transfection reagent HiperFect (Qiagen, Hilden, Germany).
Knock-down of a particular gene was independently performed by up to four siRNAs (“target sequence 1”, “target sequence 2”, “target sequence 3”, and “target sequence 4” in Table 4) specific for a particular gene.
Additional controls were included: “AllStars Negative Control siRNA” (Qiagen, Hilden, Germany, Order No. 1027280) as negative control, siRNAs directed against PKMYT (GeneID: 9088, GenBank accessionnumber: NM_{—}182687, target sequence: CTGGGAGGAACTTACCGTCTA) as positive control (cellular factor against influenza replication), siRNAs directed against PLK (GeneID: 5347, GenBank accessionnumber: BC014135, target sequence: CCGGATCAAGAAGAATGAATA) as transfection control (cytotoxic after transfection).
The infection rate of transfected A549 cells in selected wells is measured by automated microscopy to be able to dissect the inhibitory effects to early or late events during the infection process.
Results were analysed by the statistical R-package “cellHTS” software, developed by Michael Butros, Ligia Bras and Wolfgang Huber, using the B-score normalisation method (based on “Allstars Negative Control siRNA” transfected control wells).
Read-out is inhibition of virus replication.

The siRNAs and corresponding genes that showed a strong antiviral activity (z-scores <−2.0) are listed in Table 4.

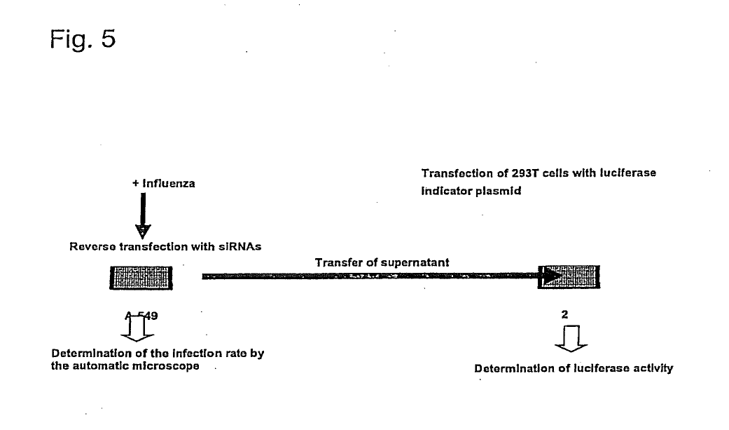

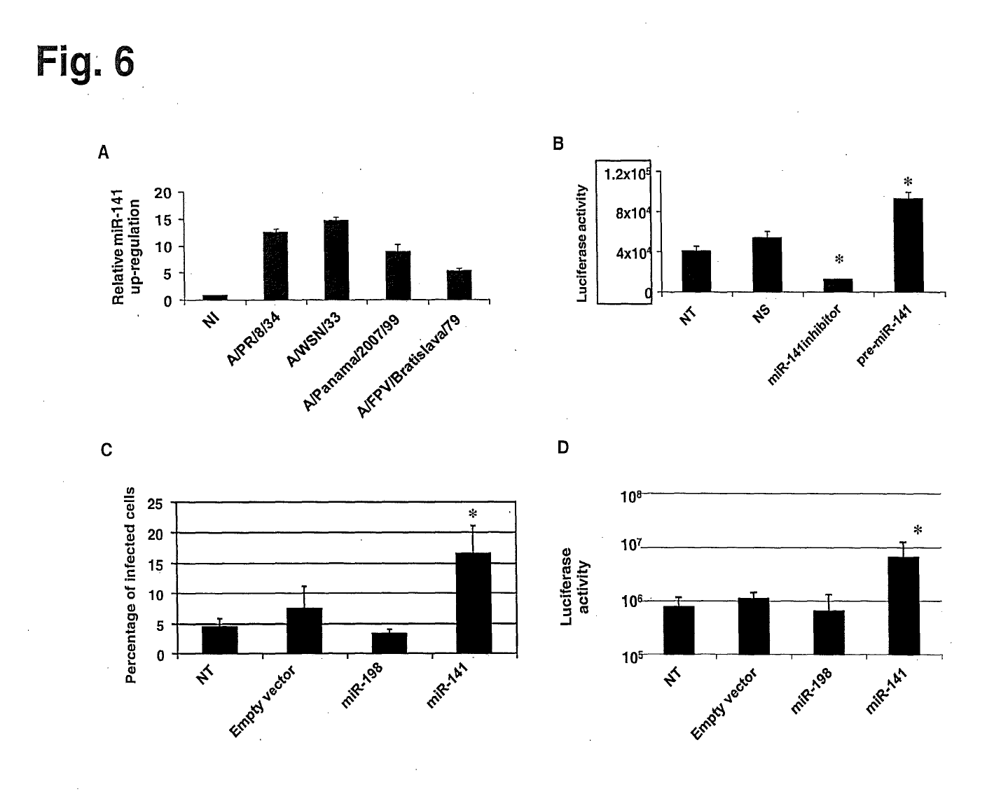

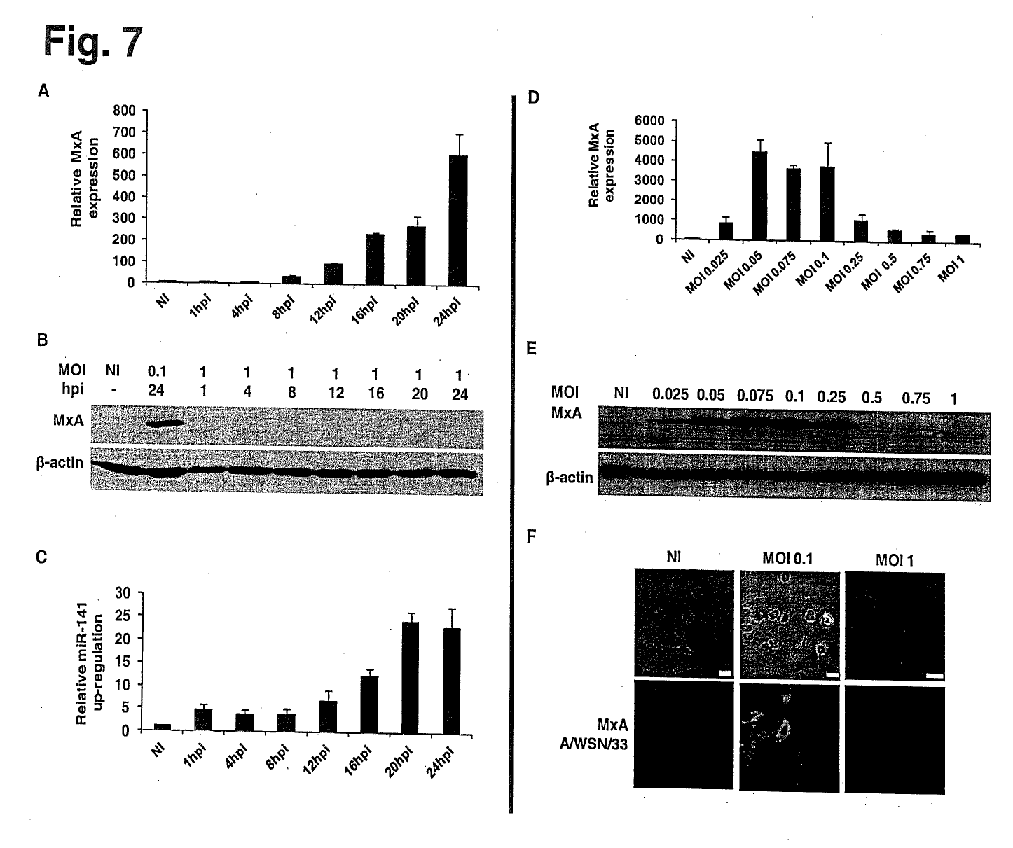

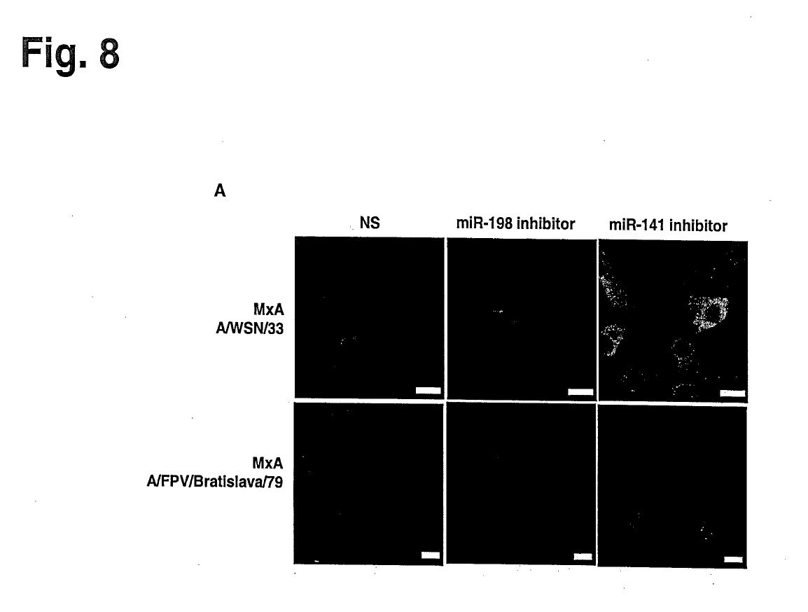

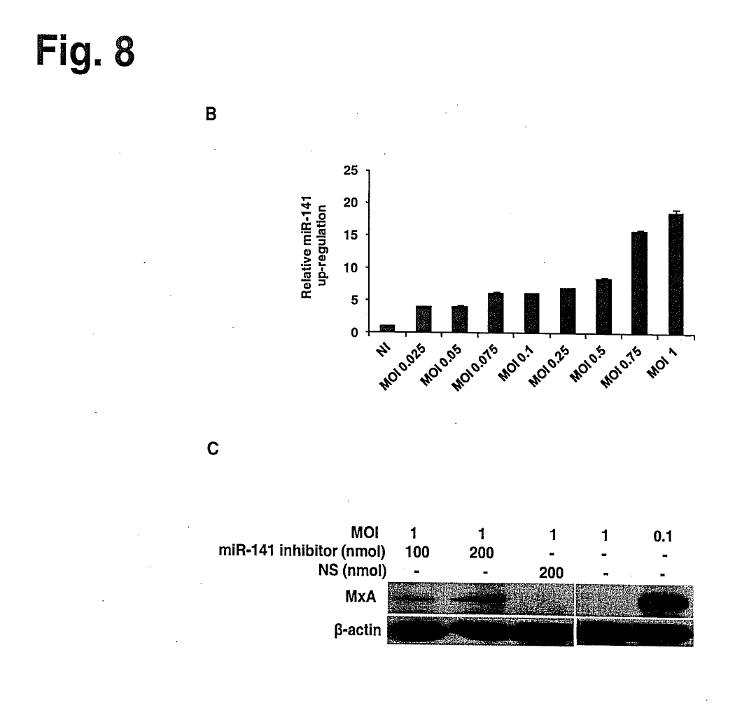

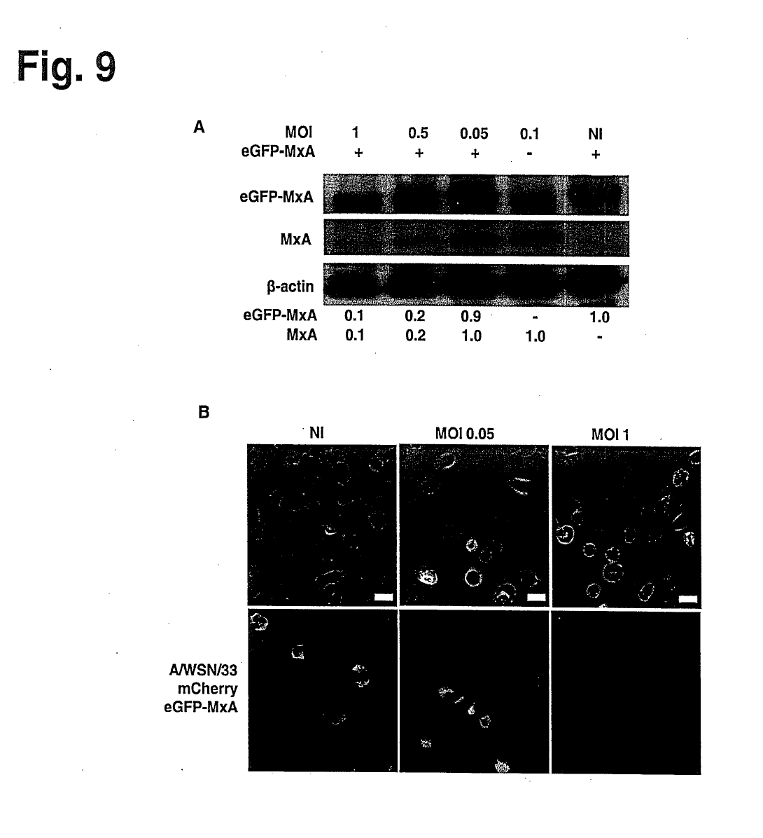

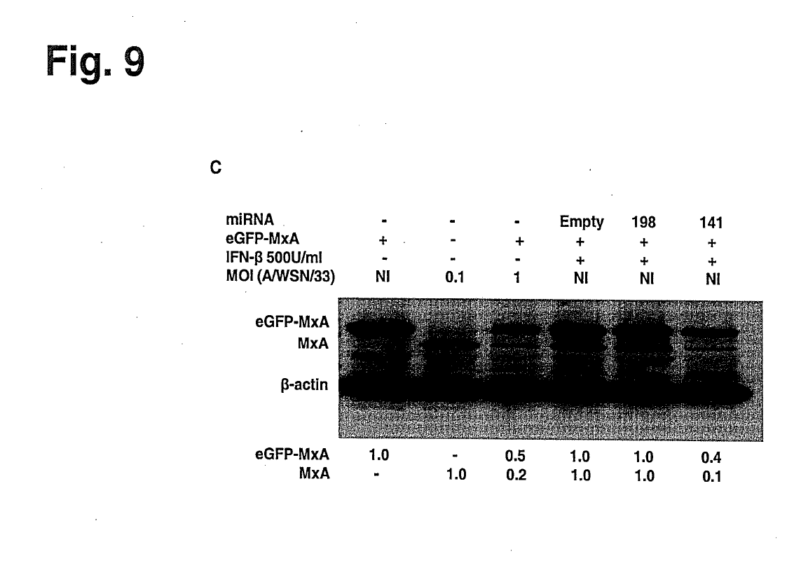

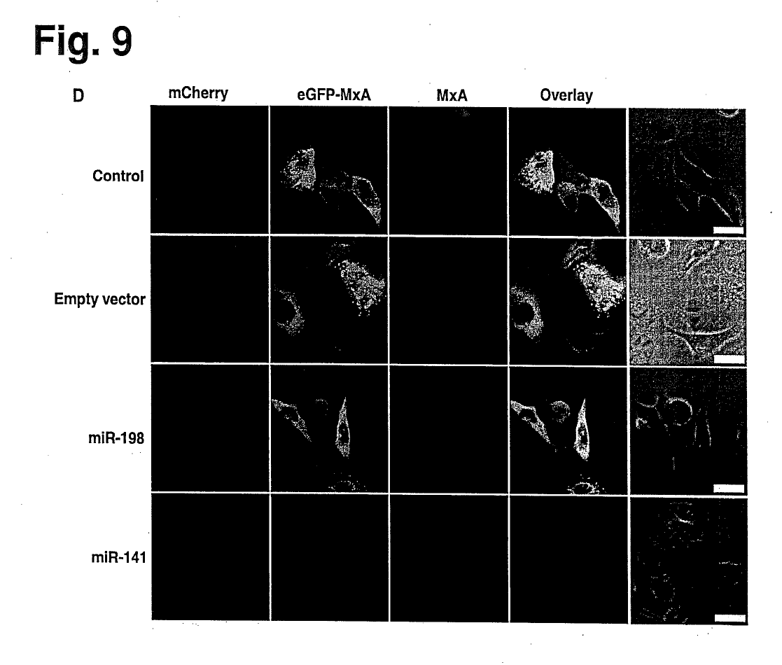

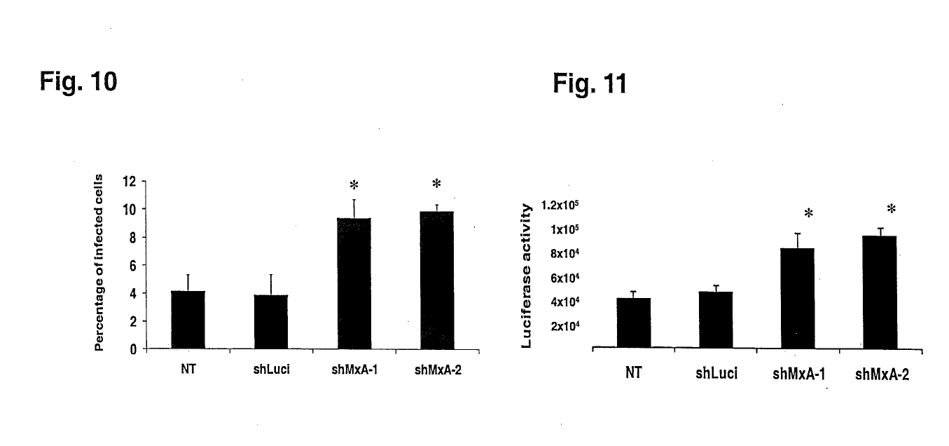

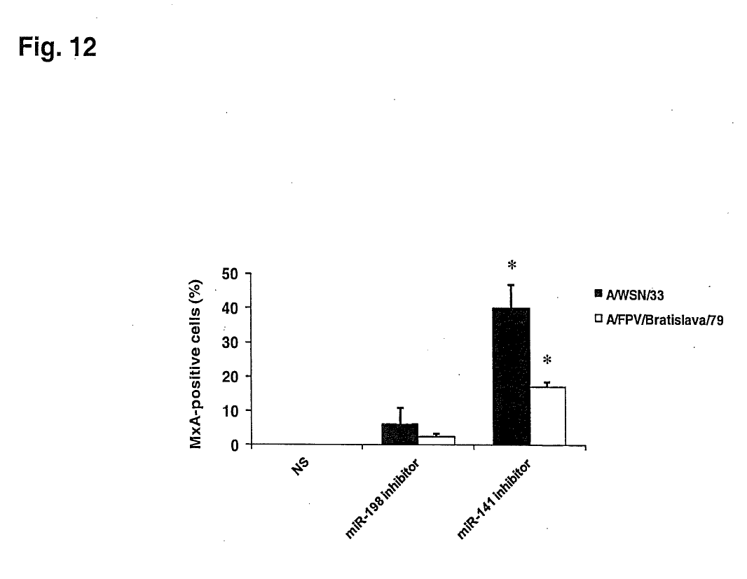

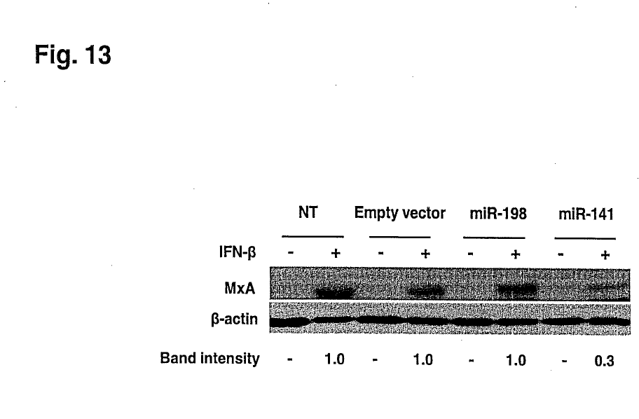

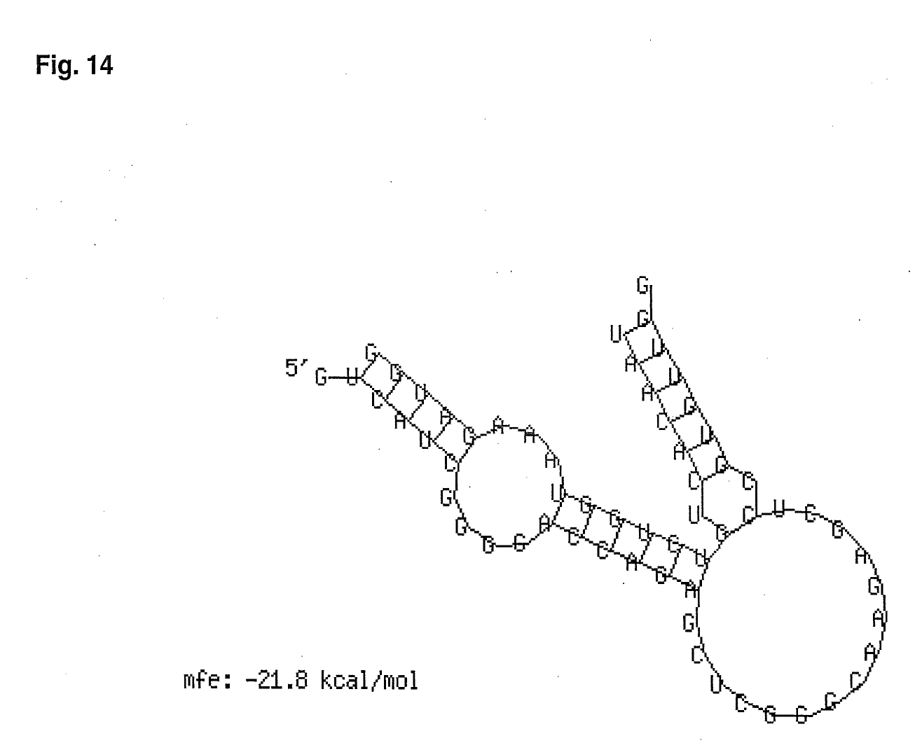

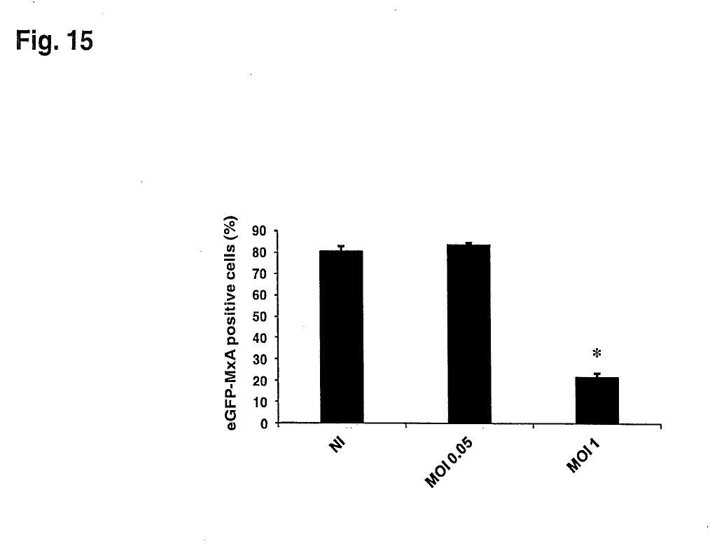

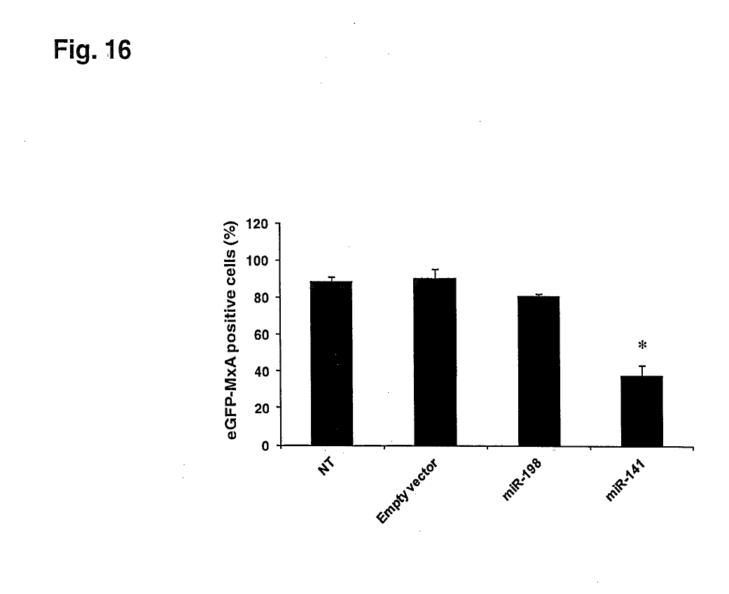

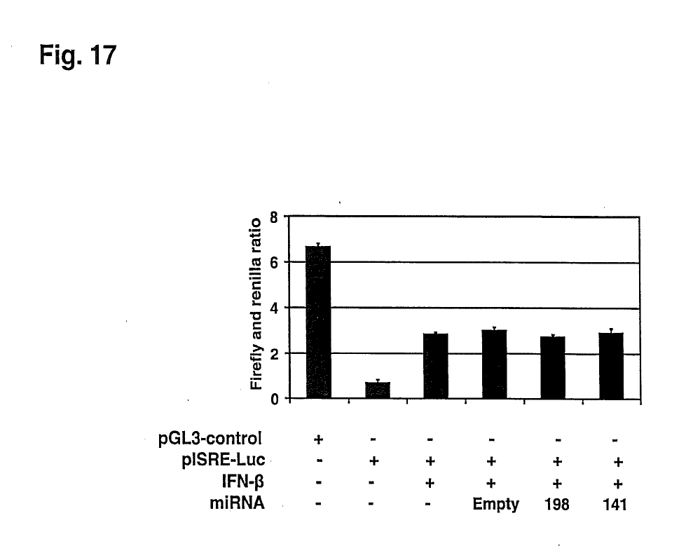

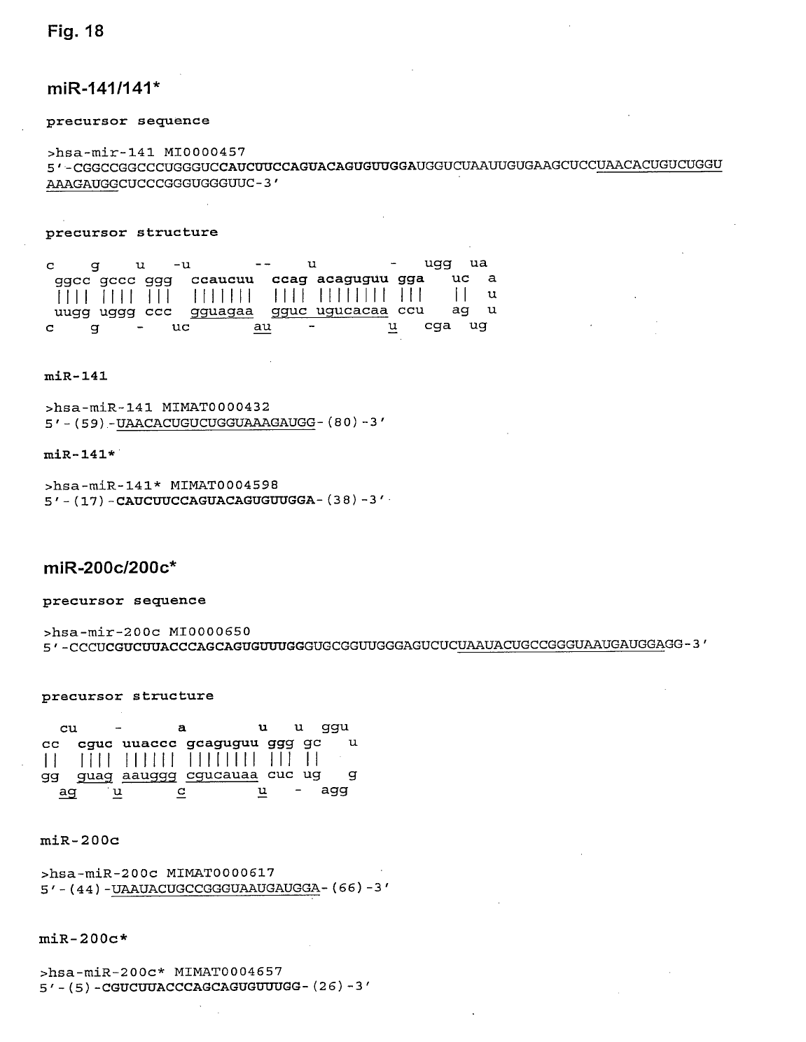

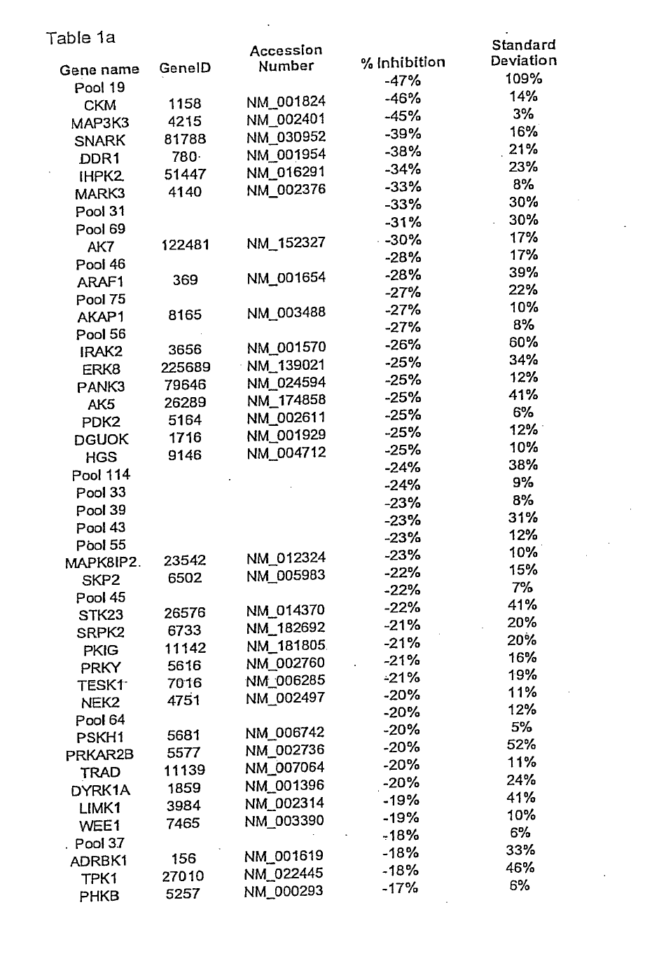

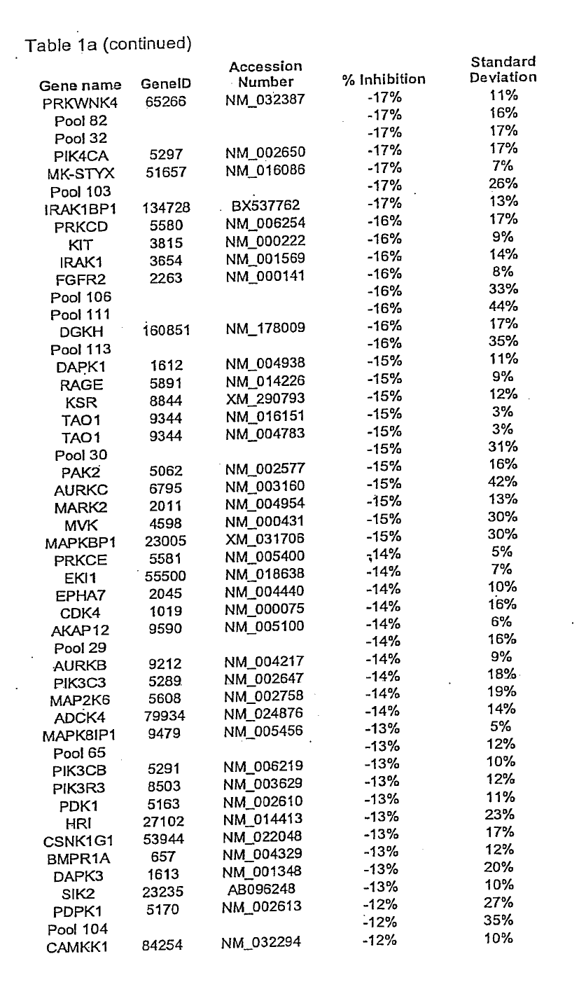

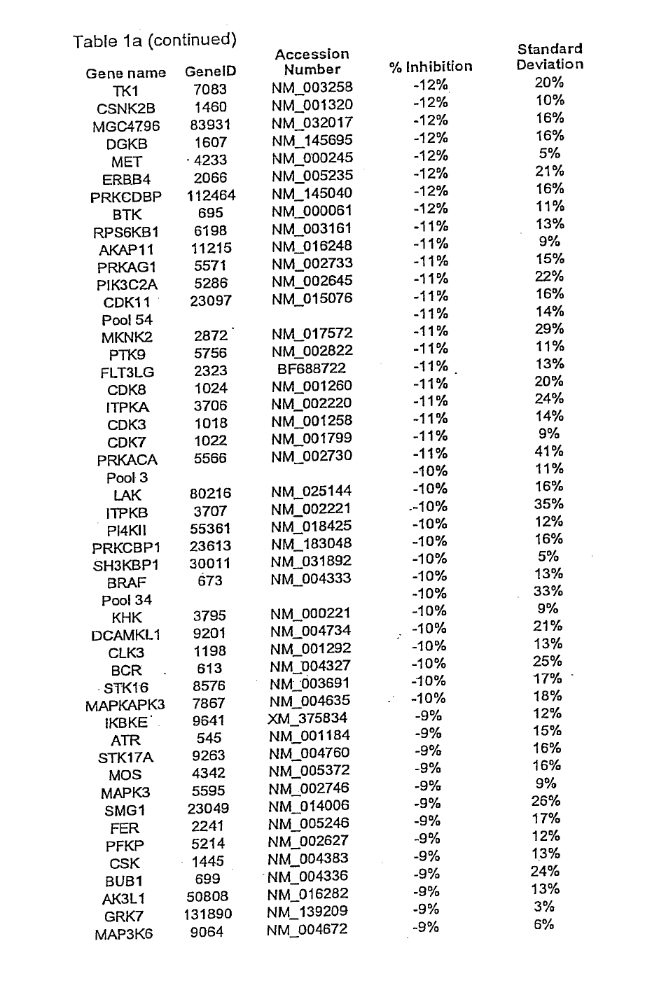

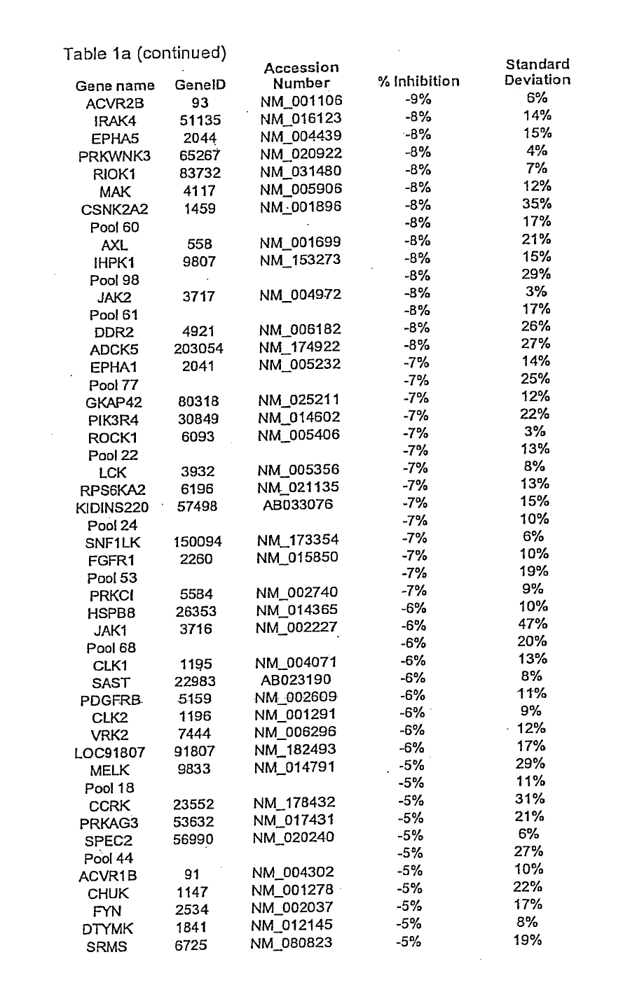

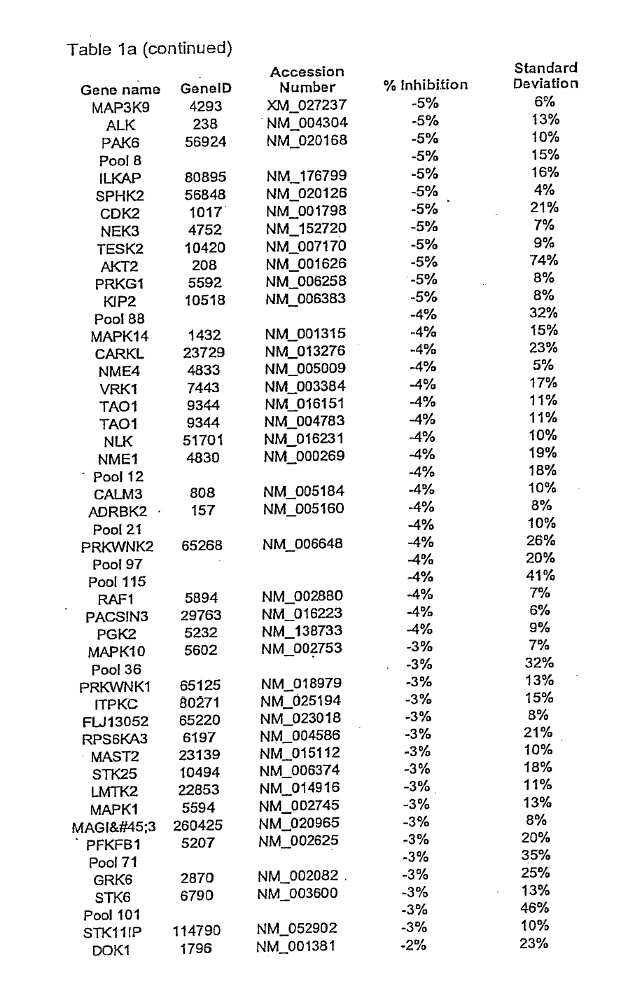

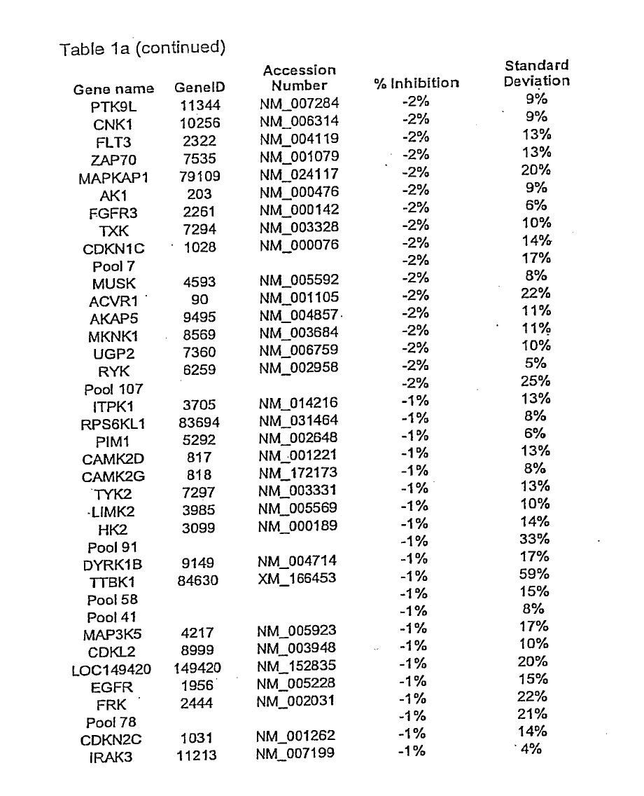

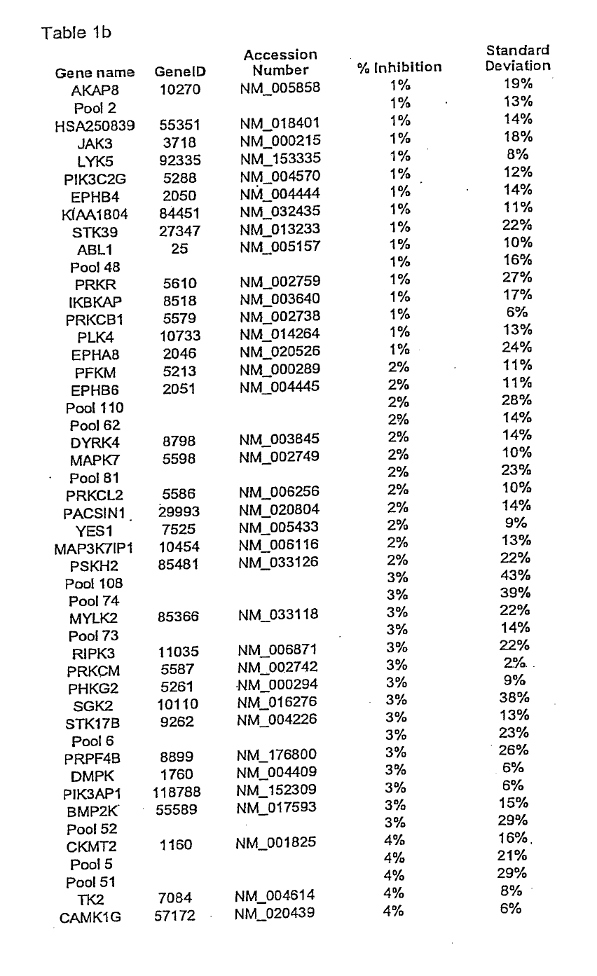

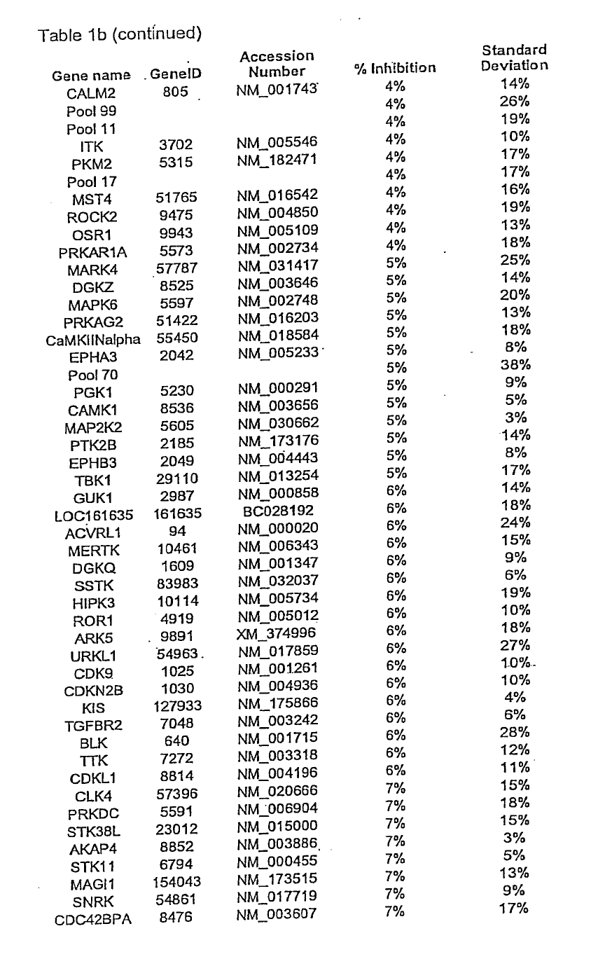

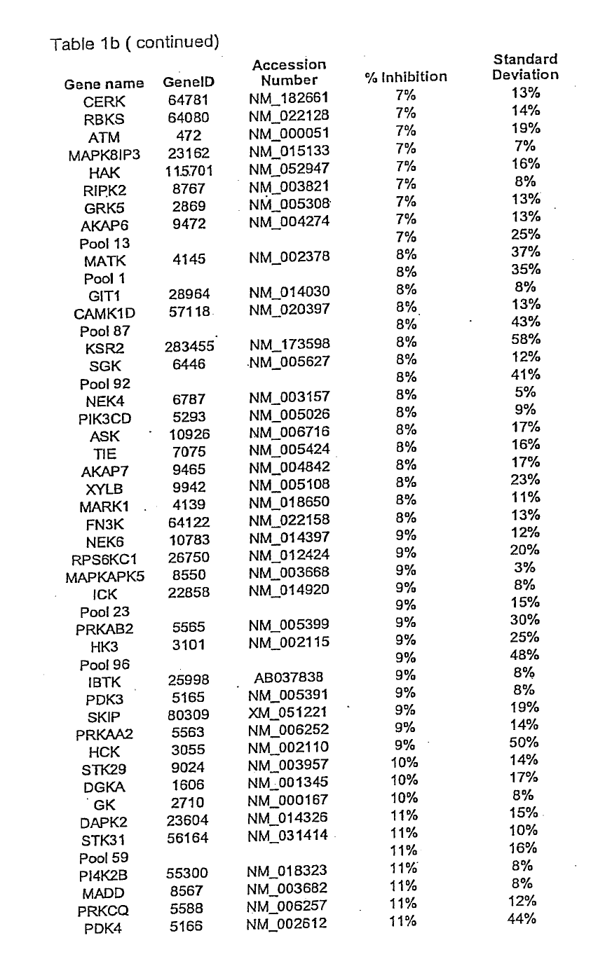

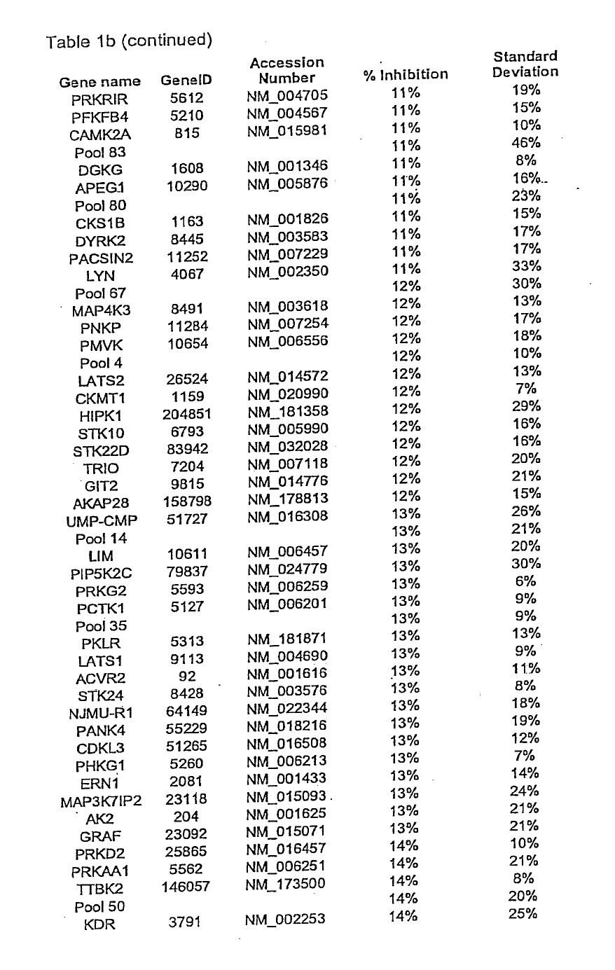

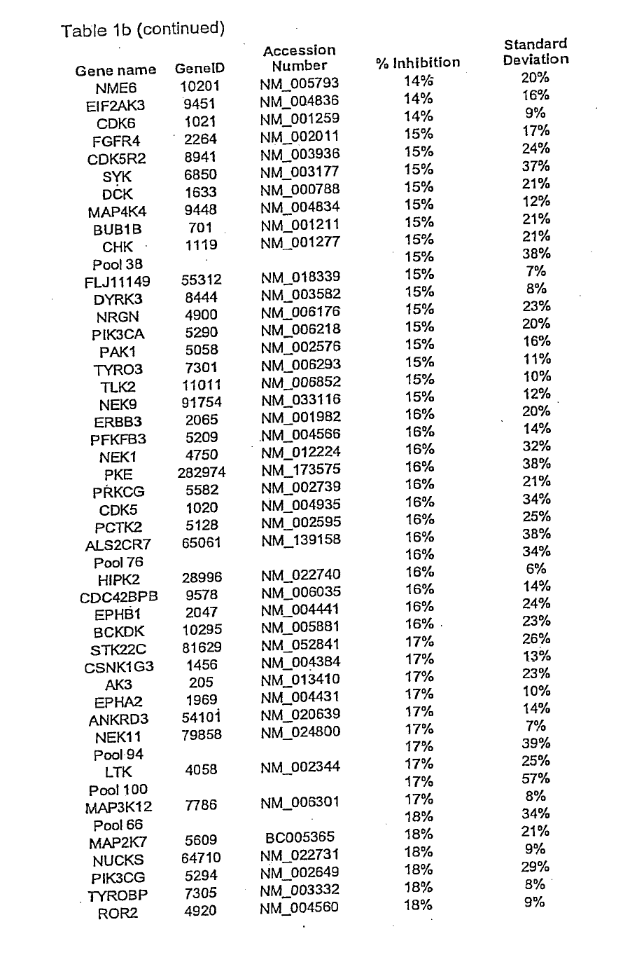

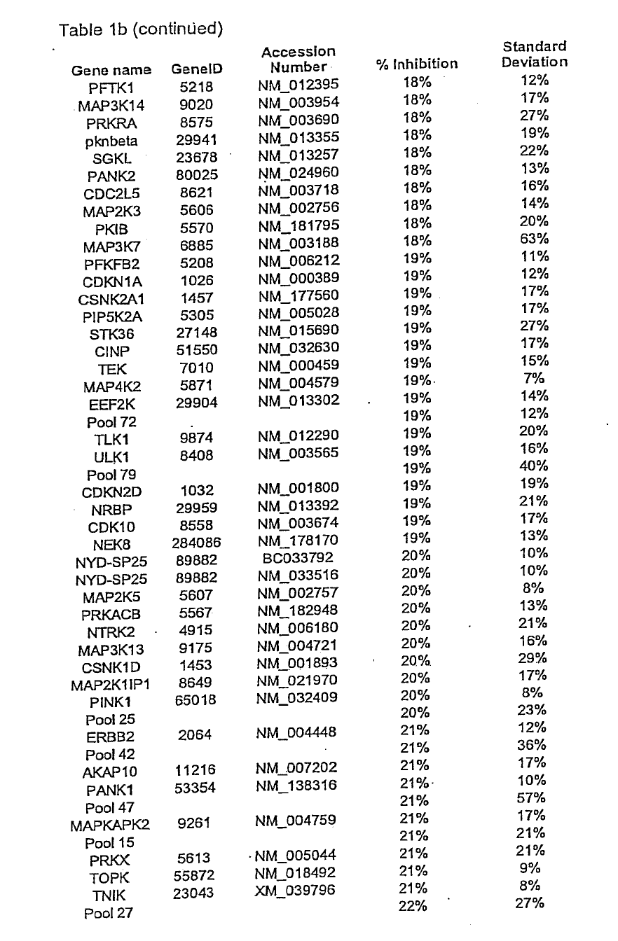

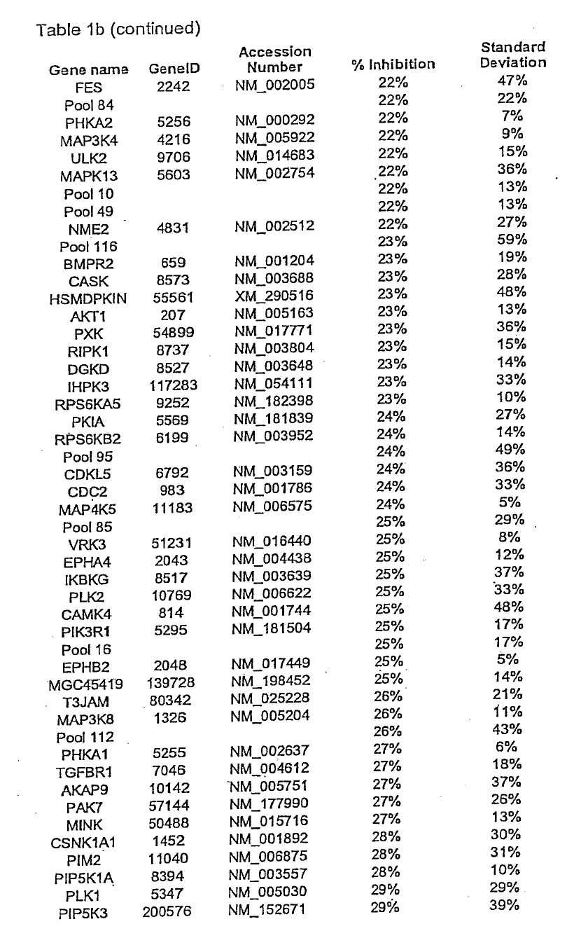

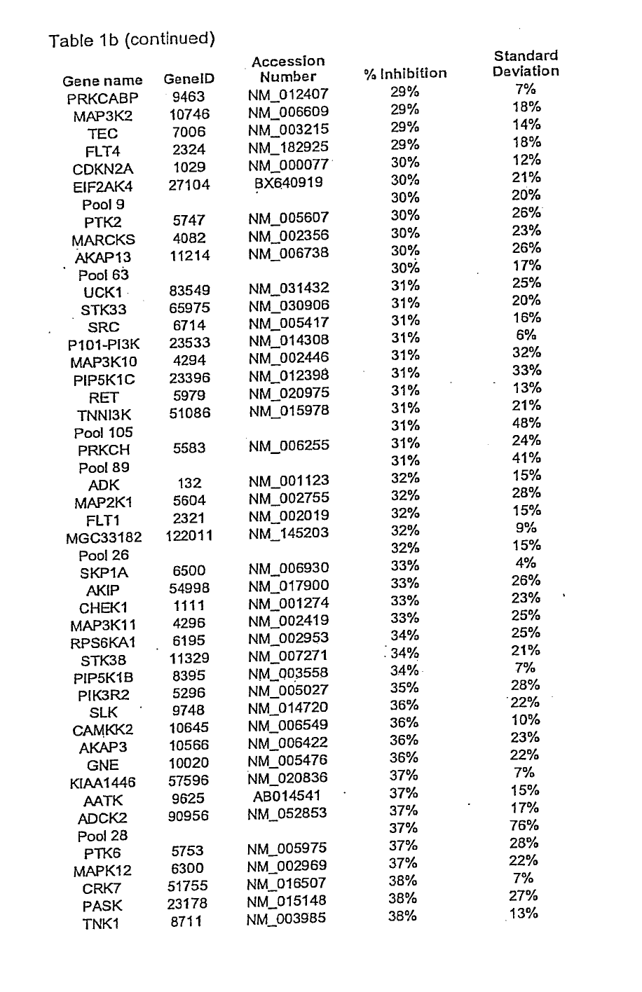

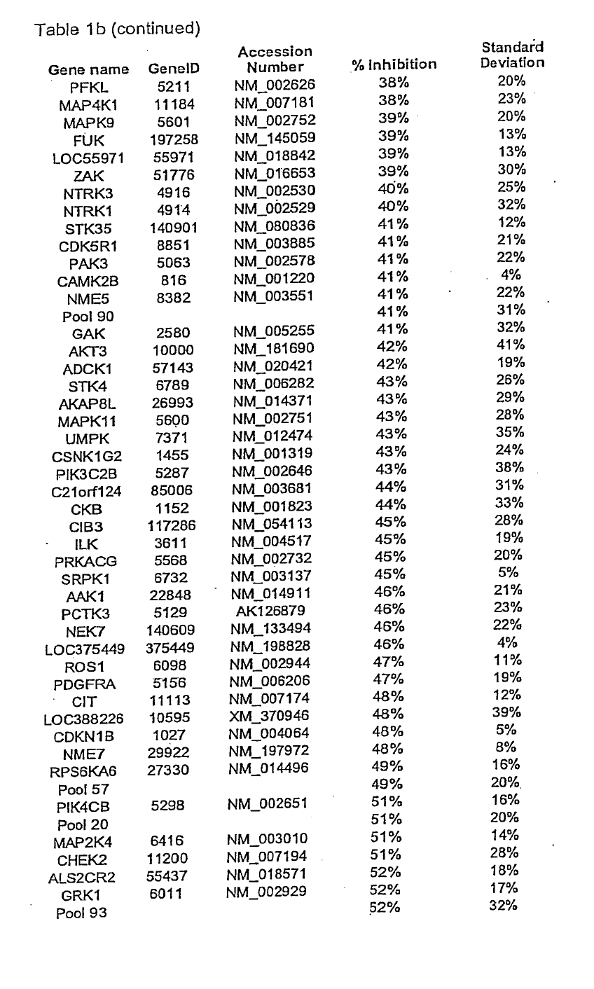

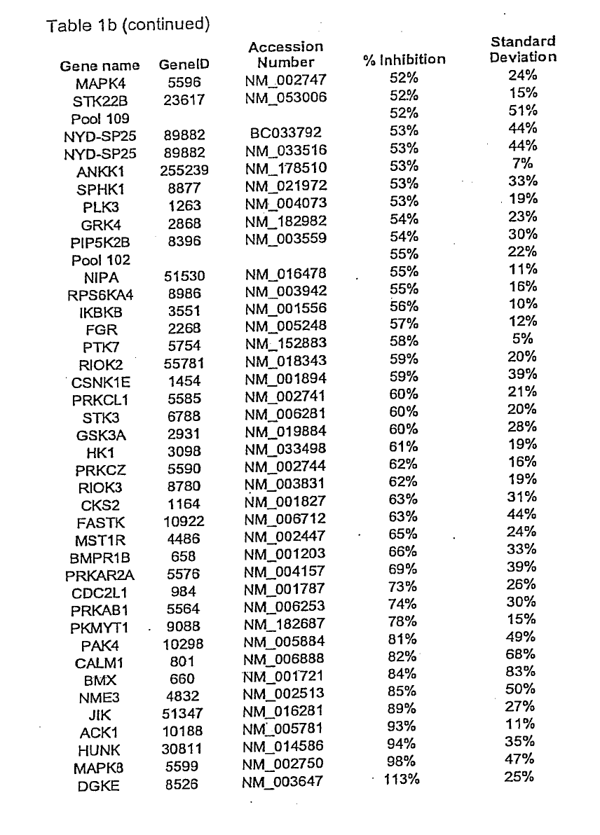

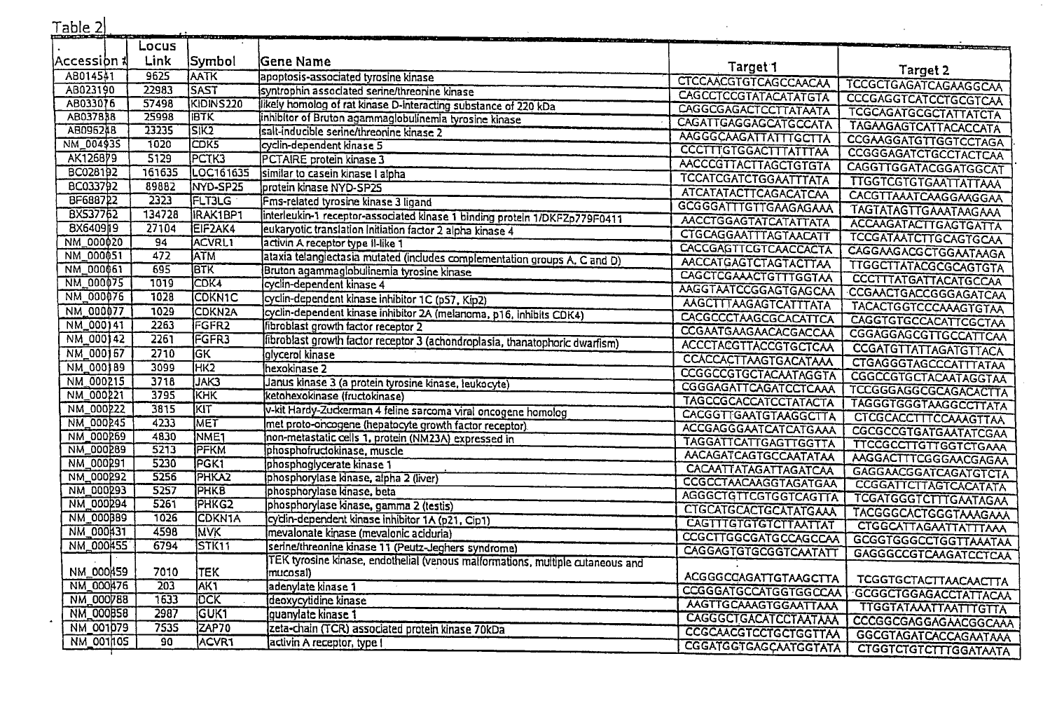

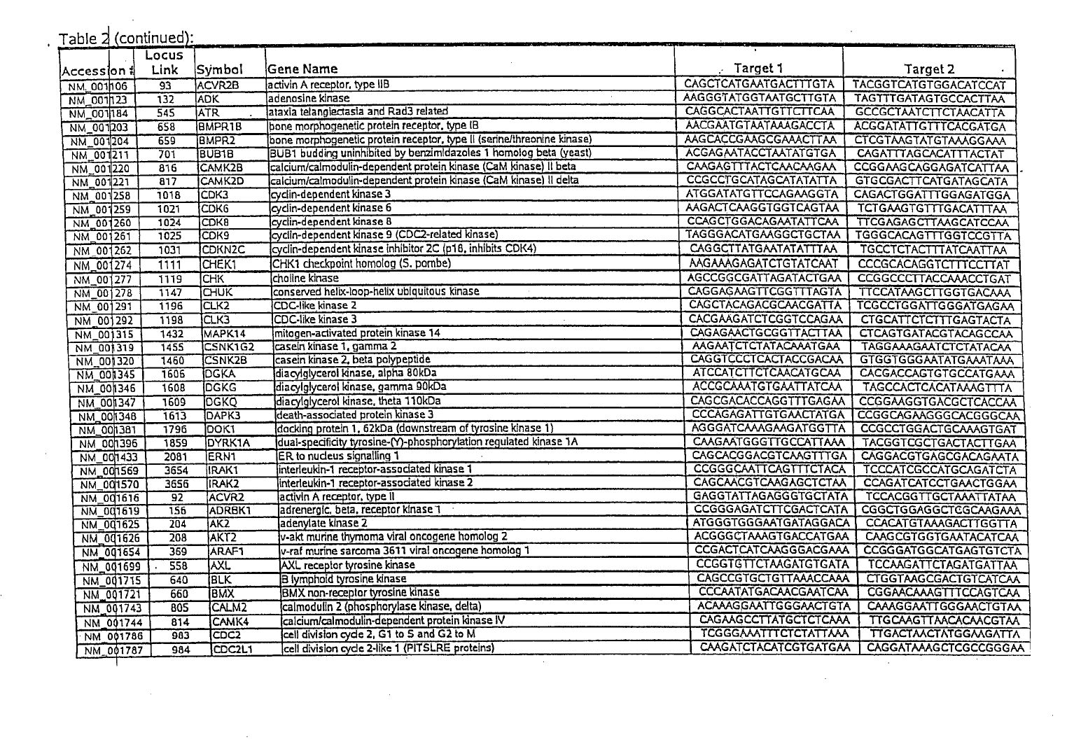

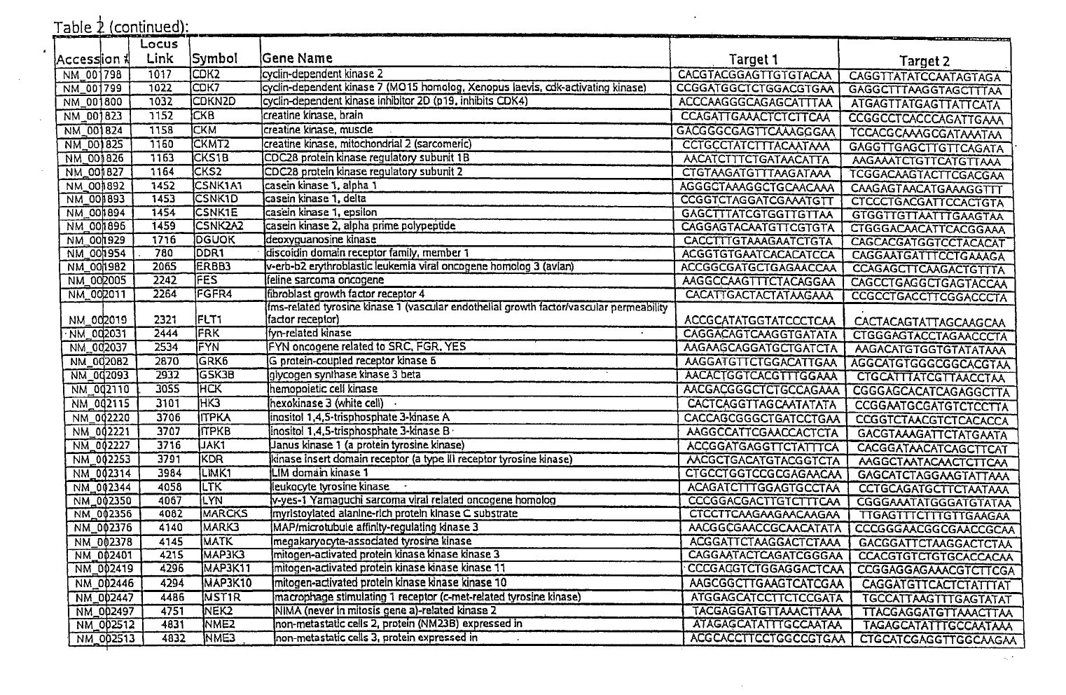

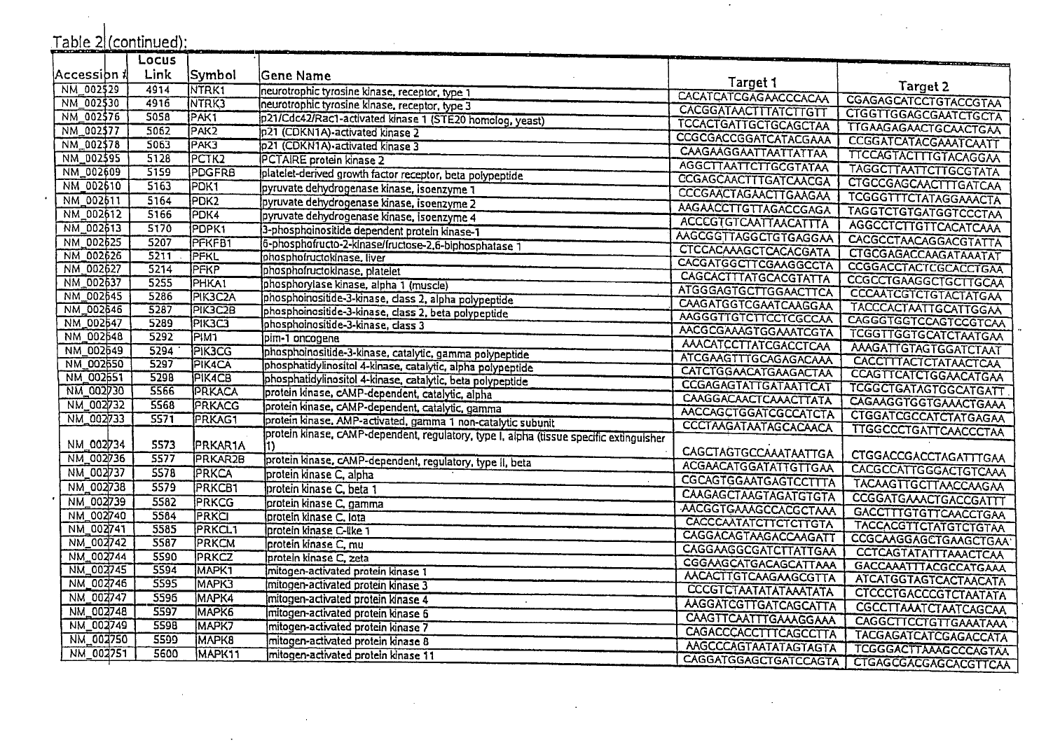

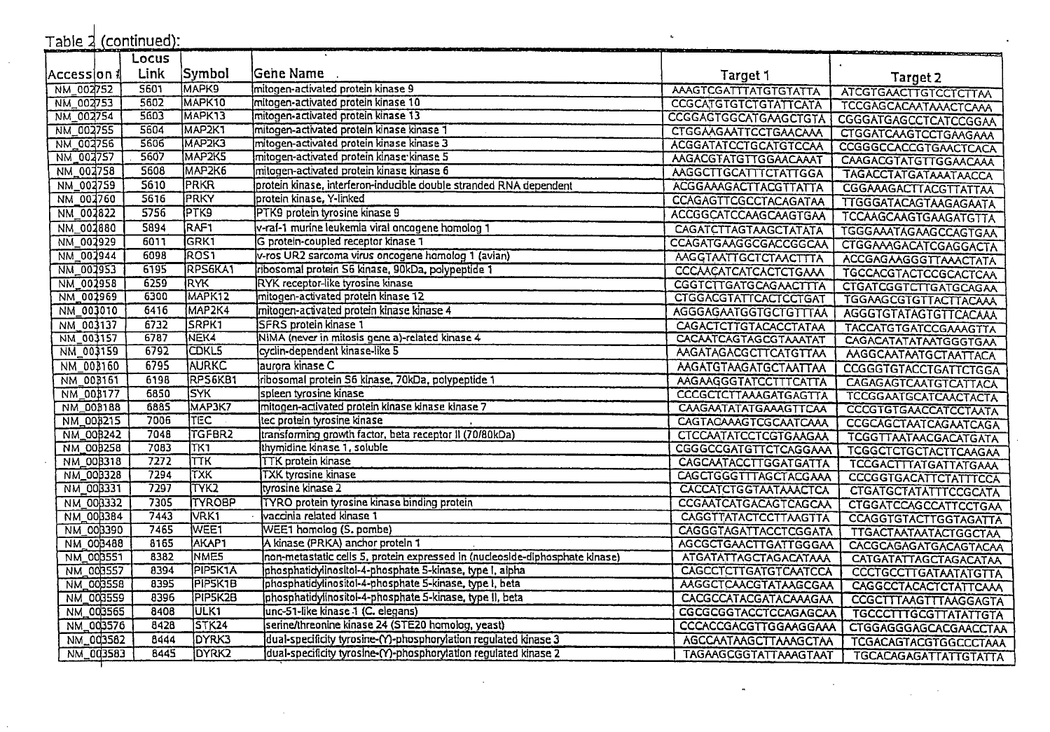

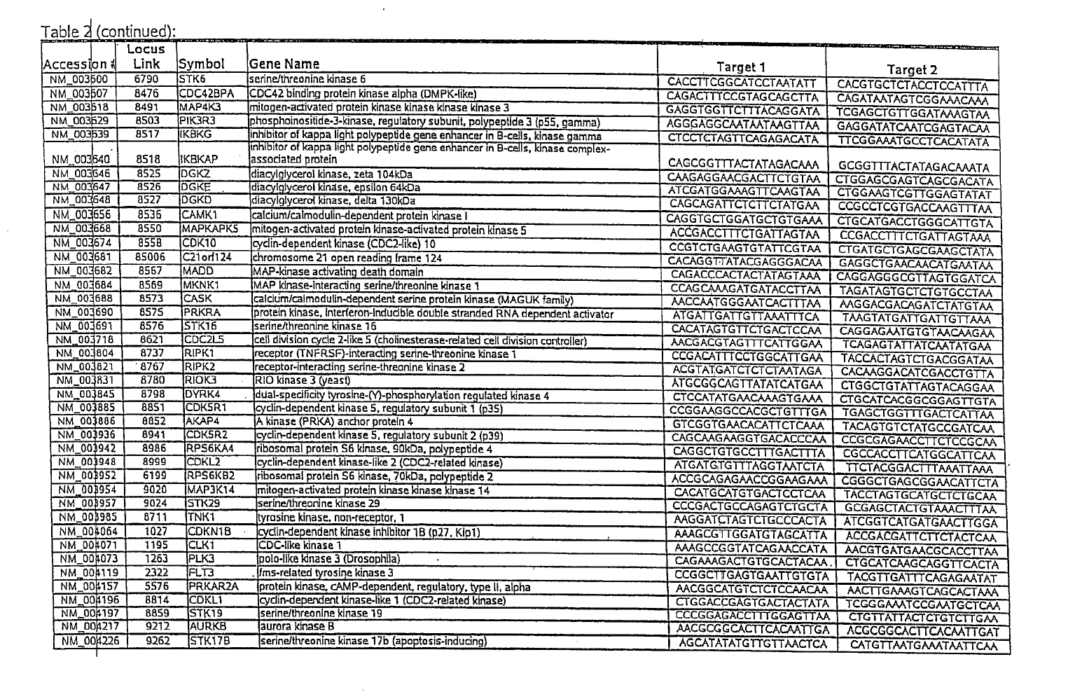

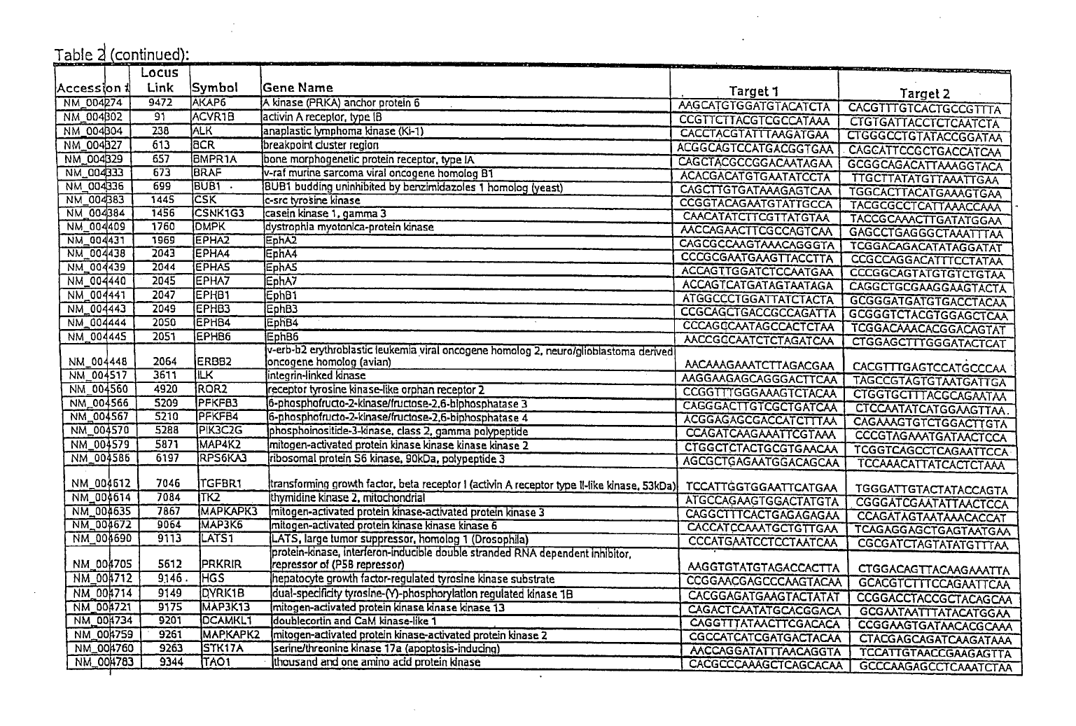

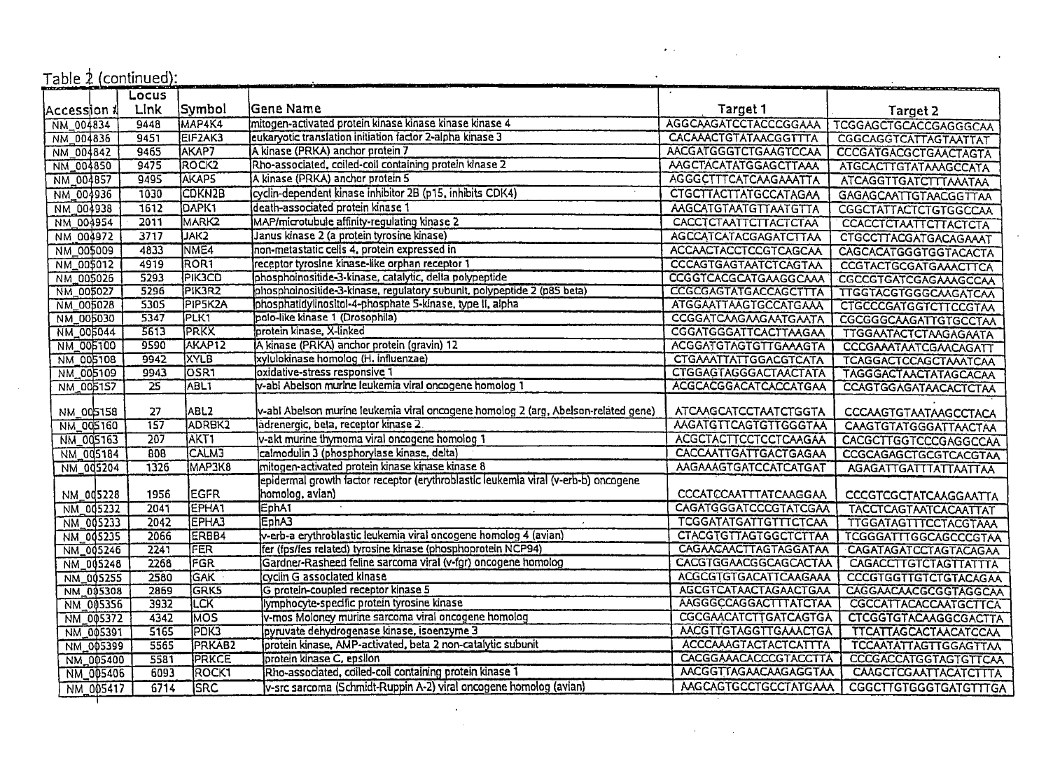

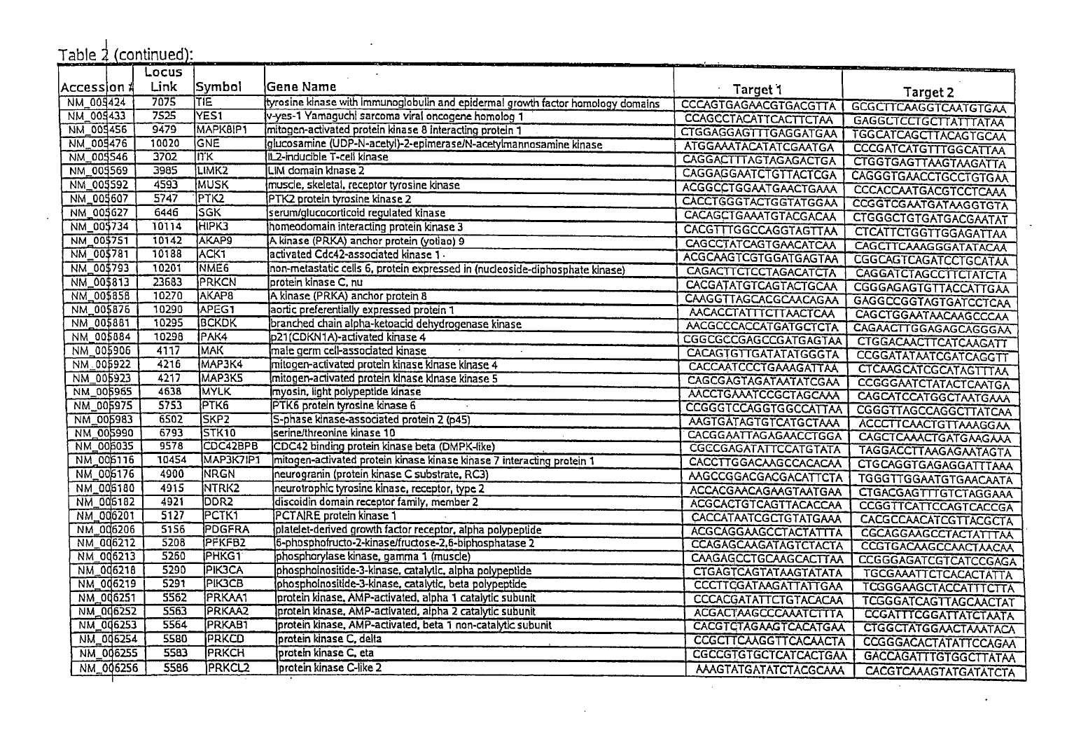

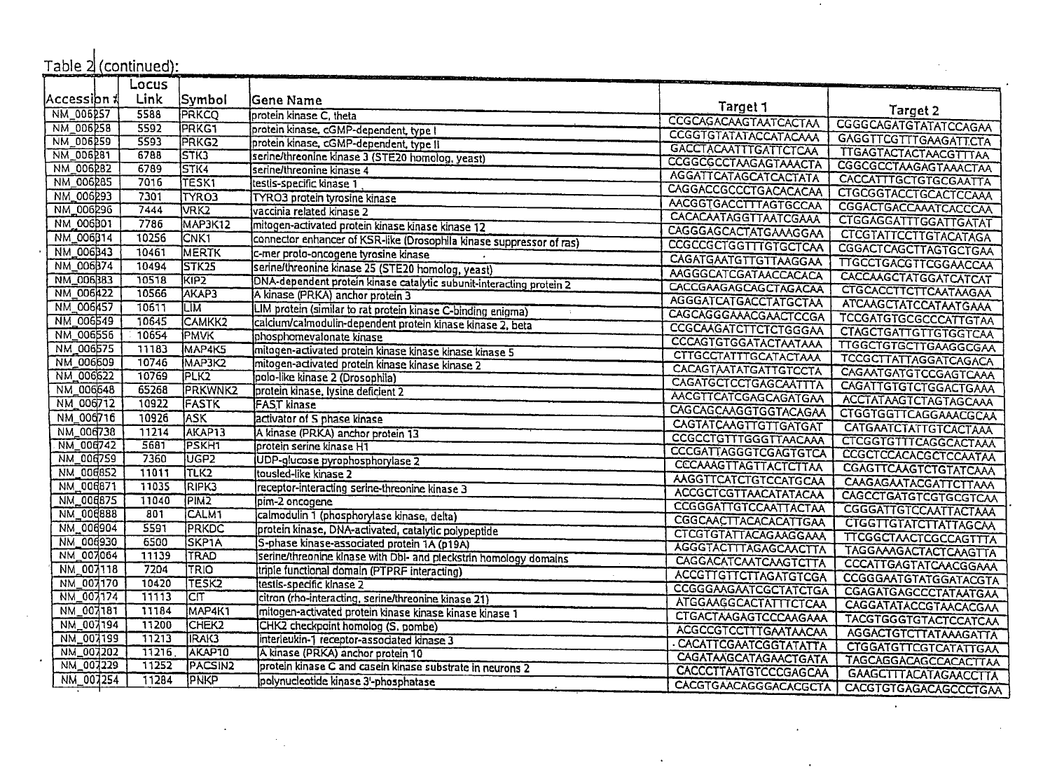

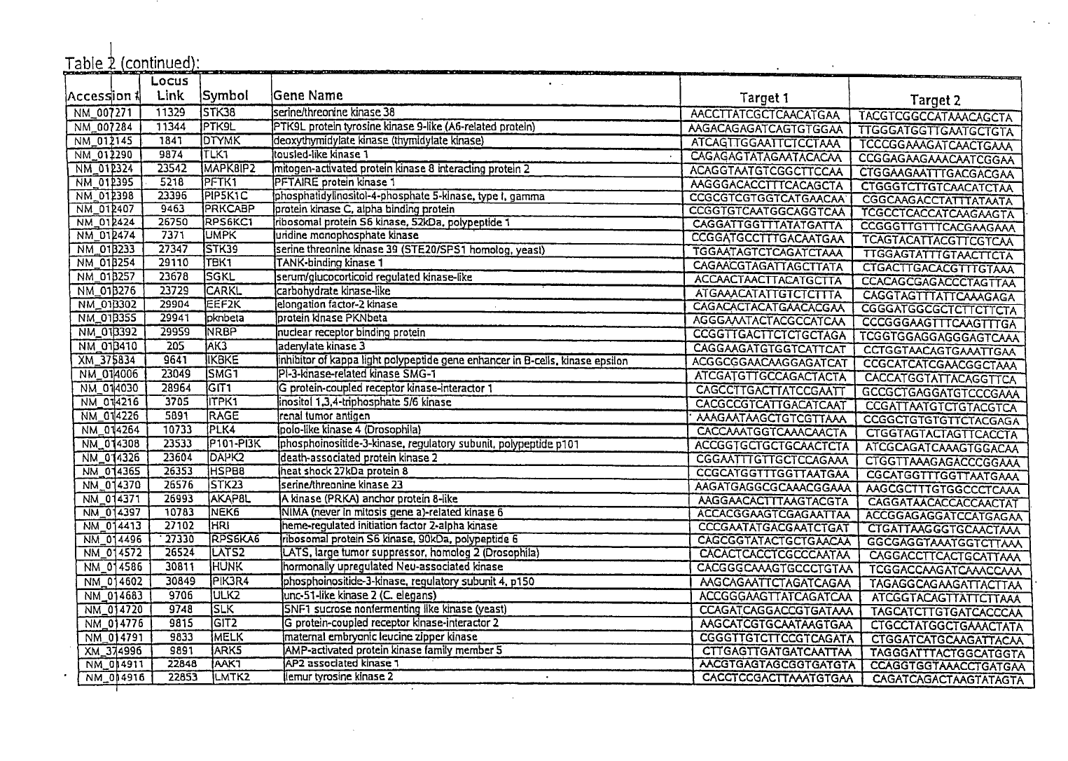

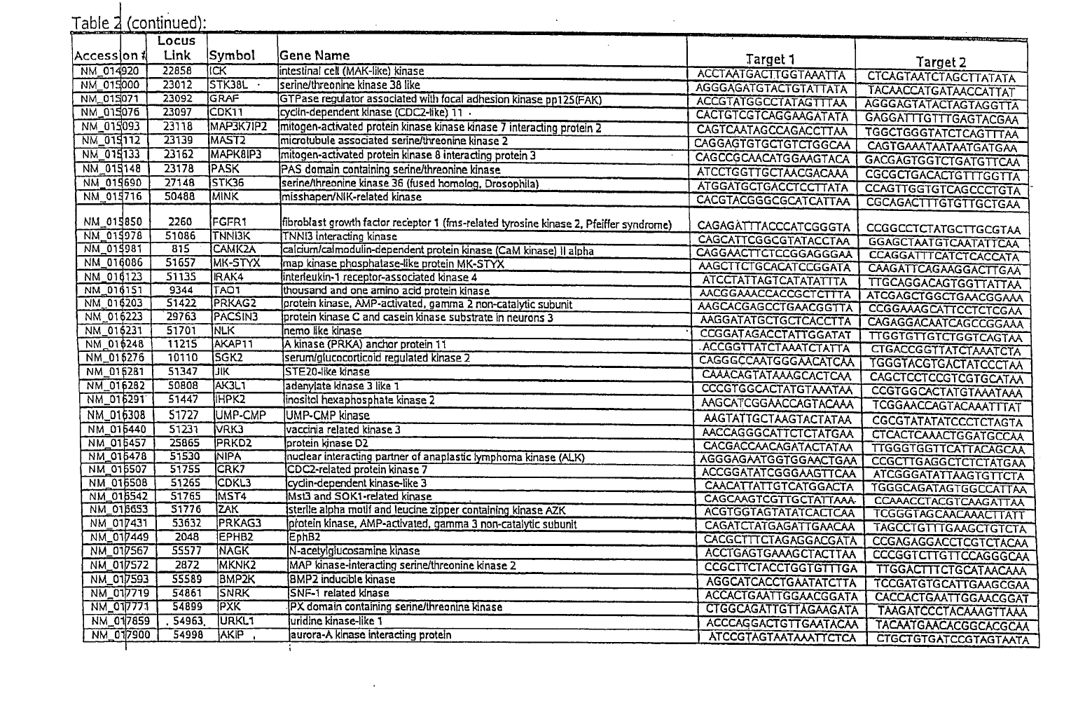

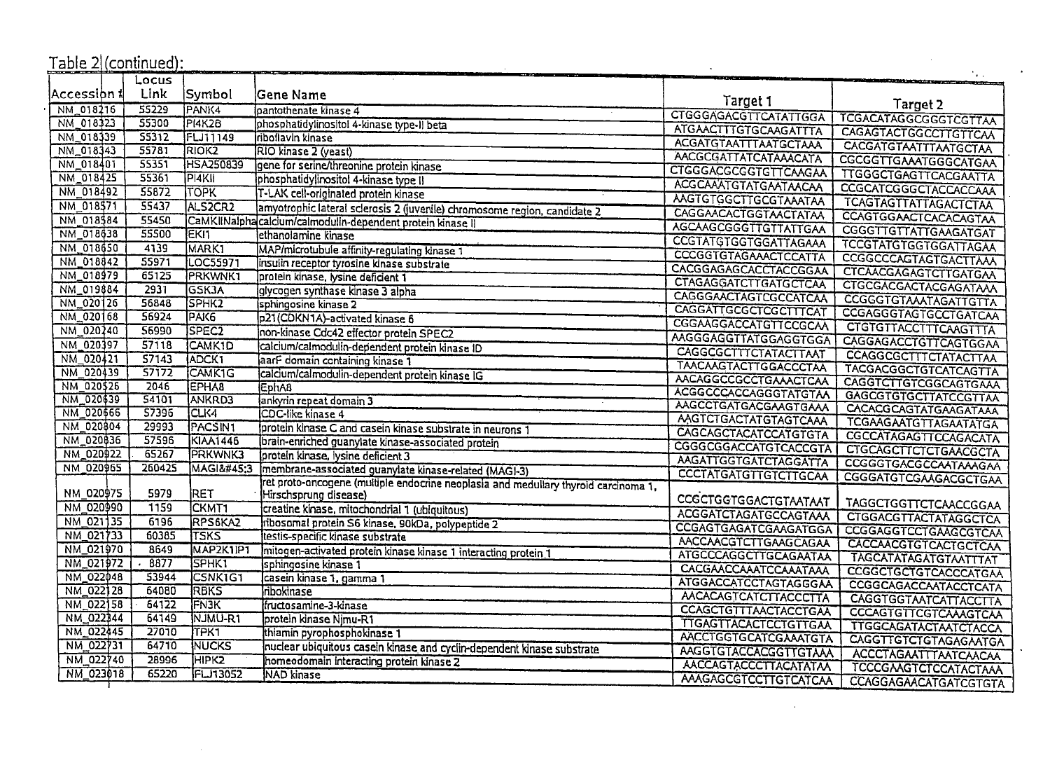

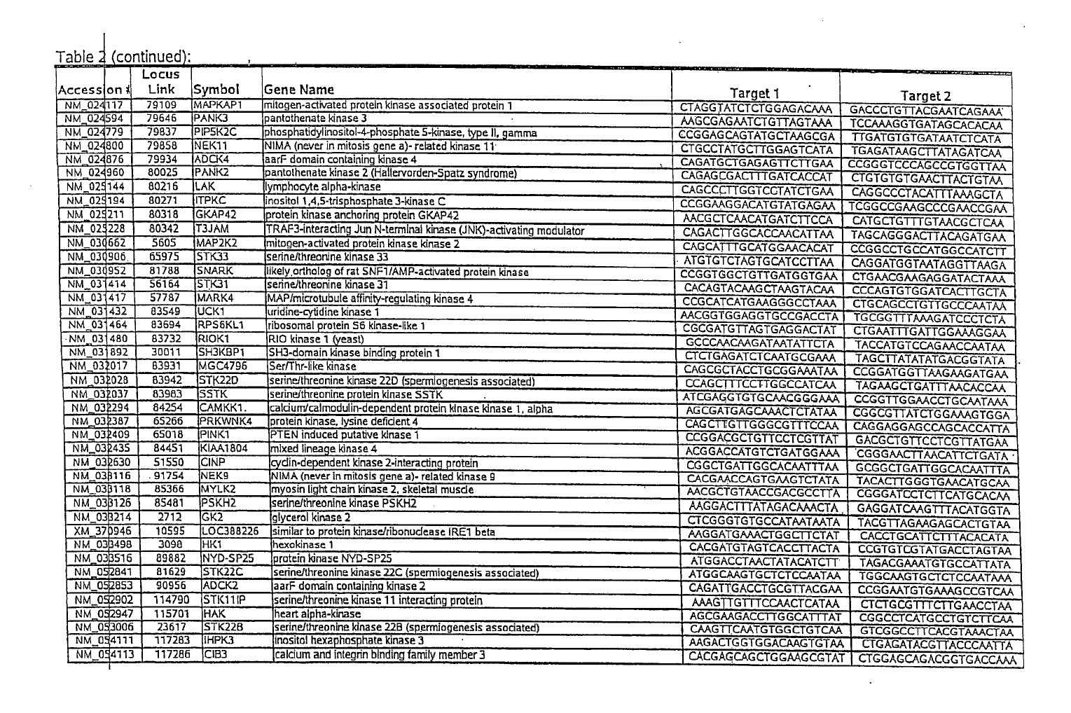

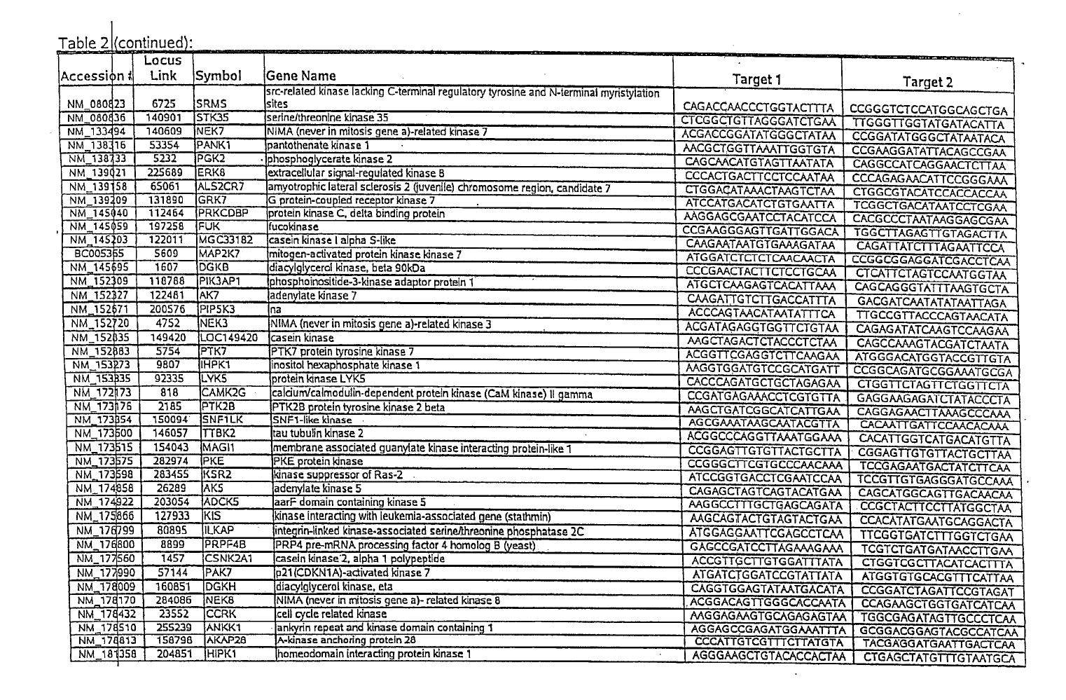

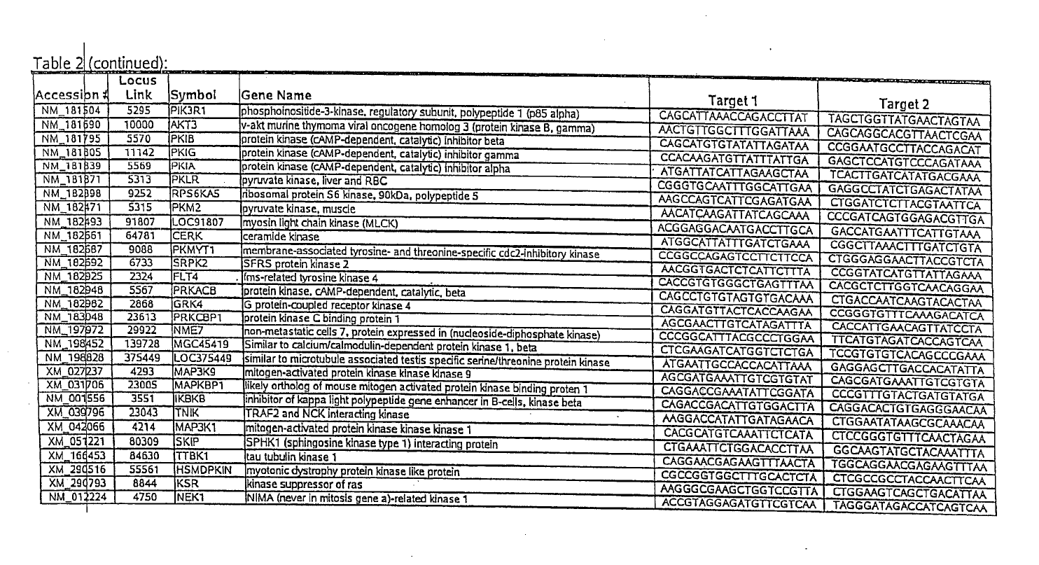

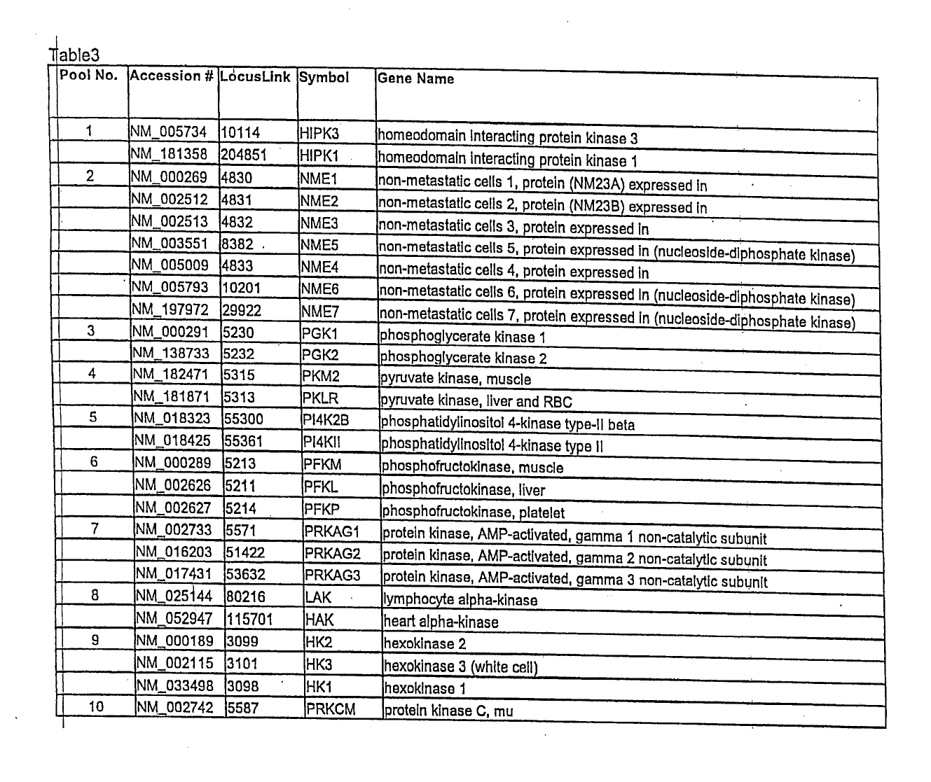

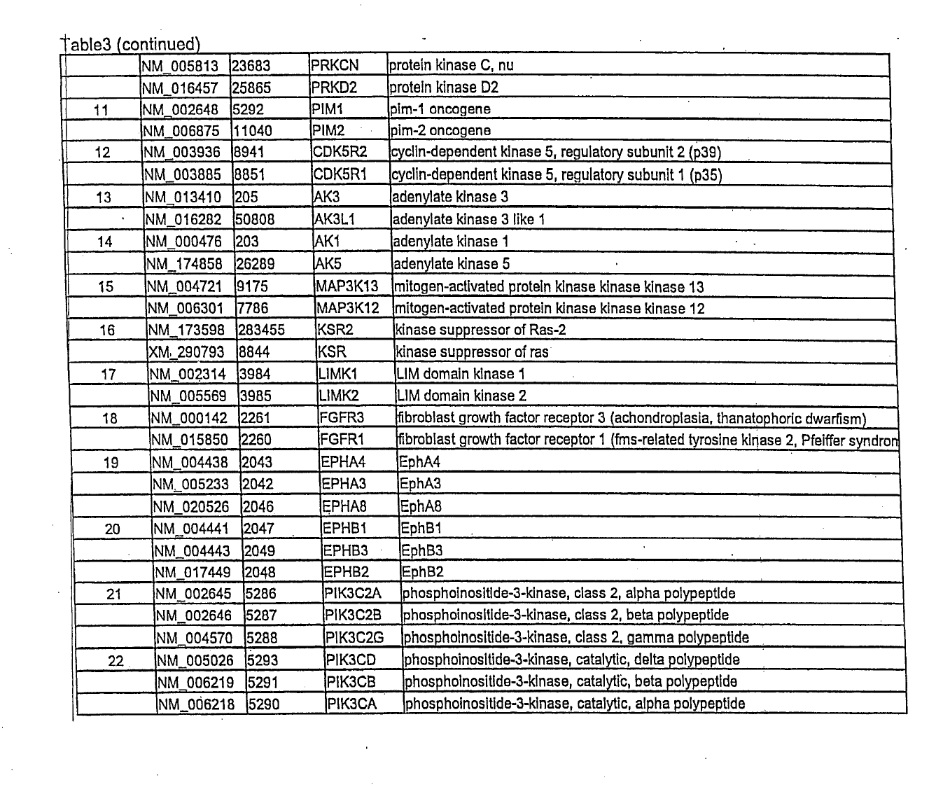

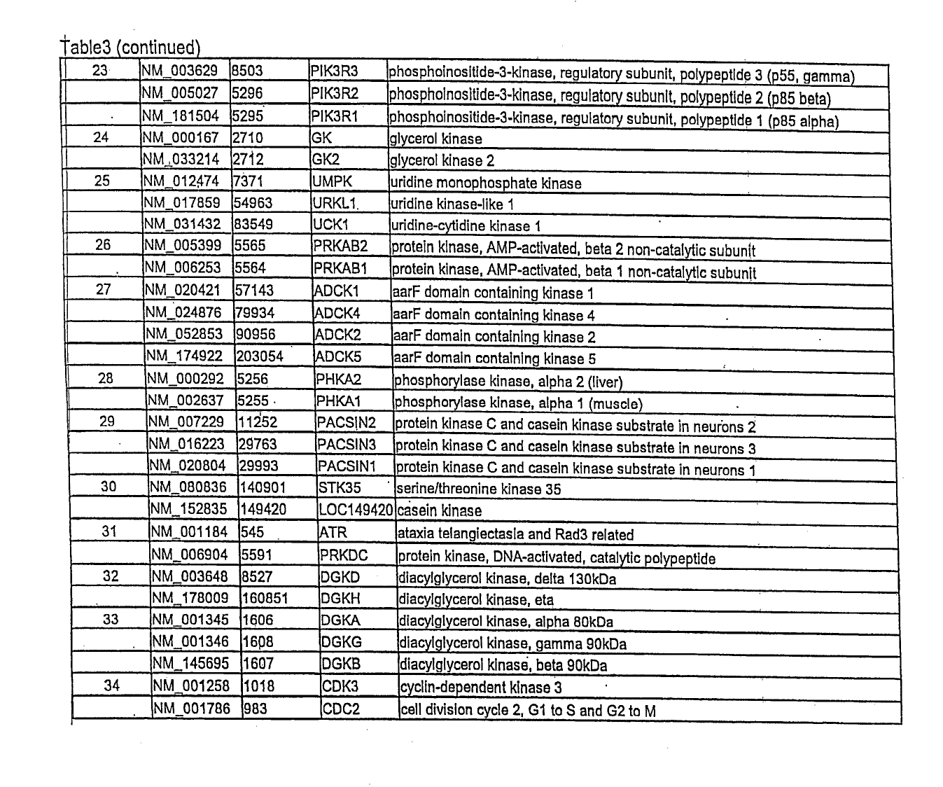

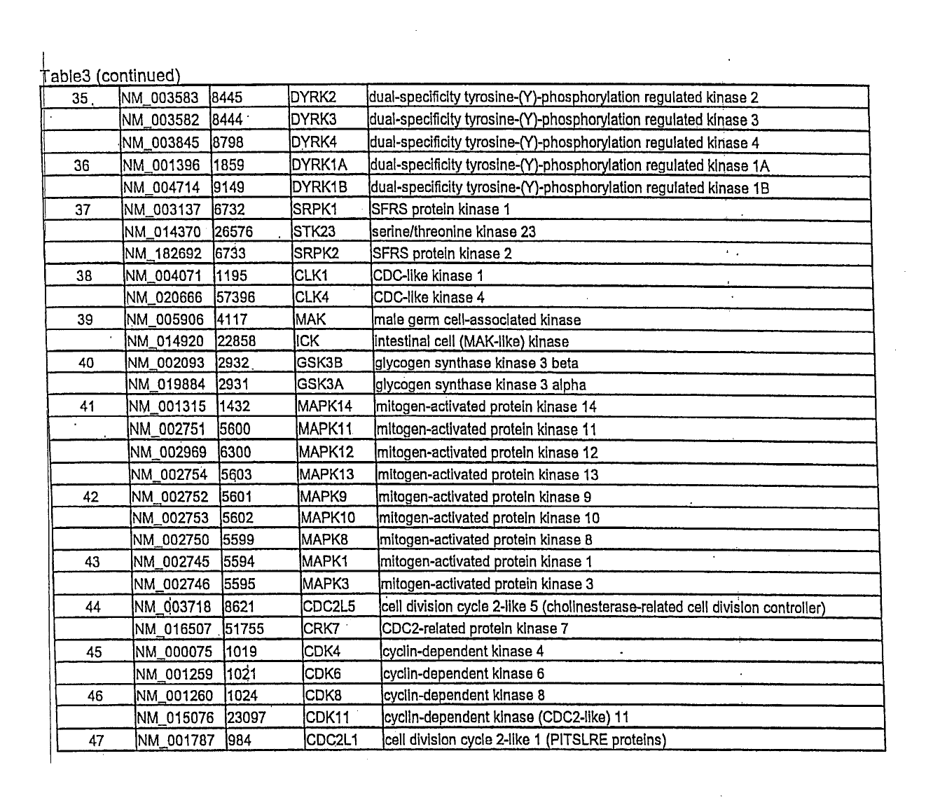

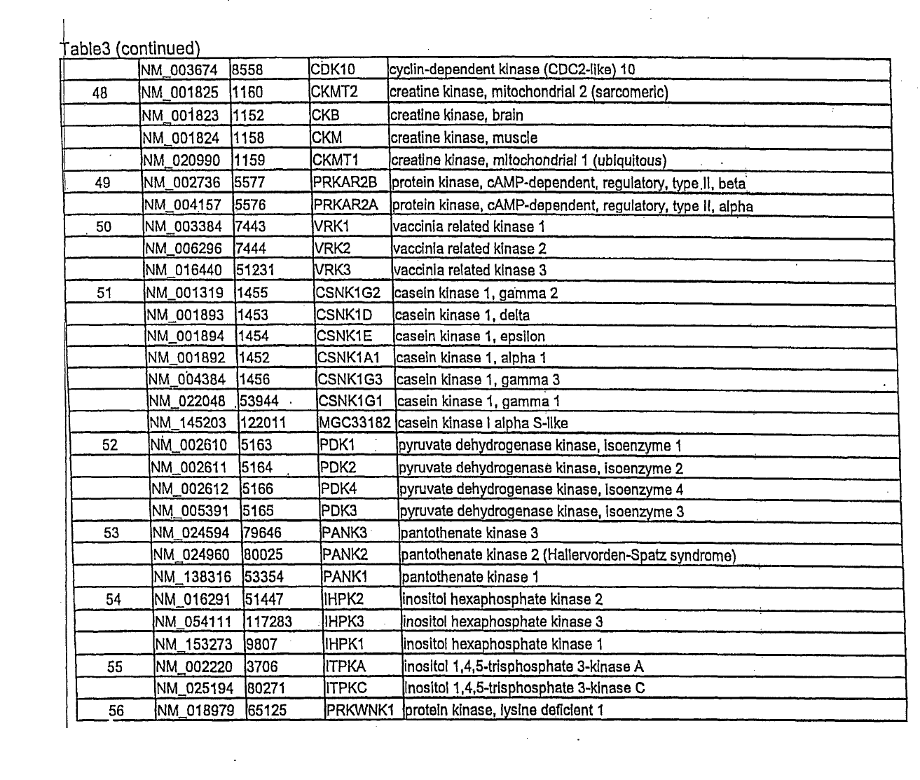

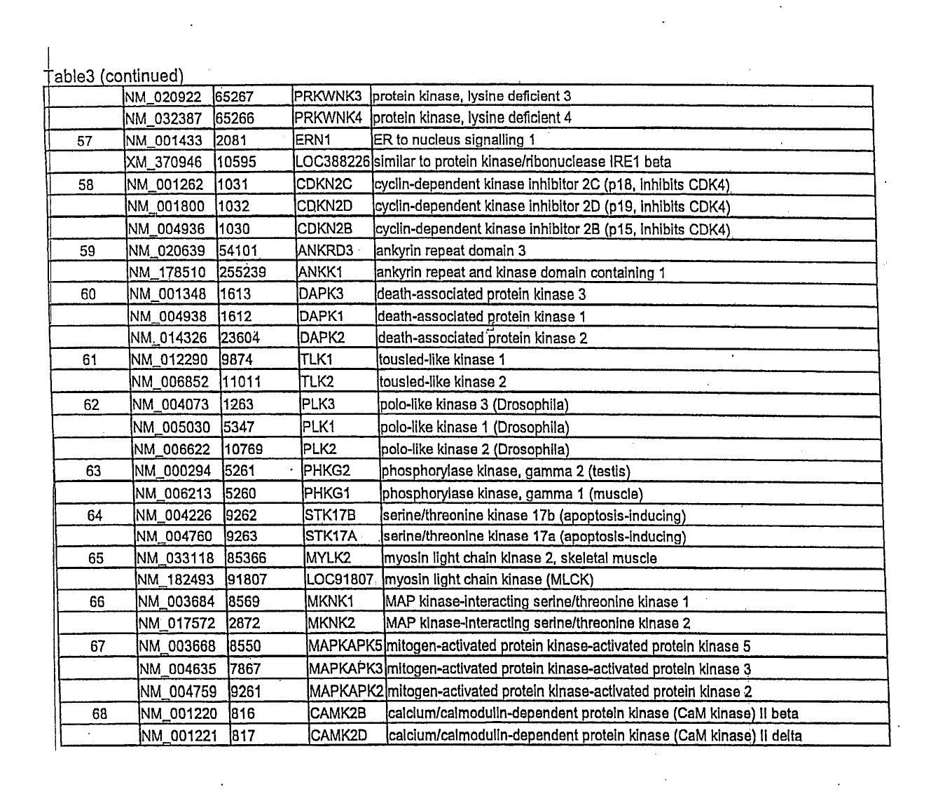

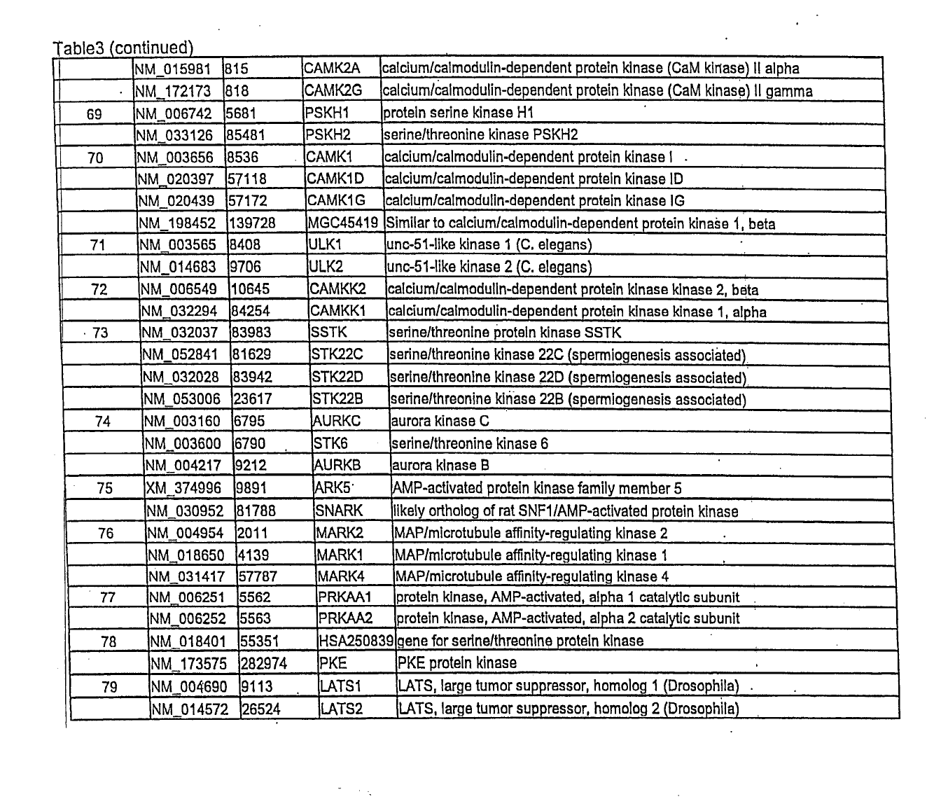

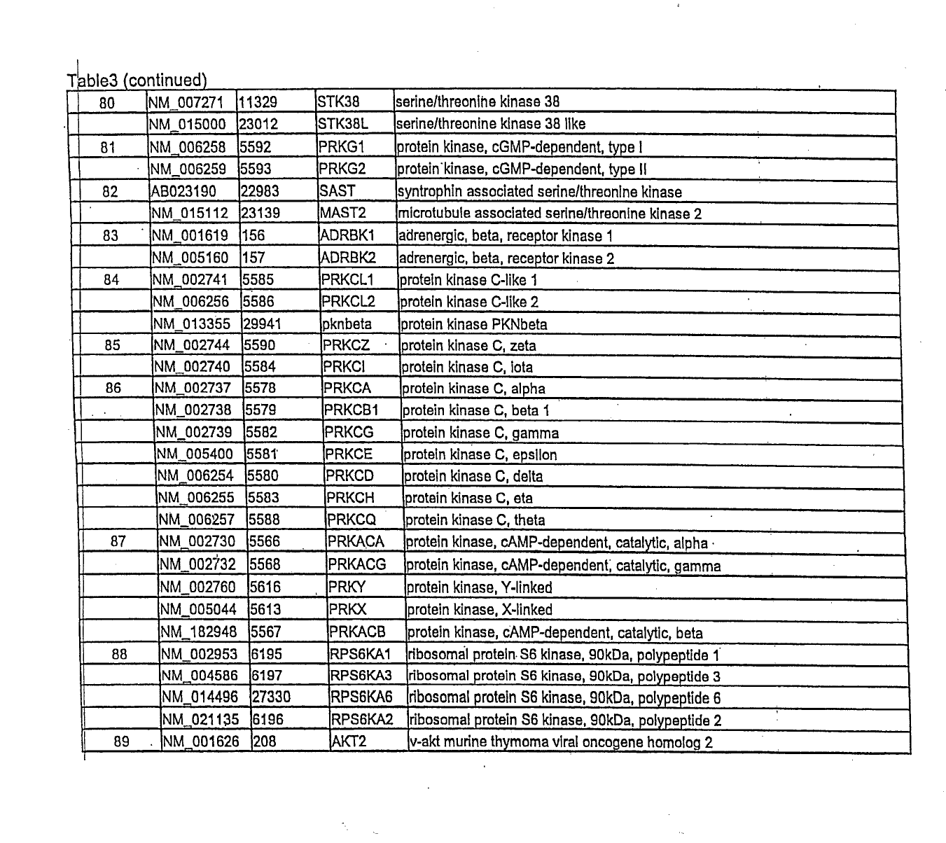

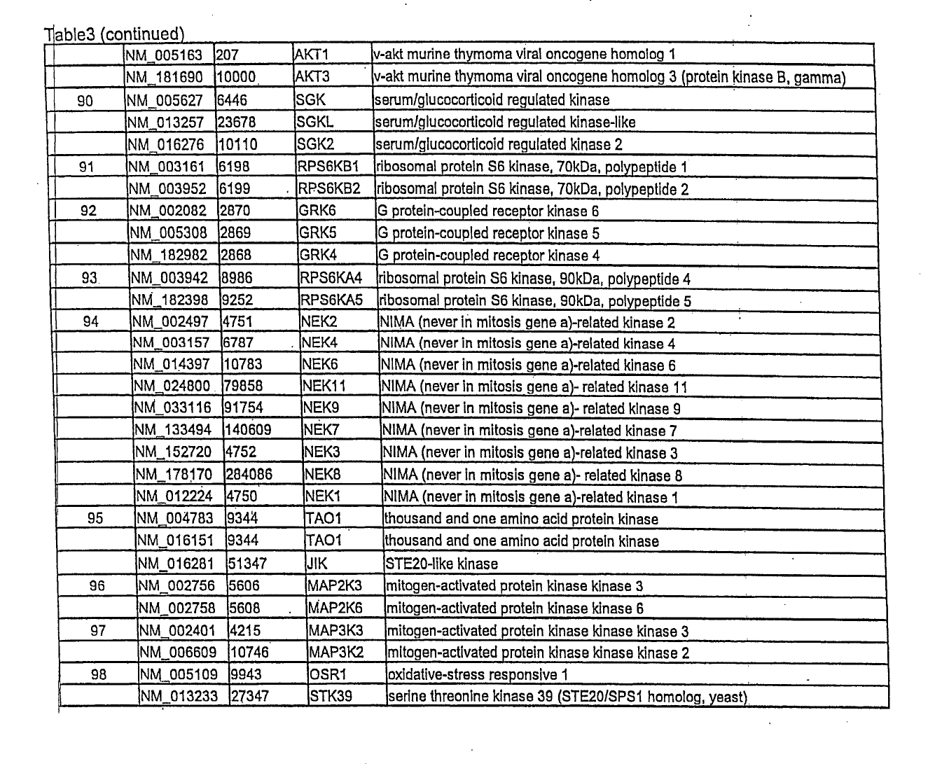

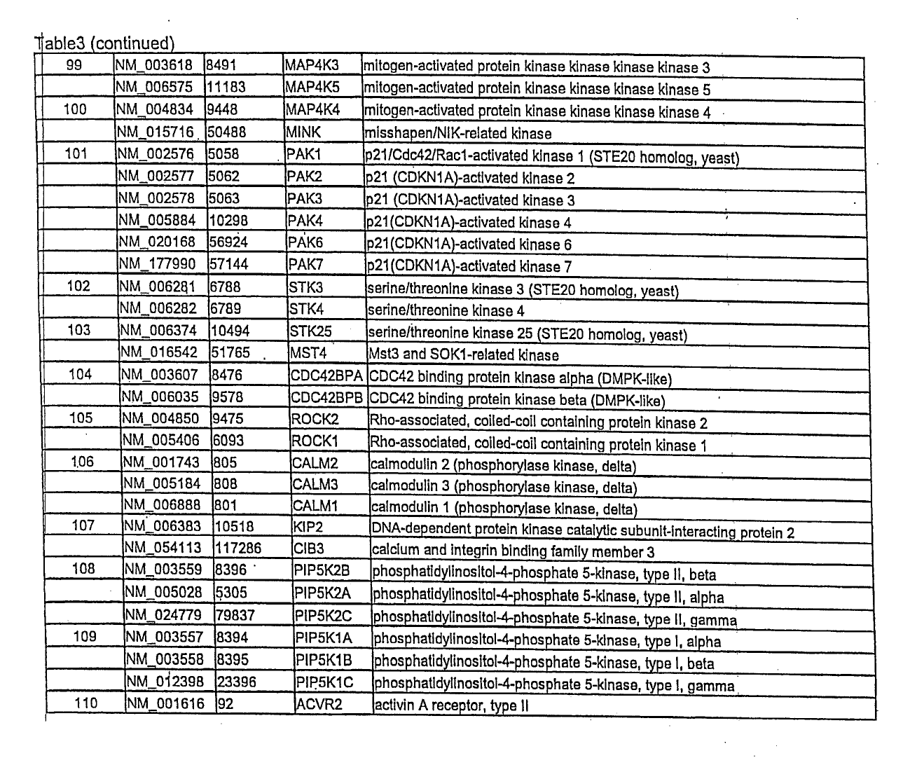

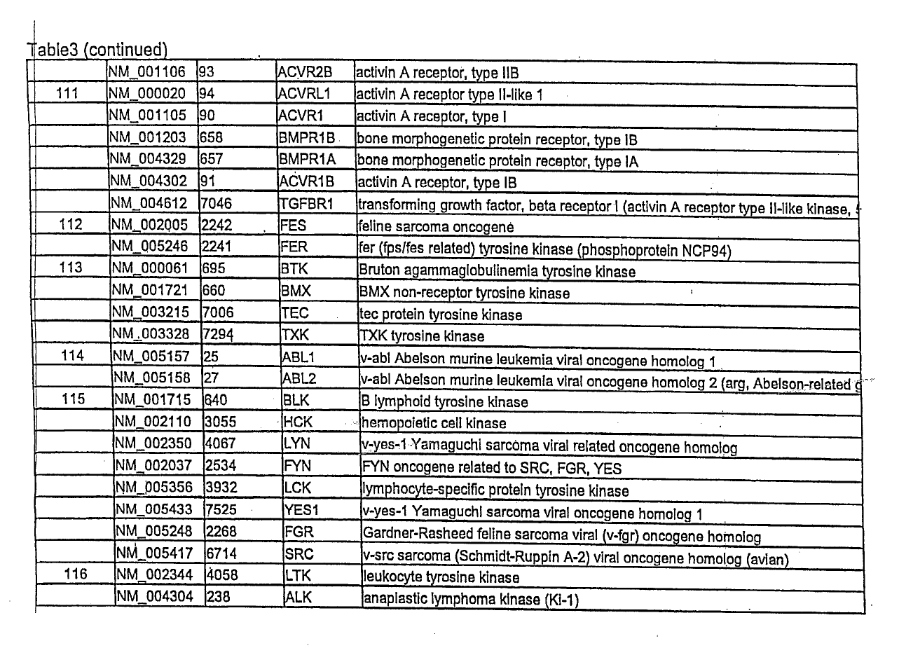

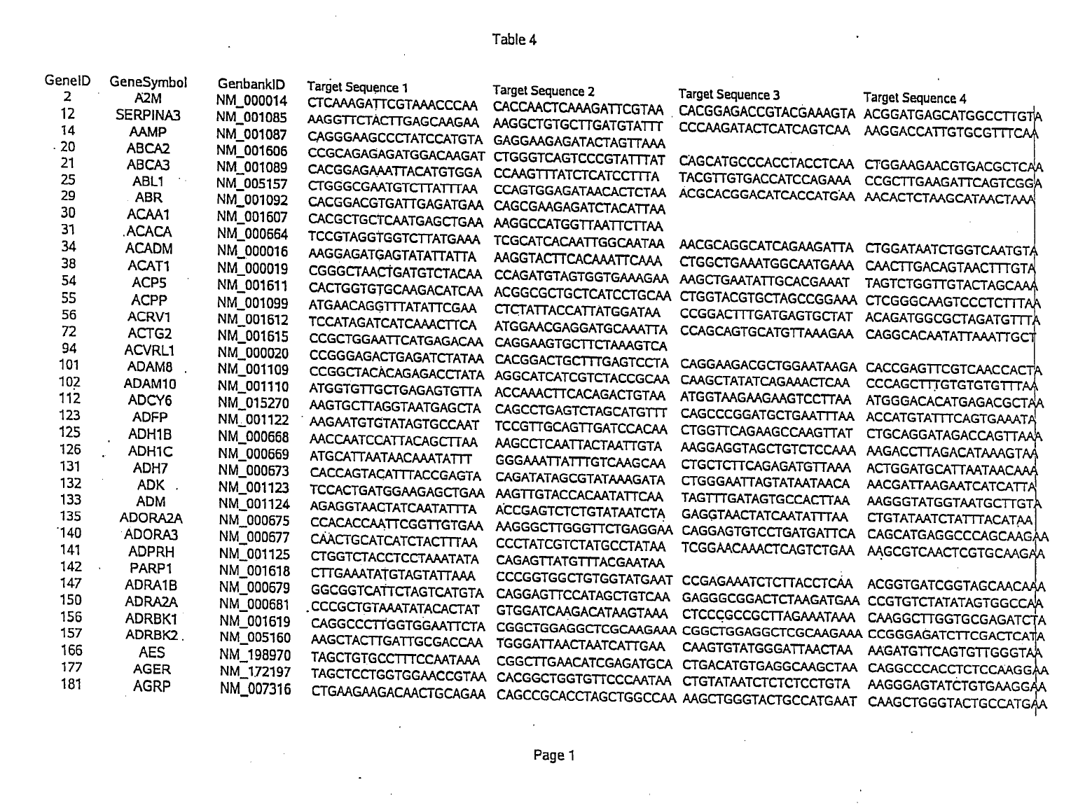

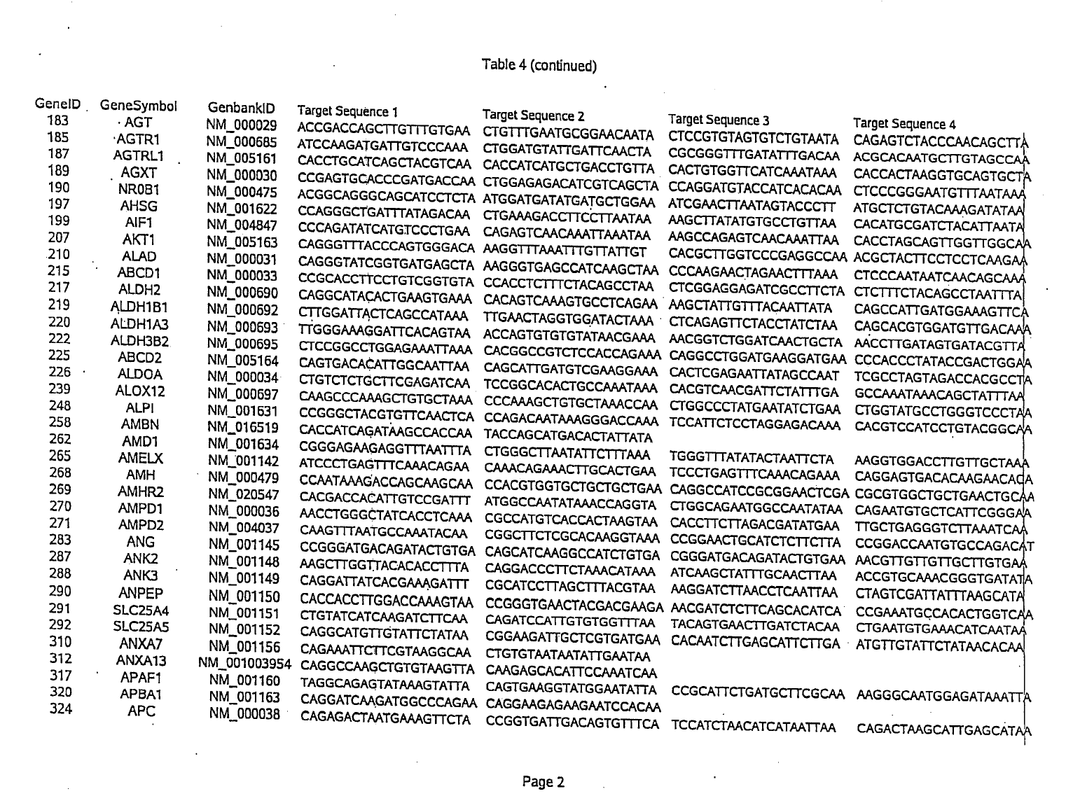

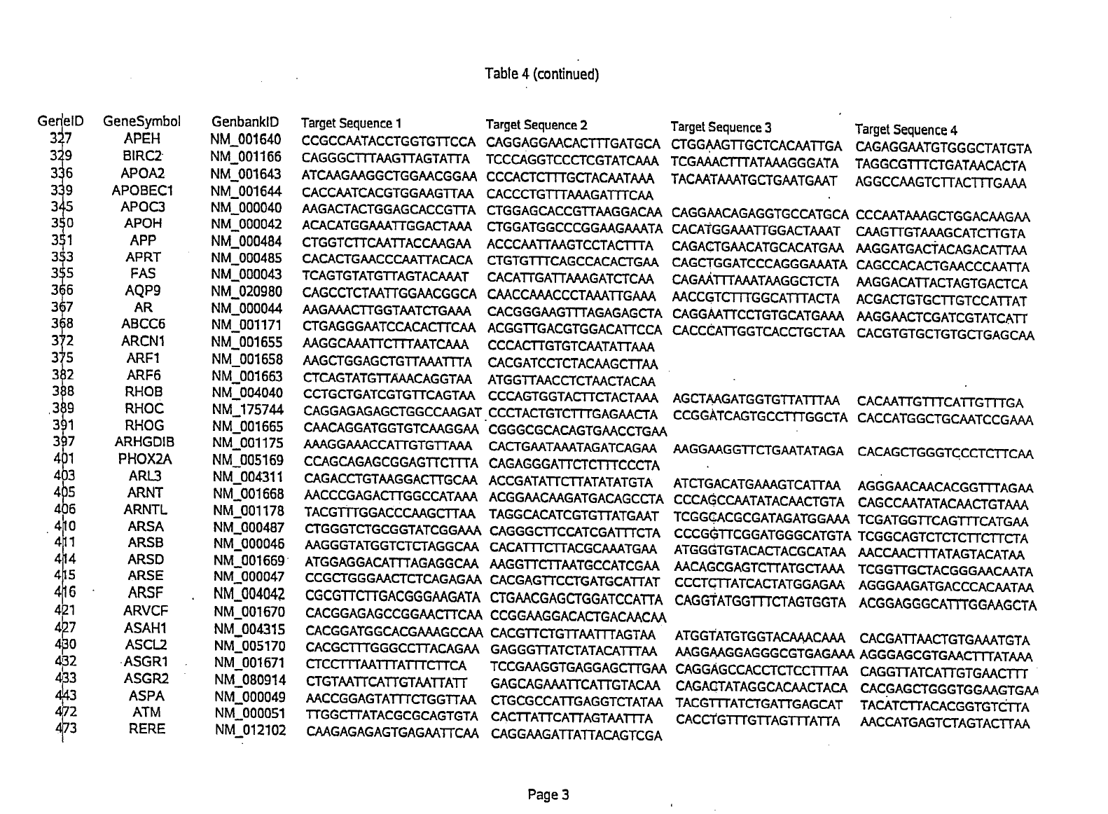

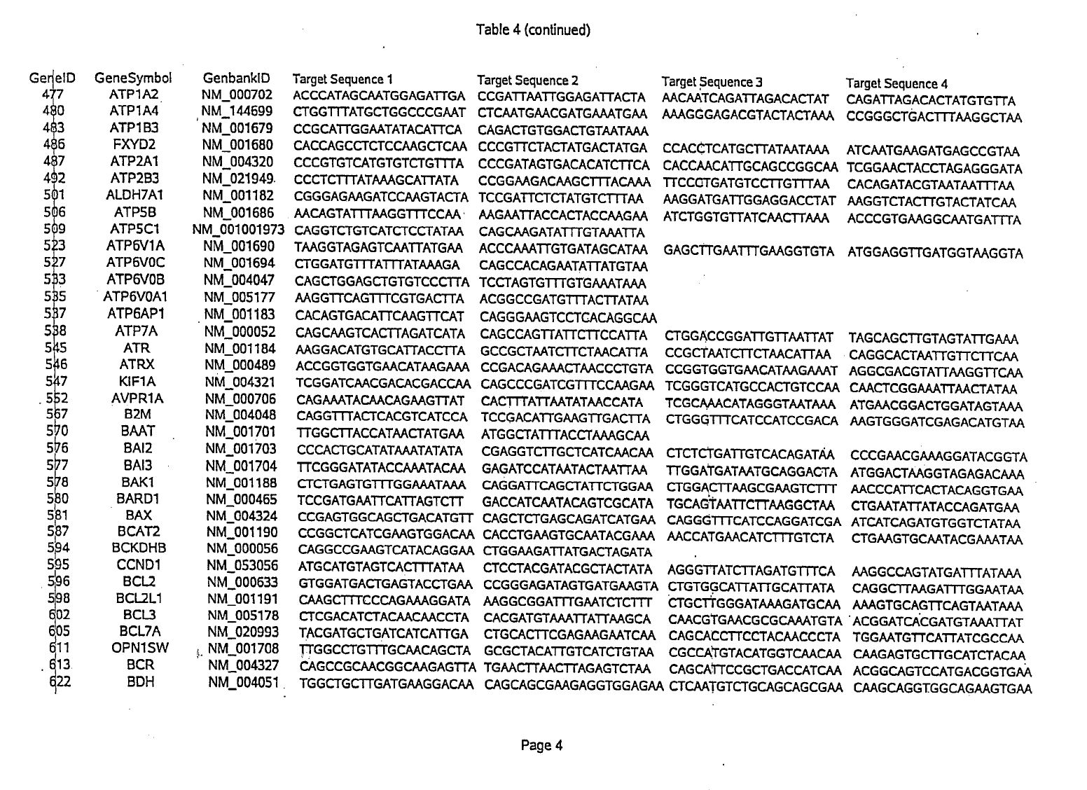

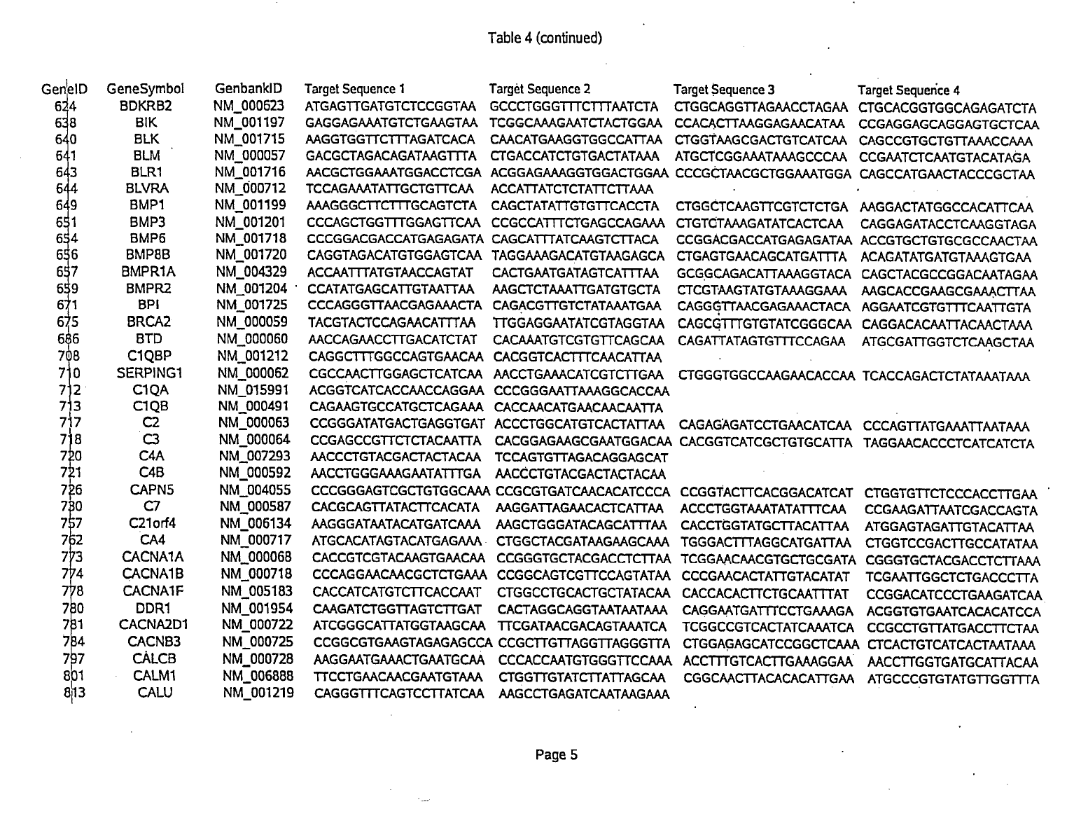

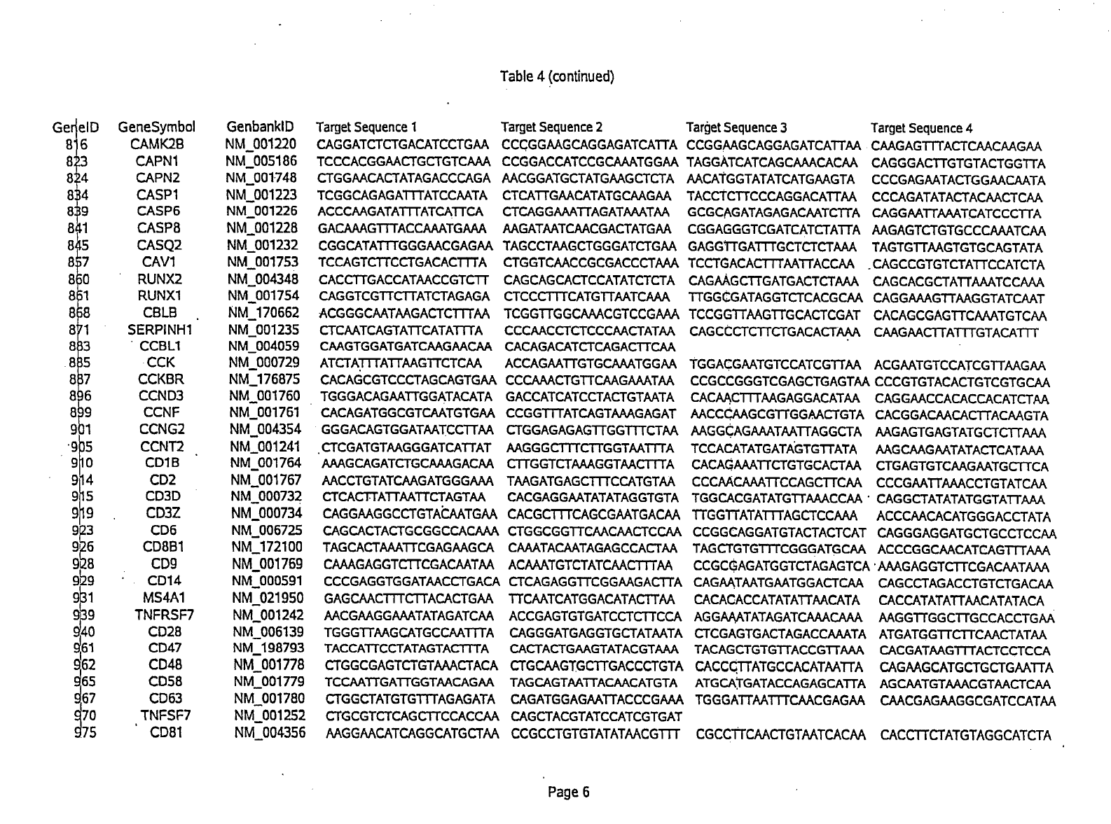

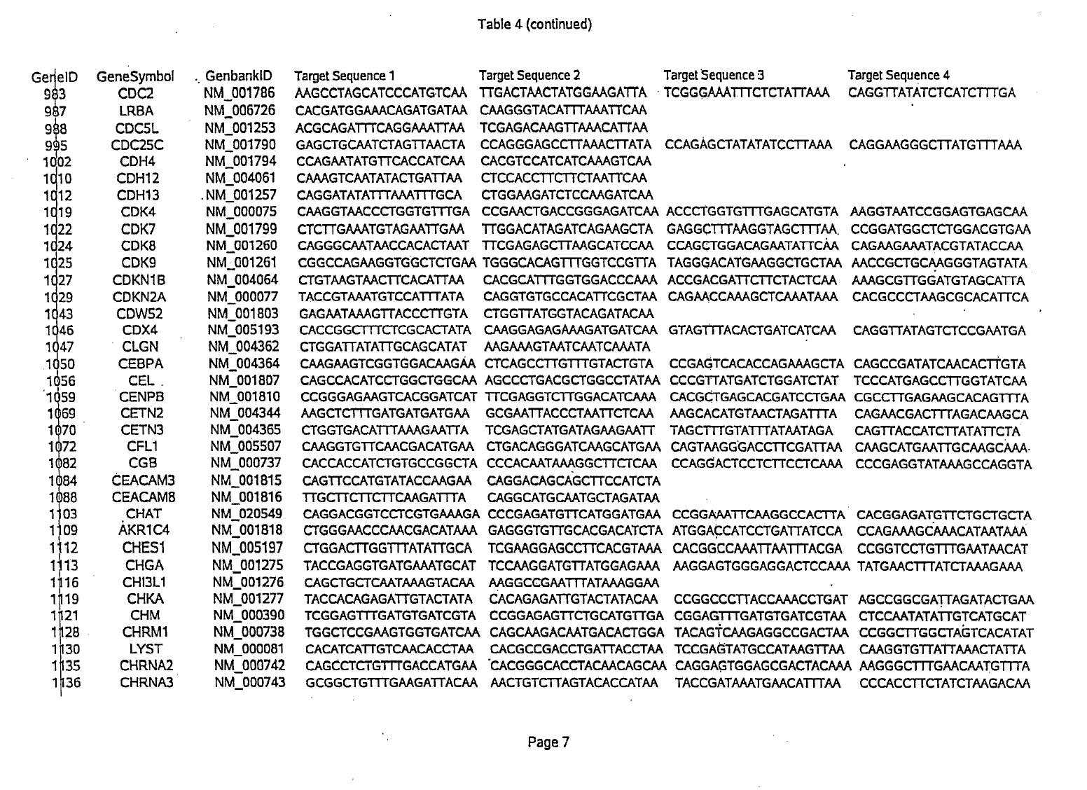

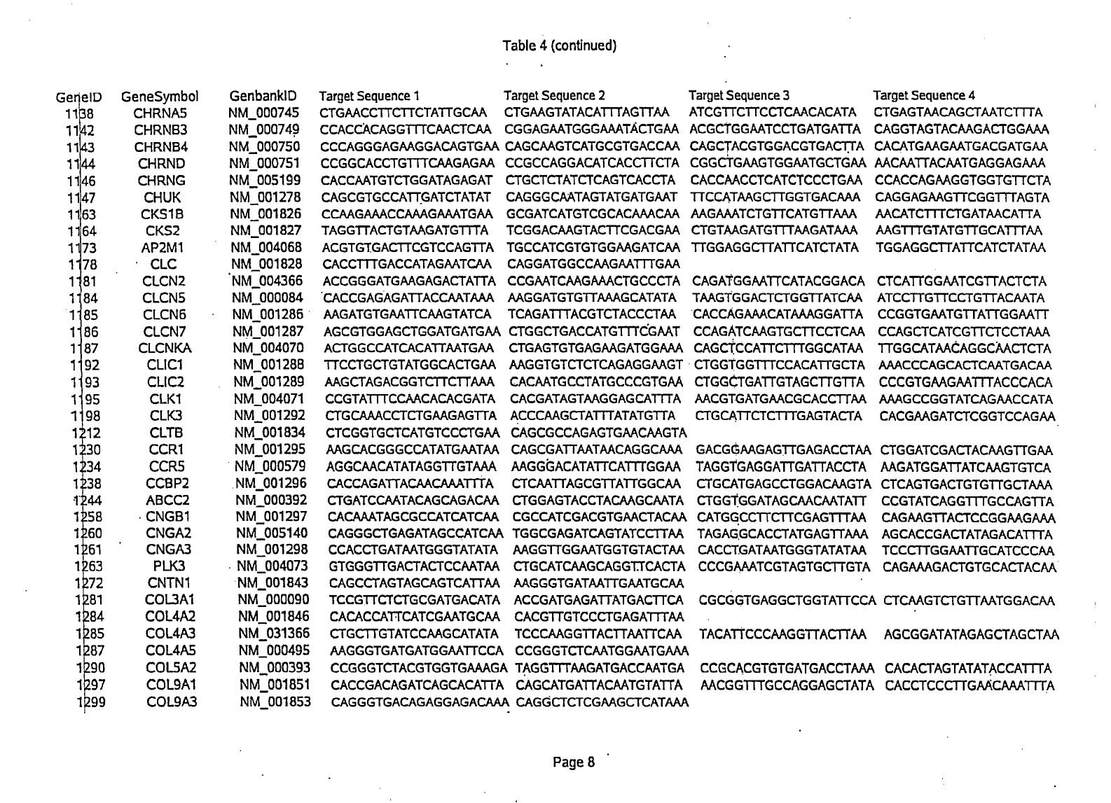

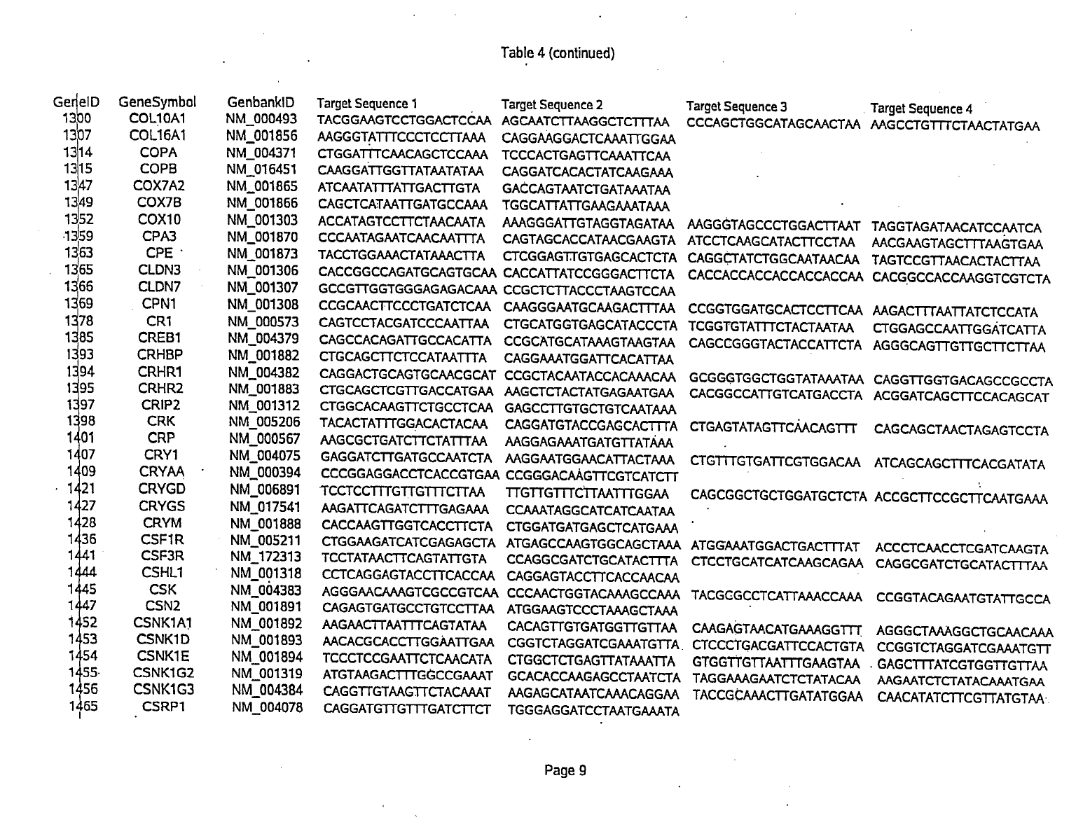

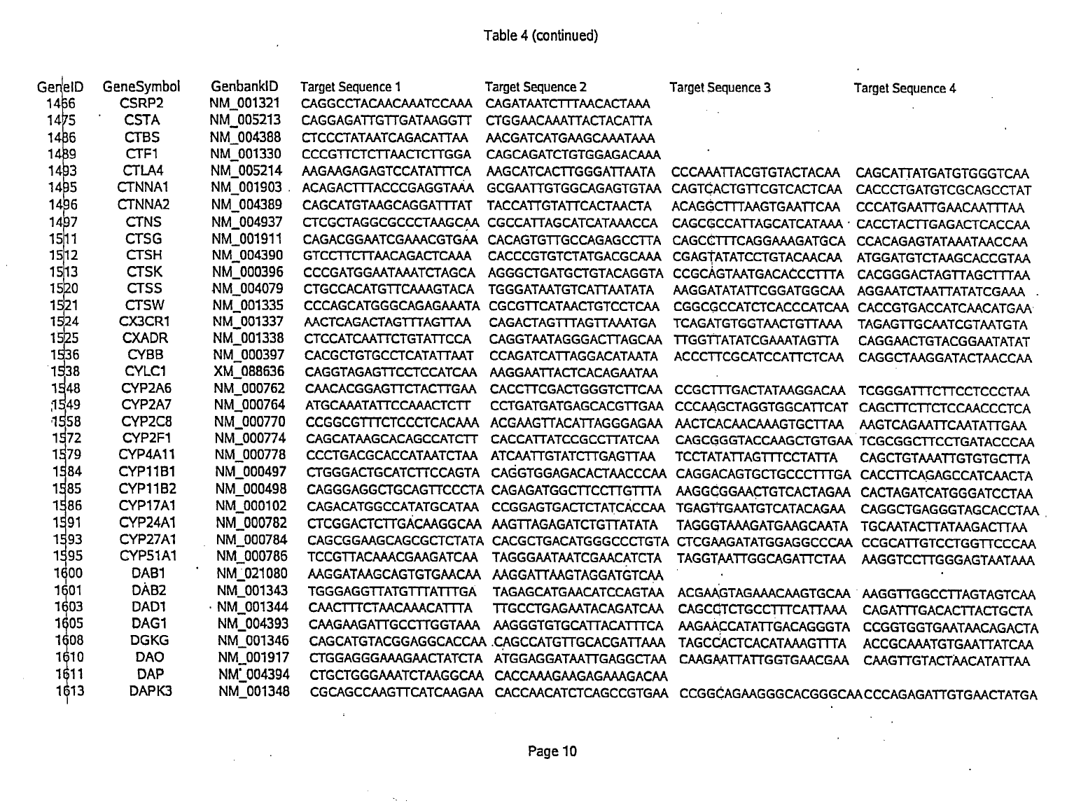

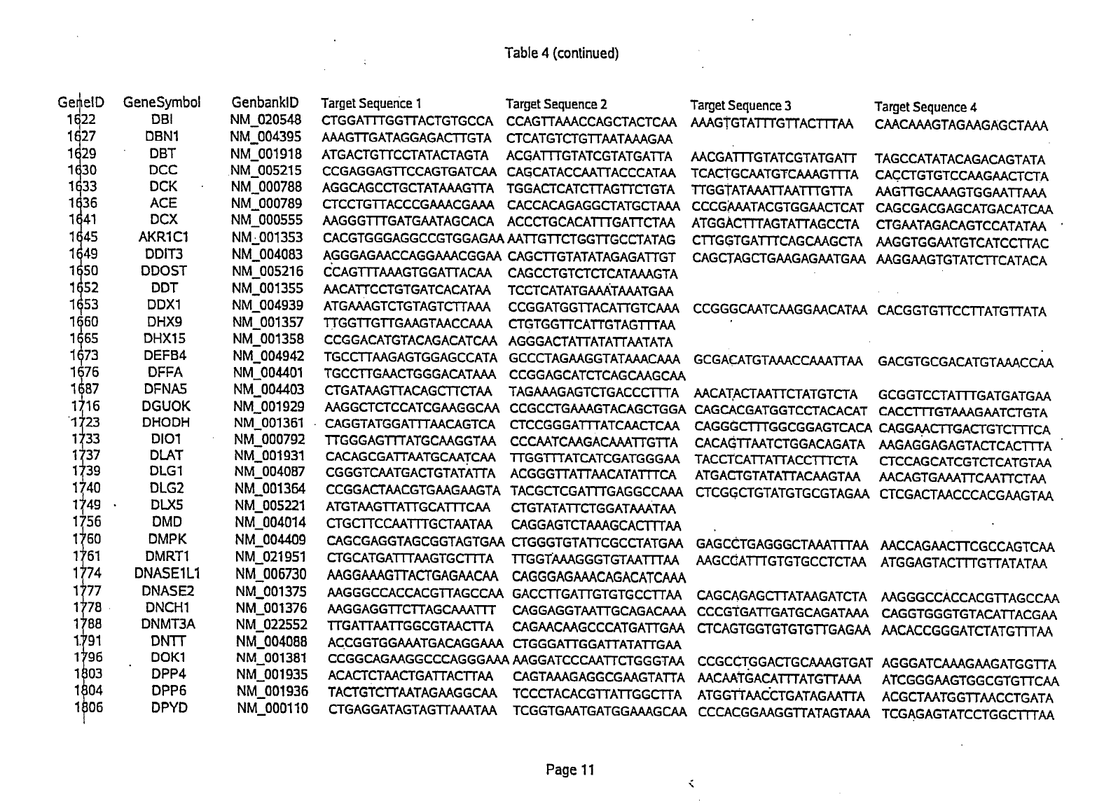

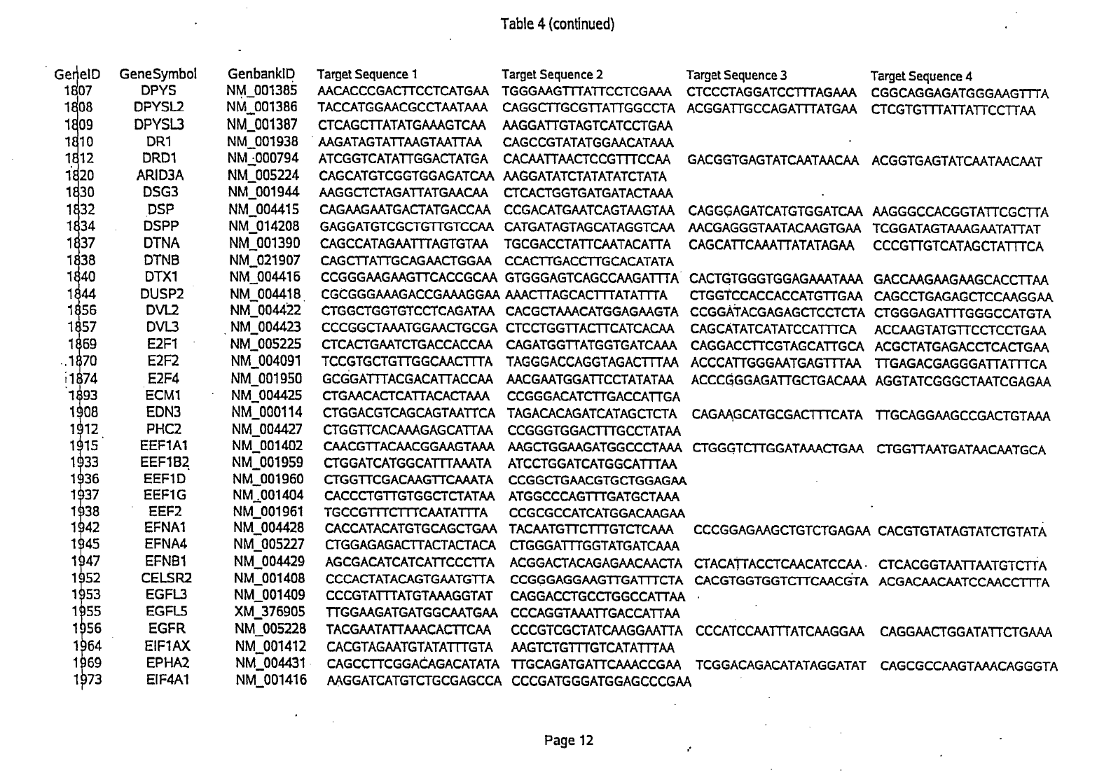

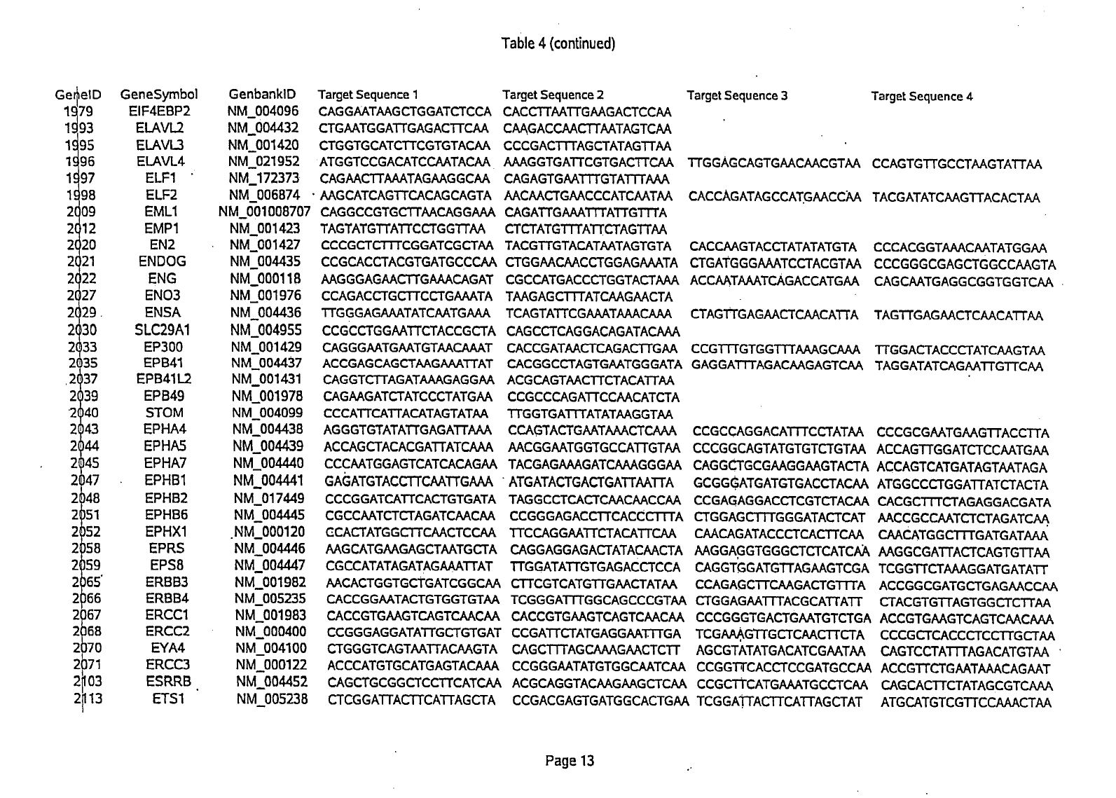

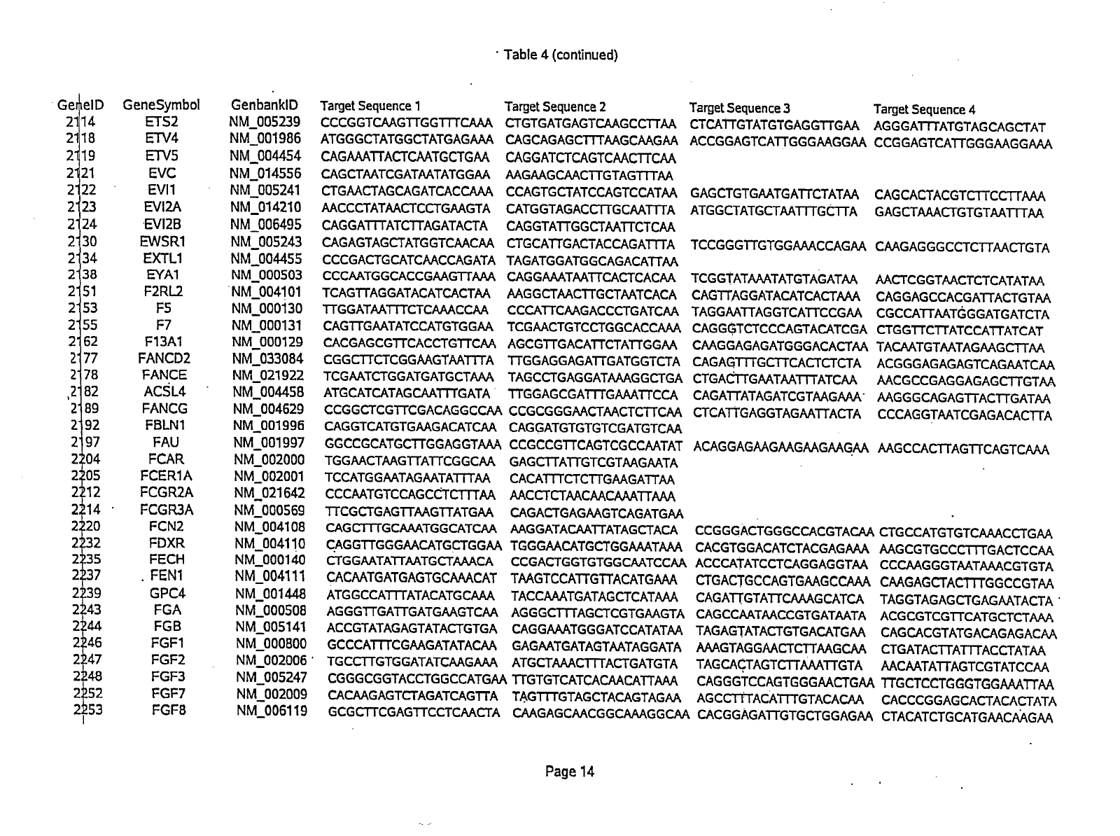

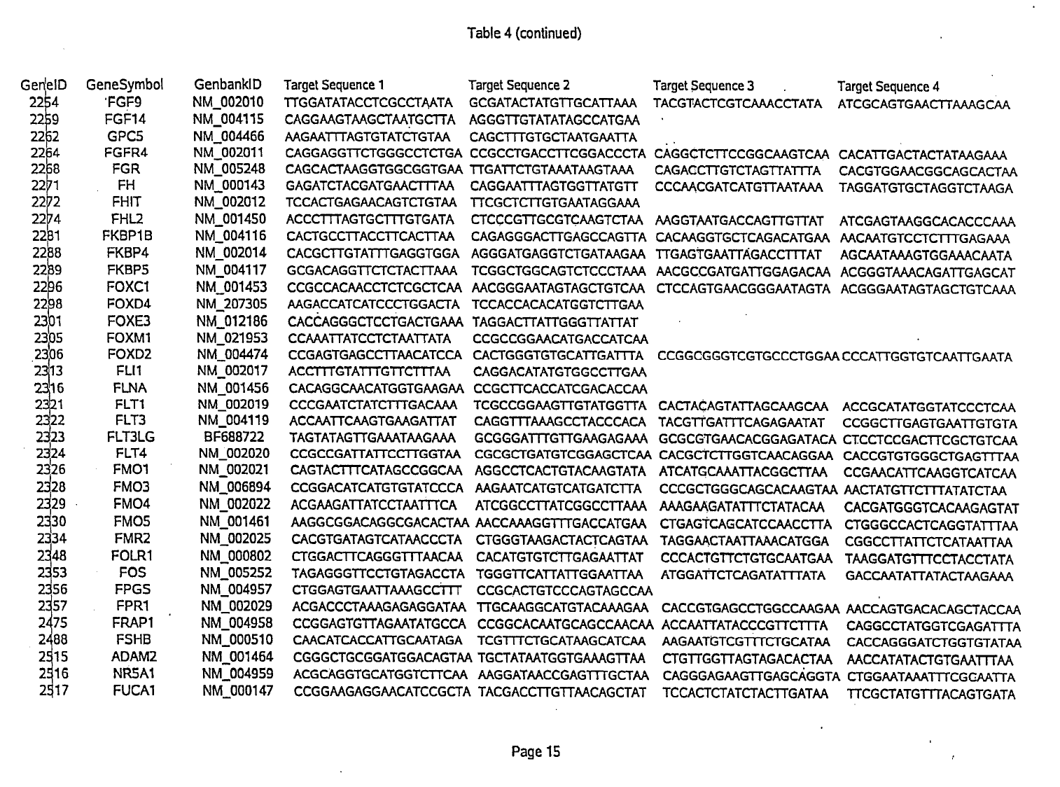

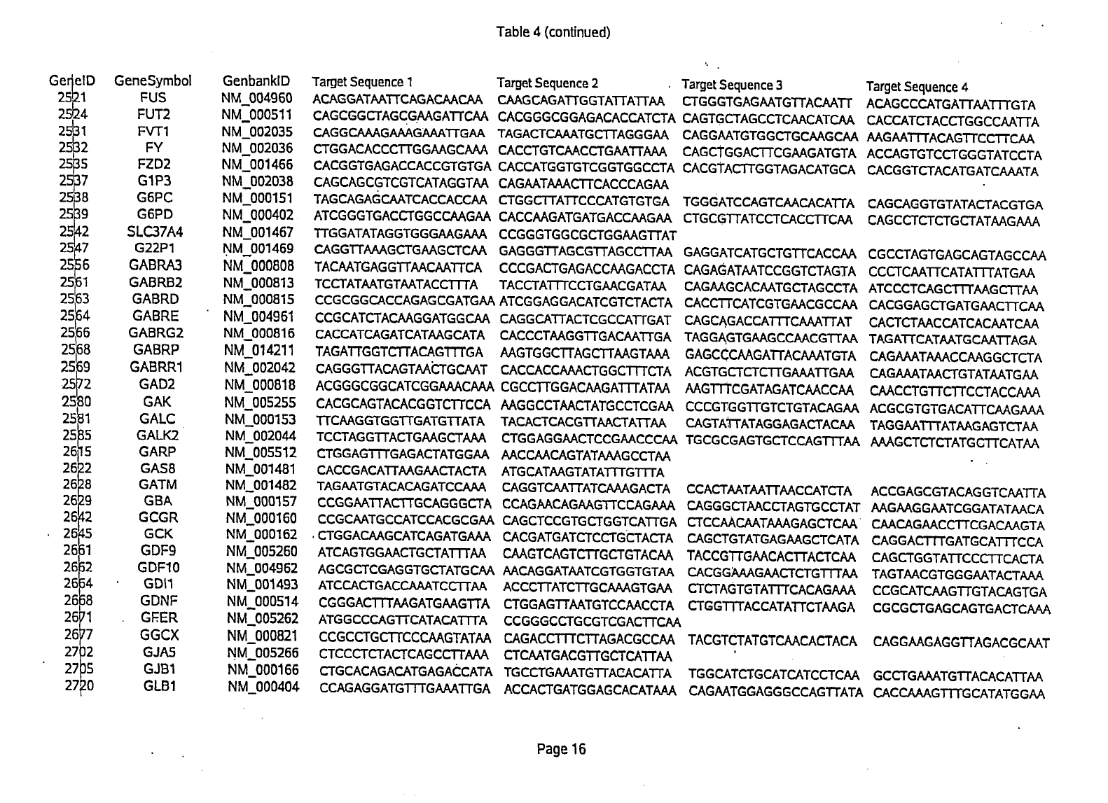

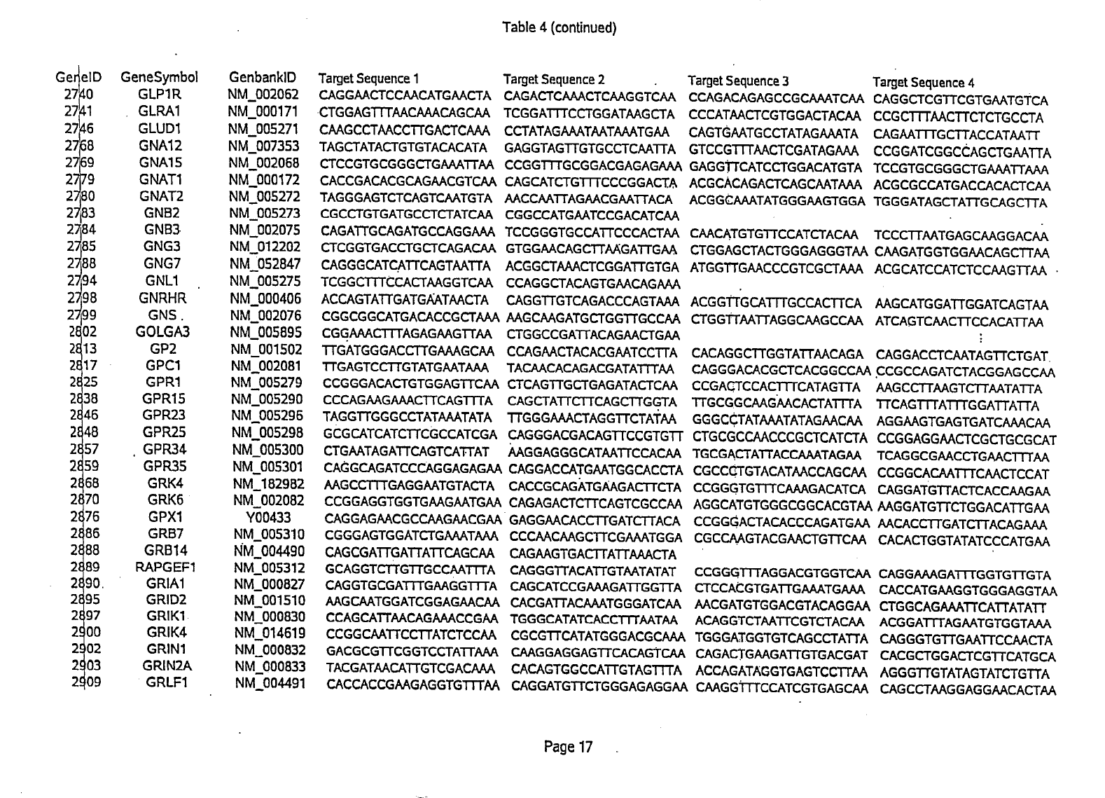

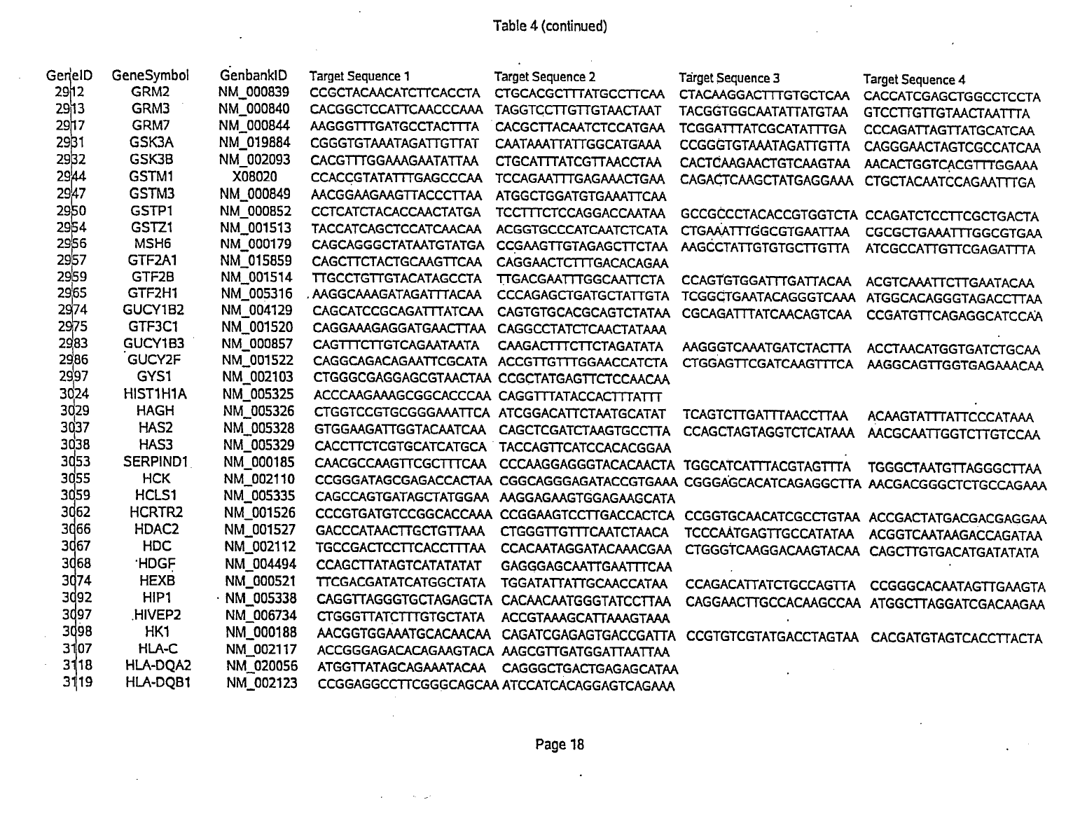

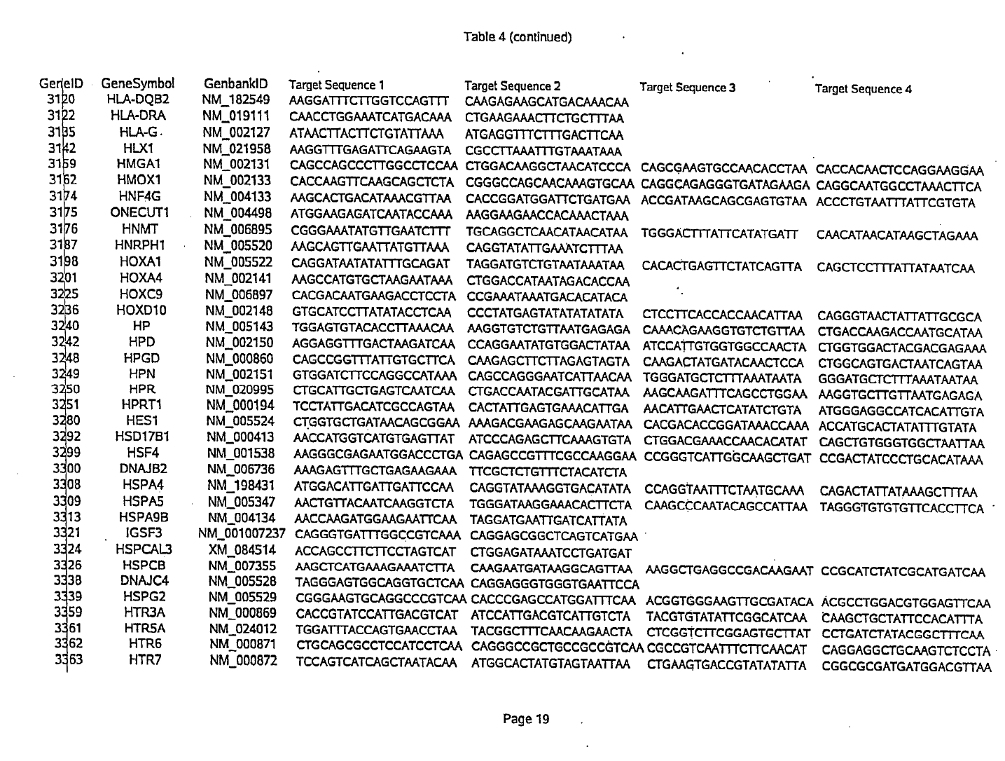

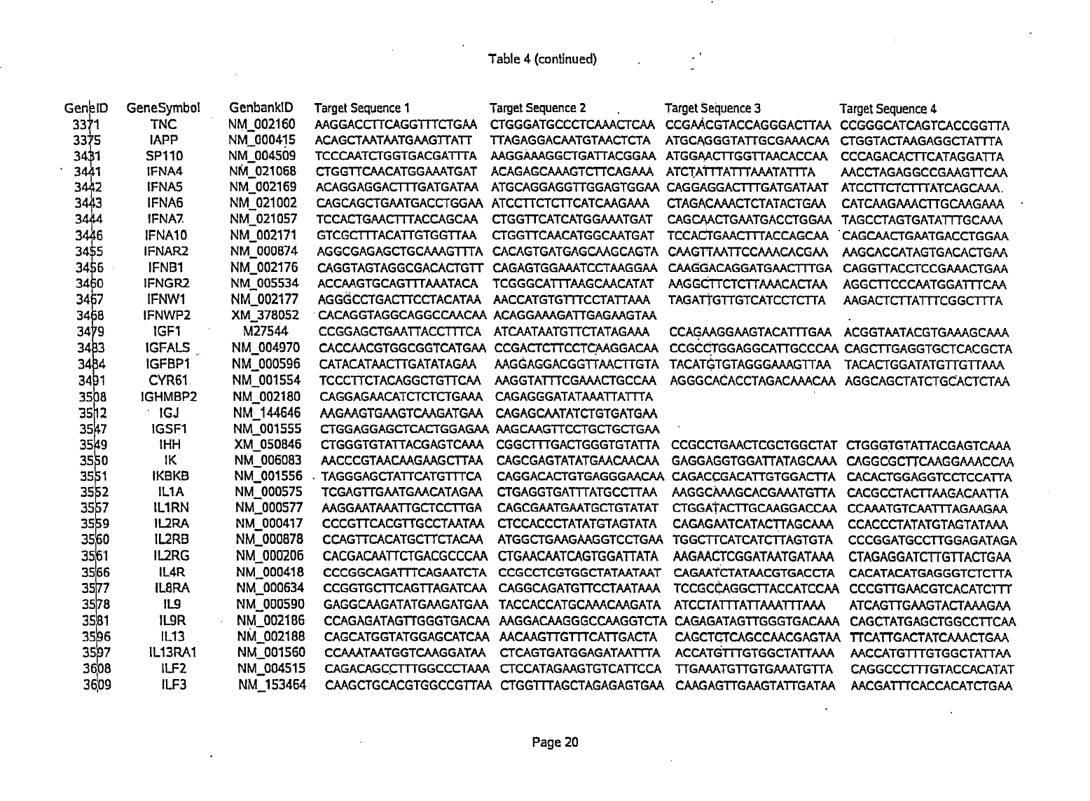

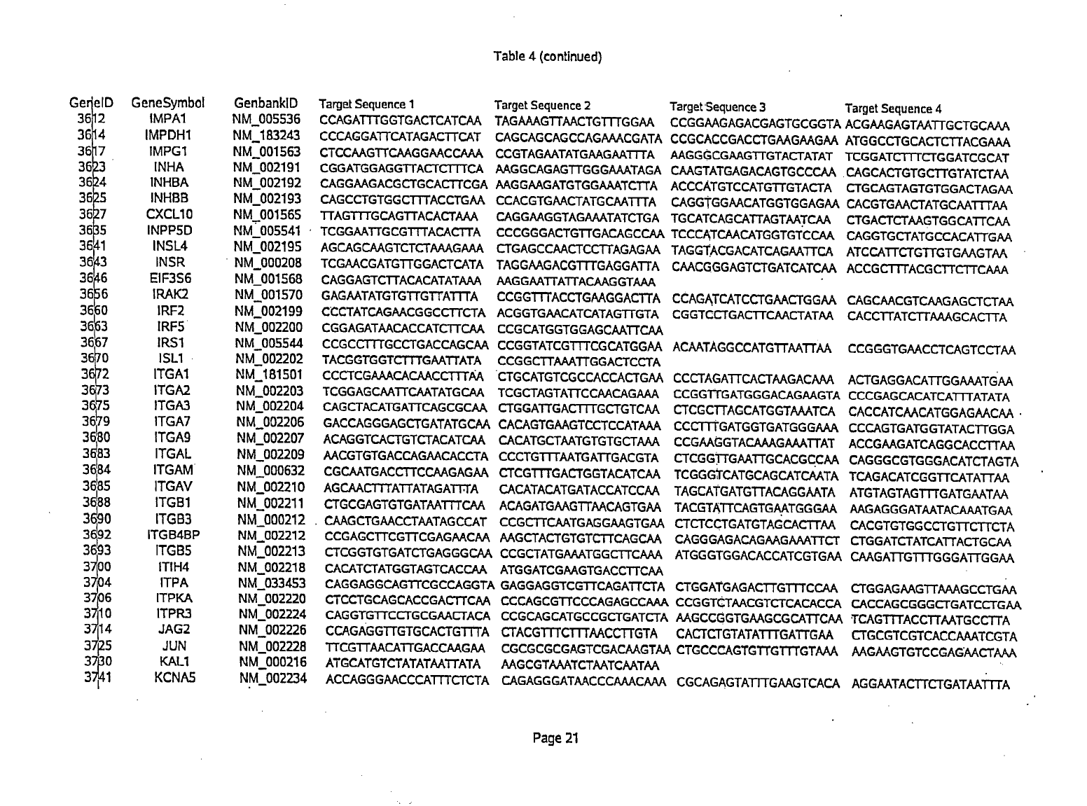

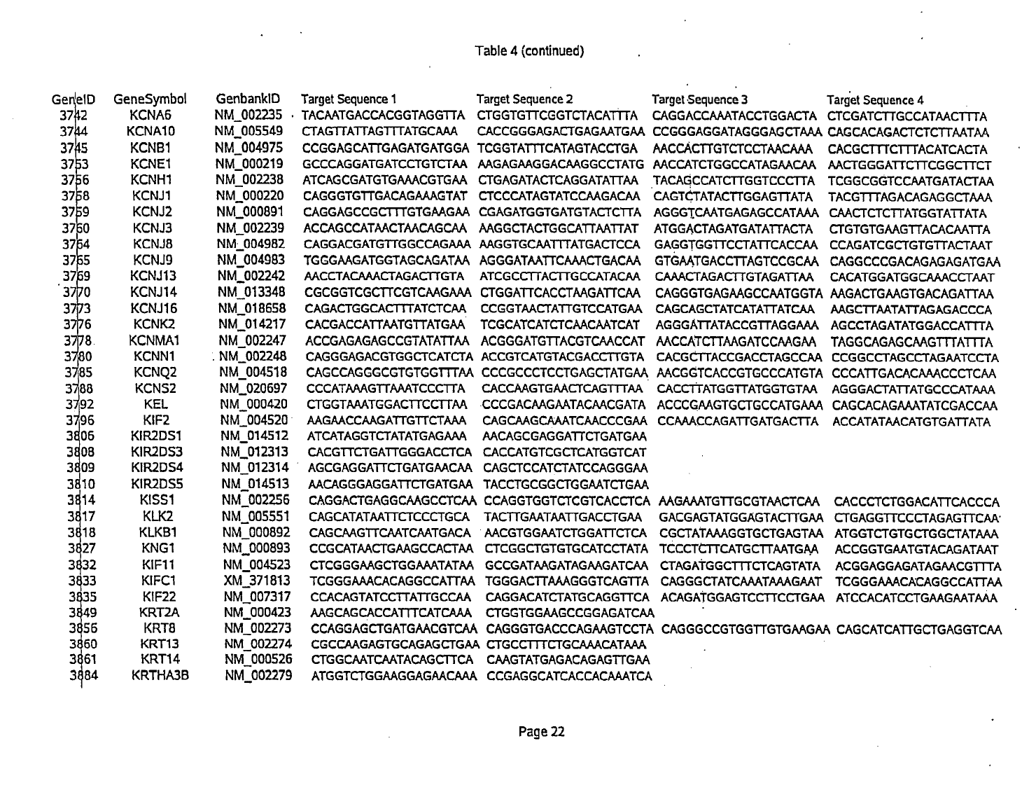

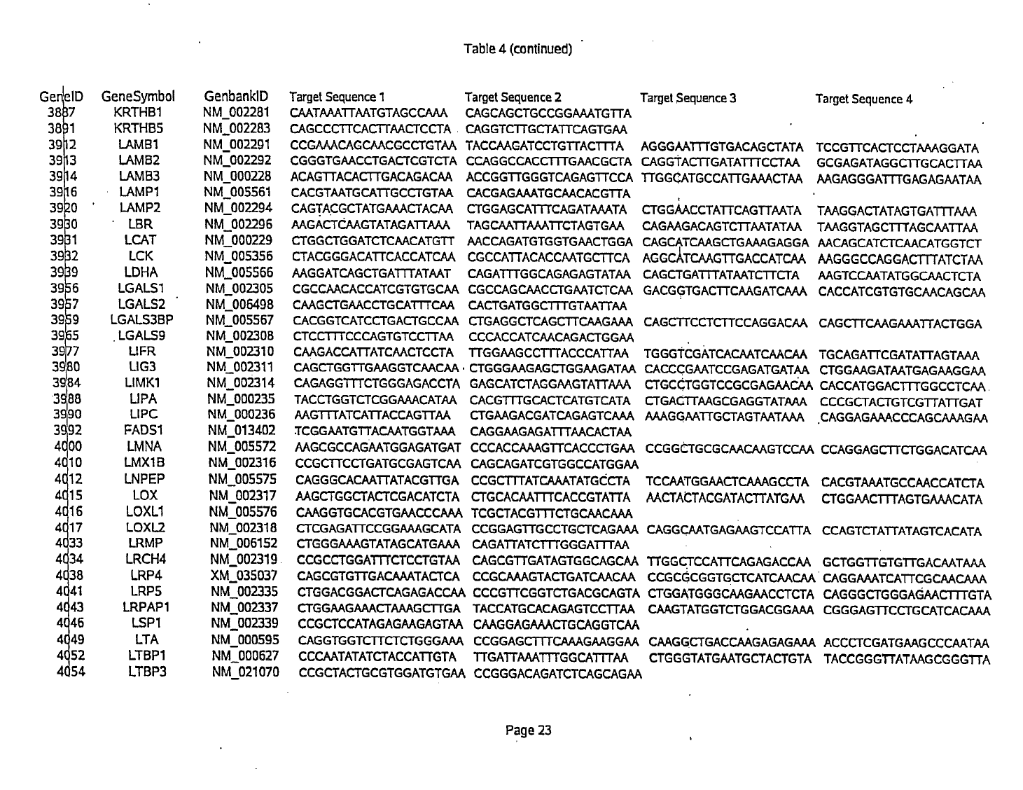

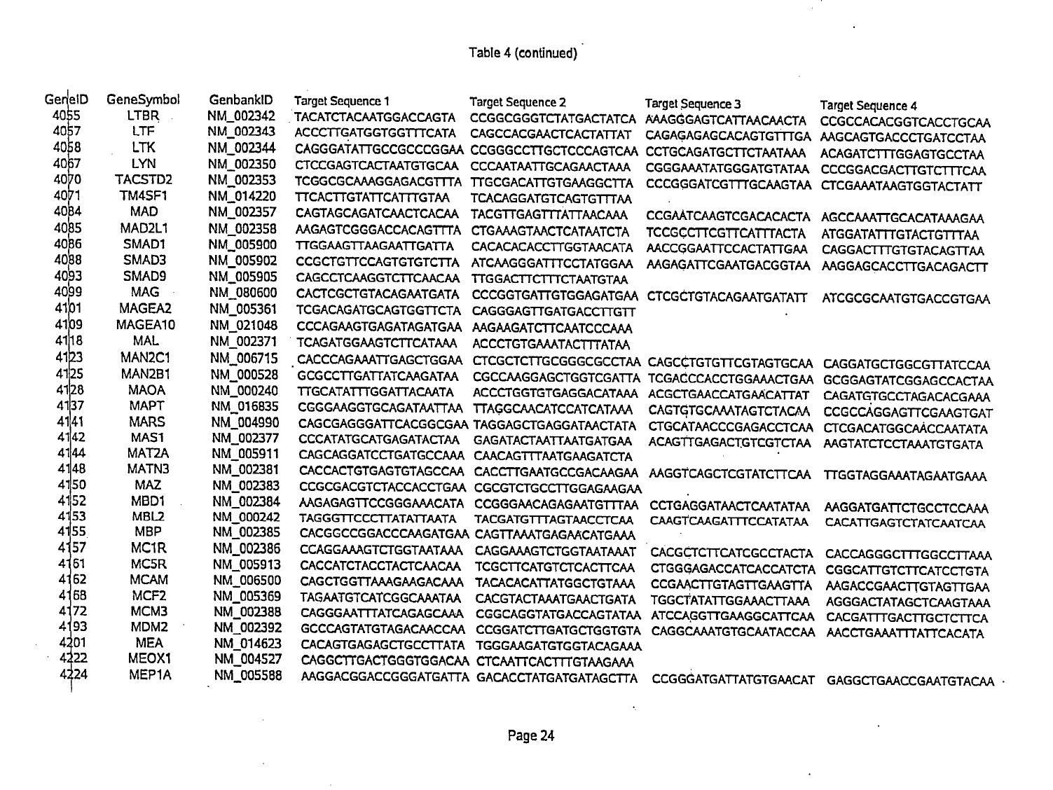

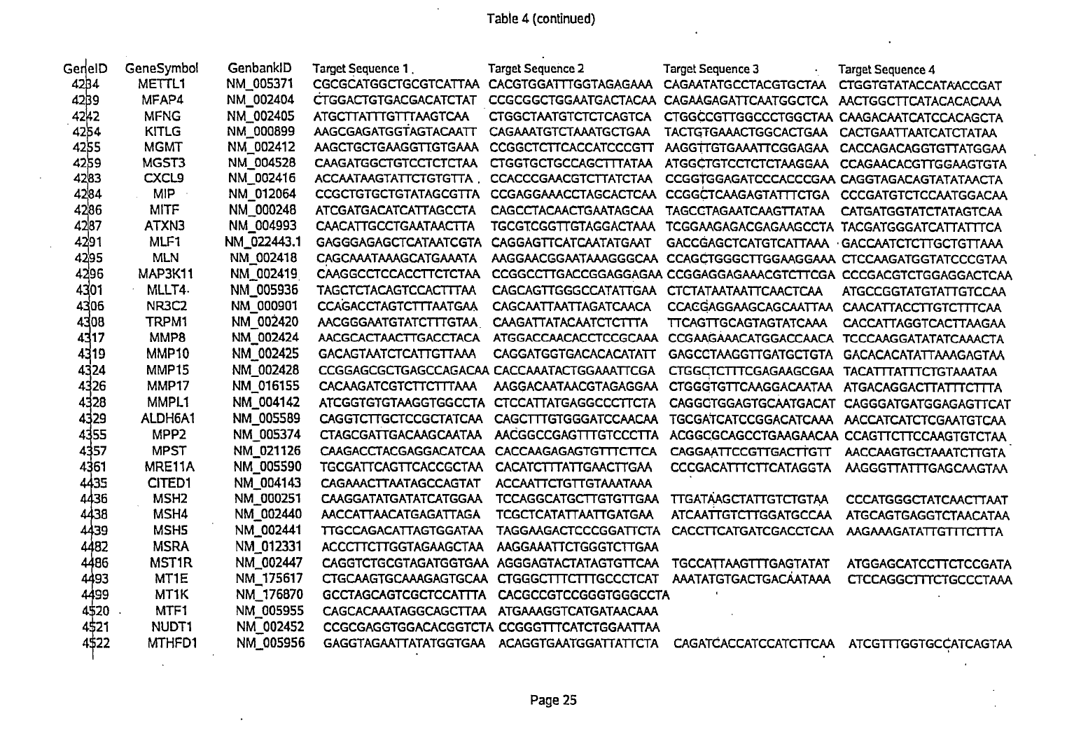

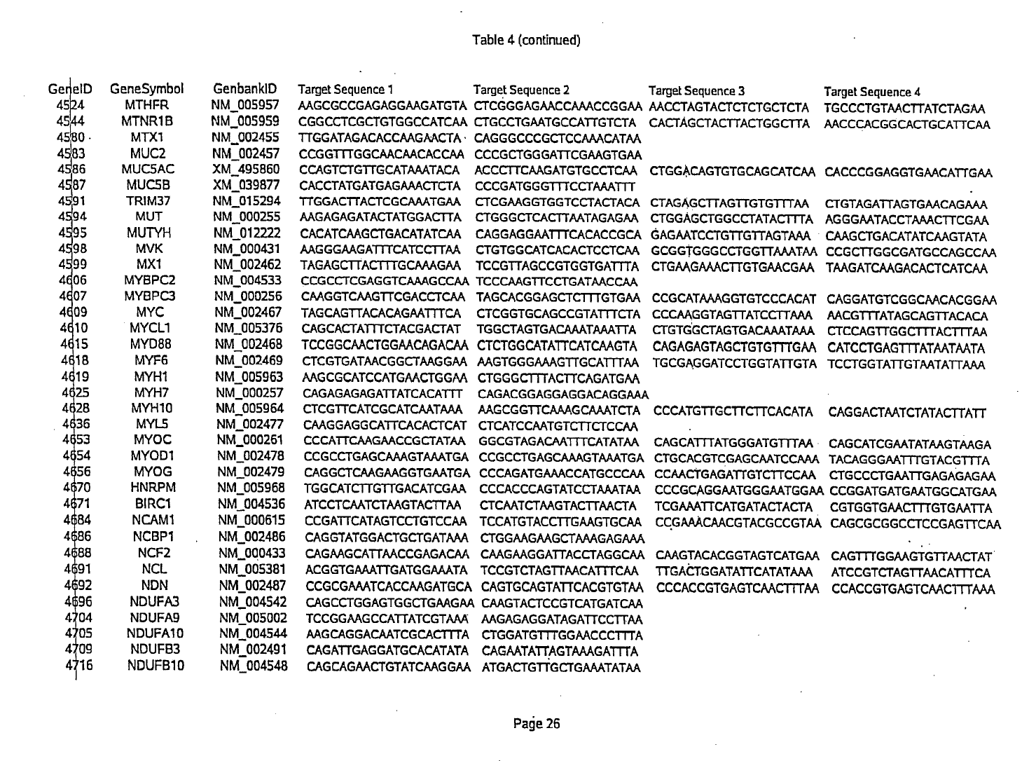

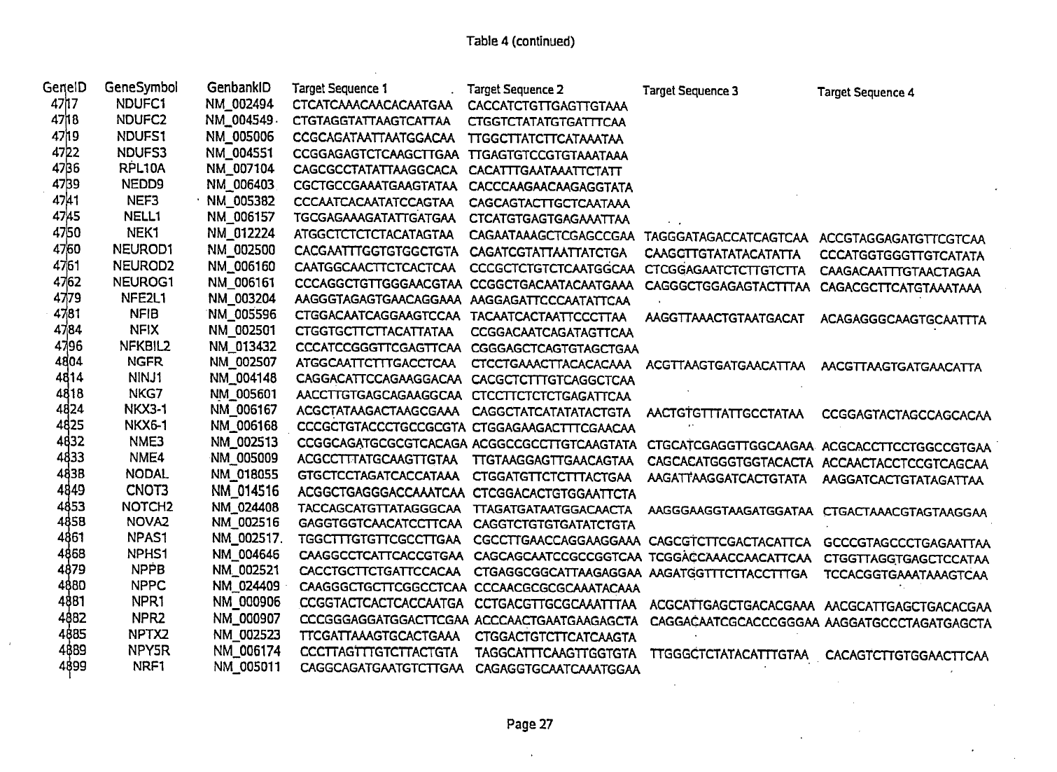

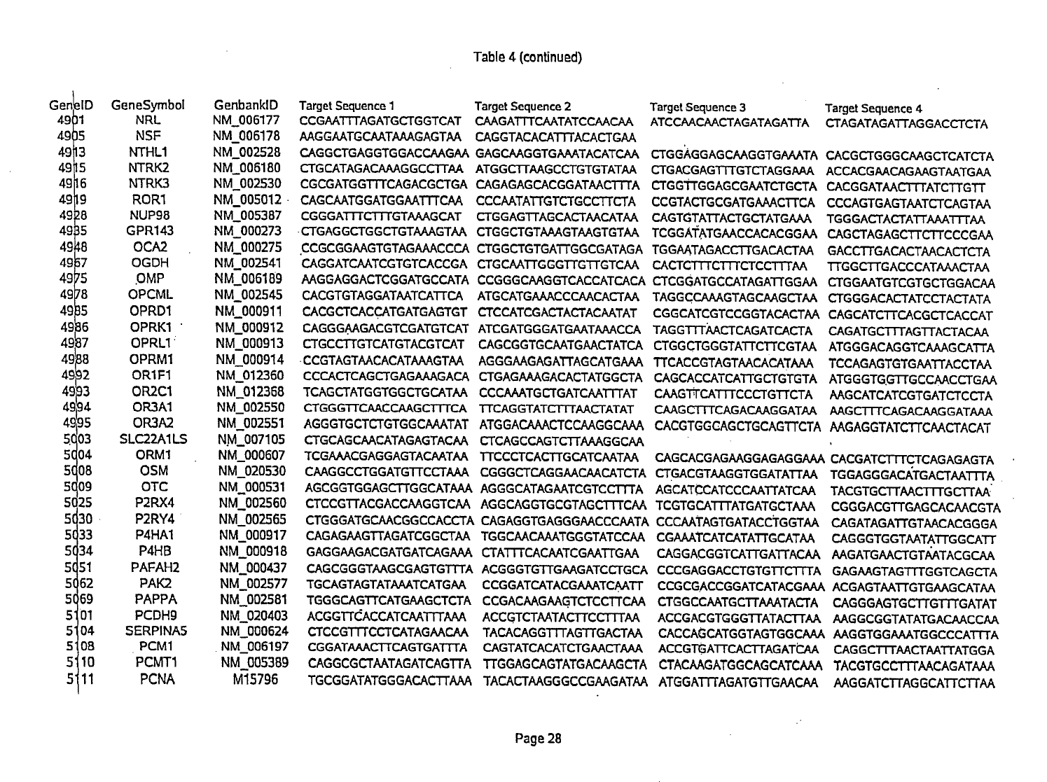

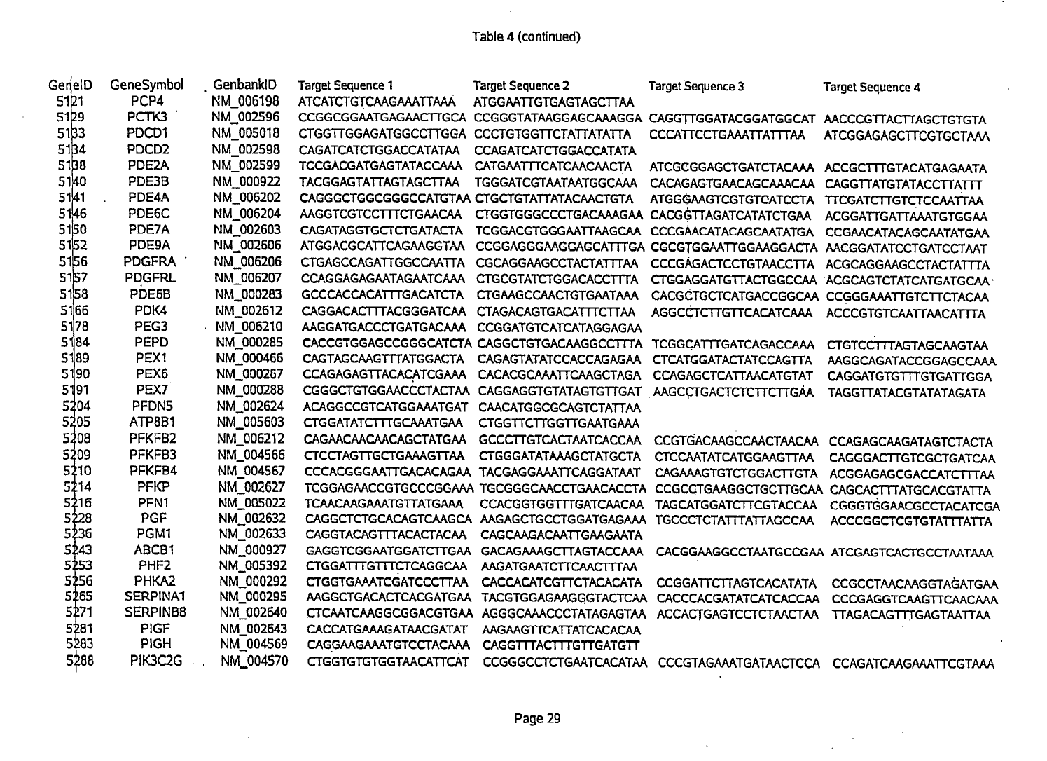

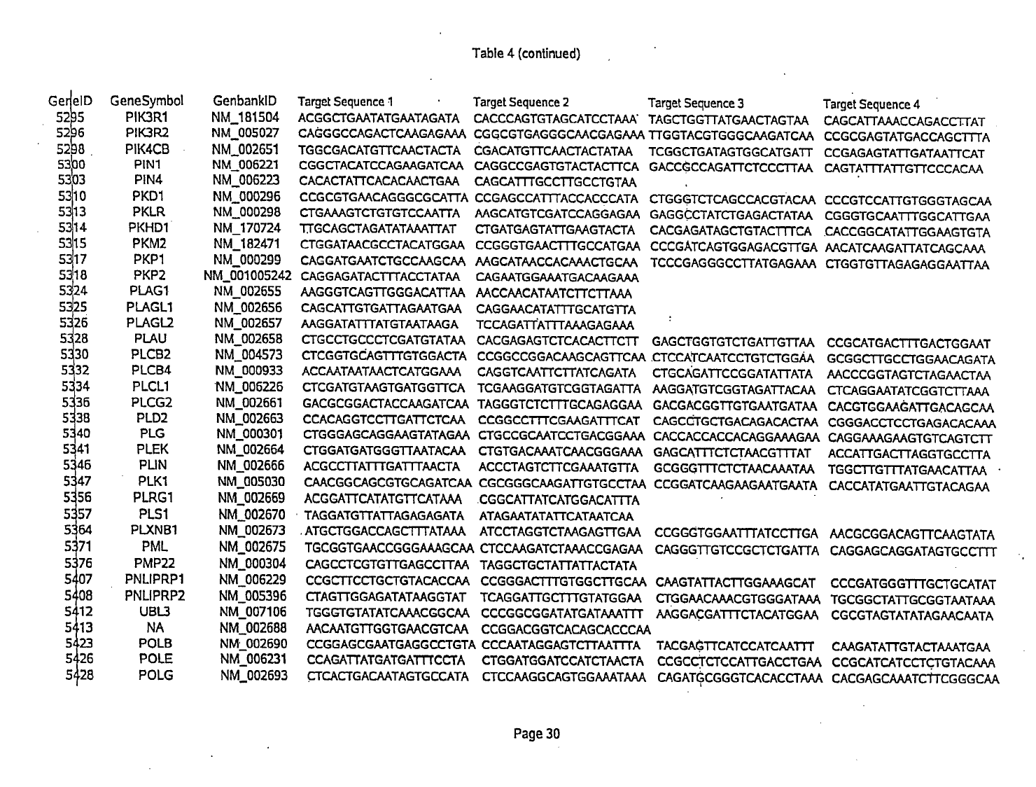

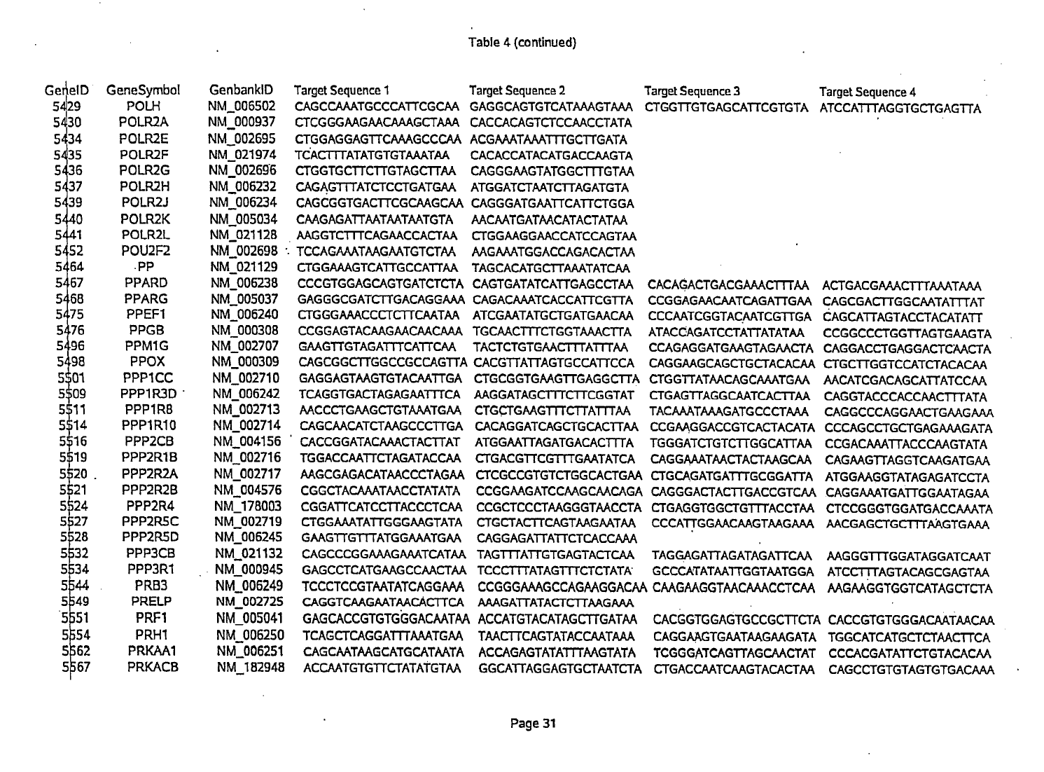

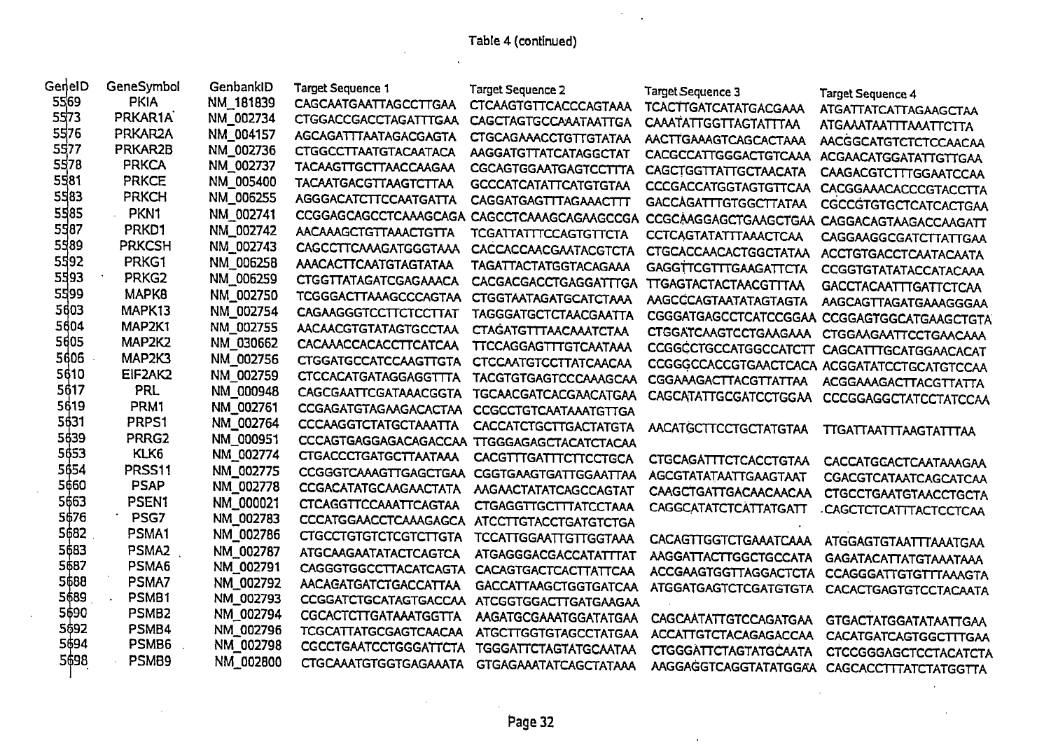

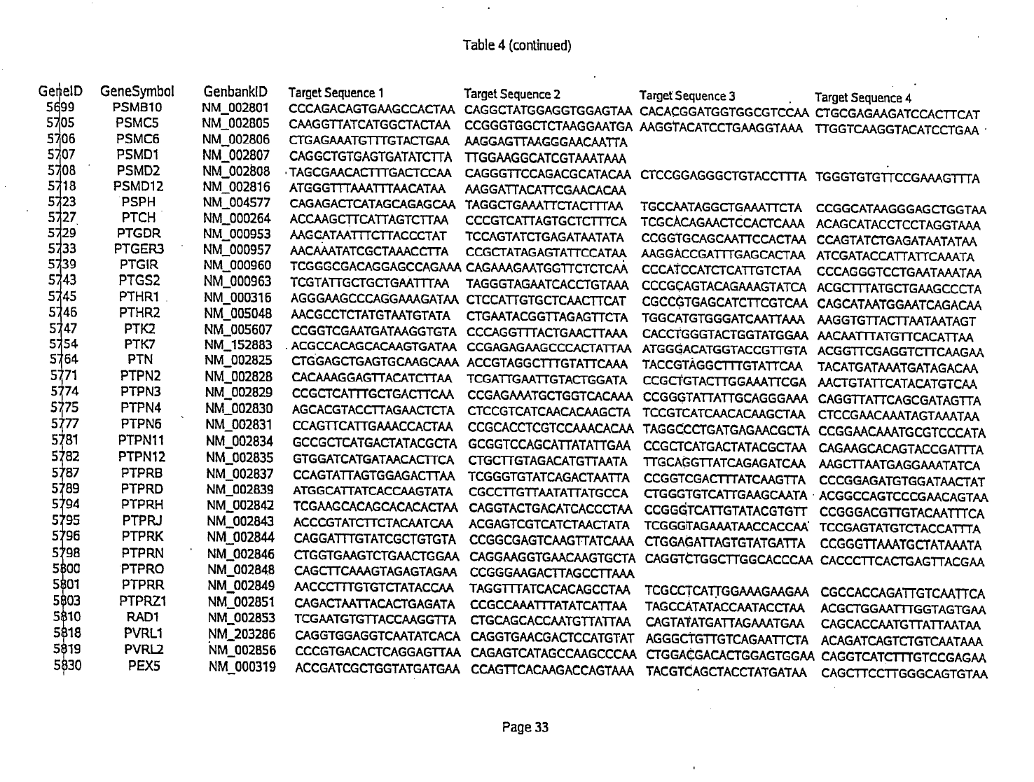

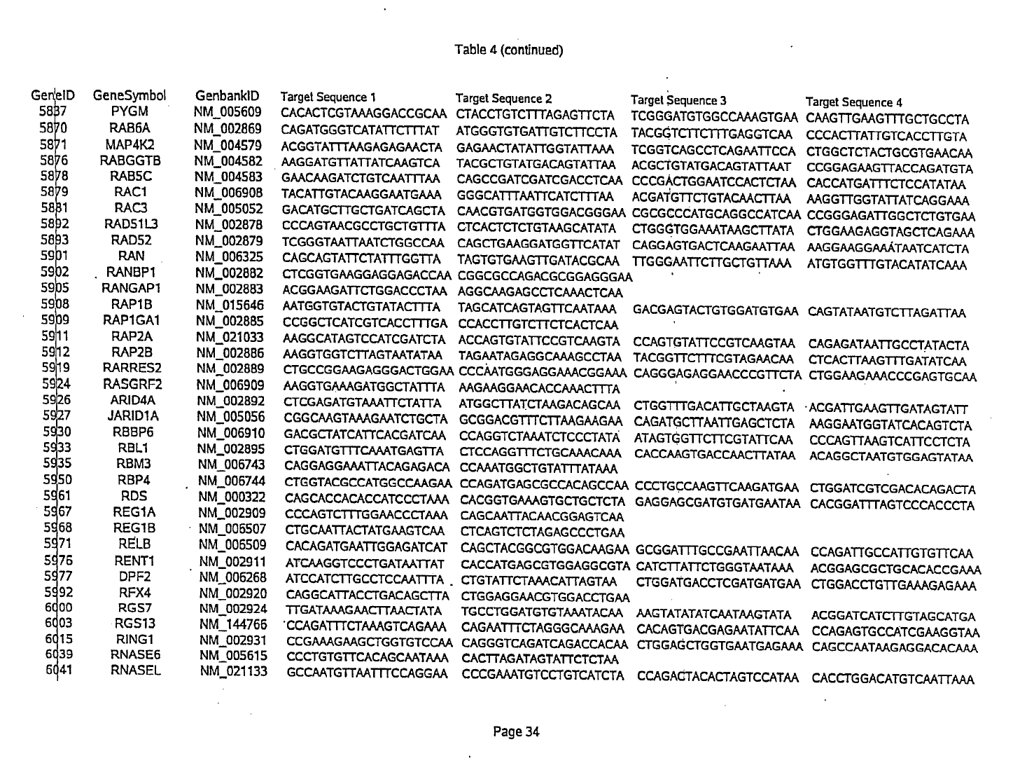

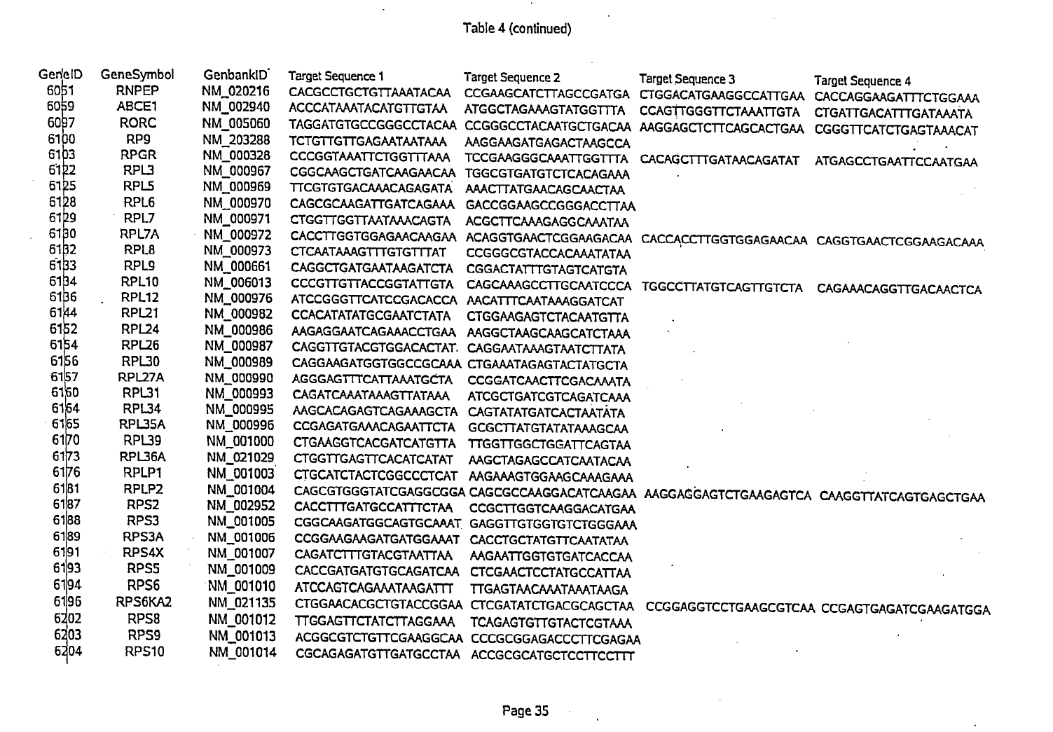

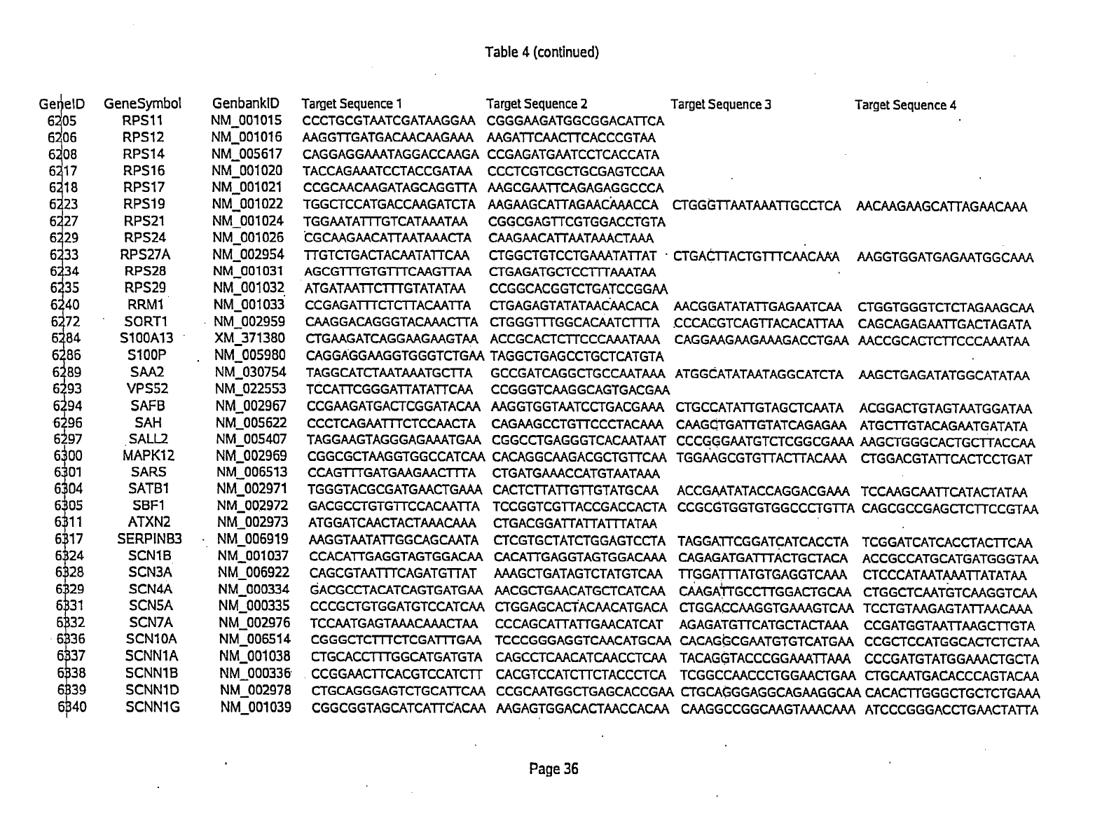

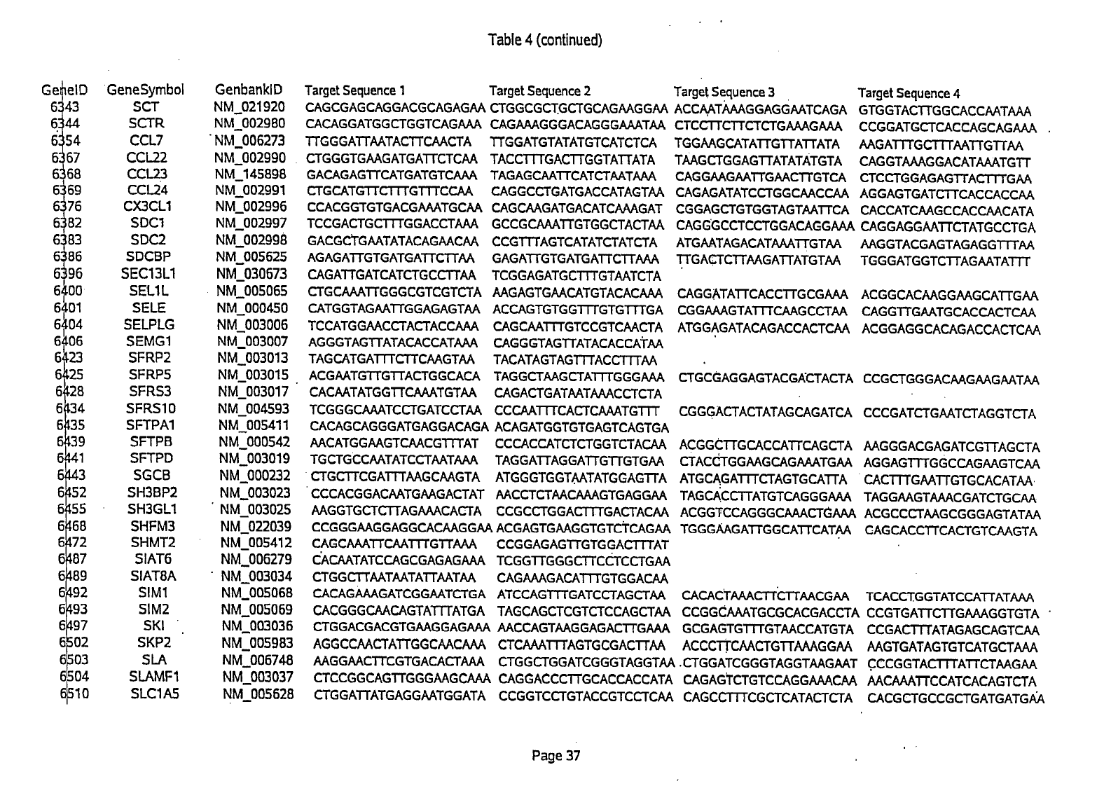

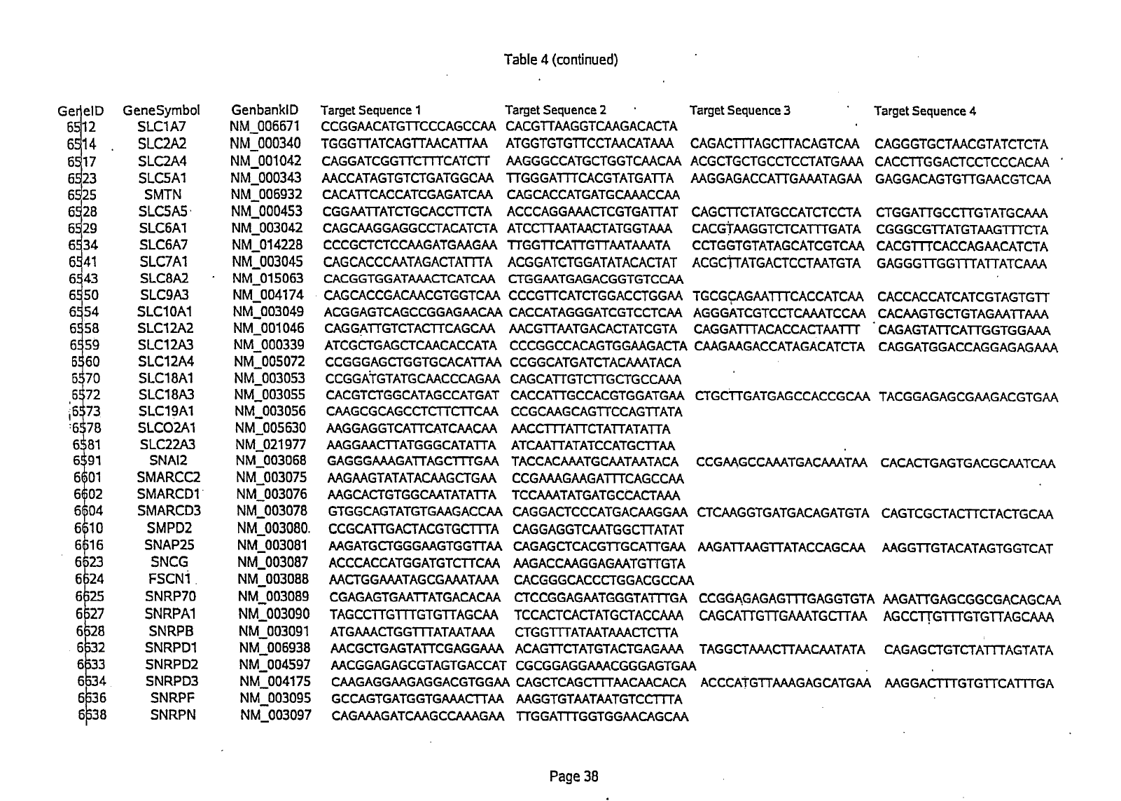

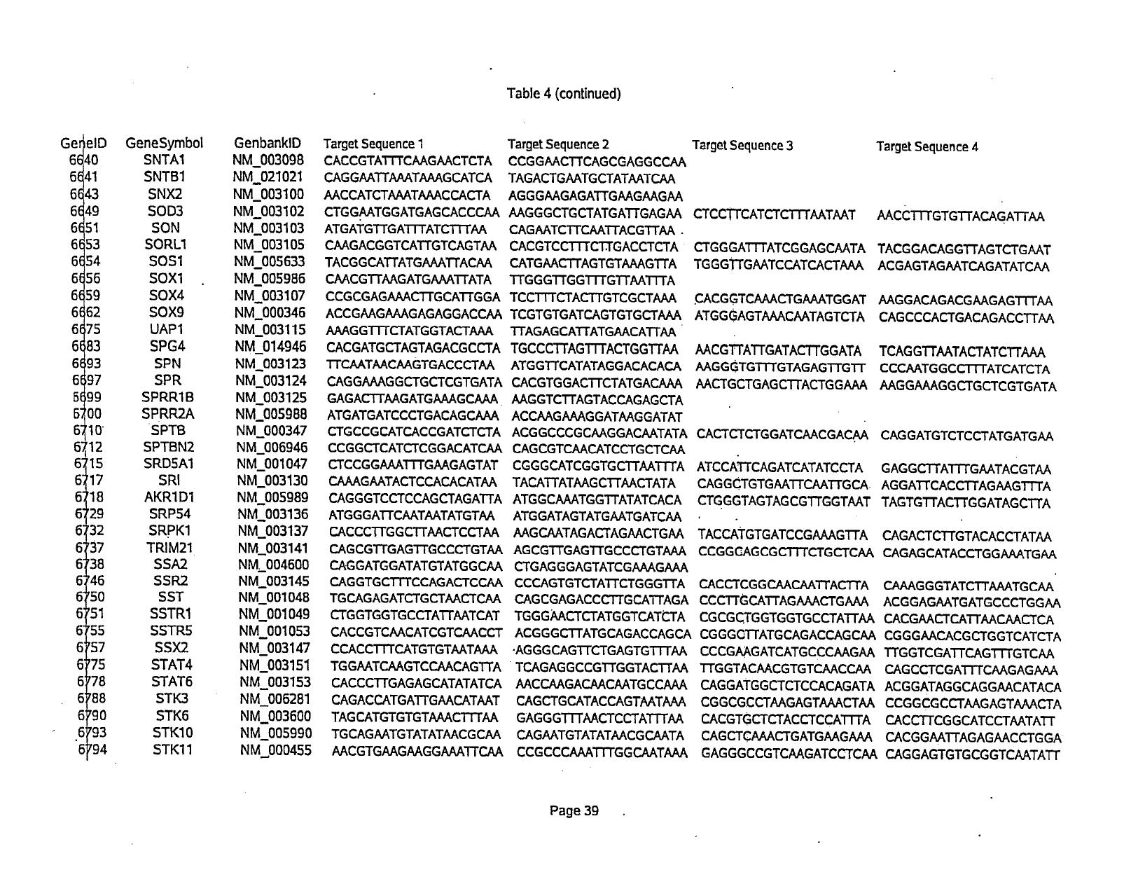

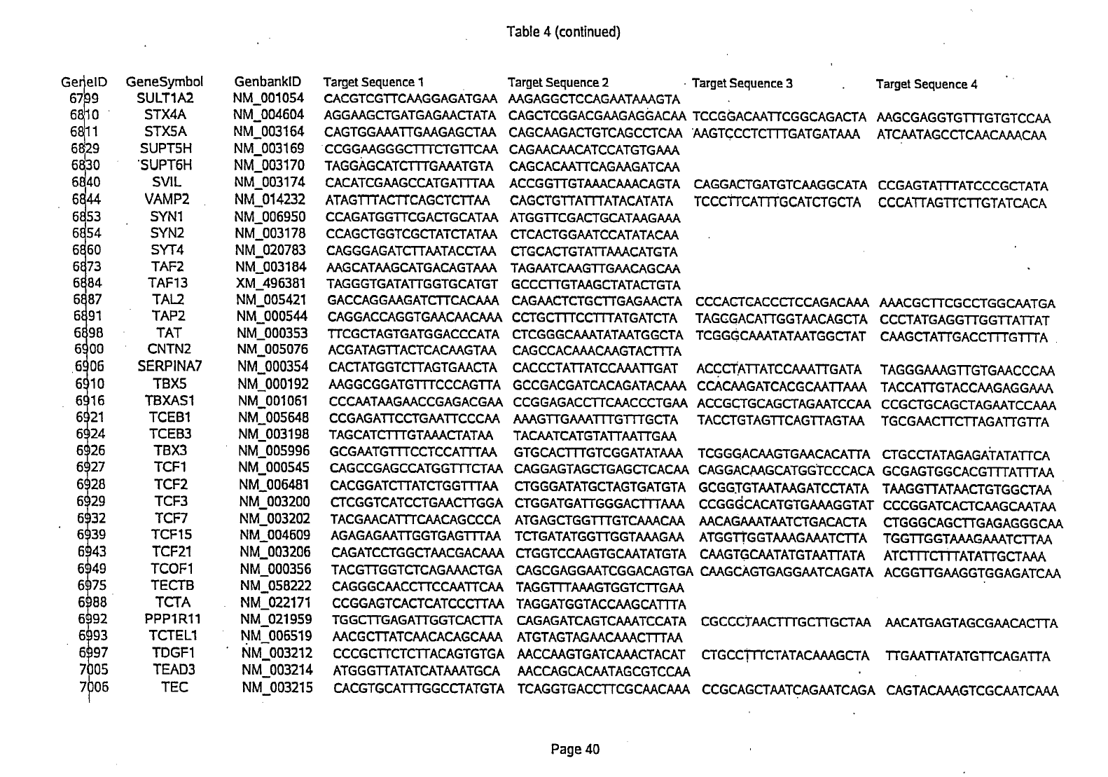

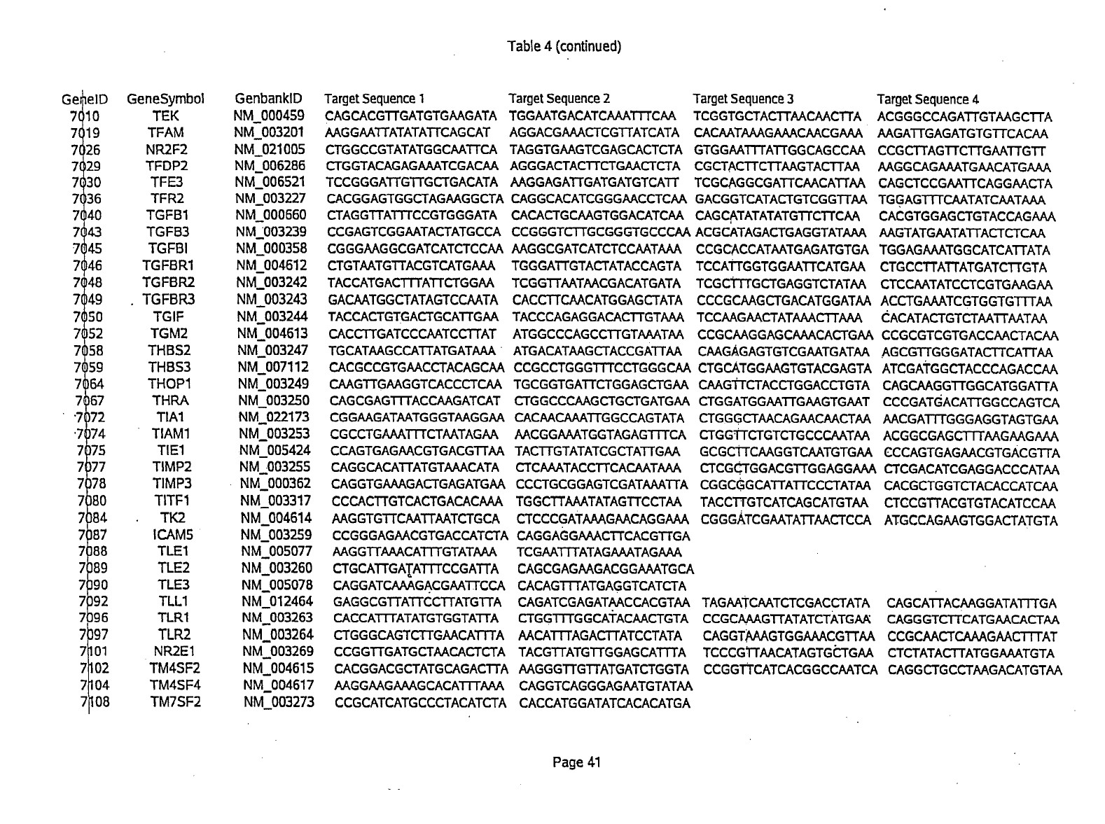

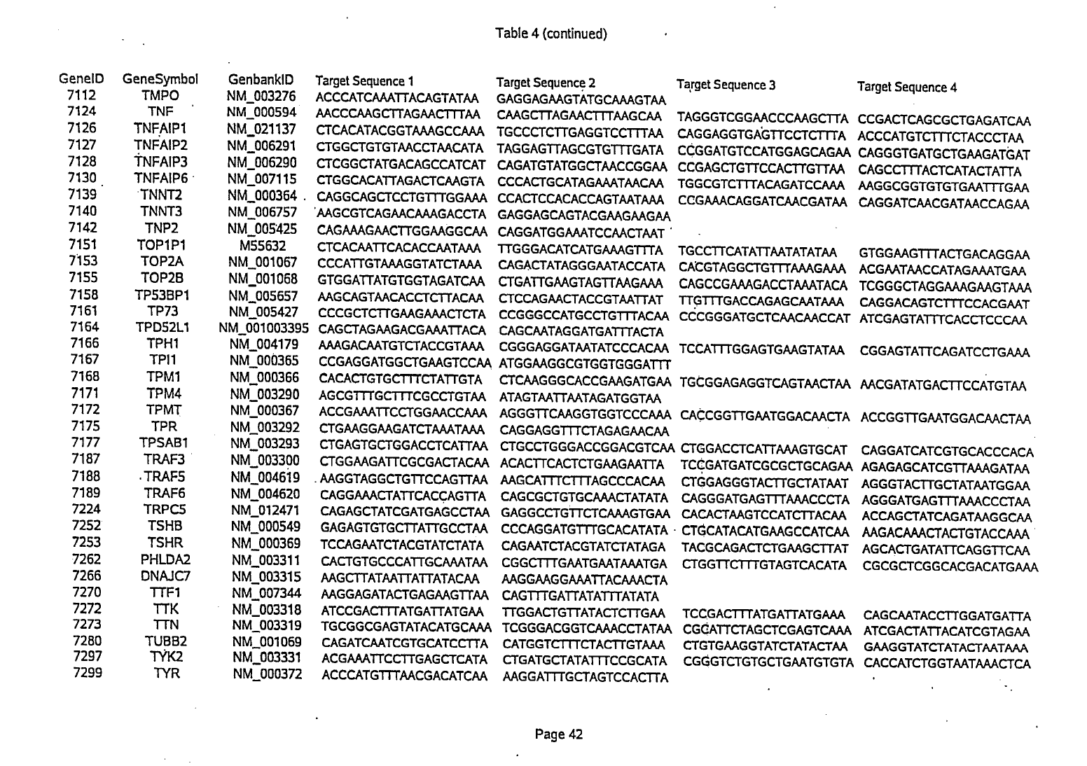

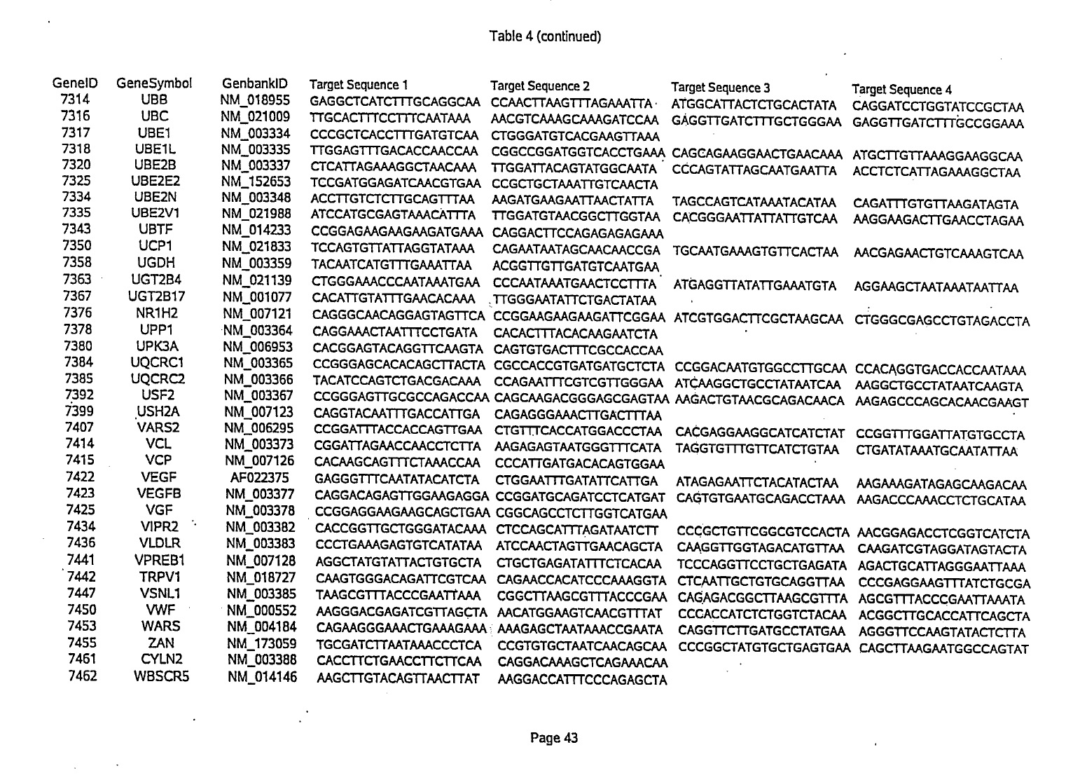

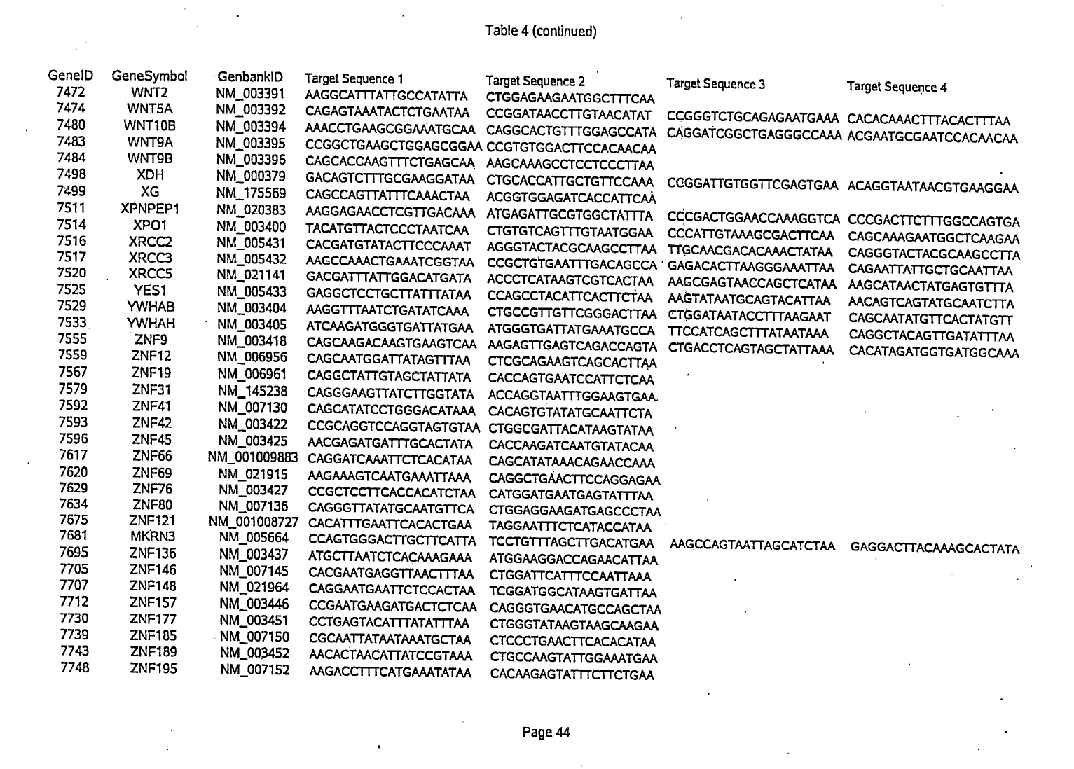

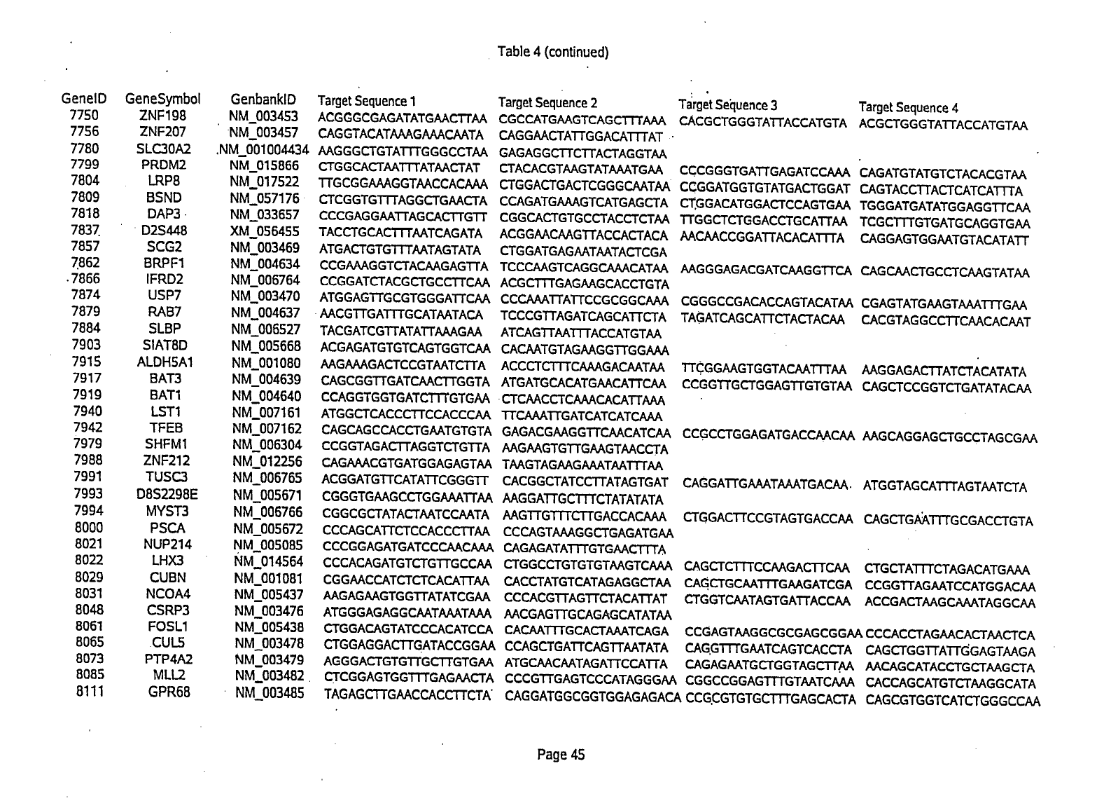

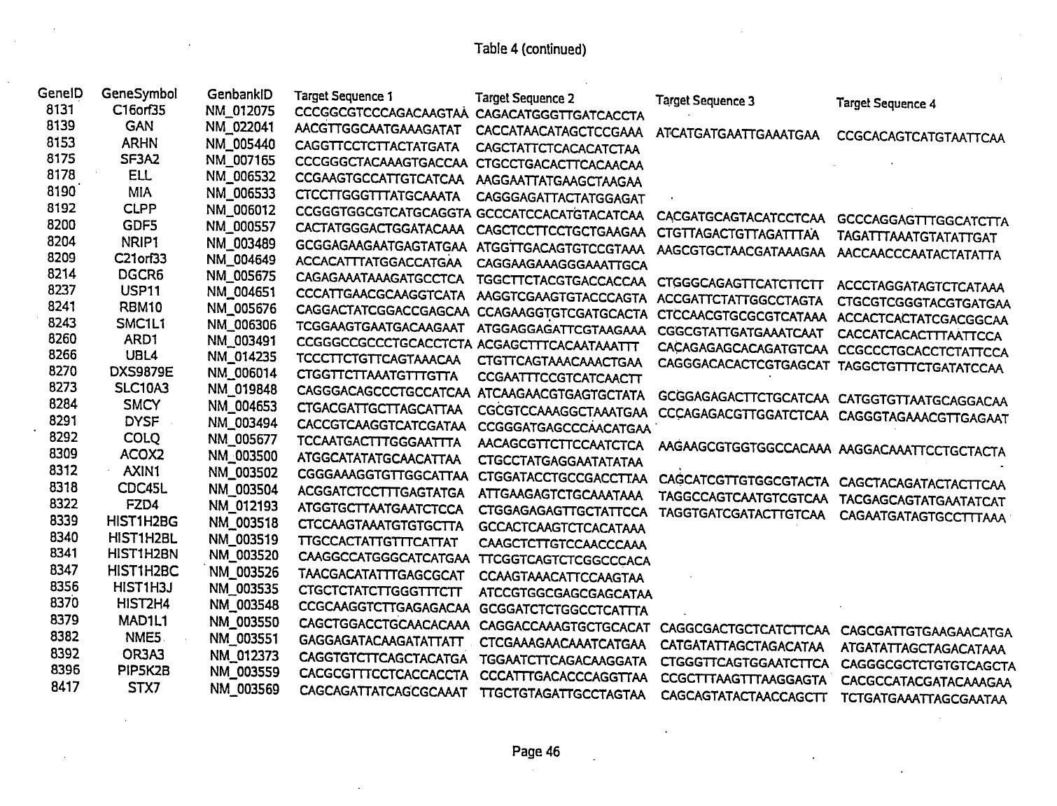

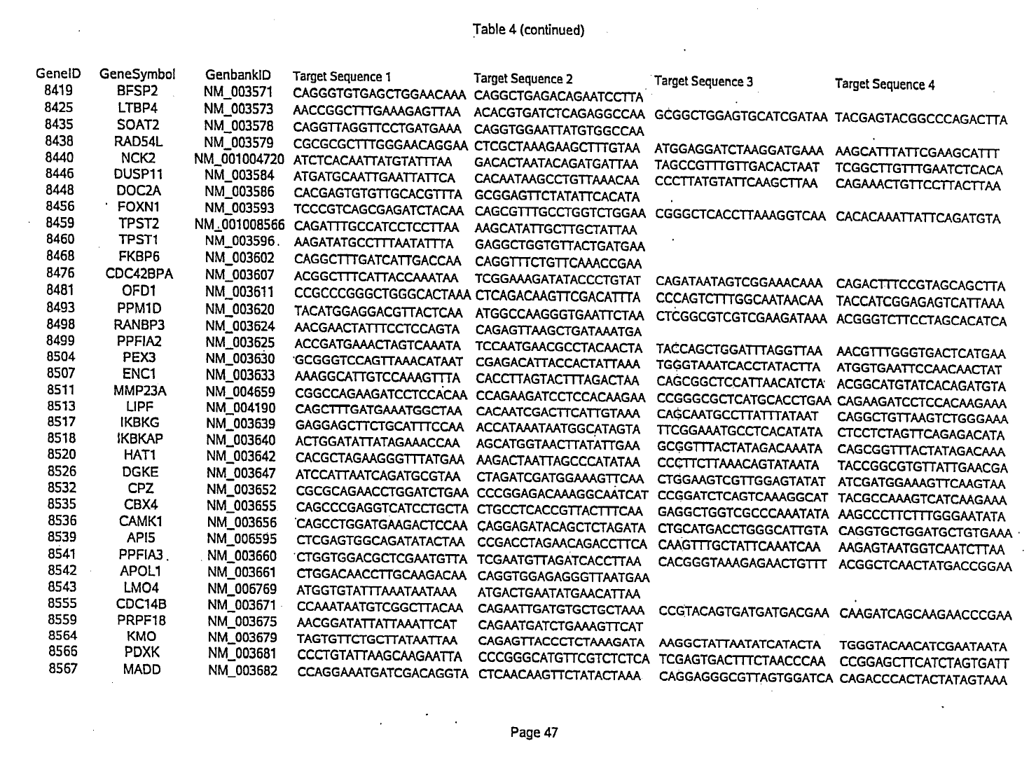

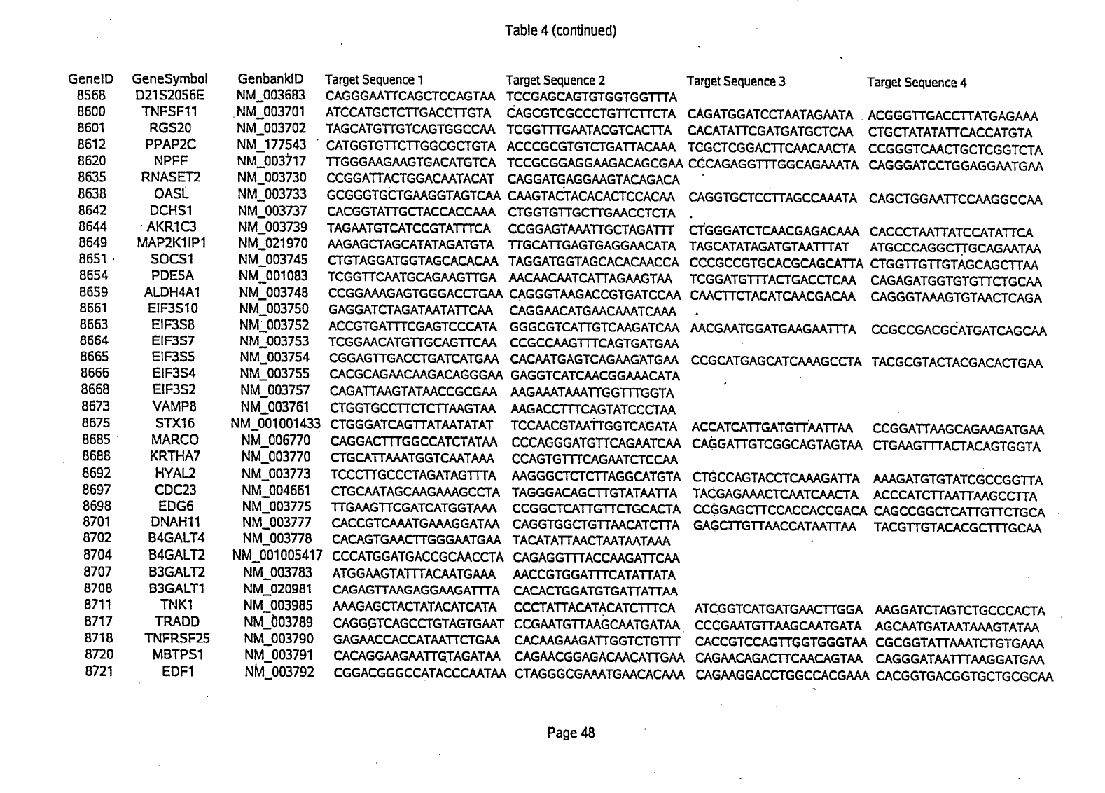

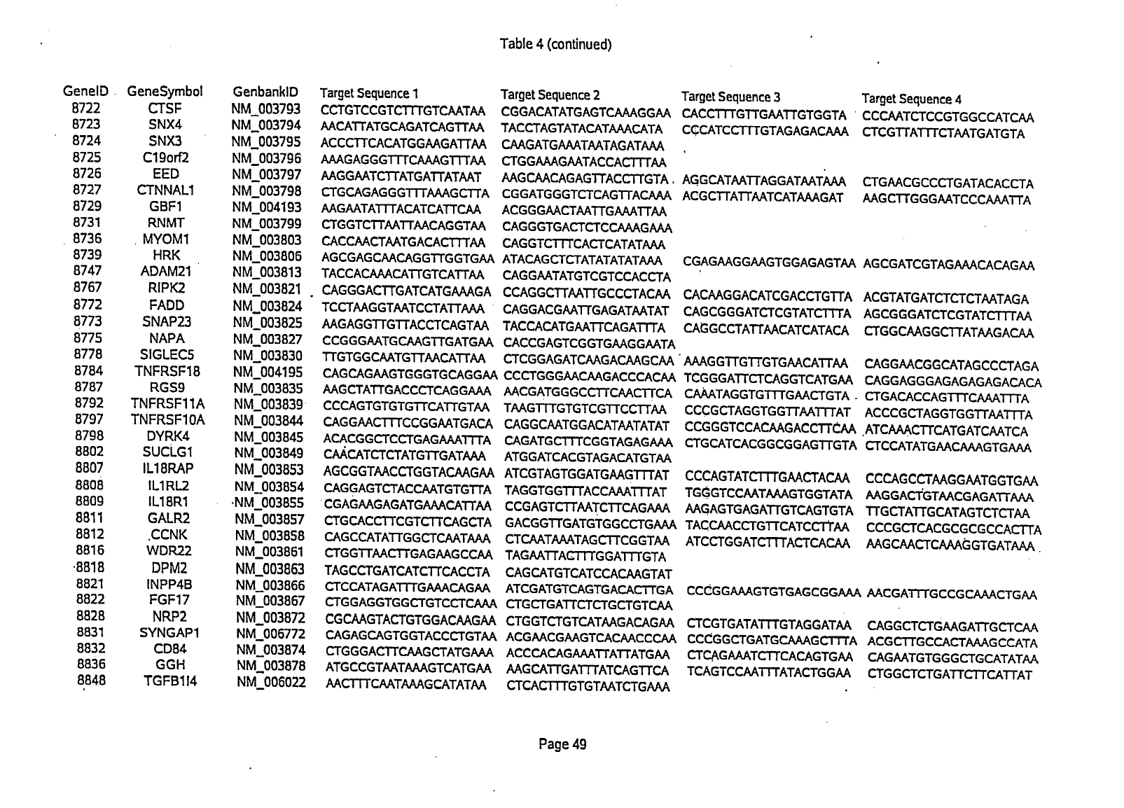

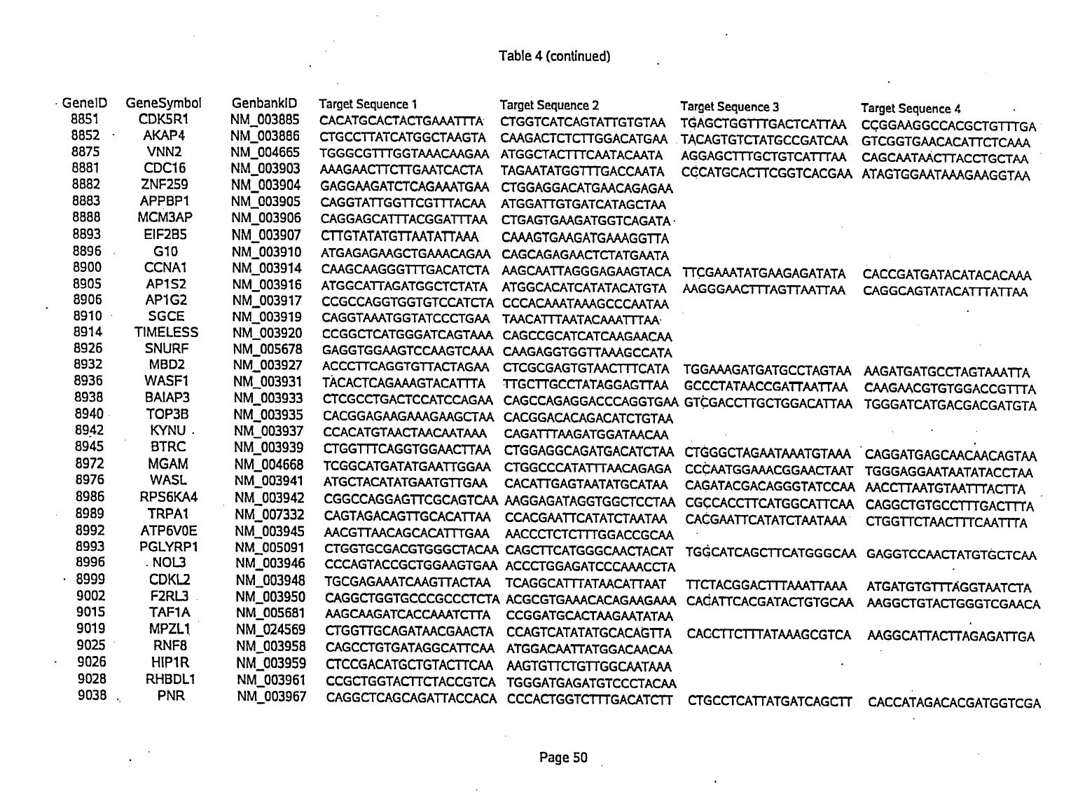

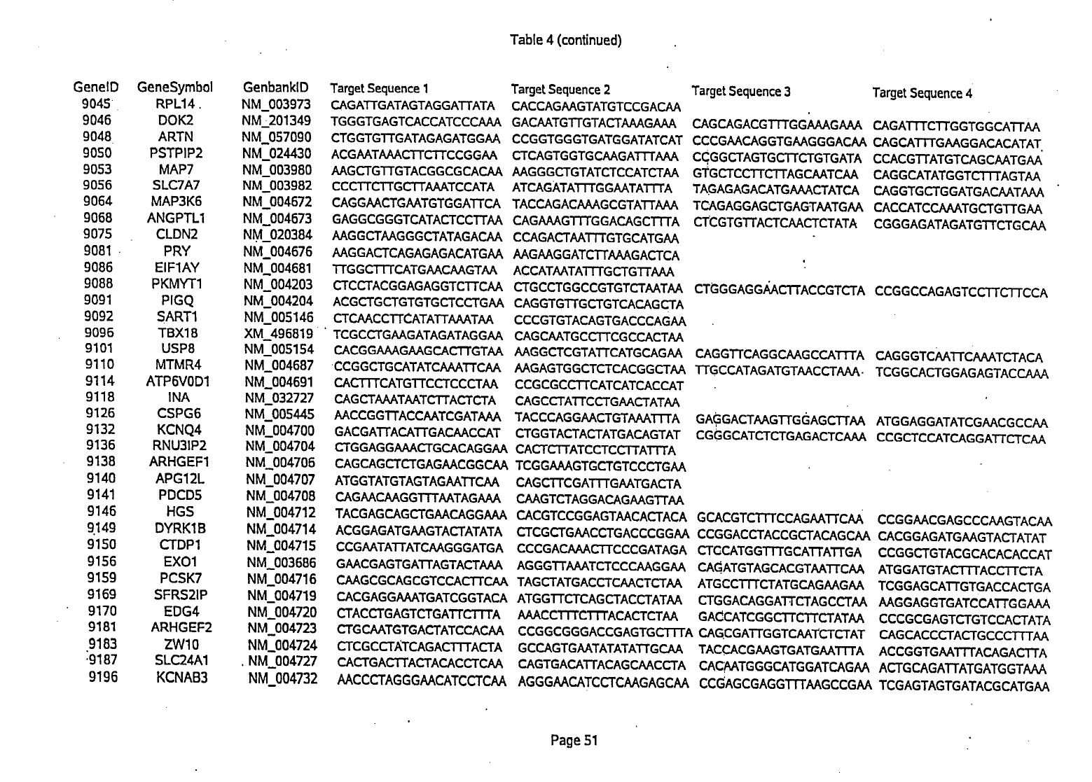

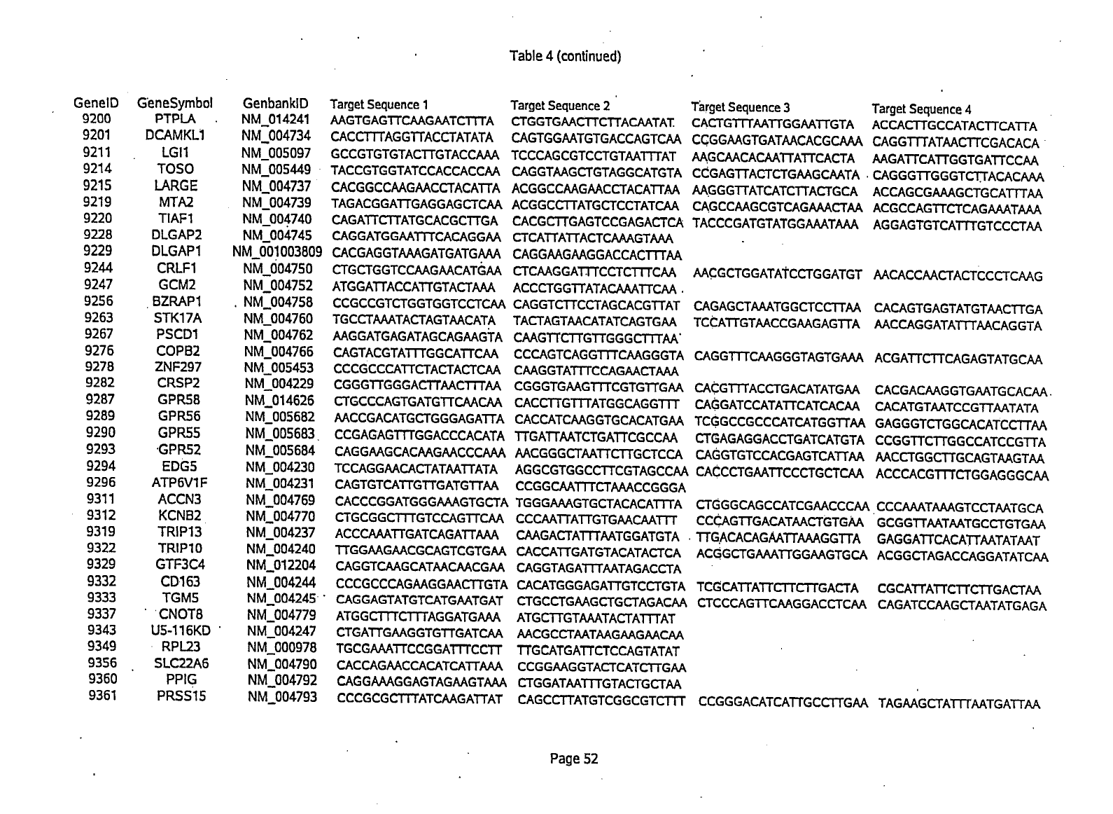

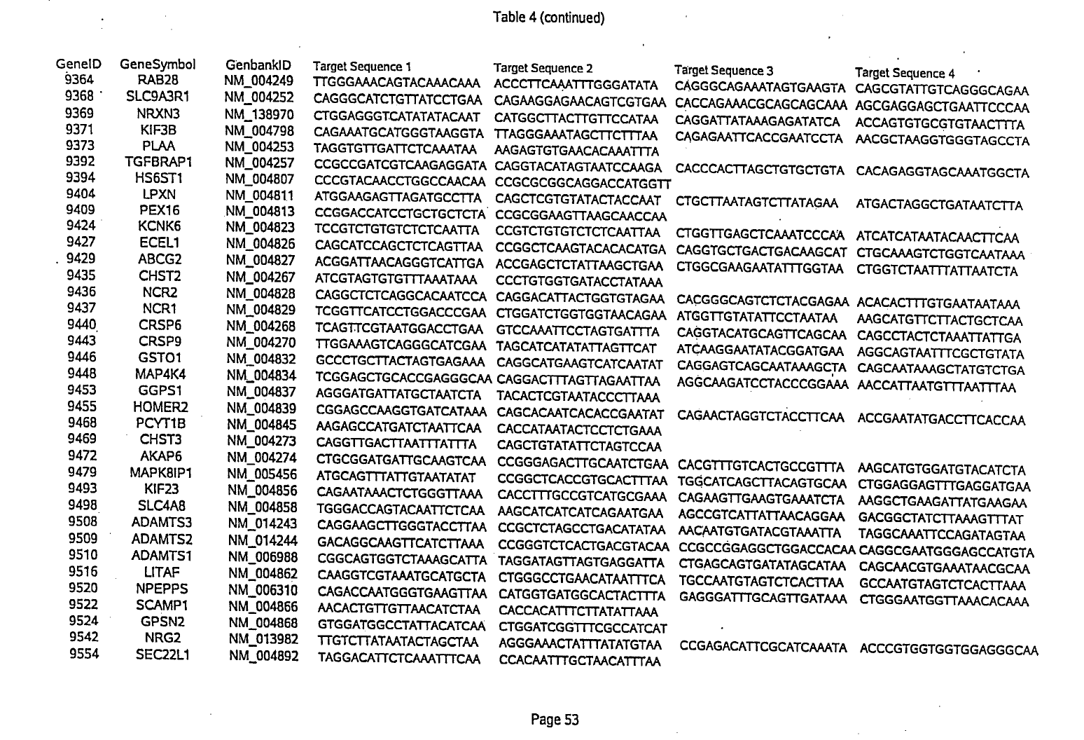

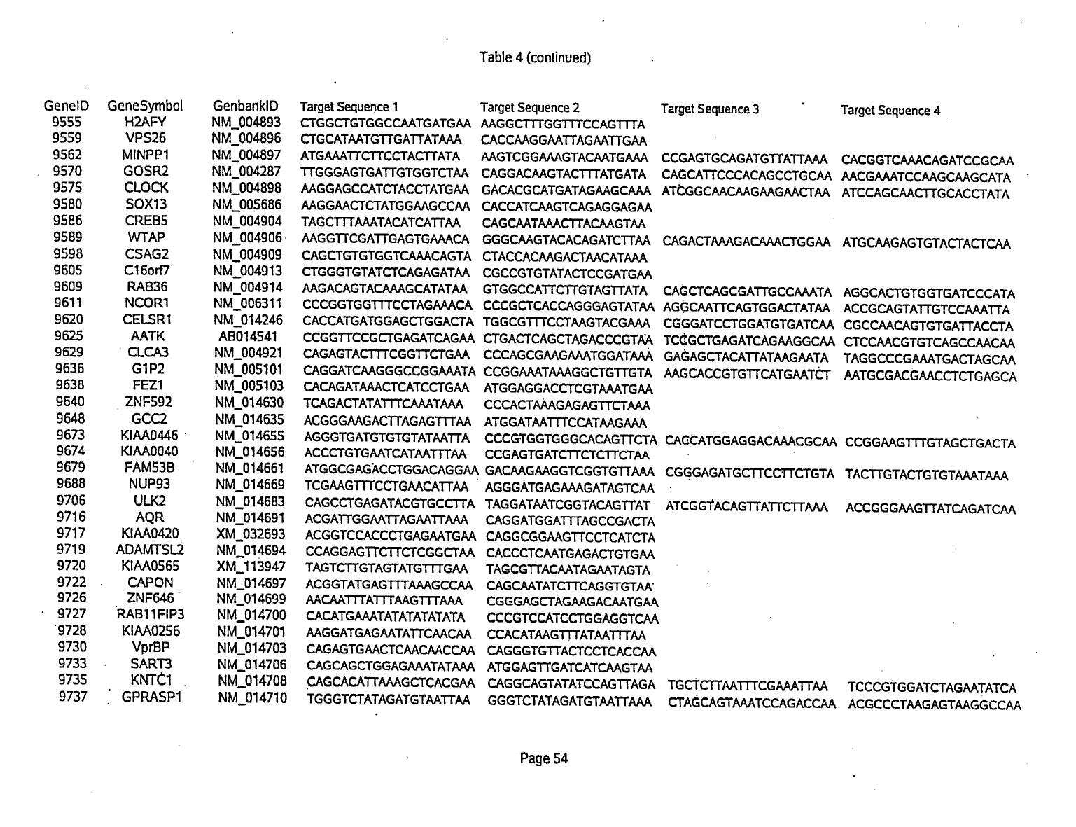

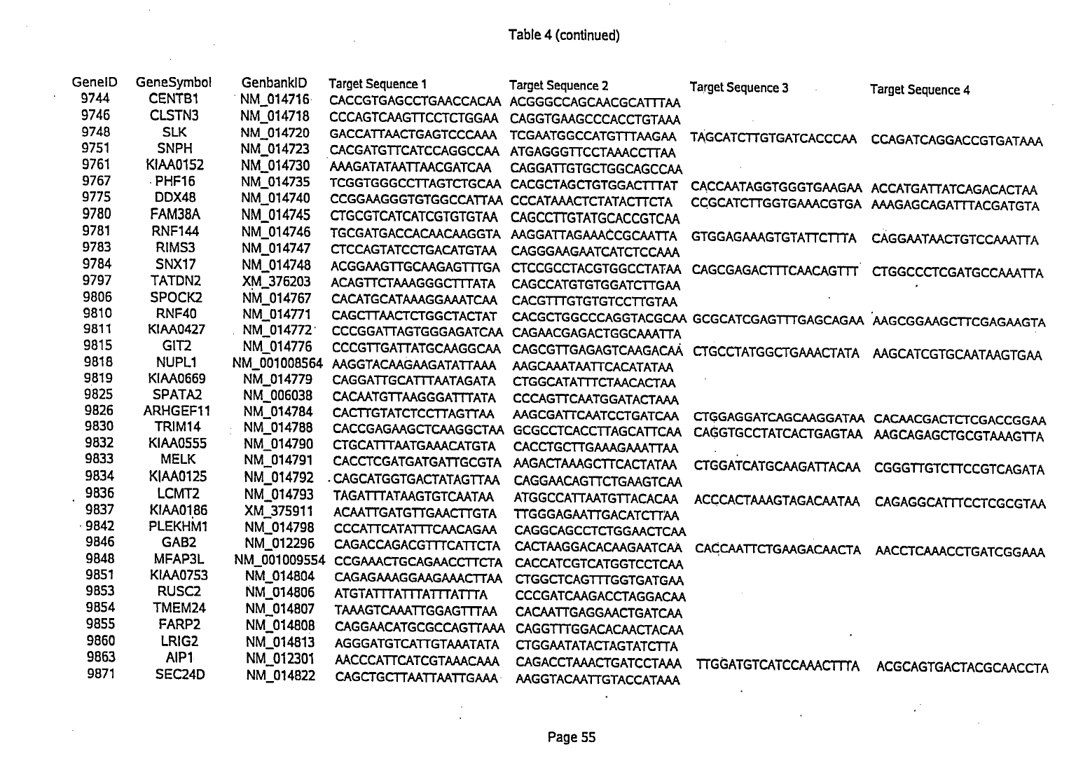

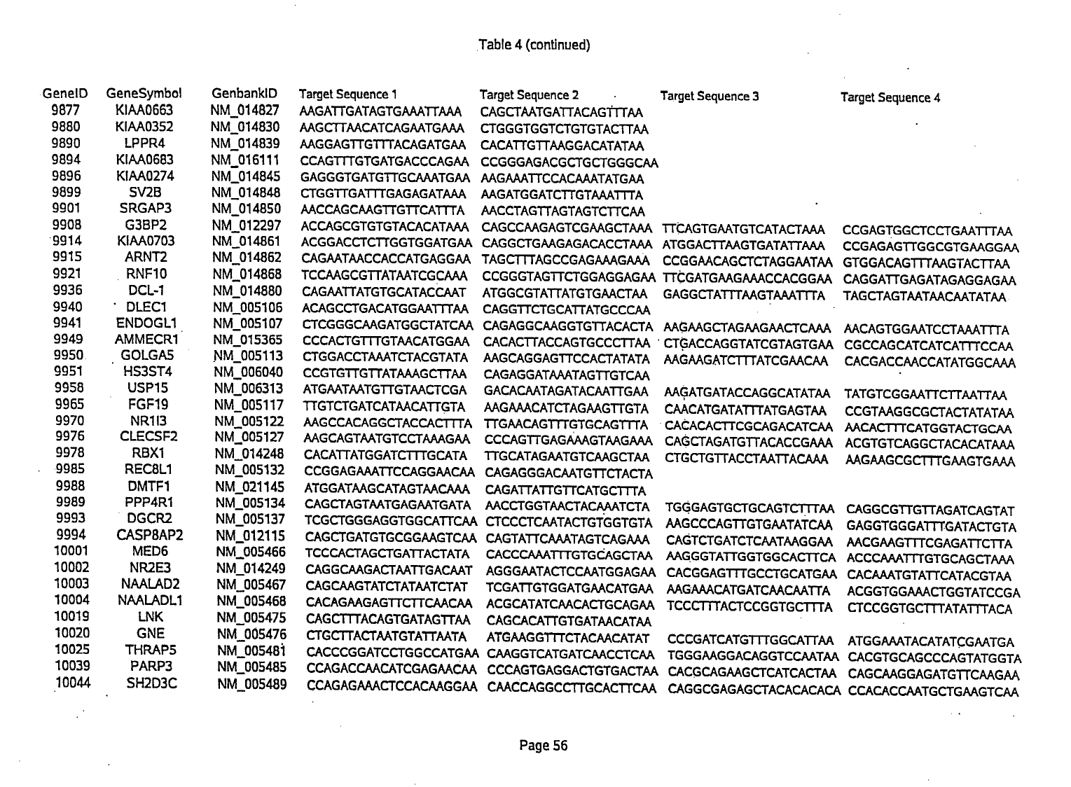

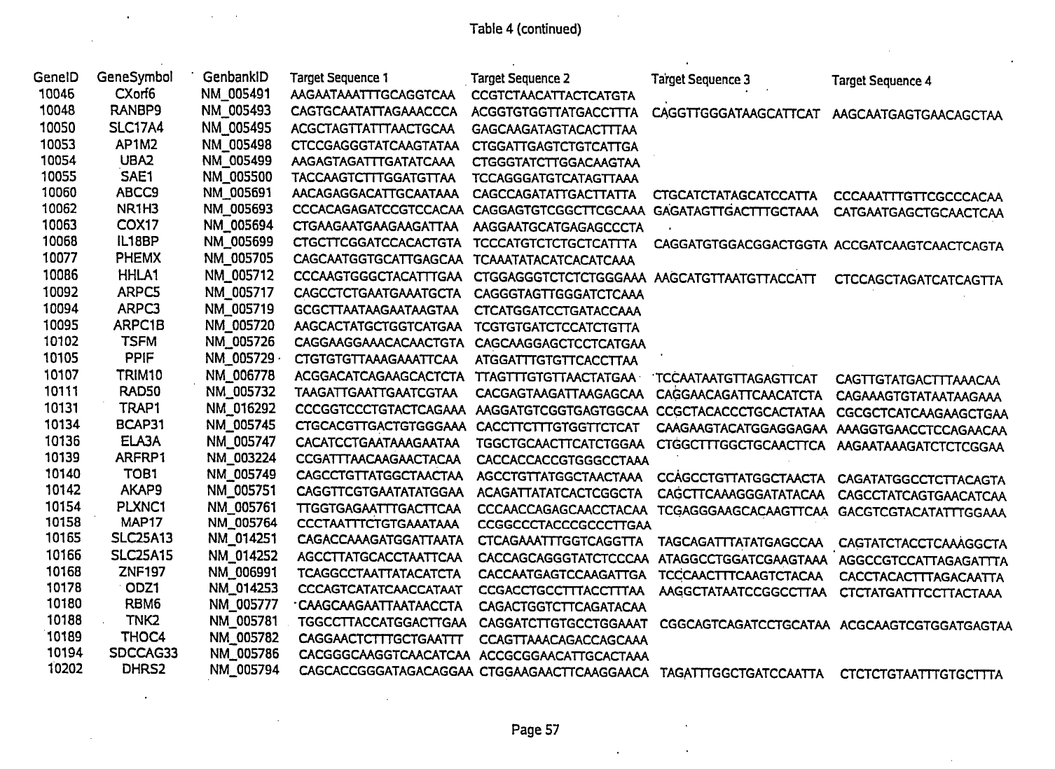

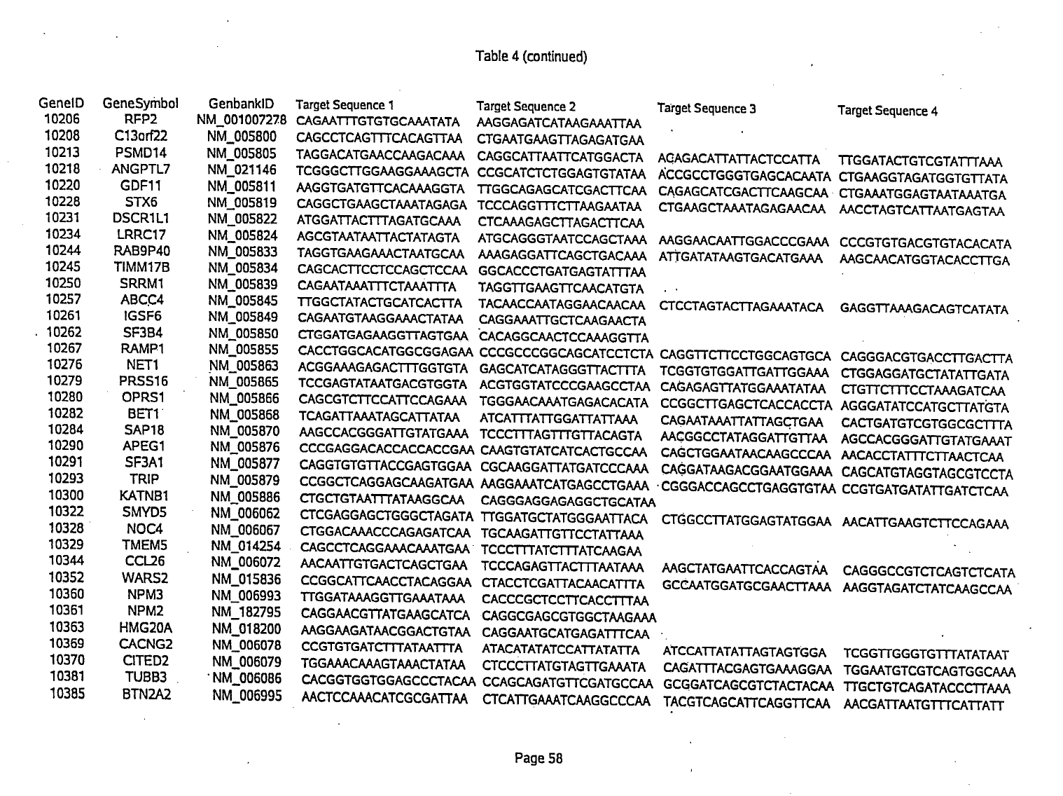

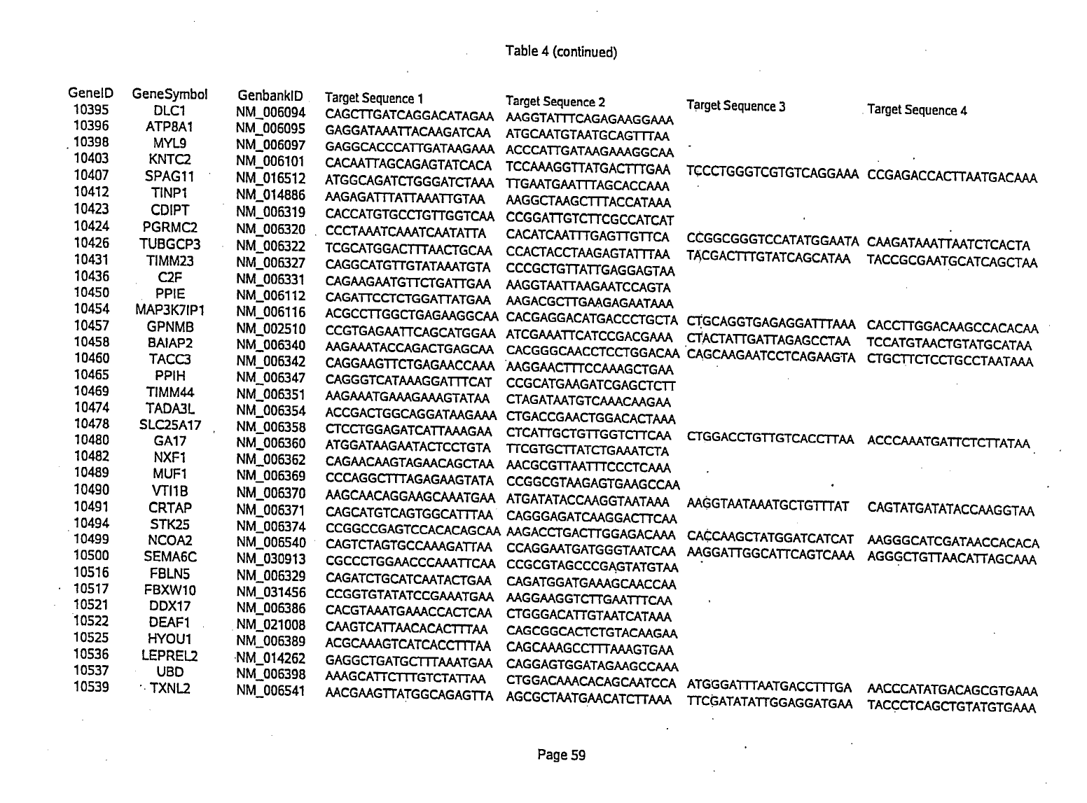

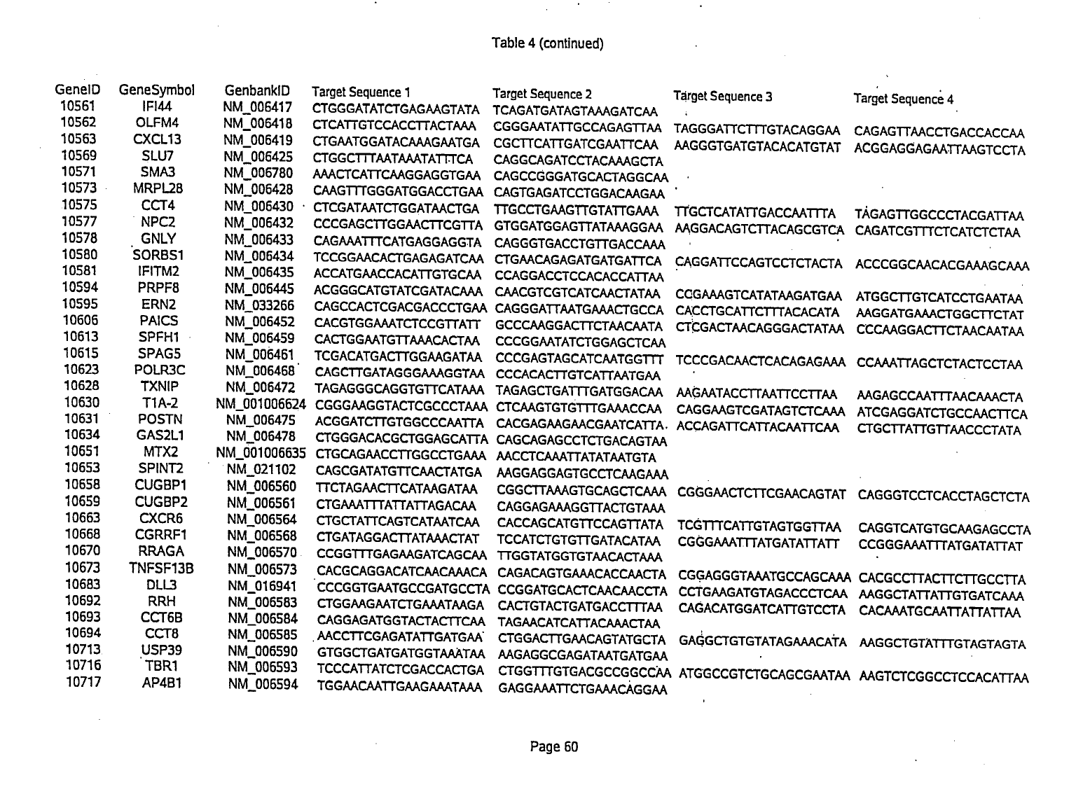

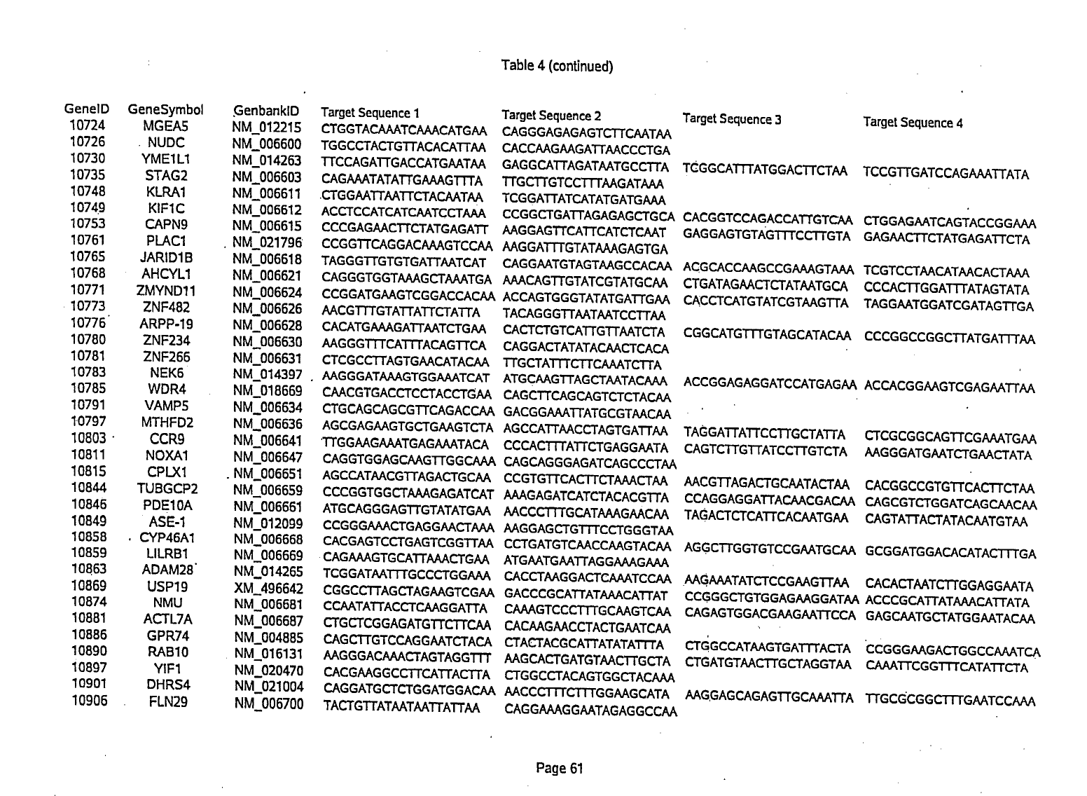

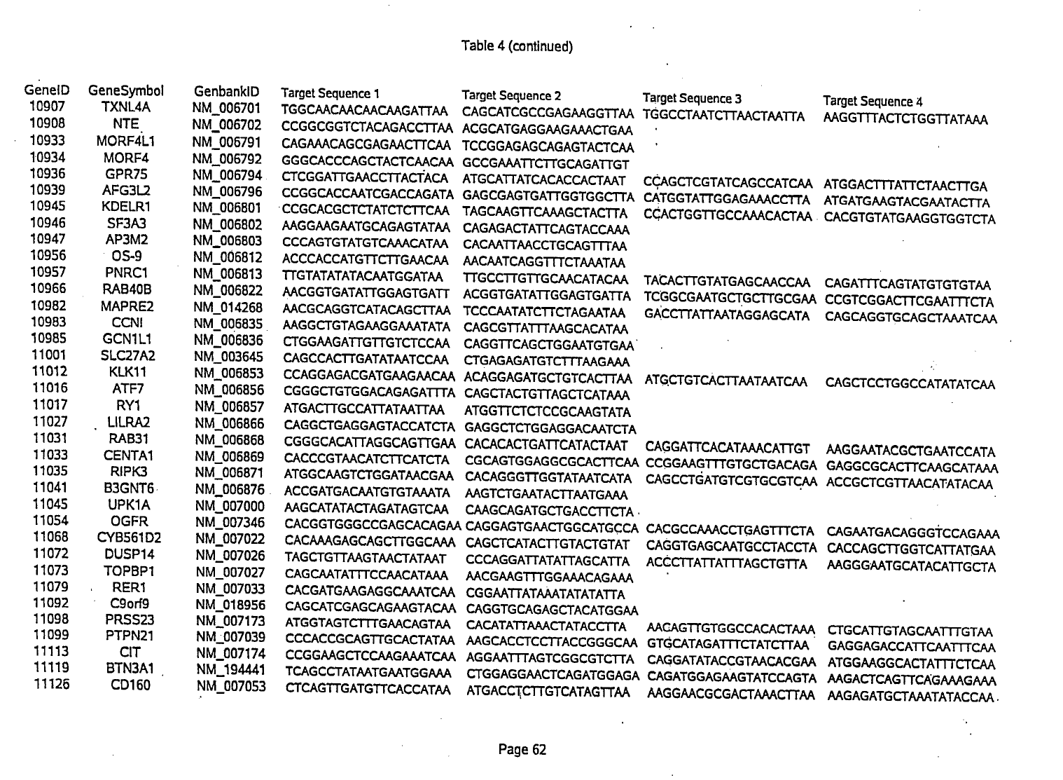

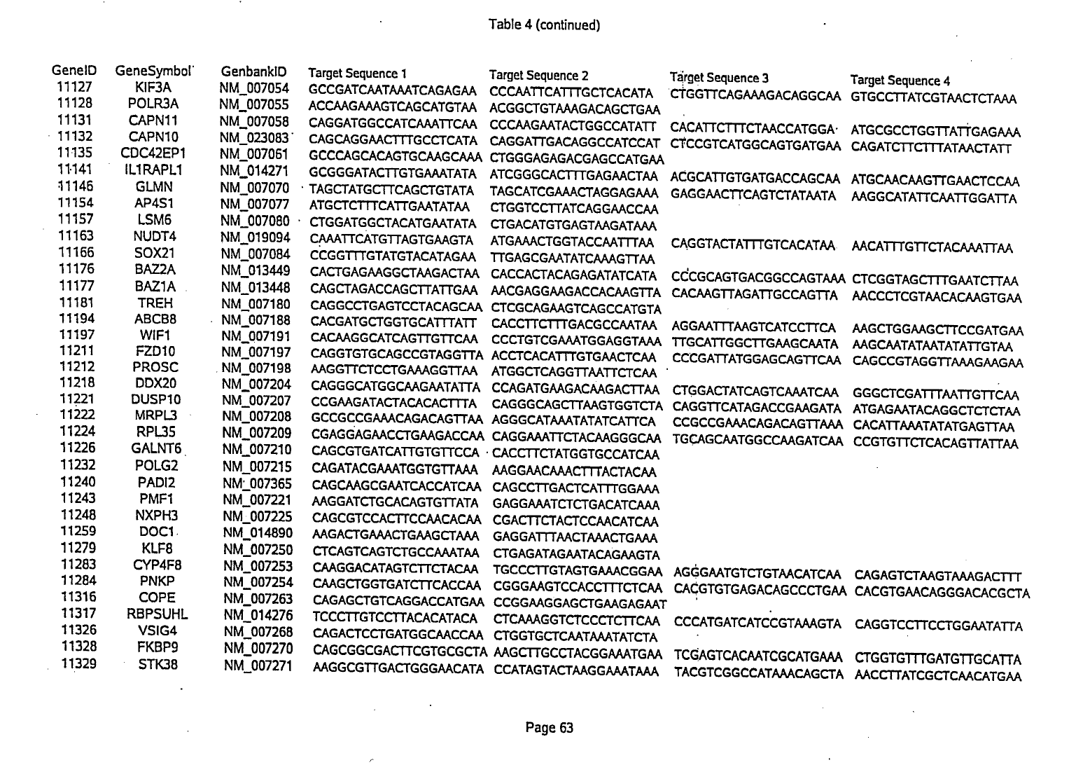

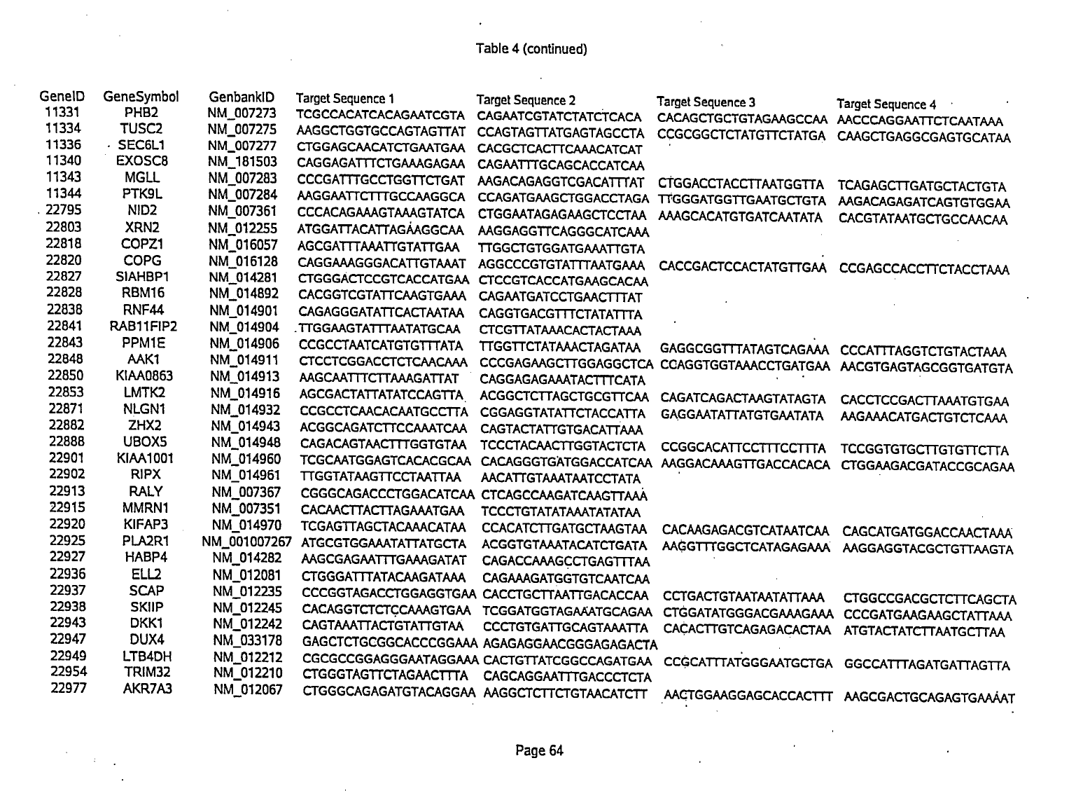

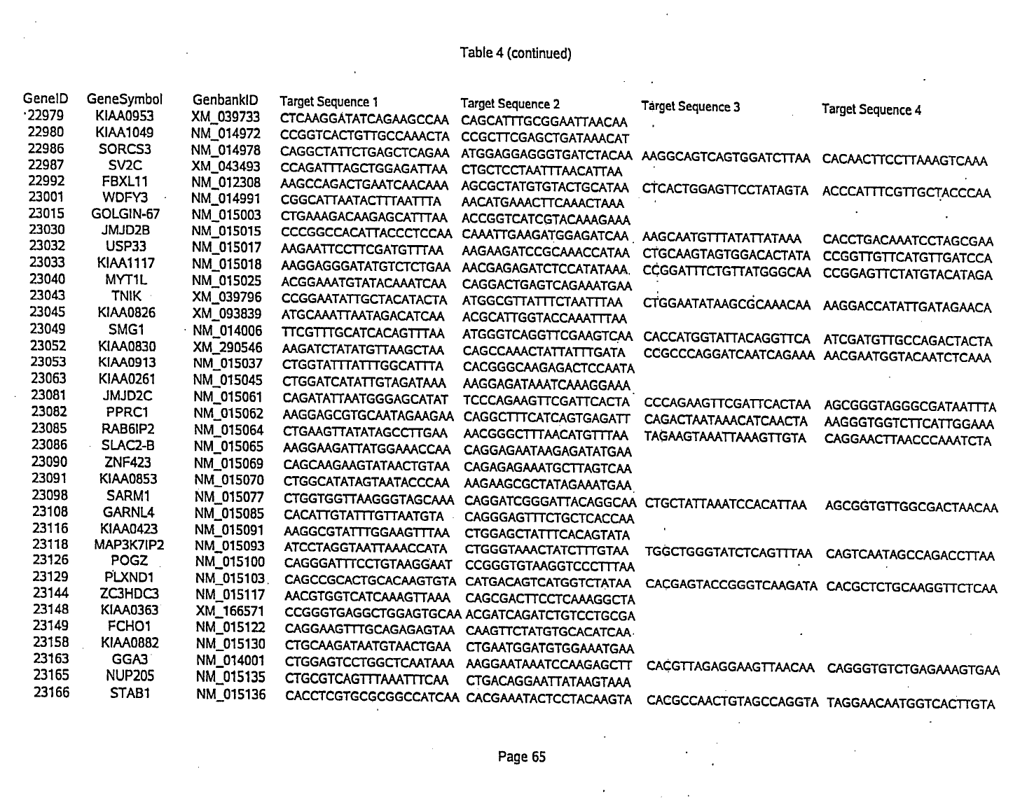

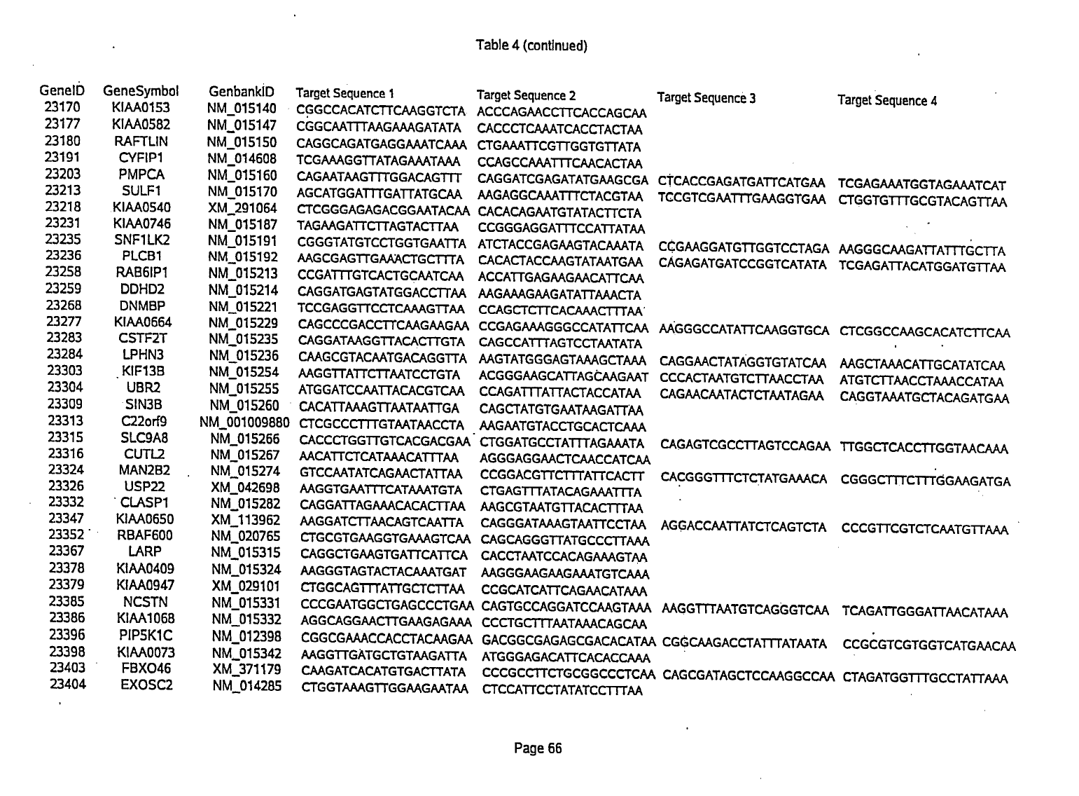

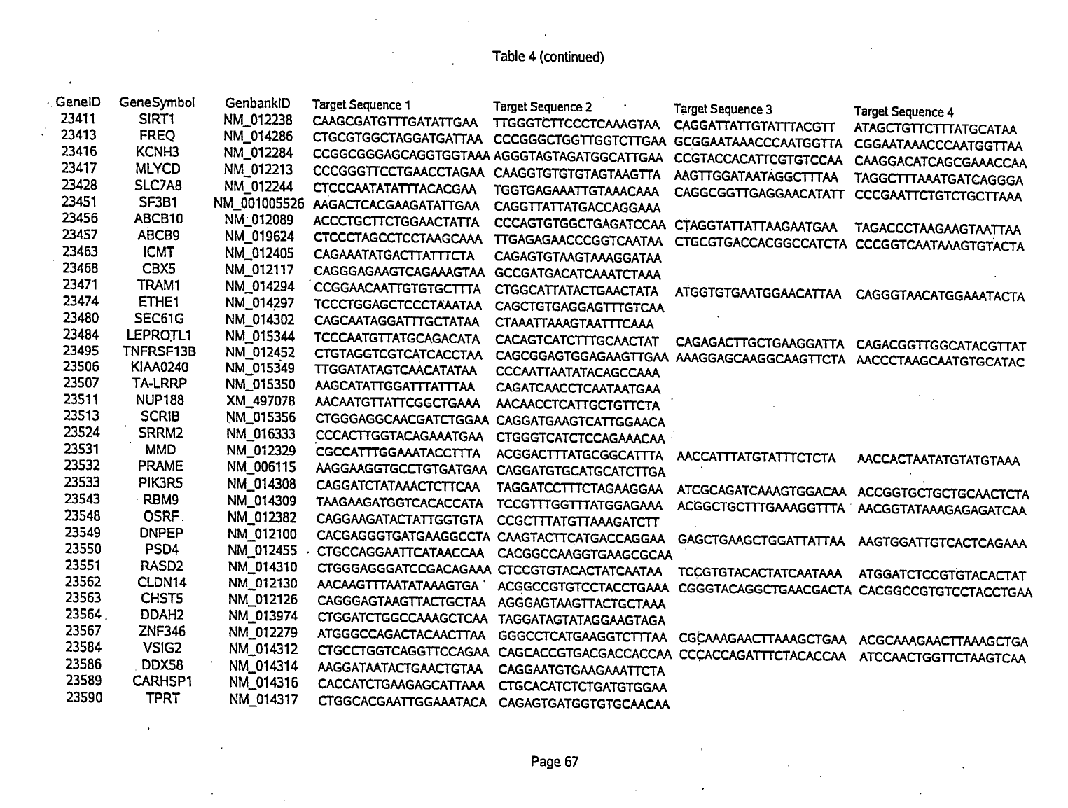

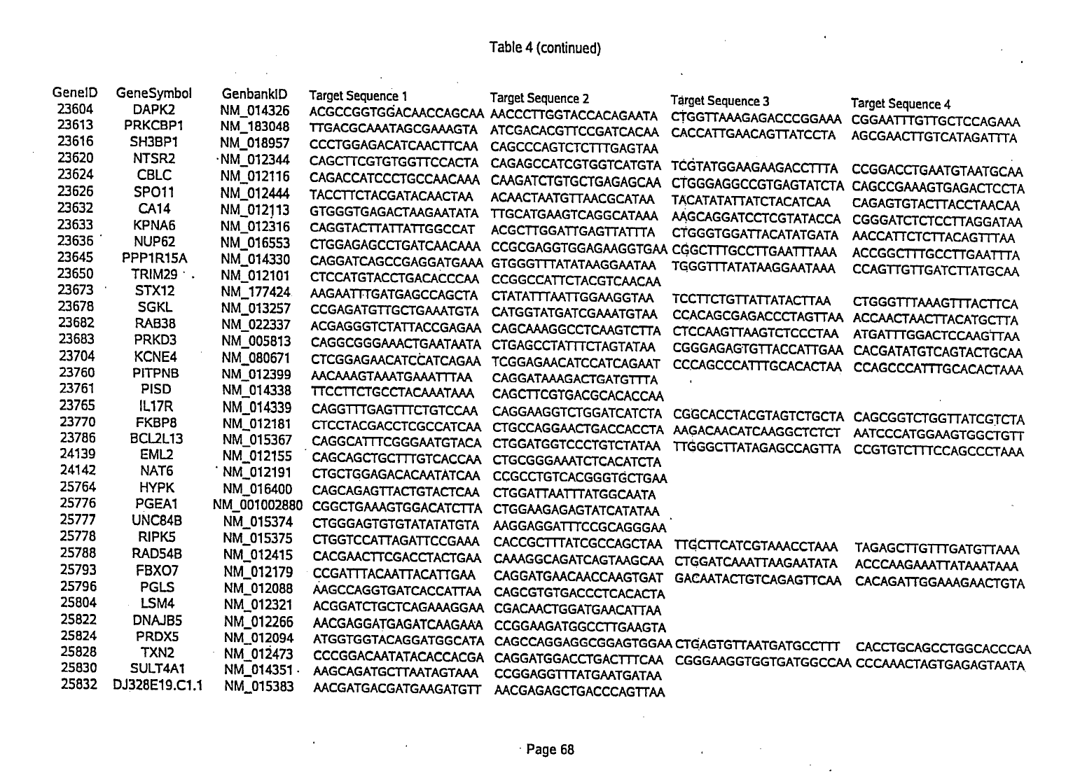

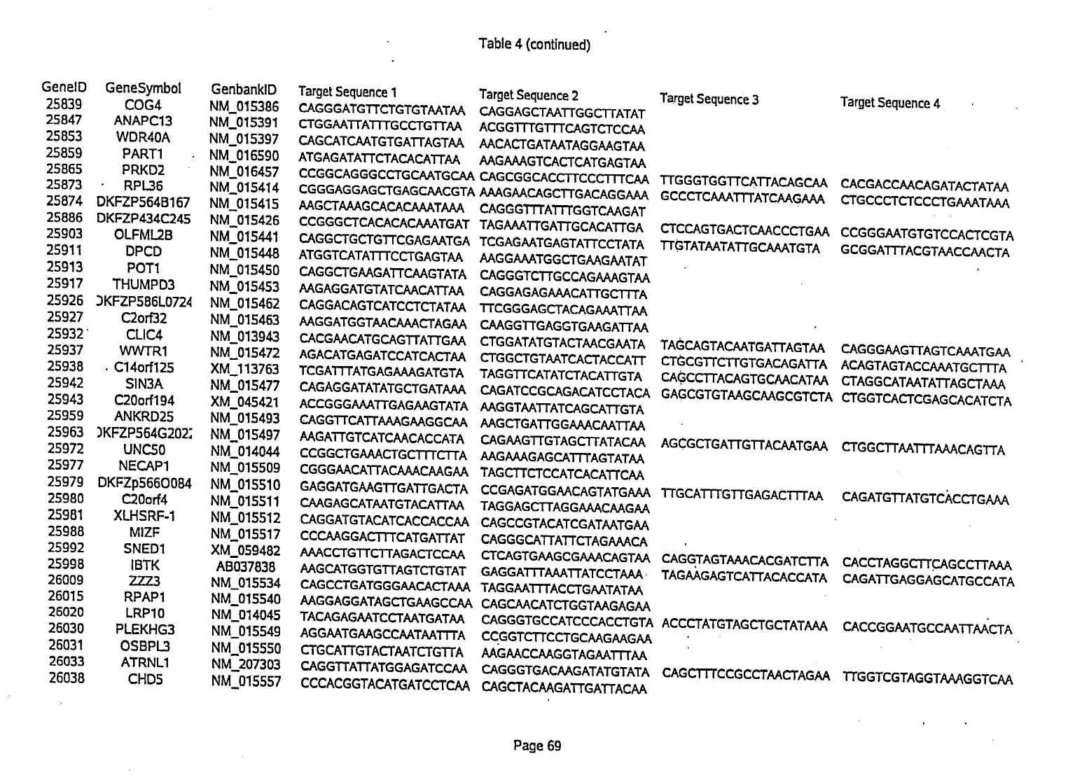

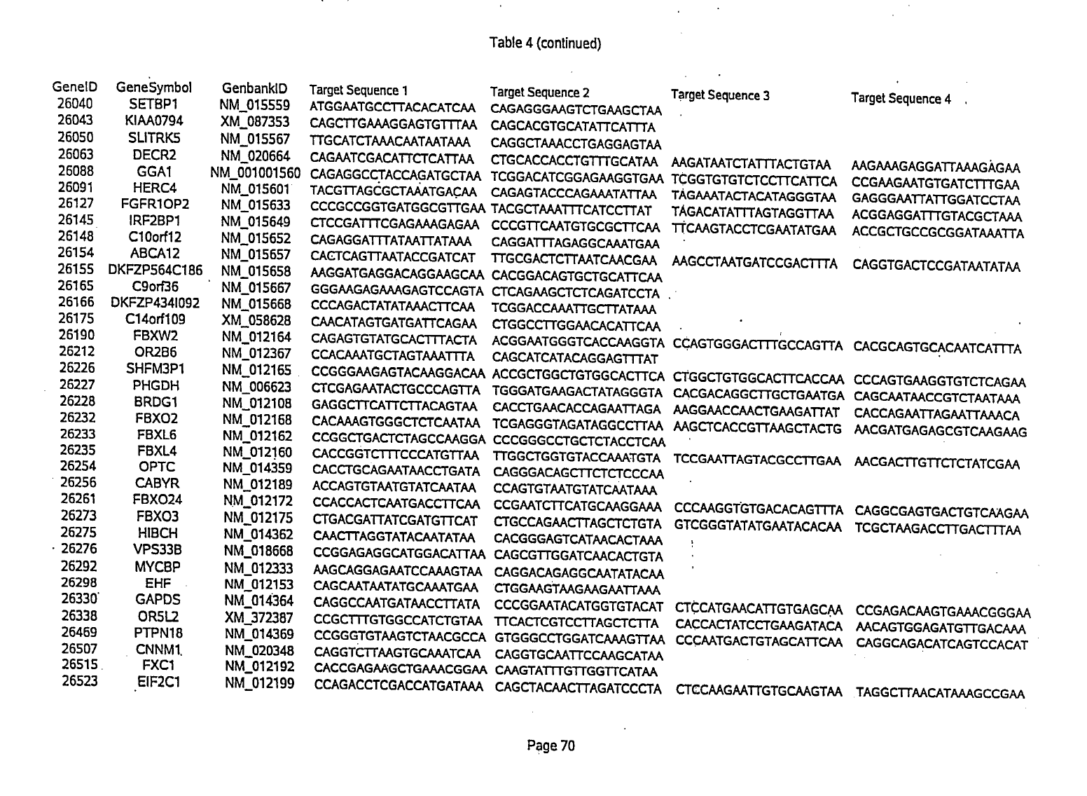

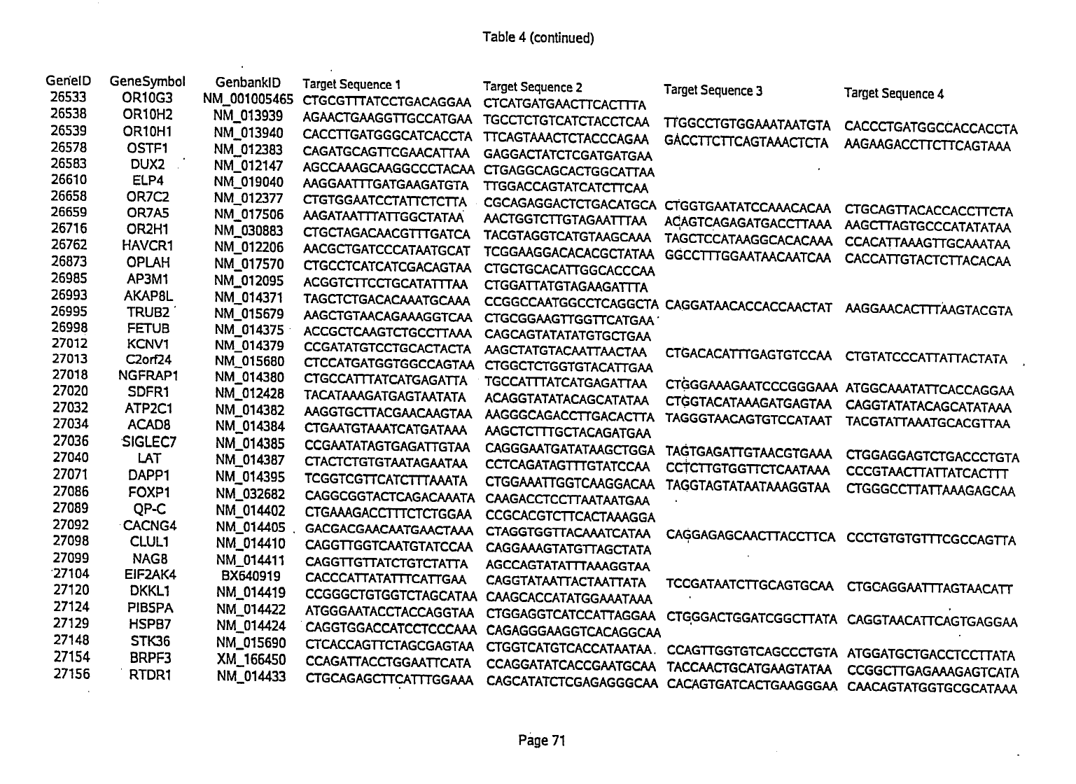

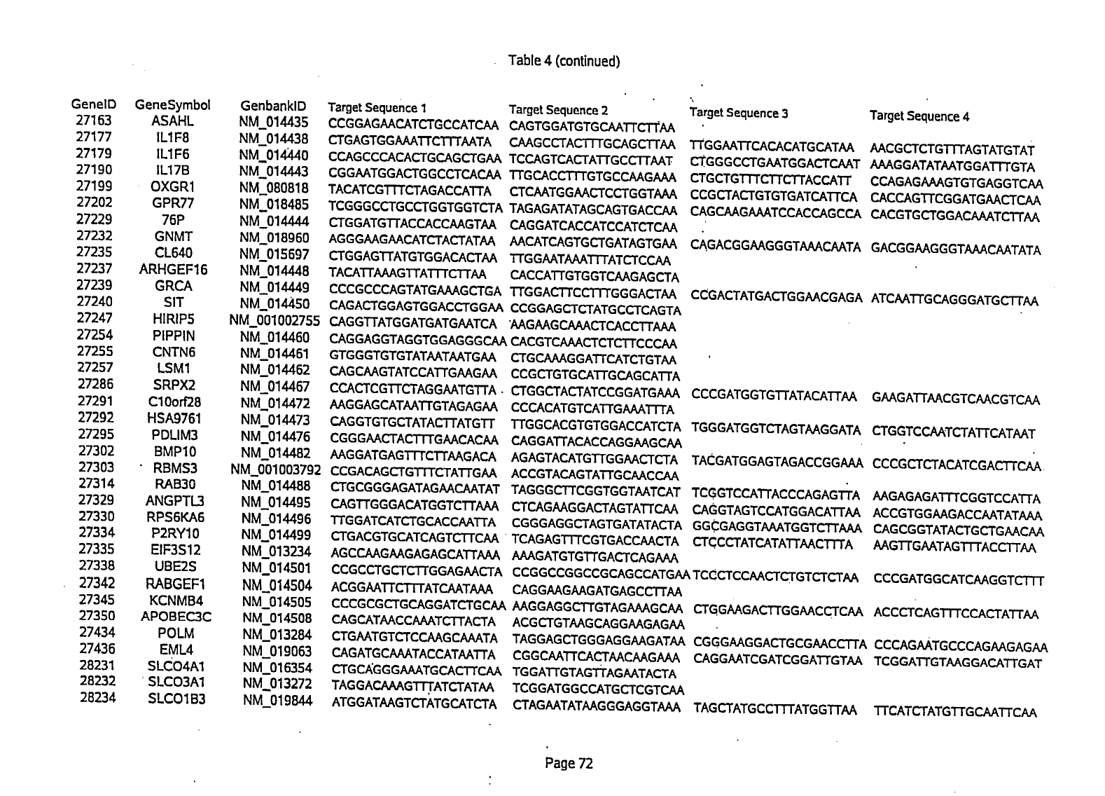

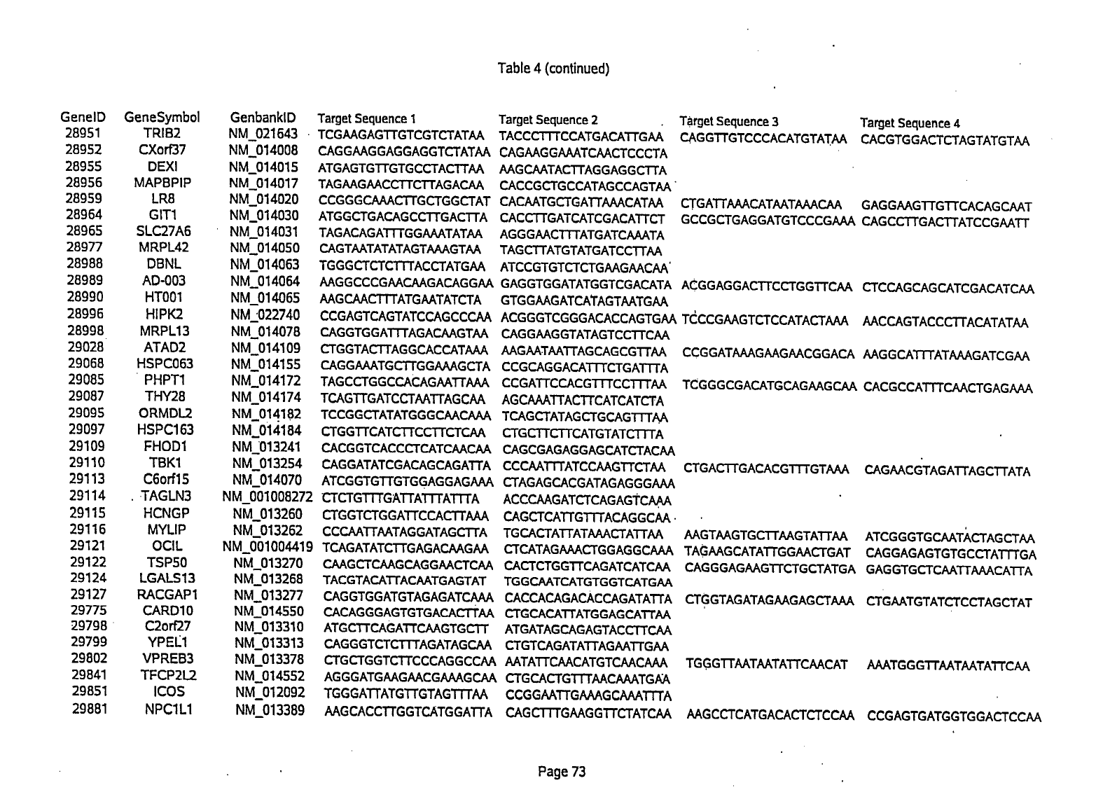

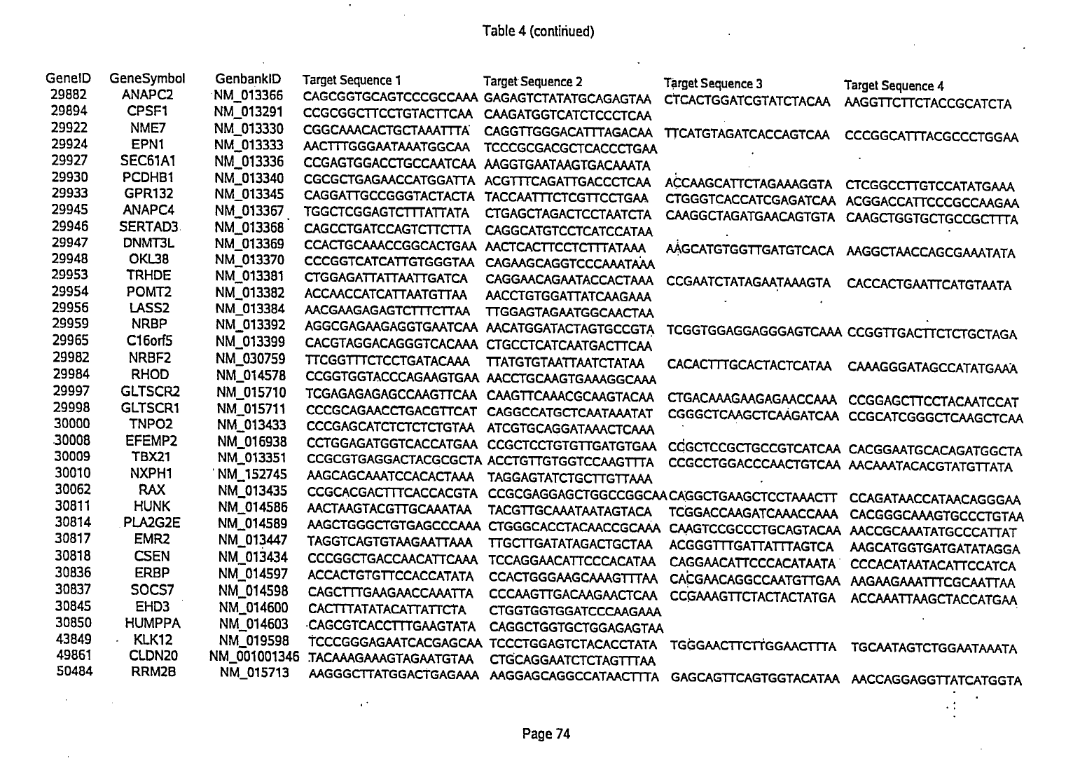

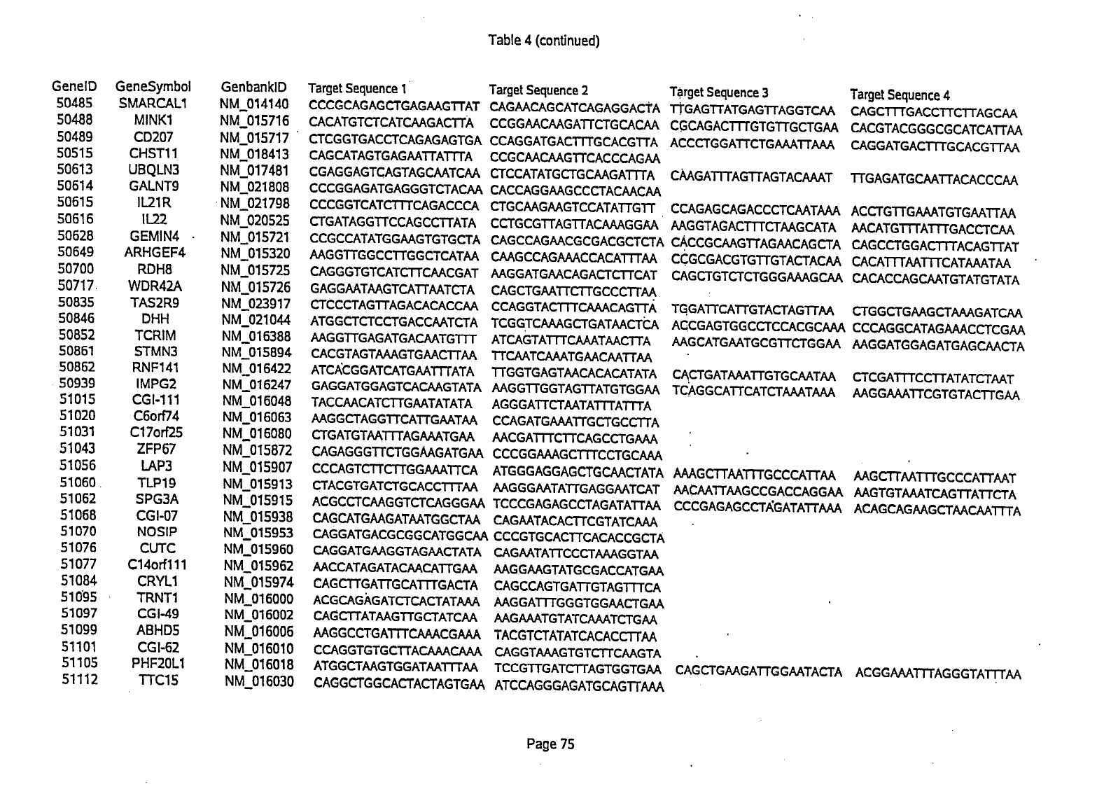

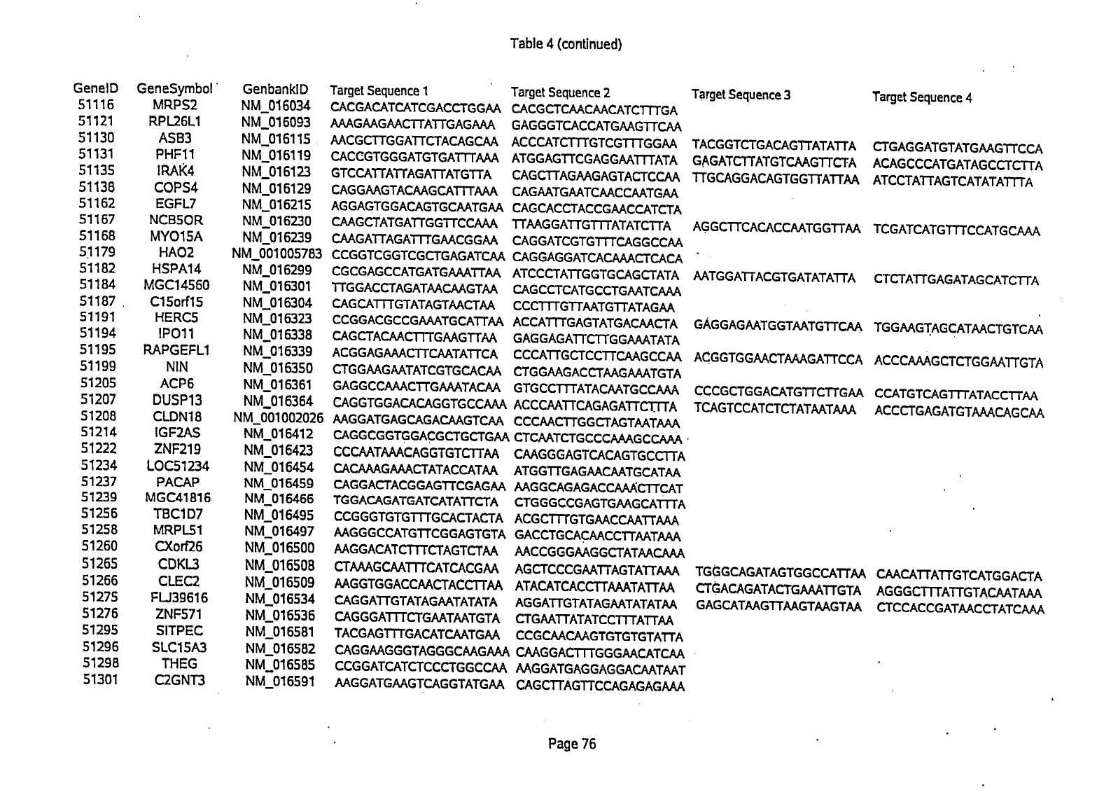

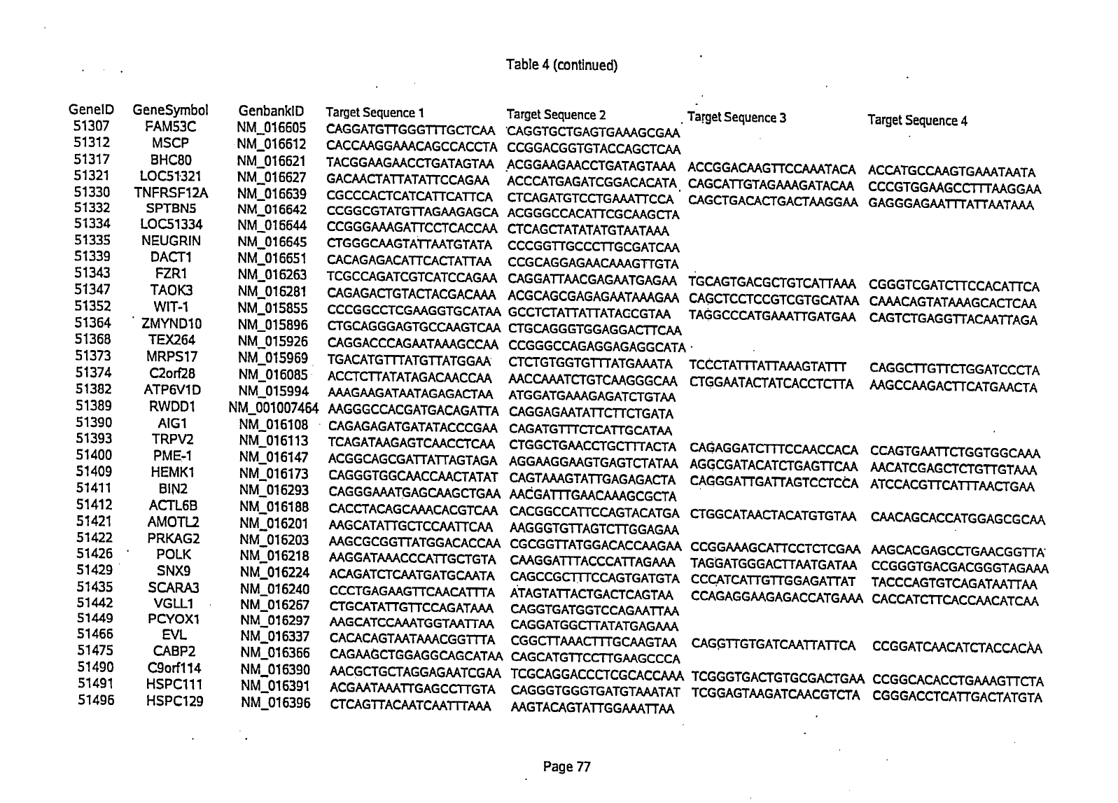

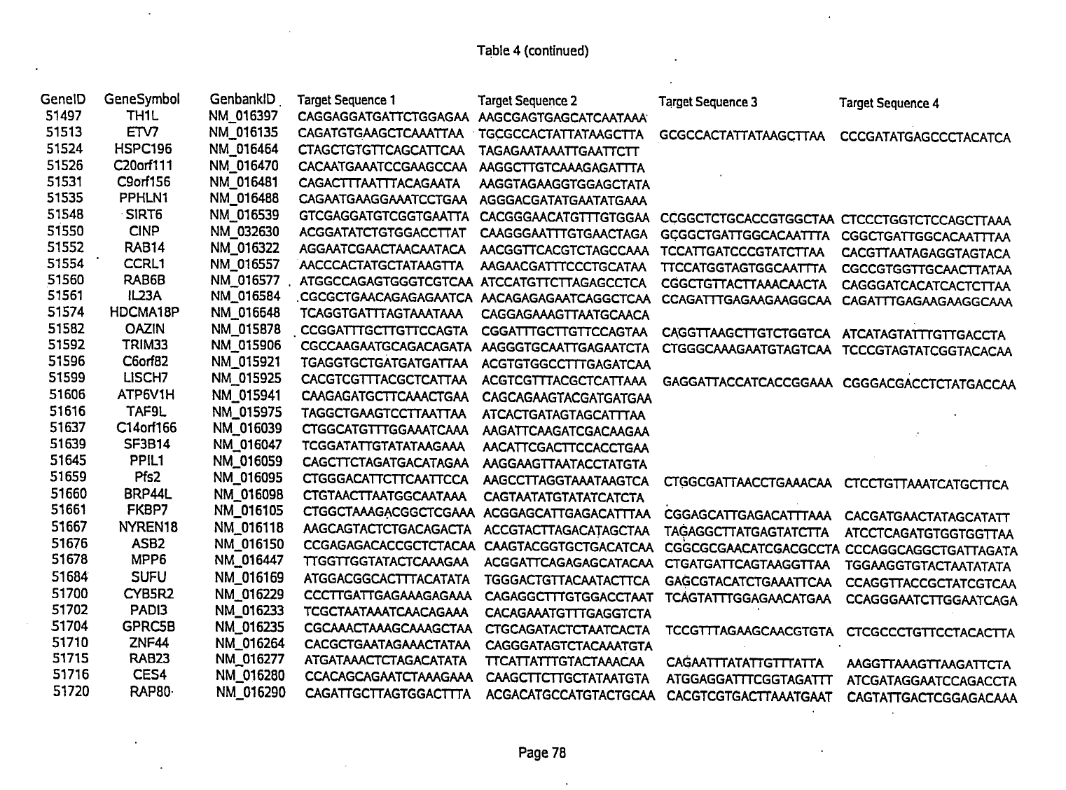

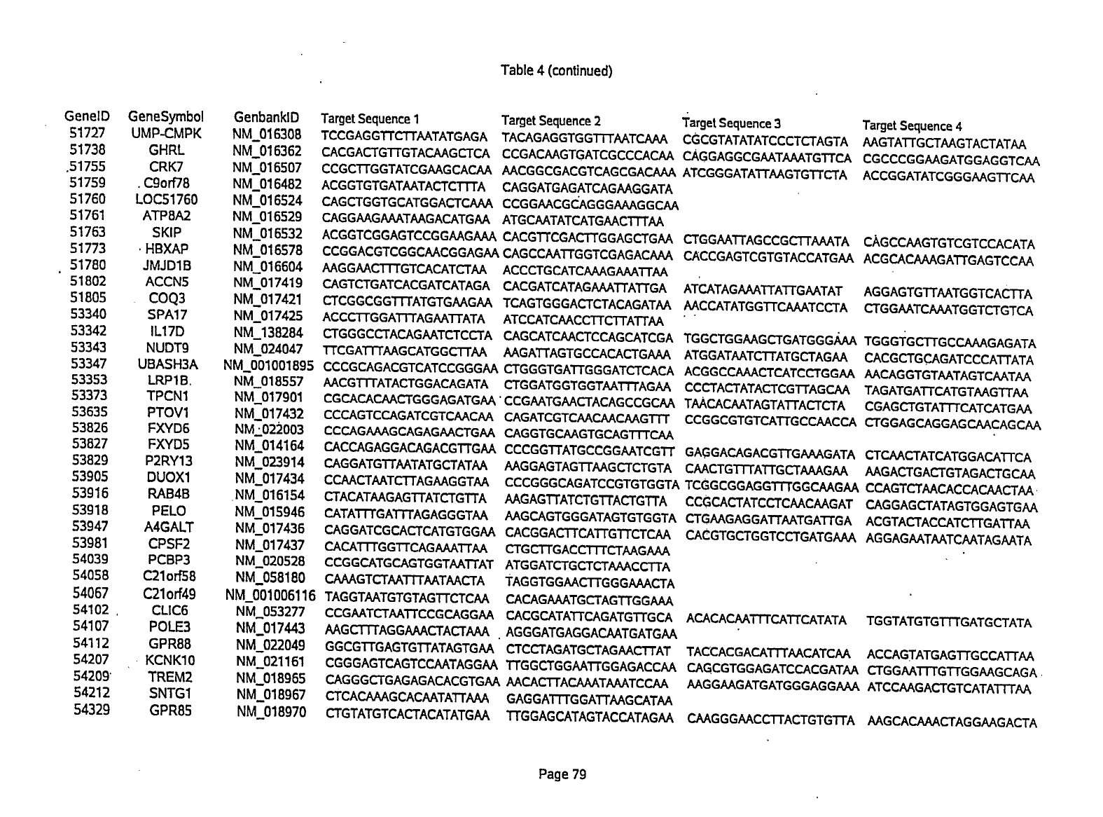

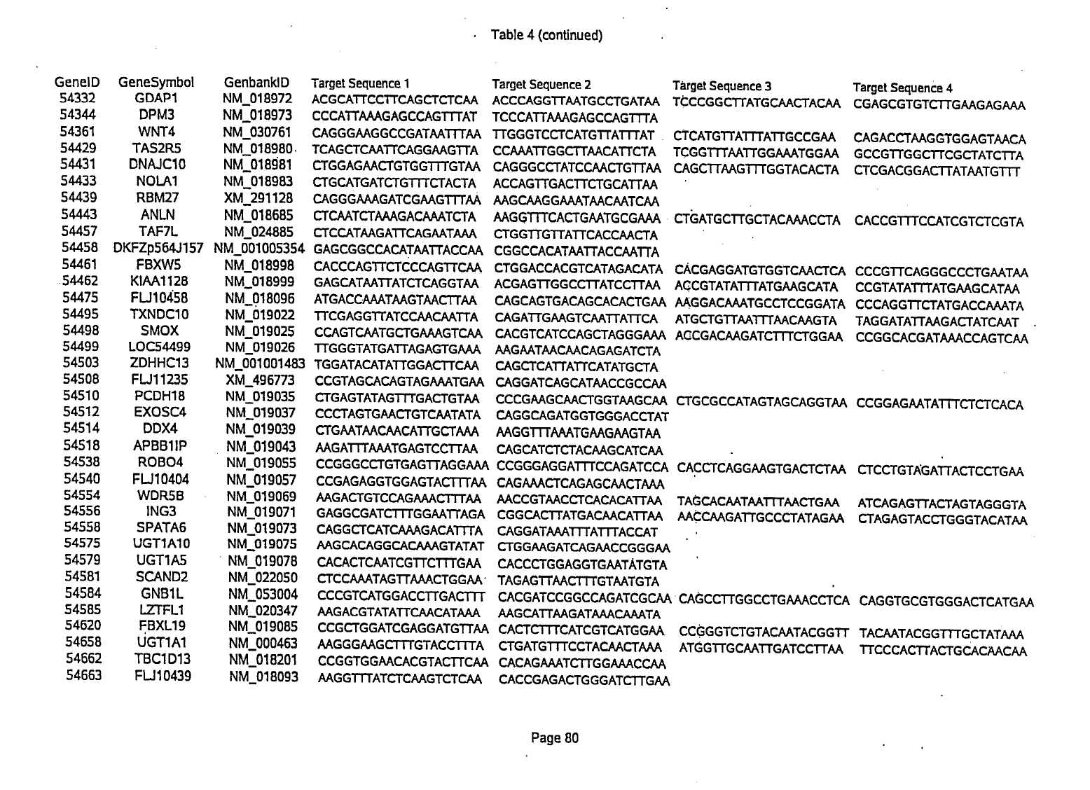

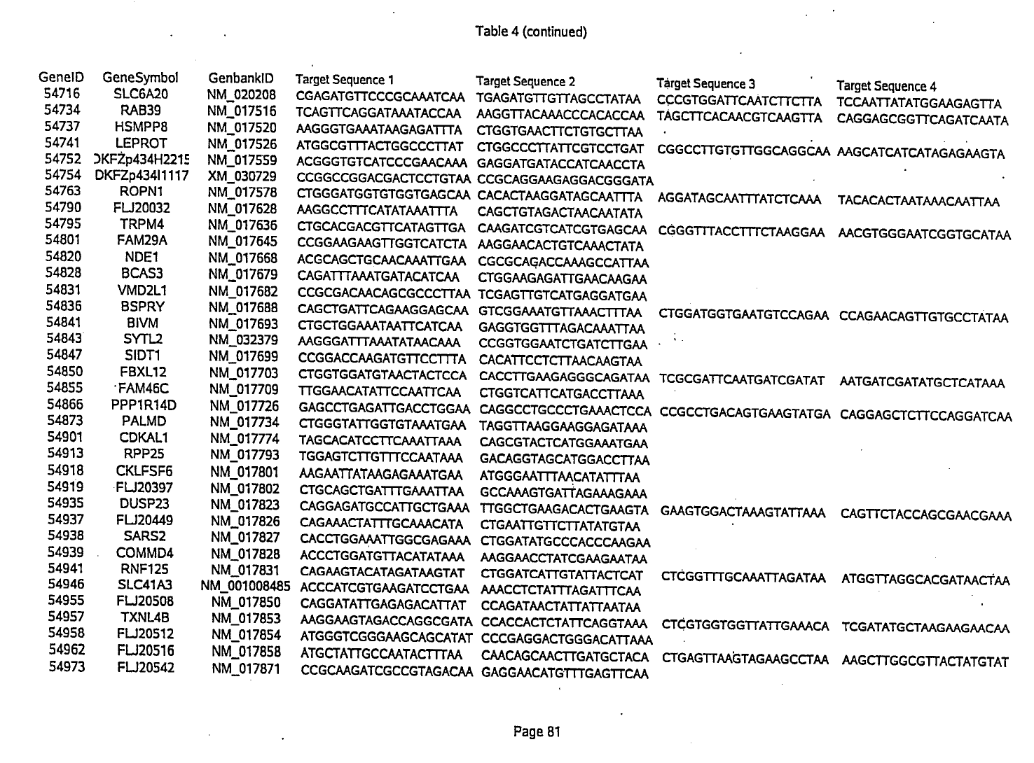

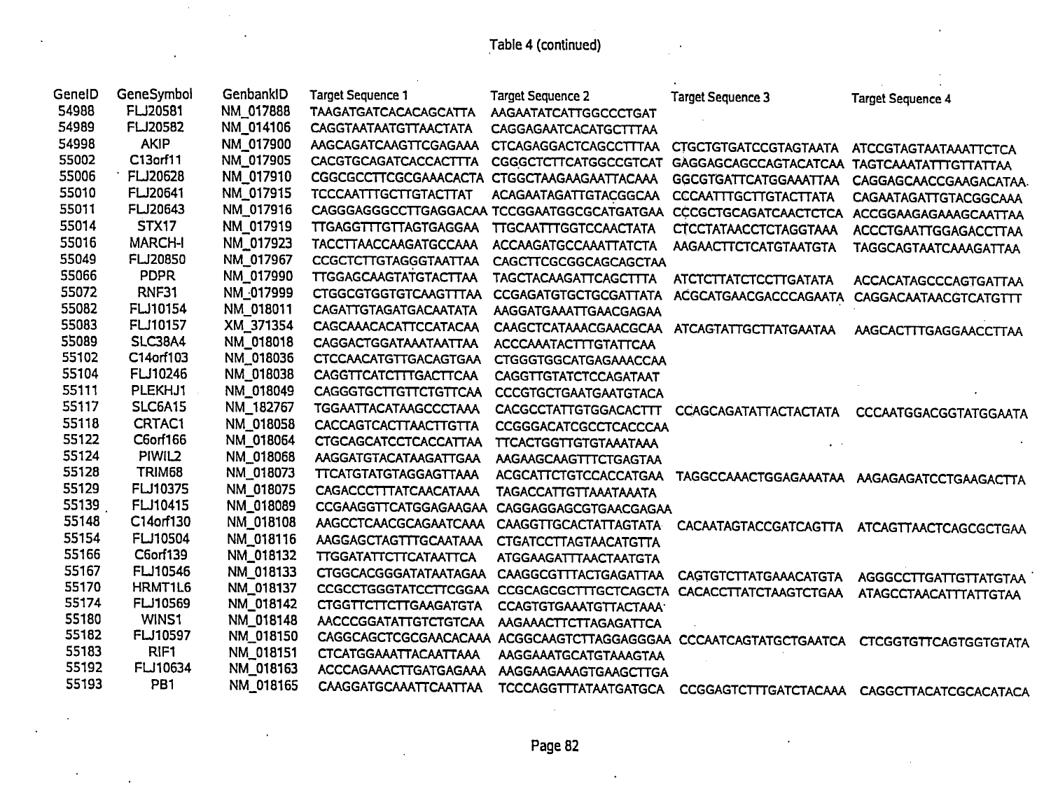

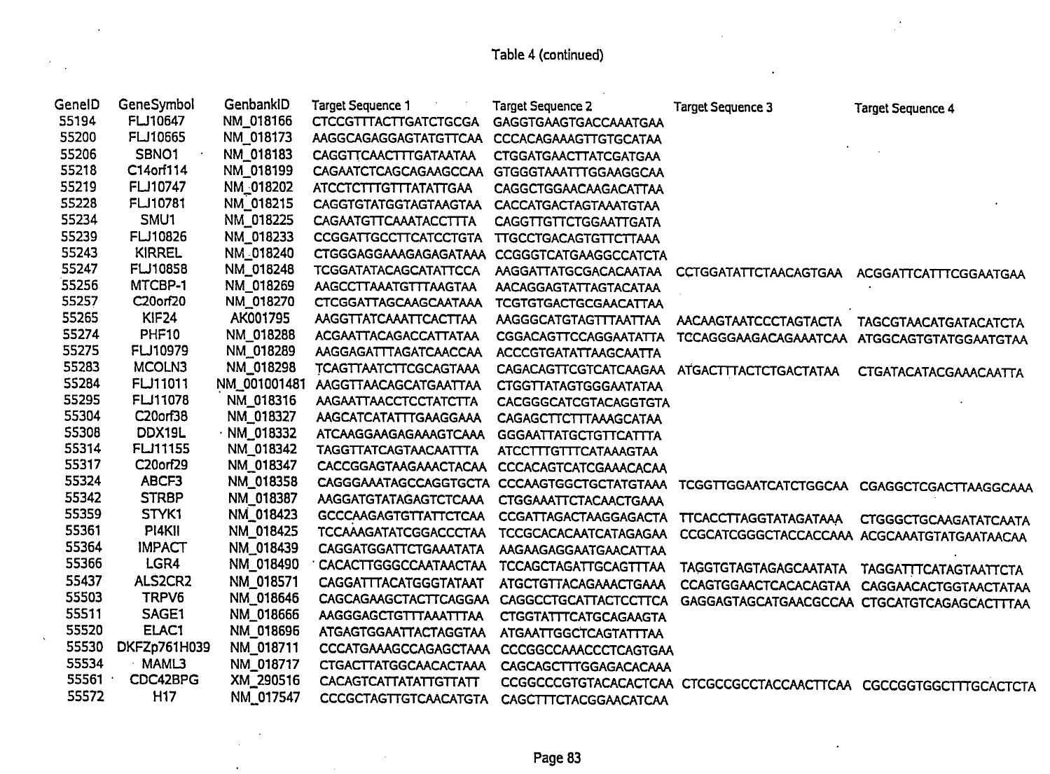

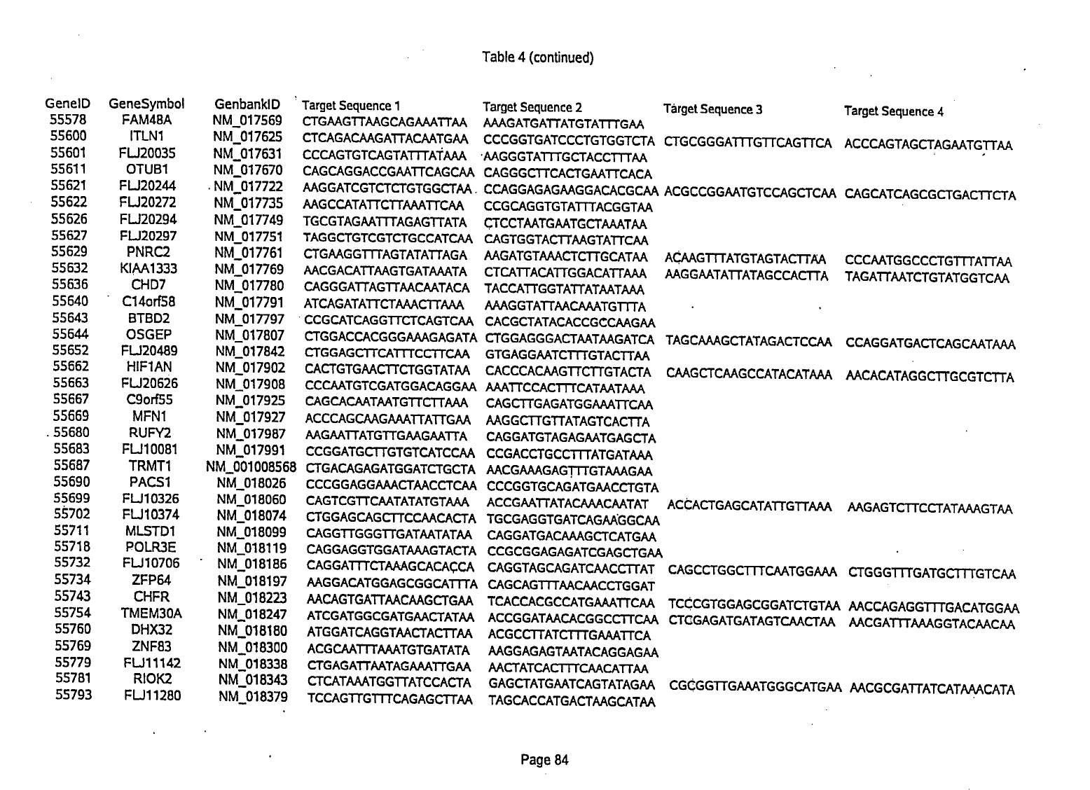

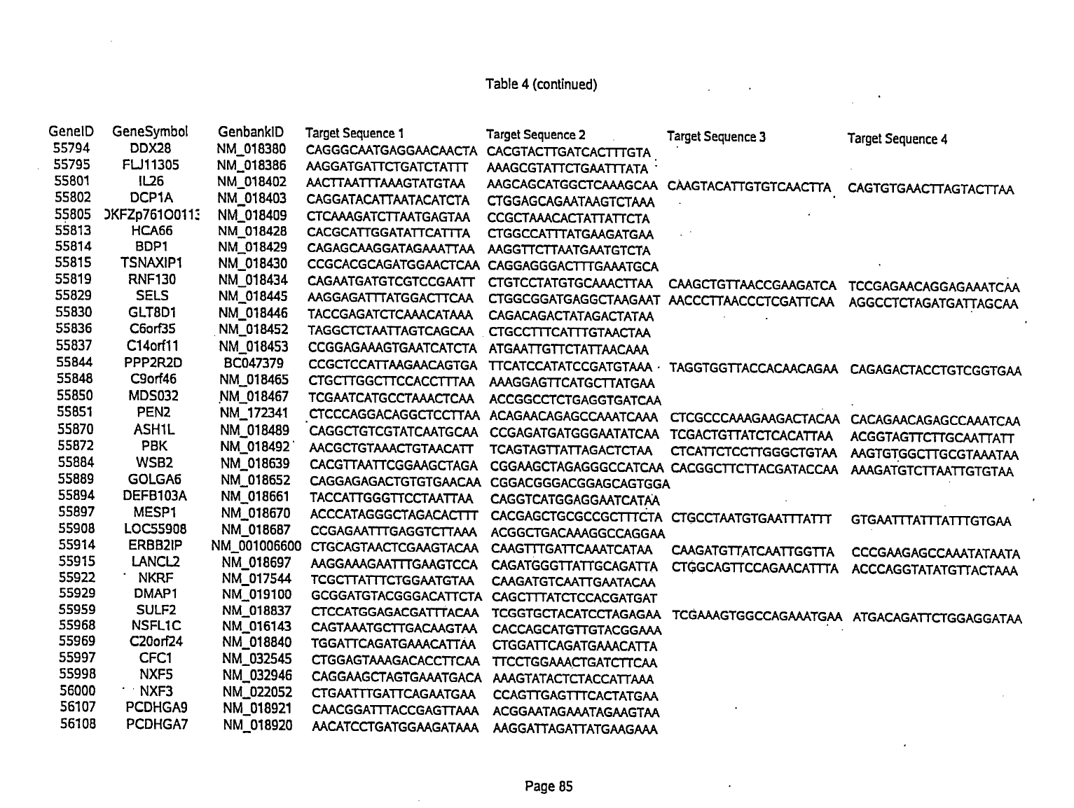

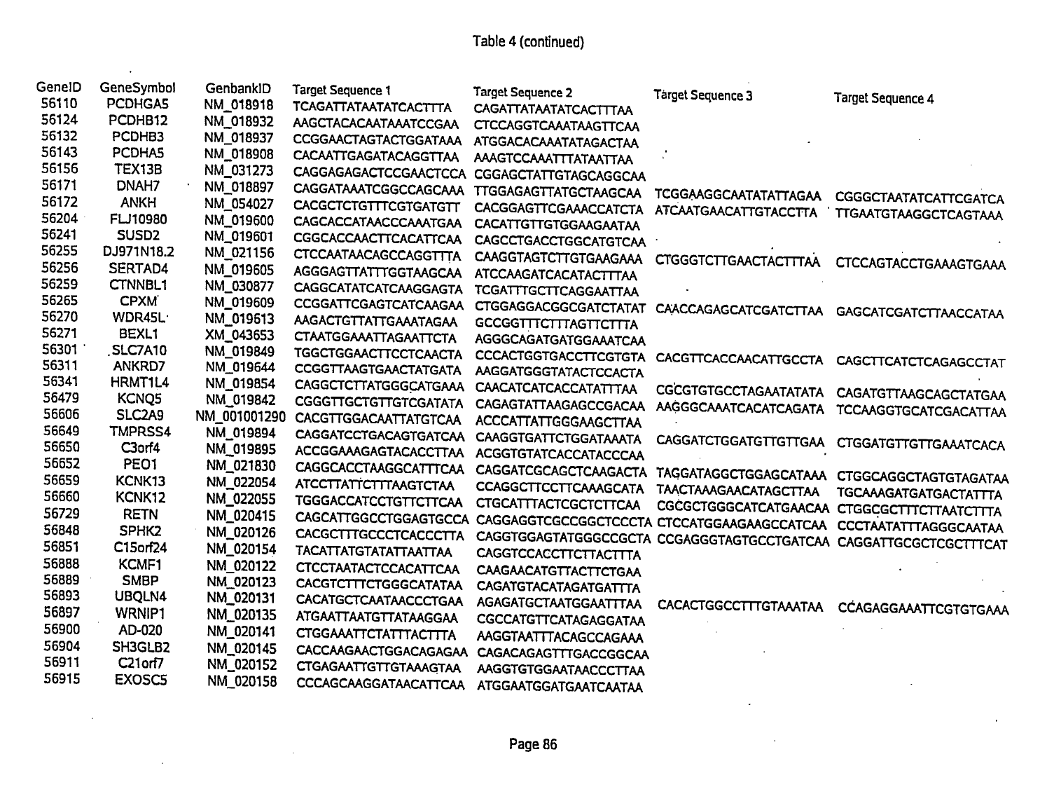

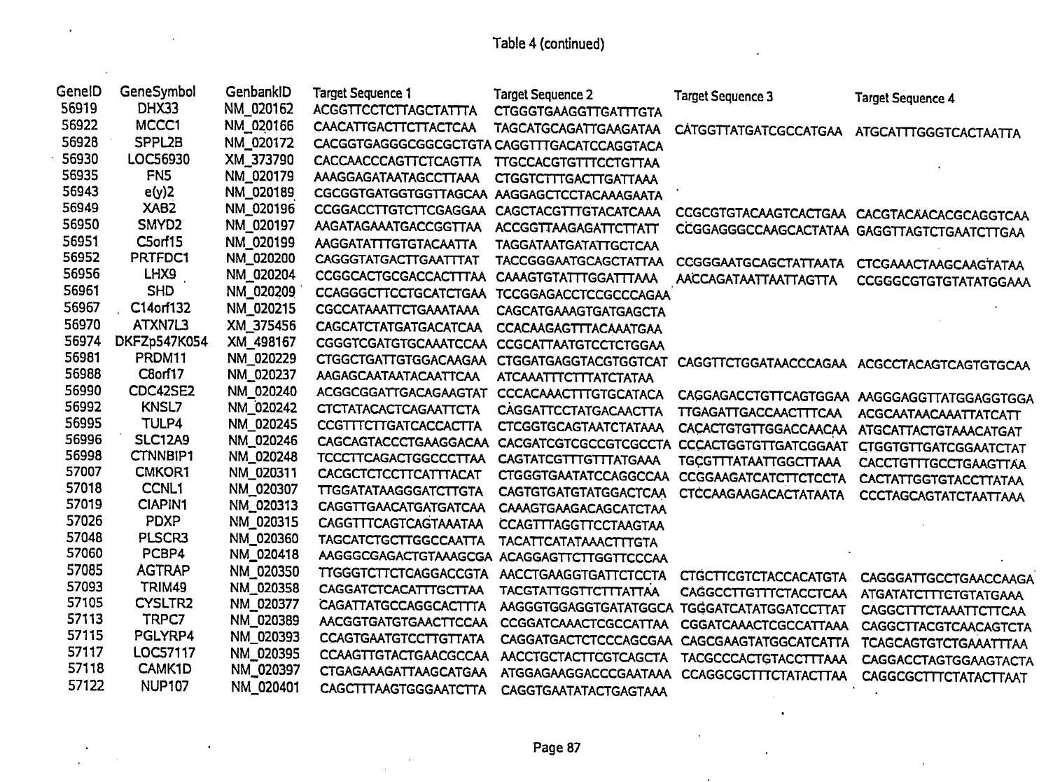

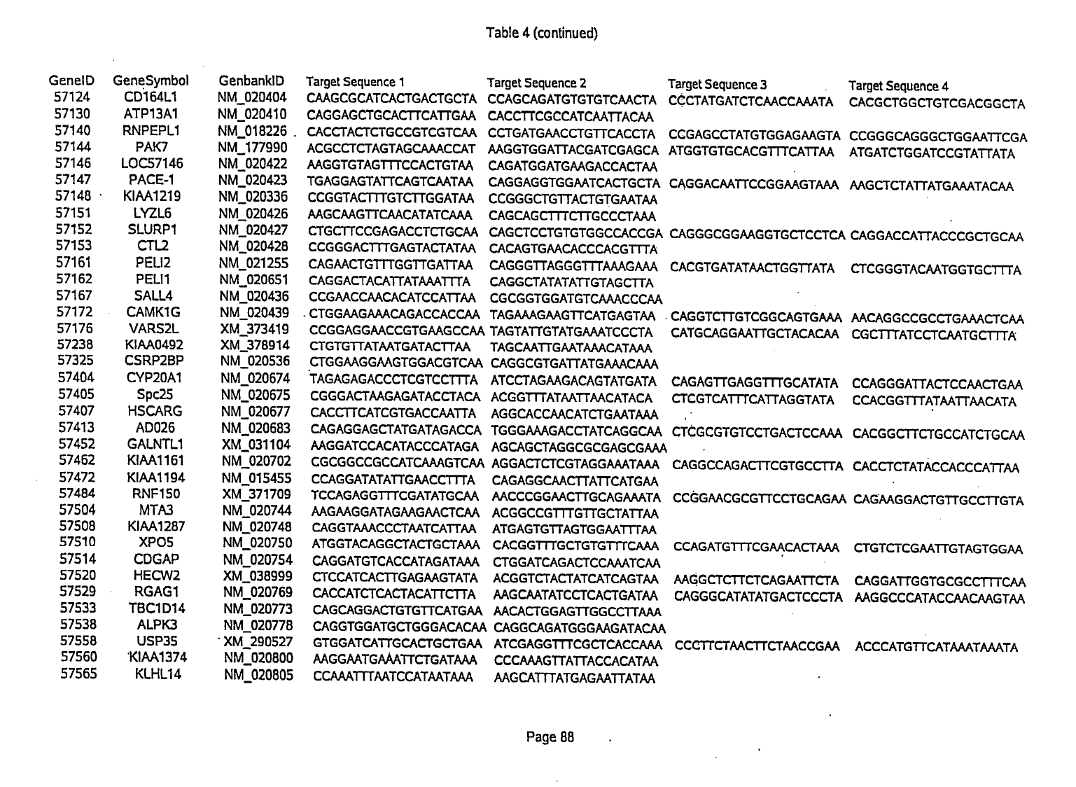

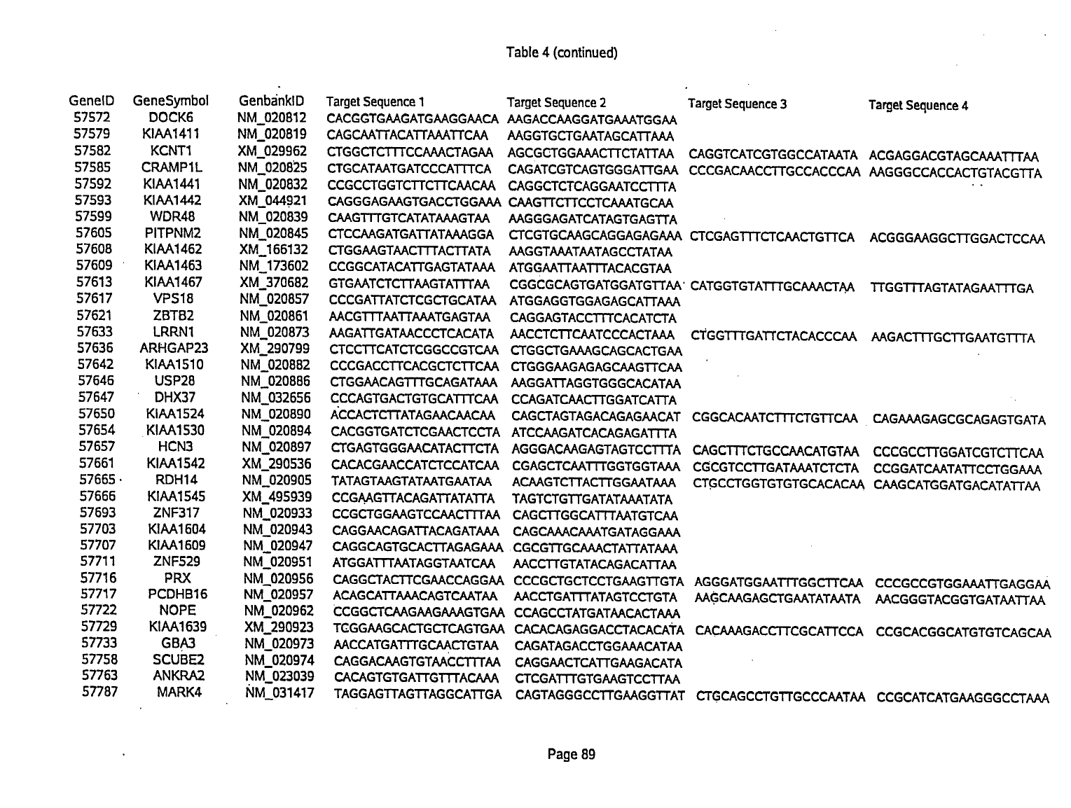

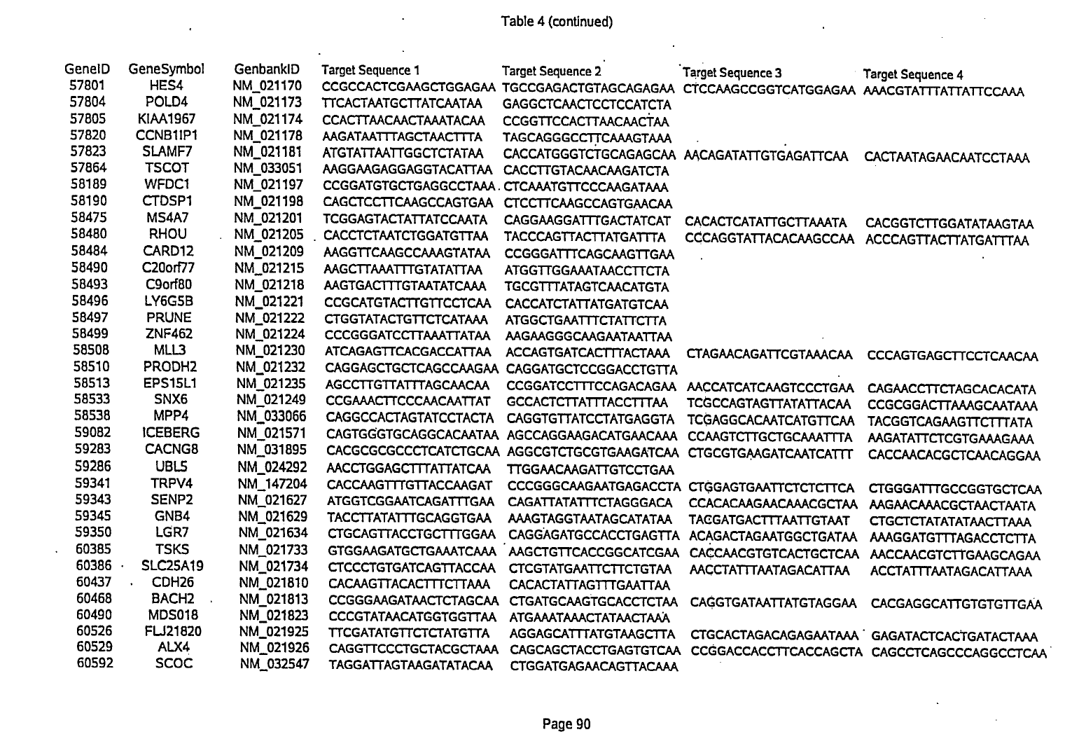

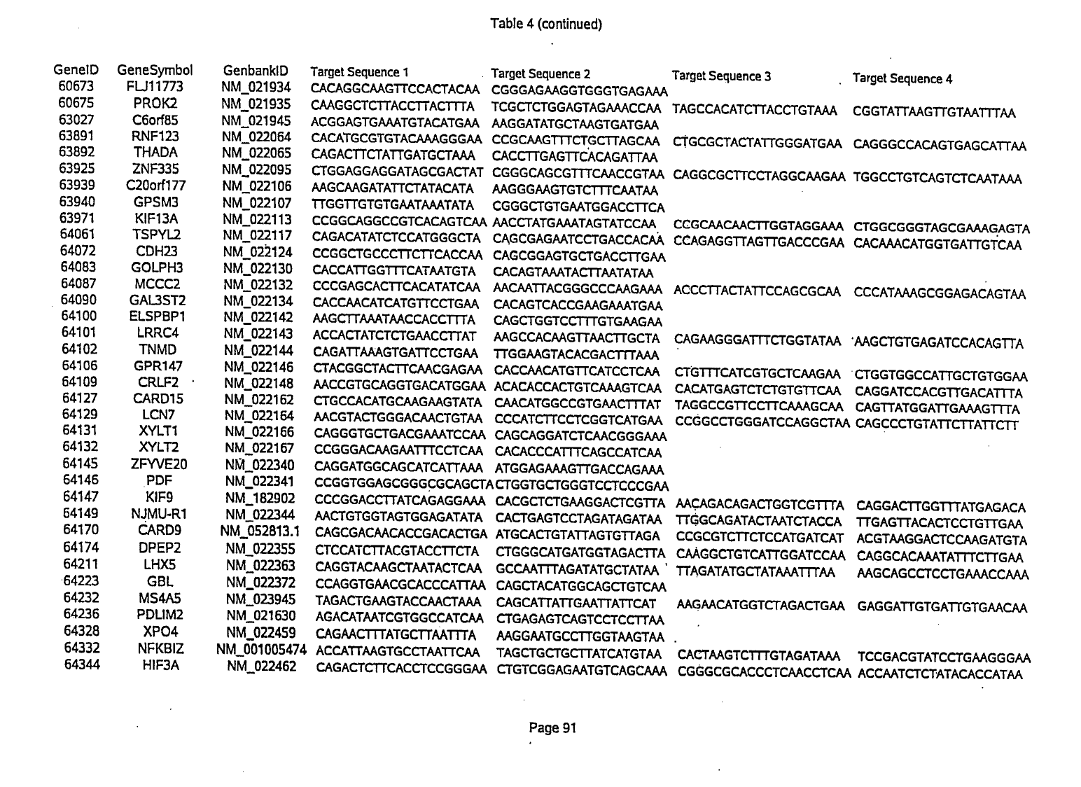

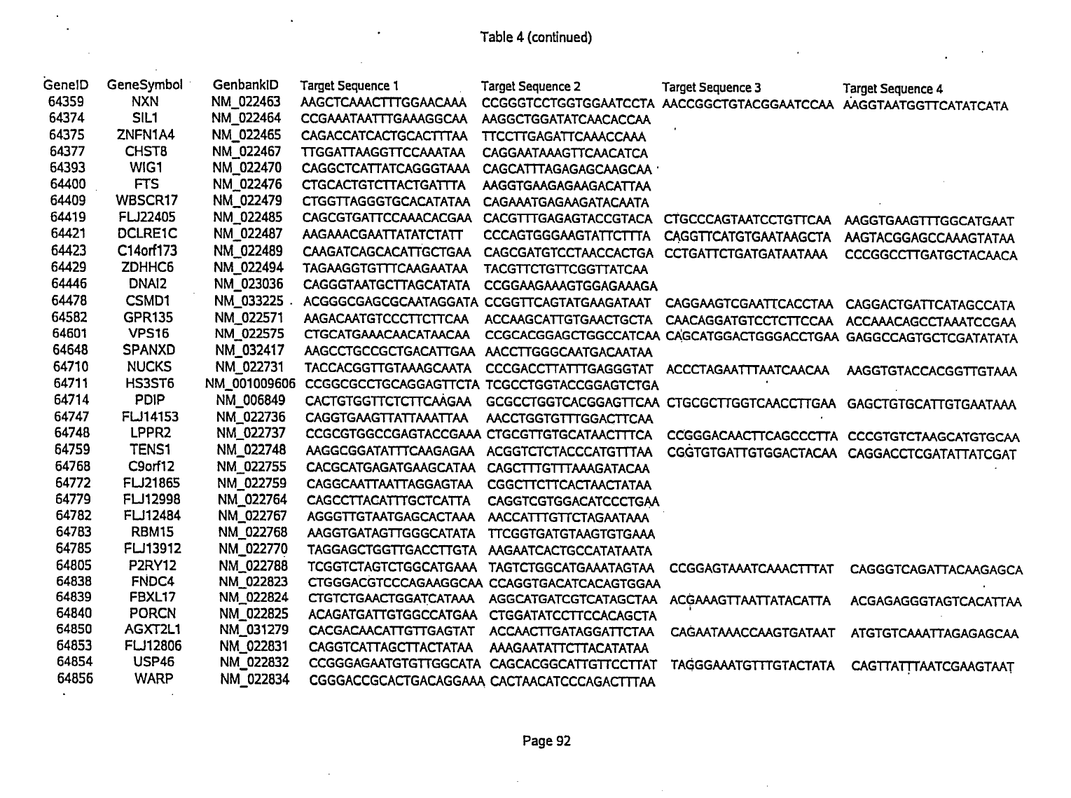

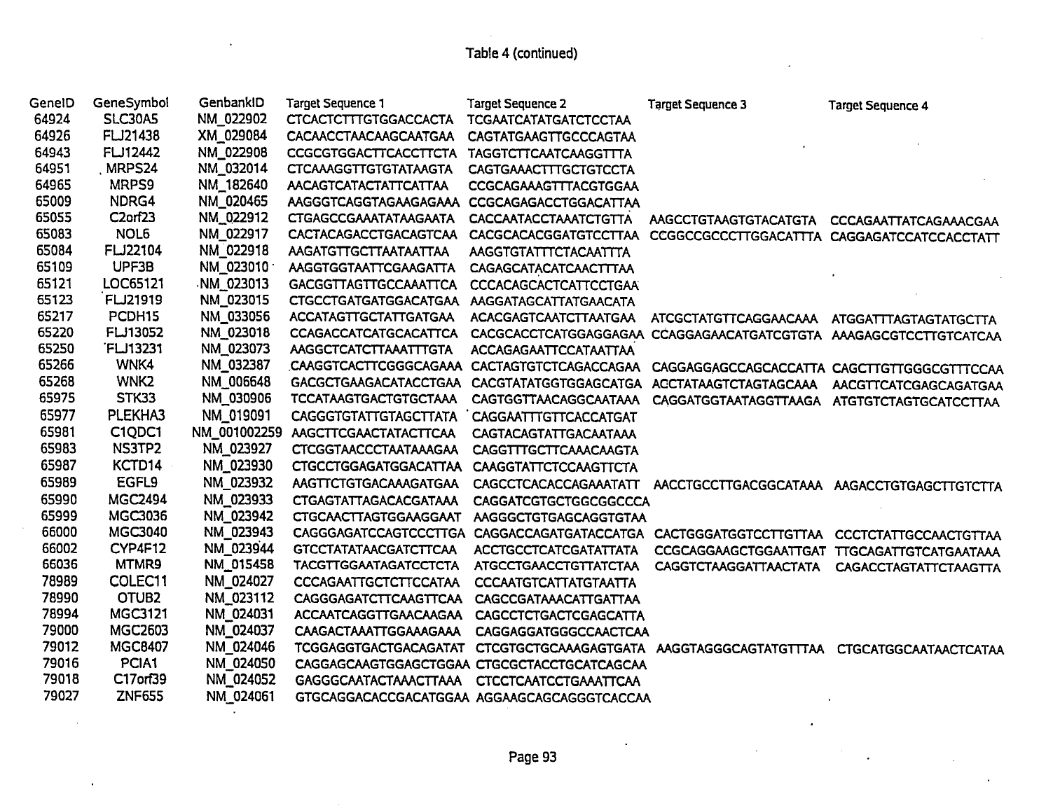

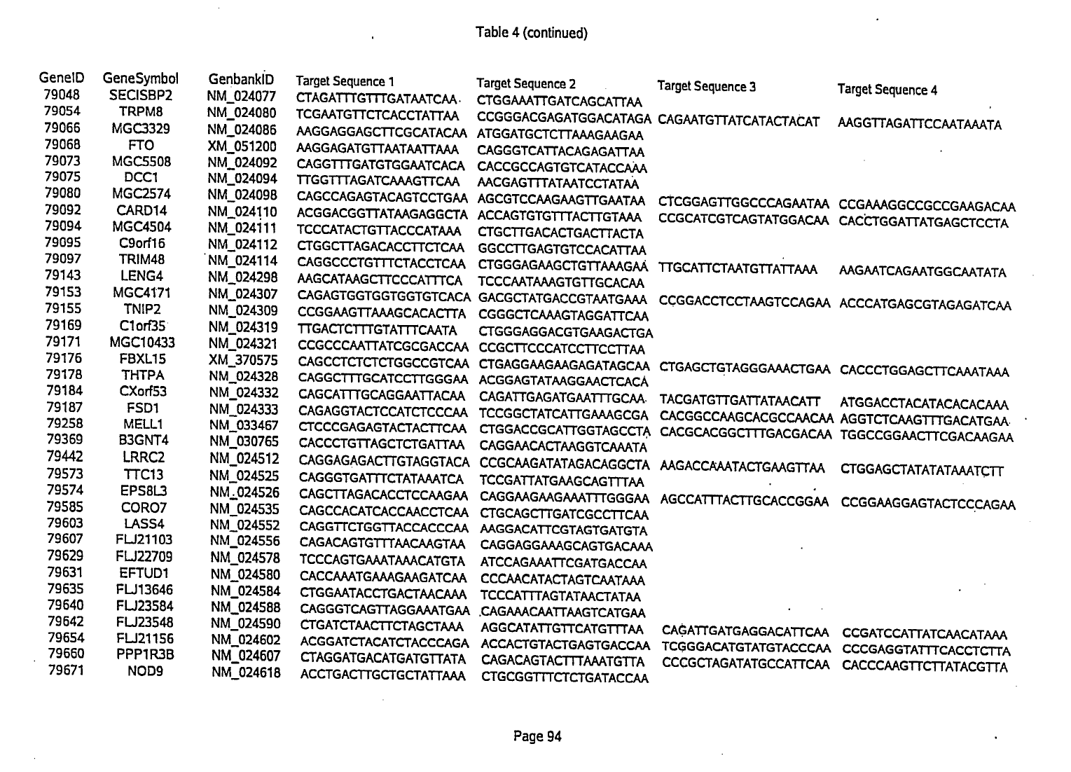

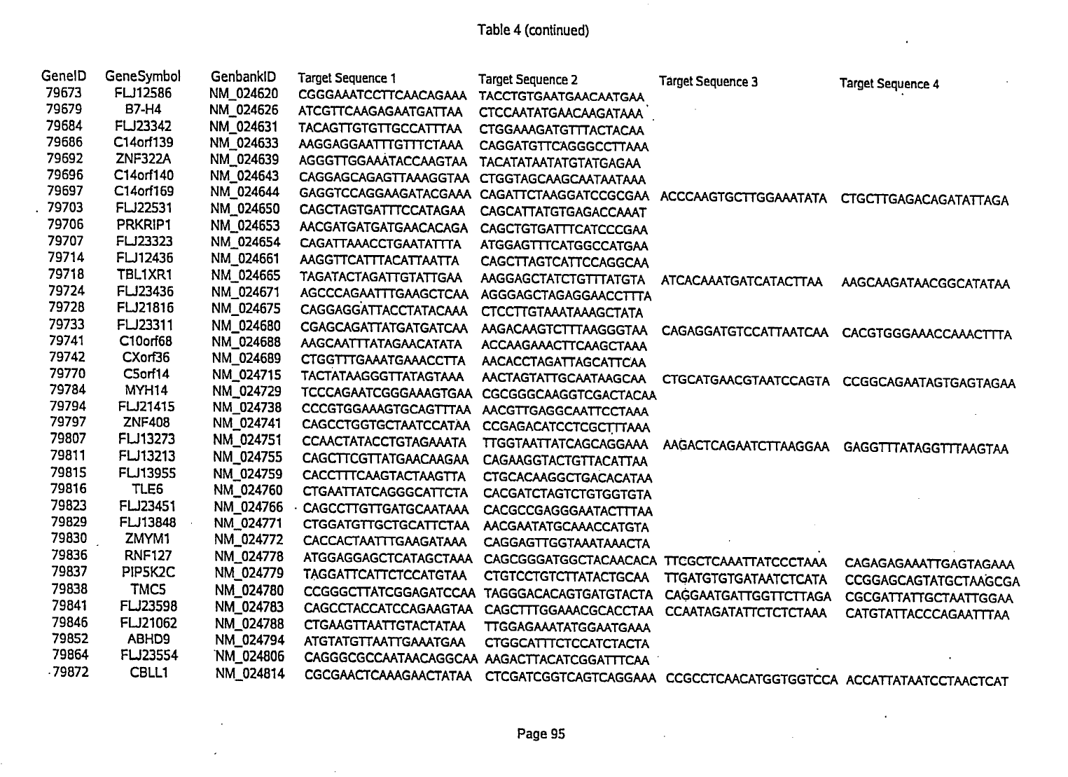

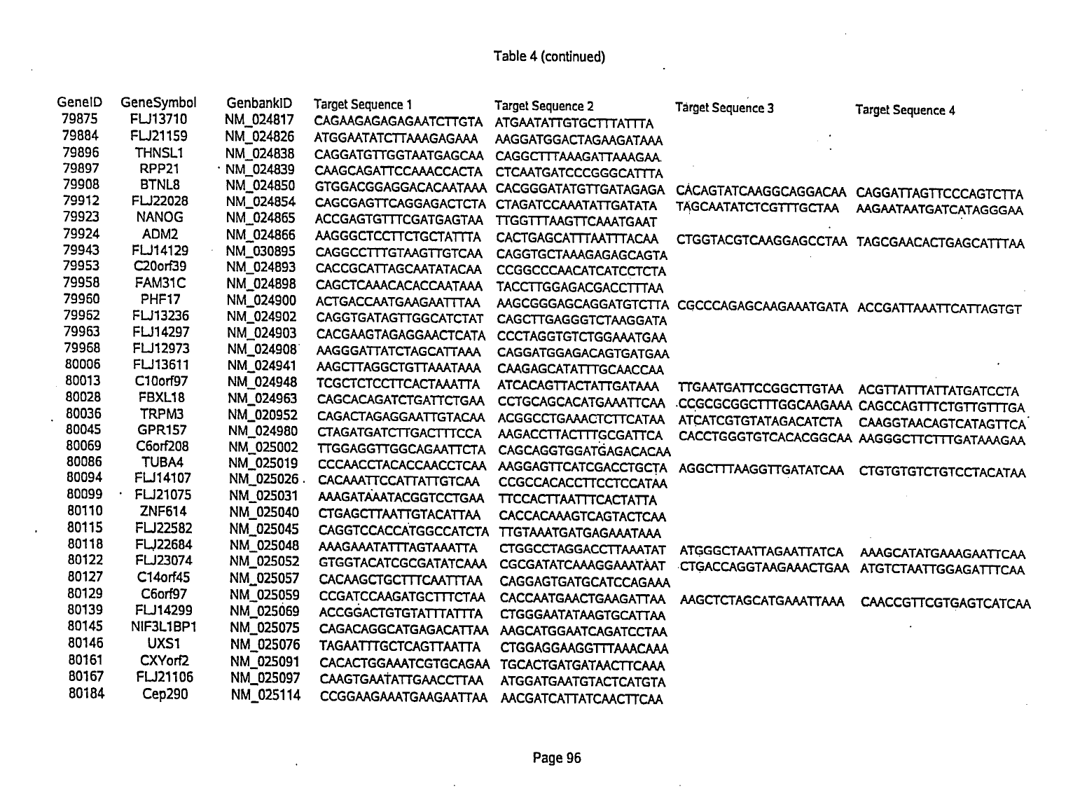

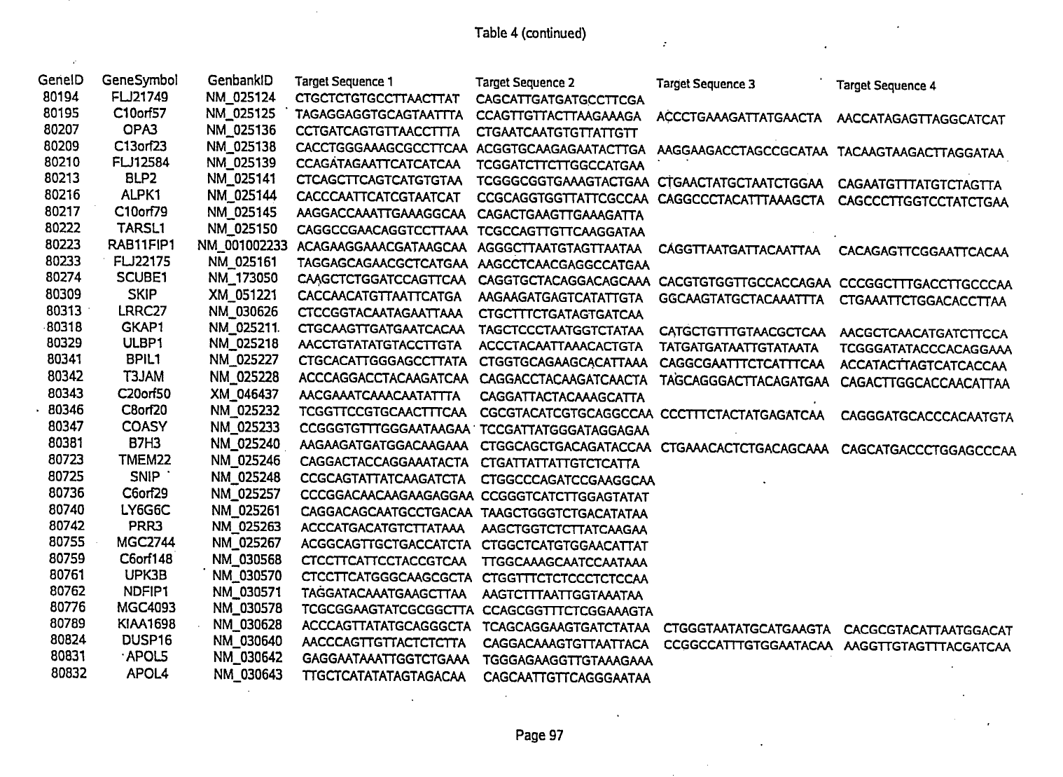

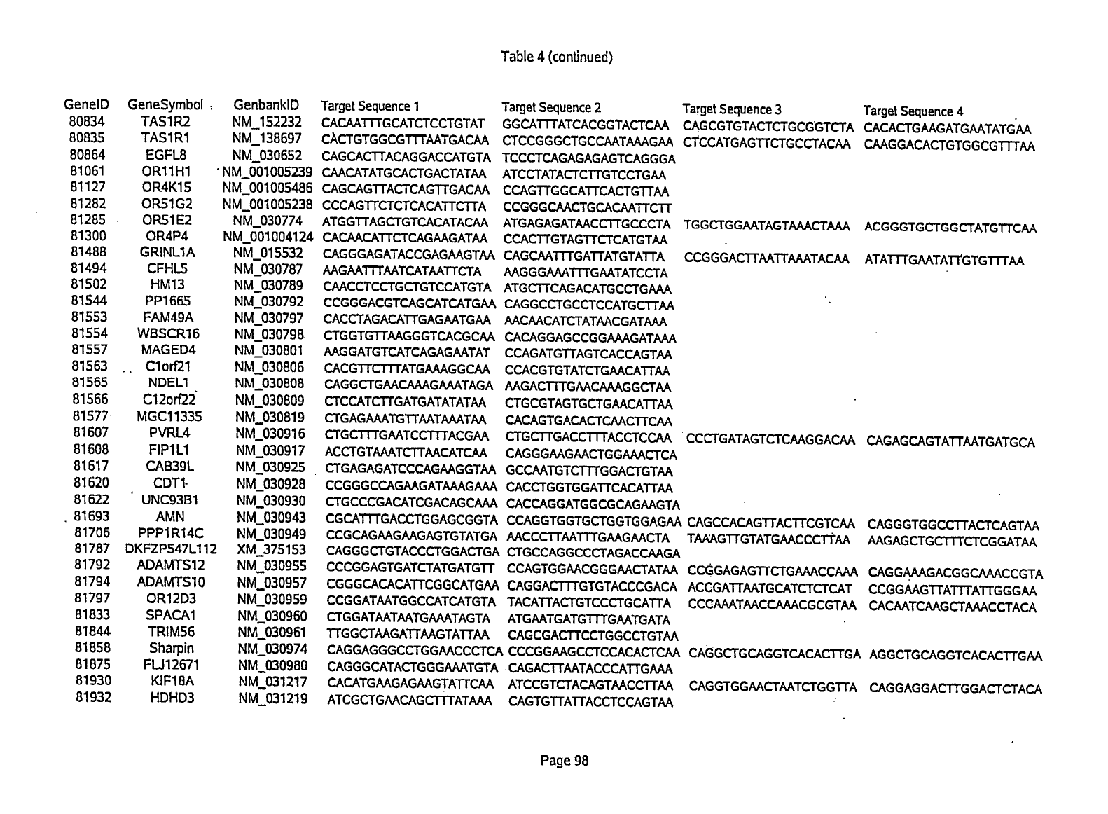

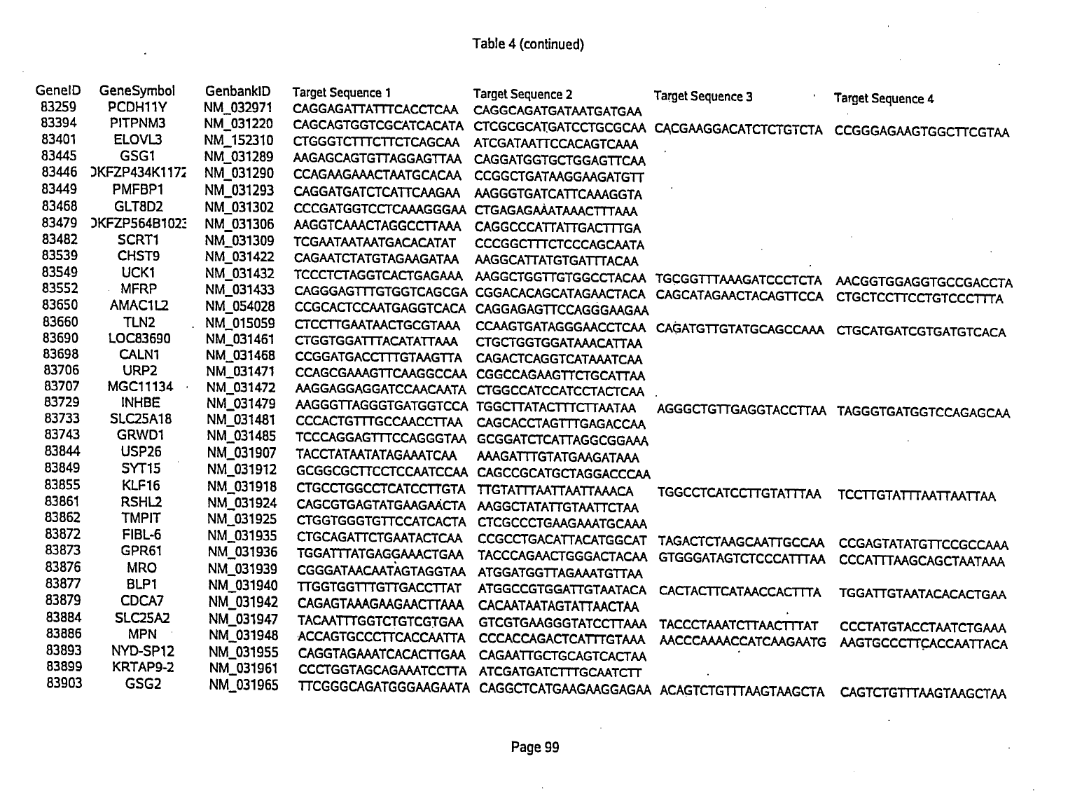

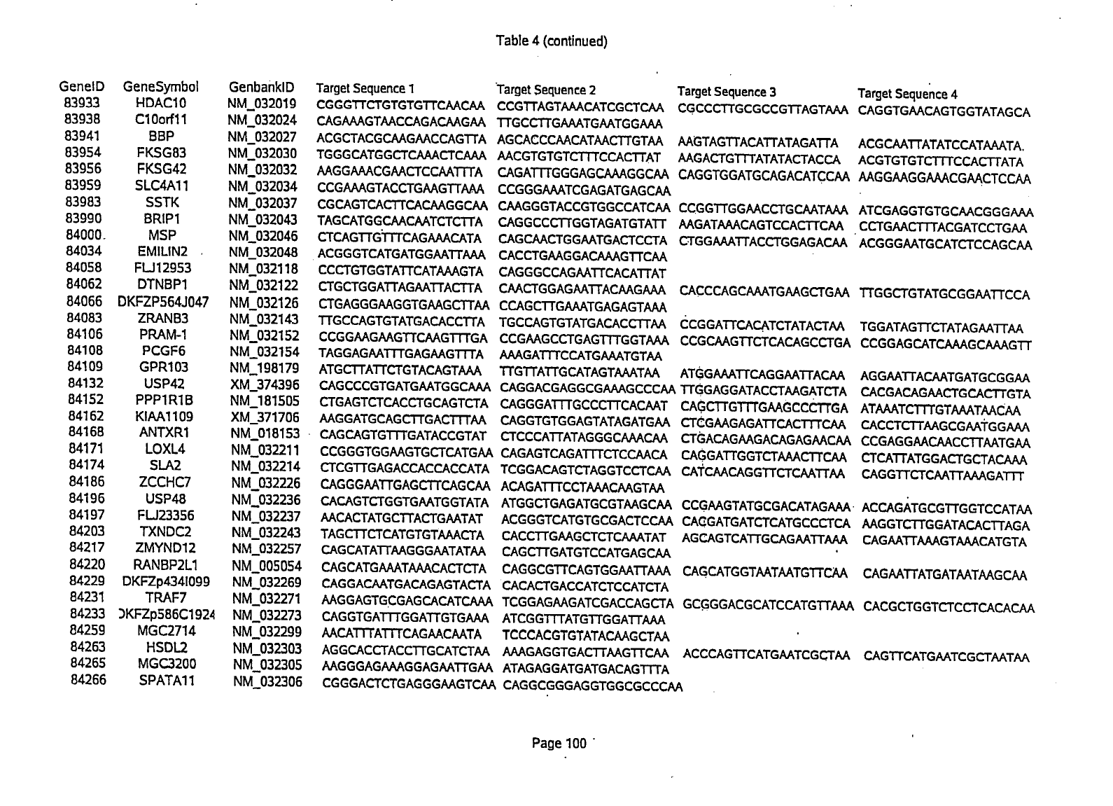

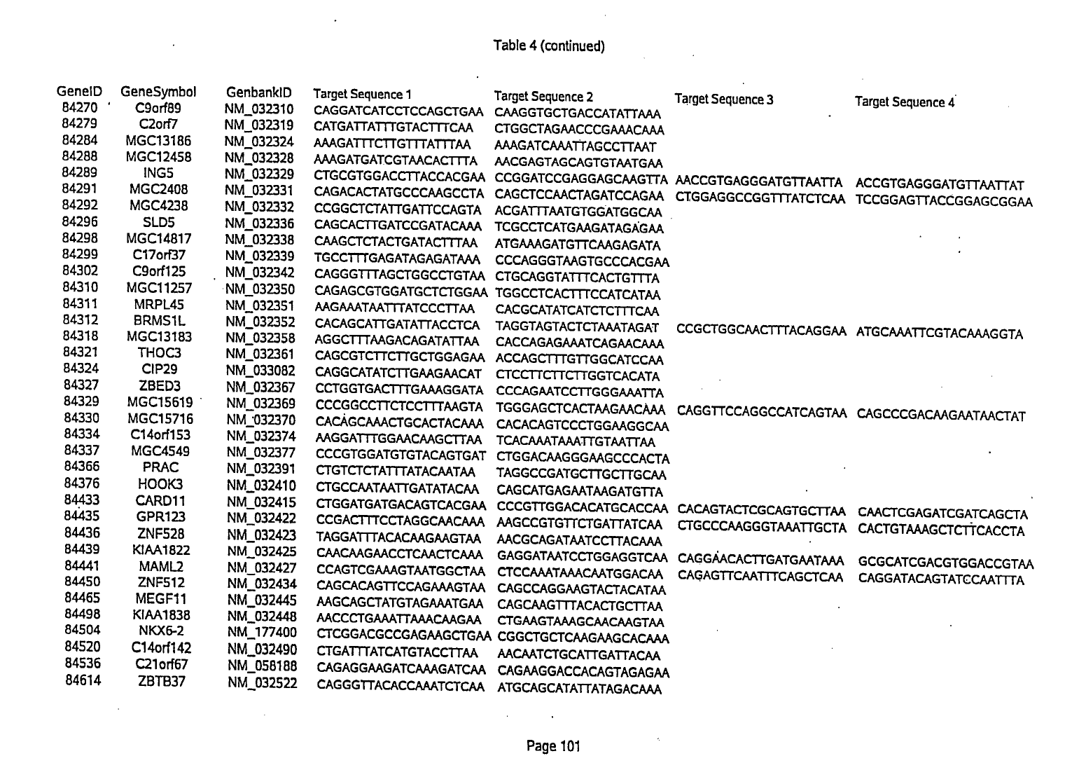

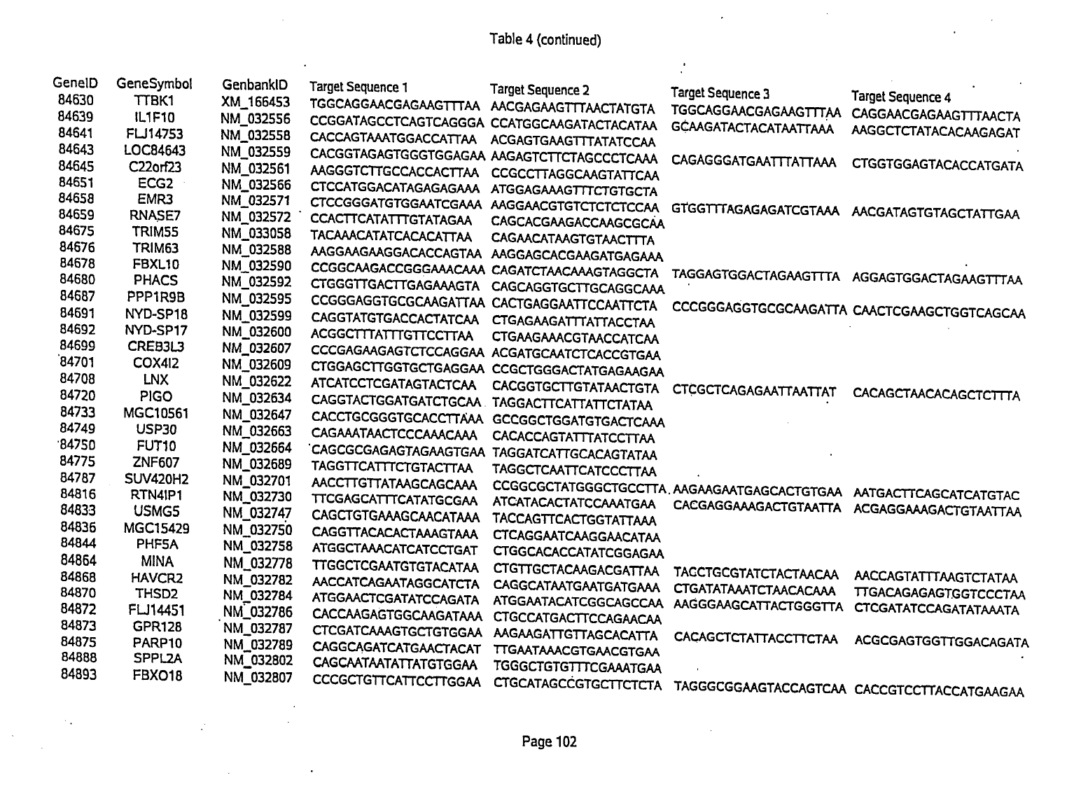

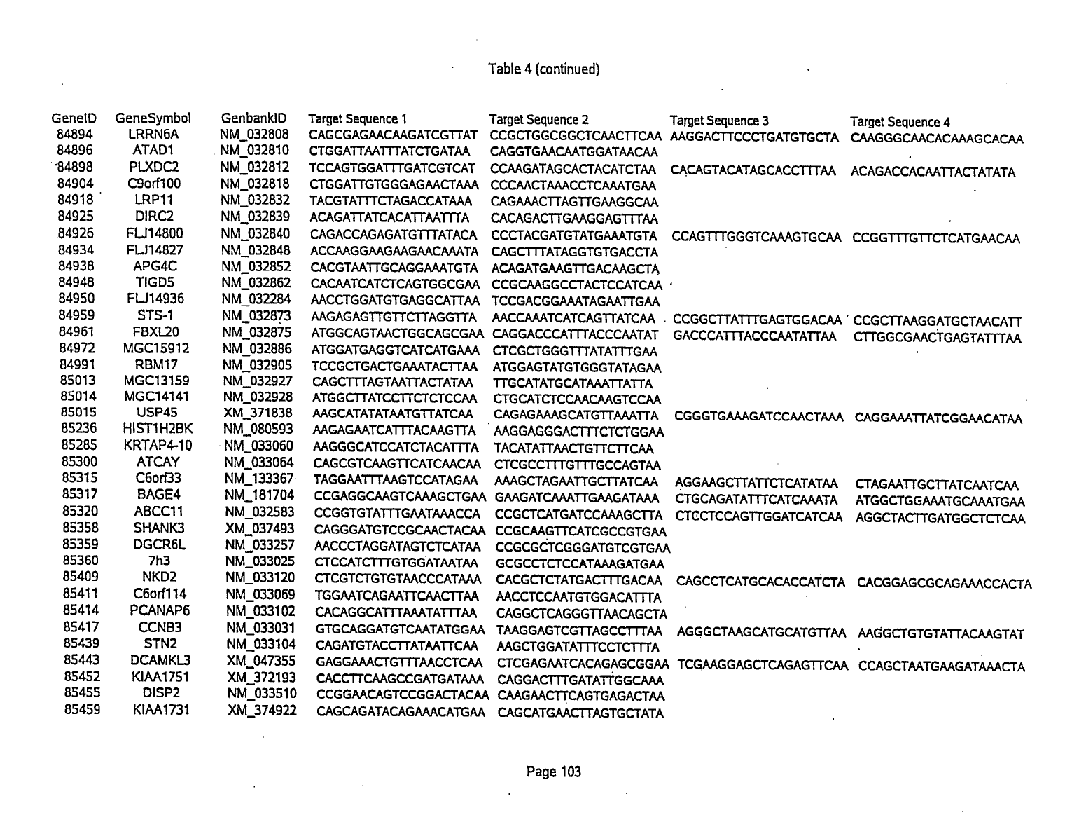

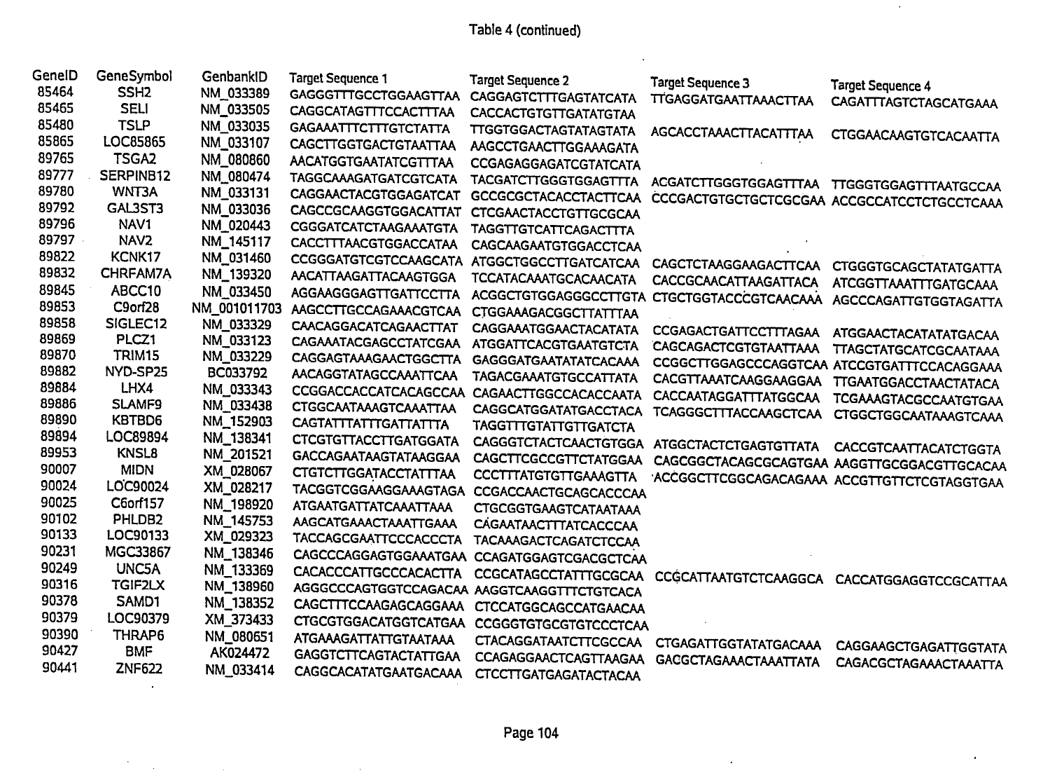

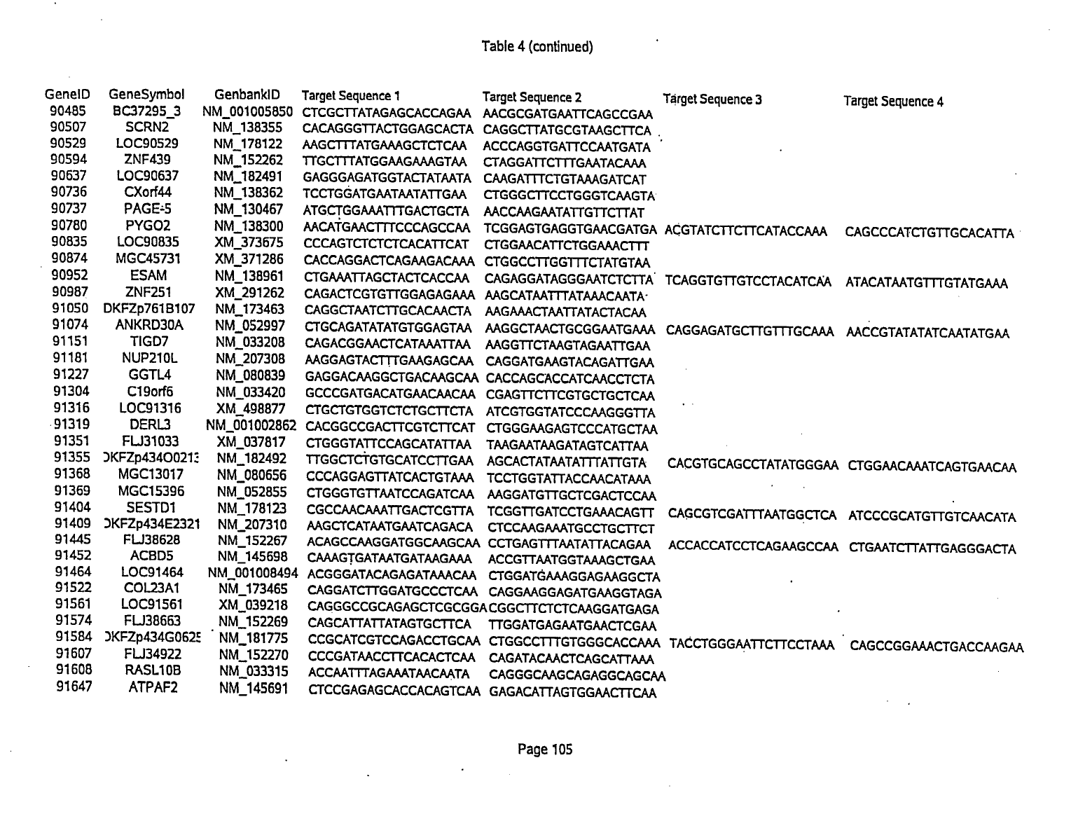

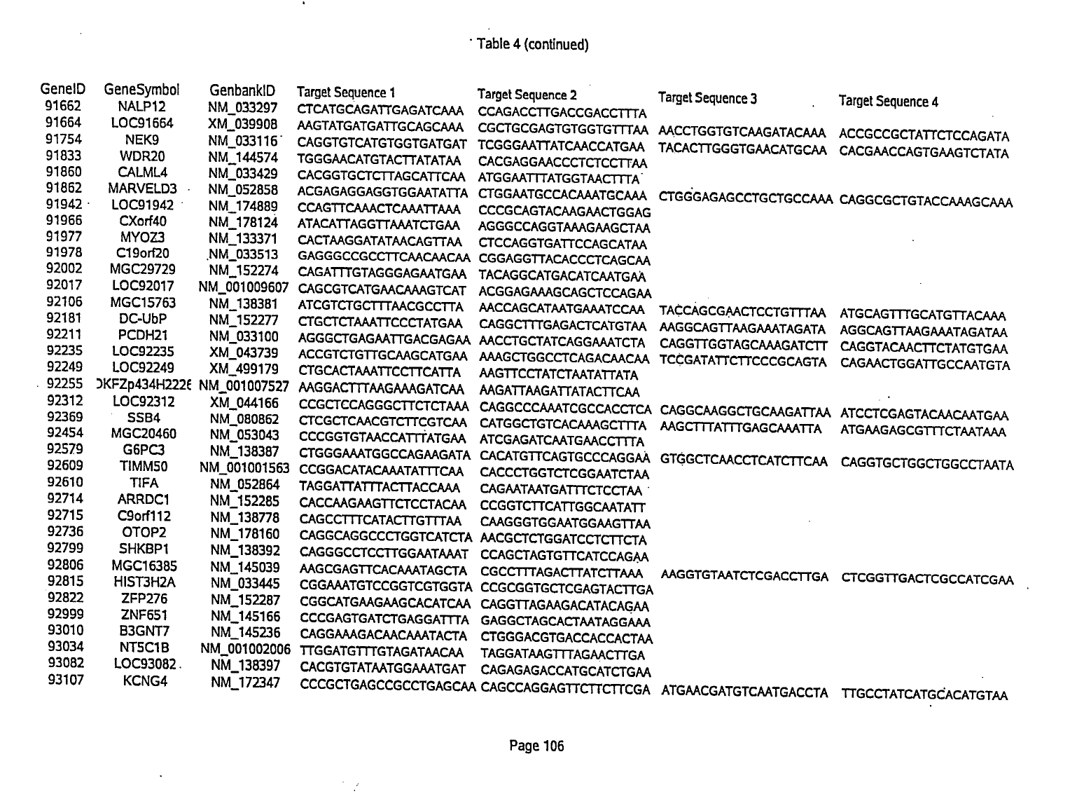

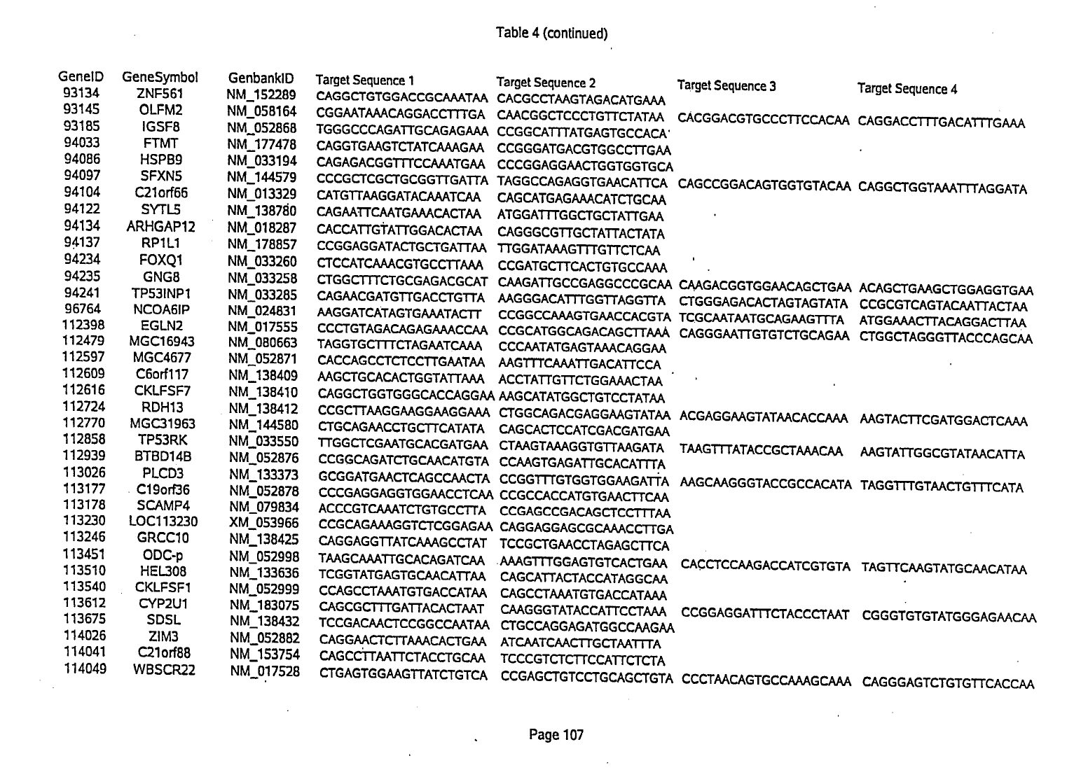

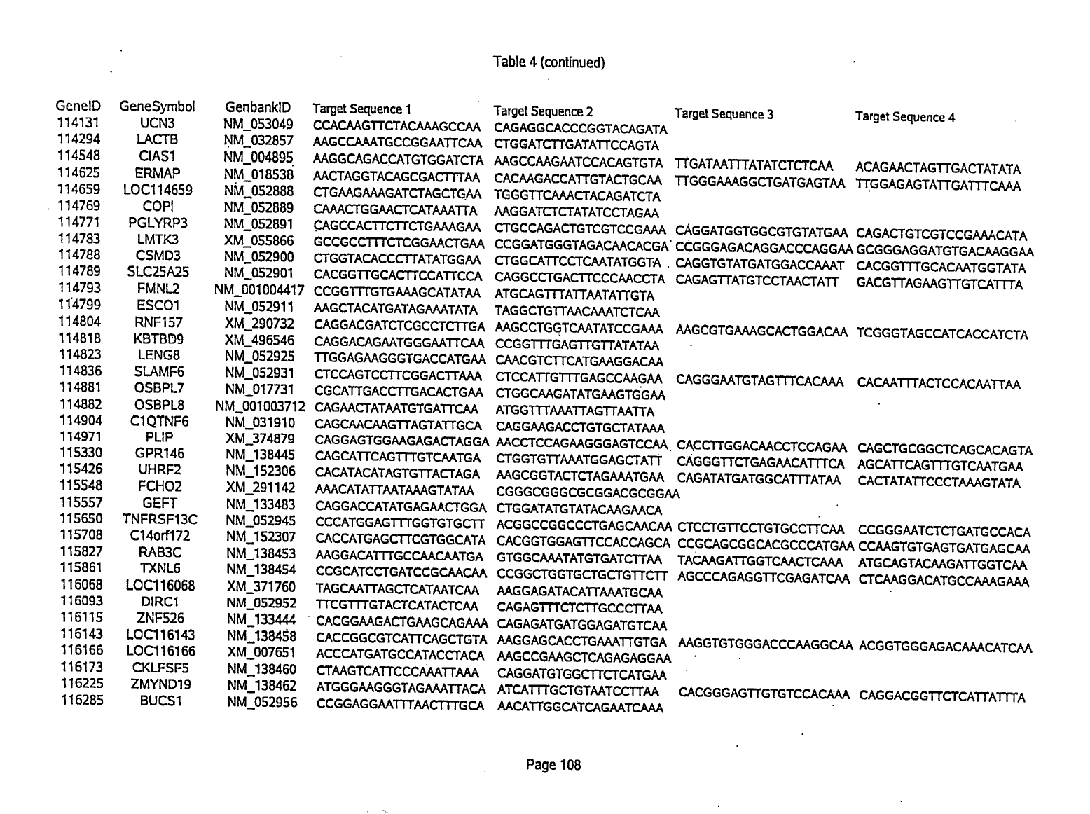

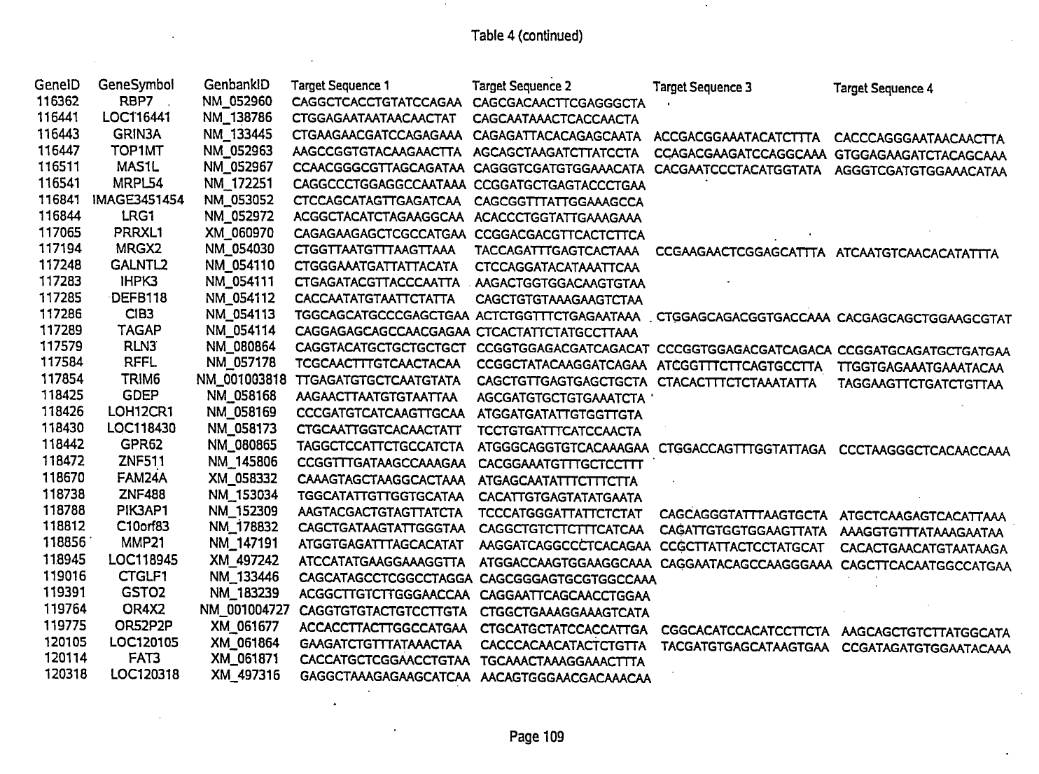

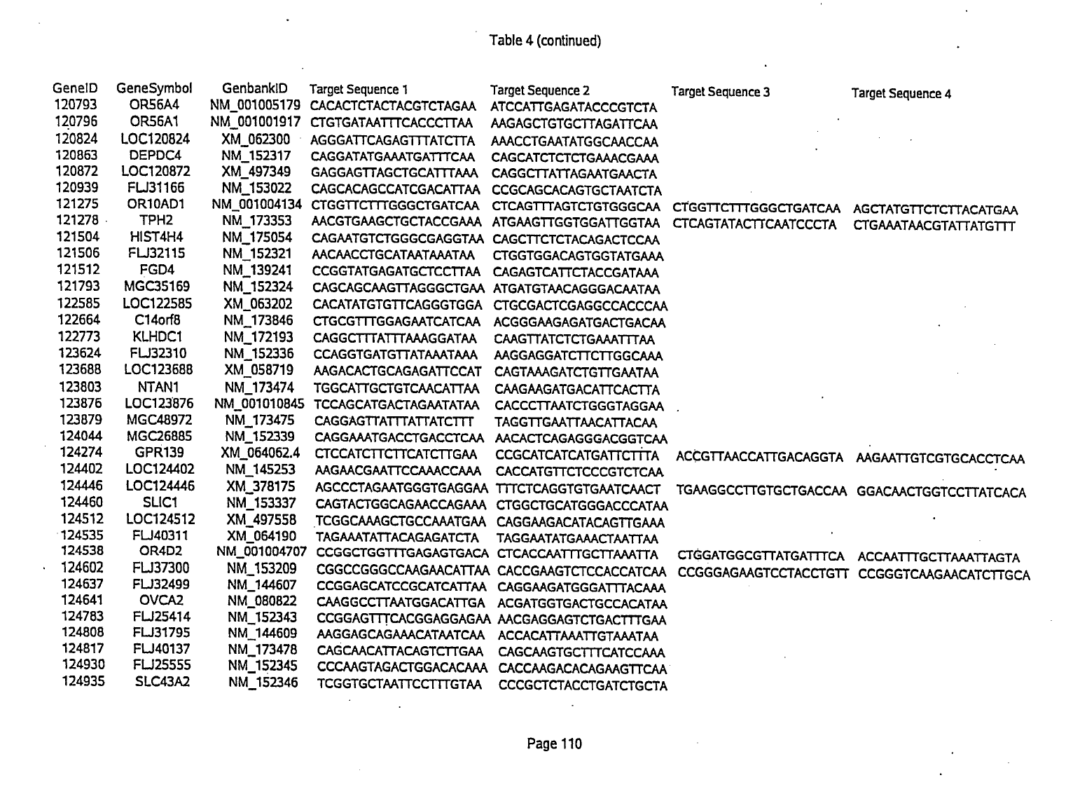

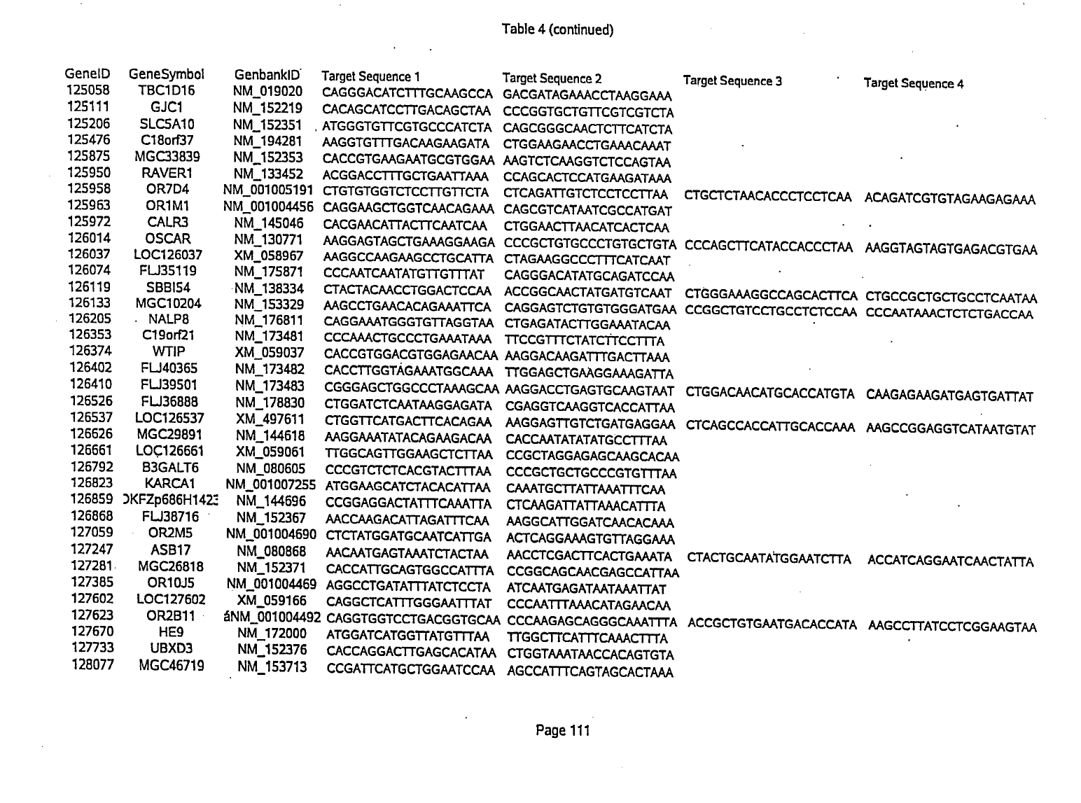

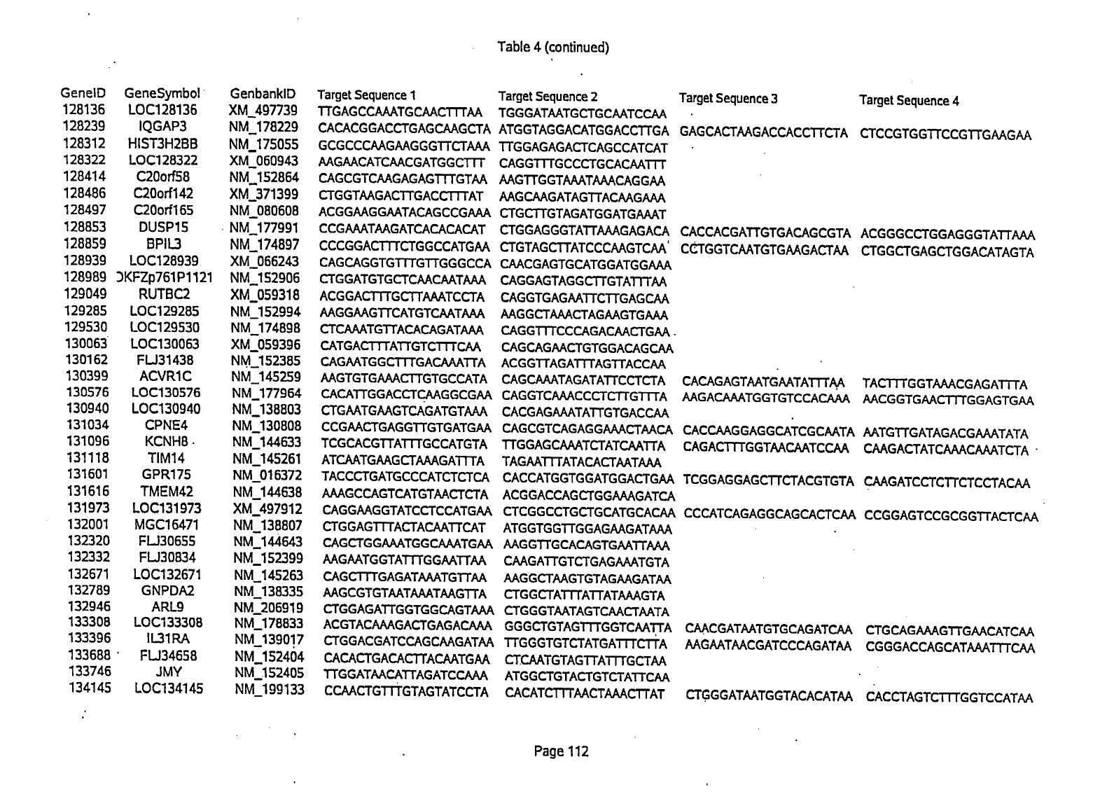

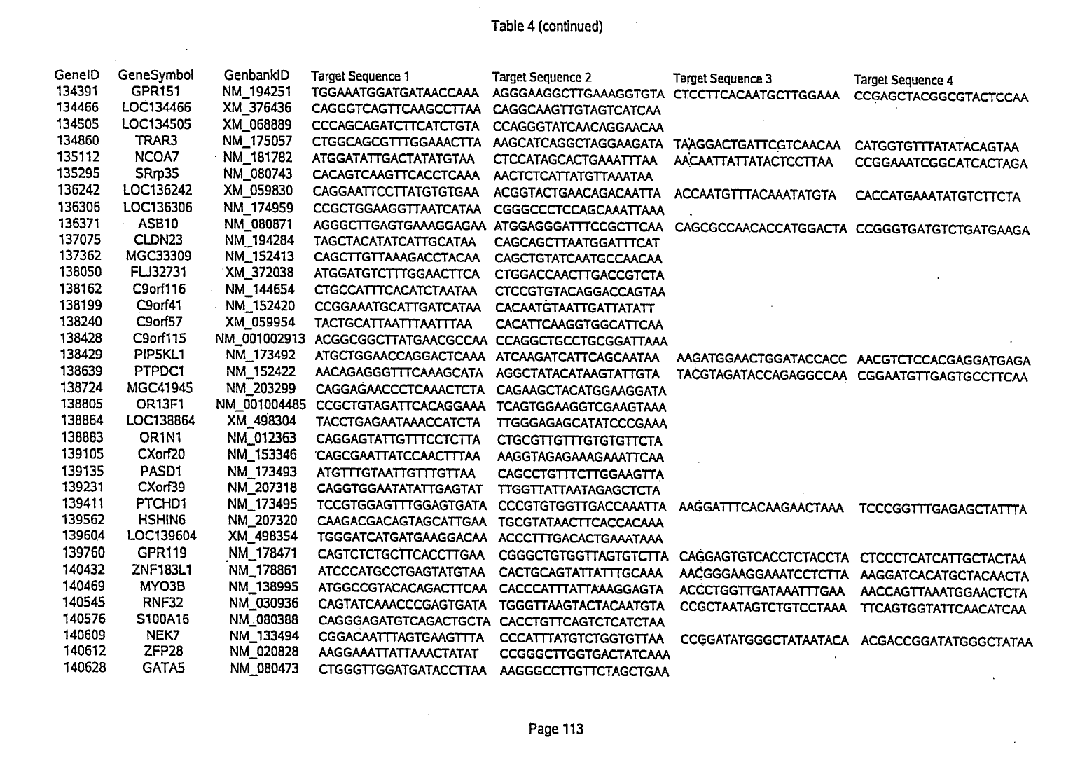

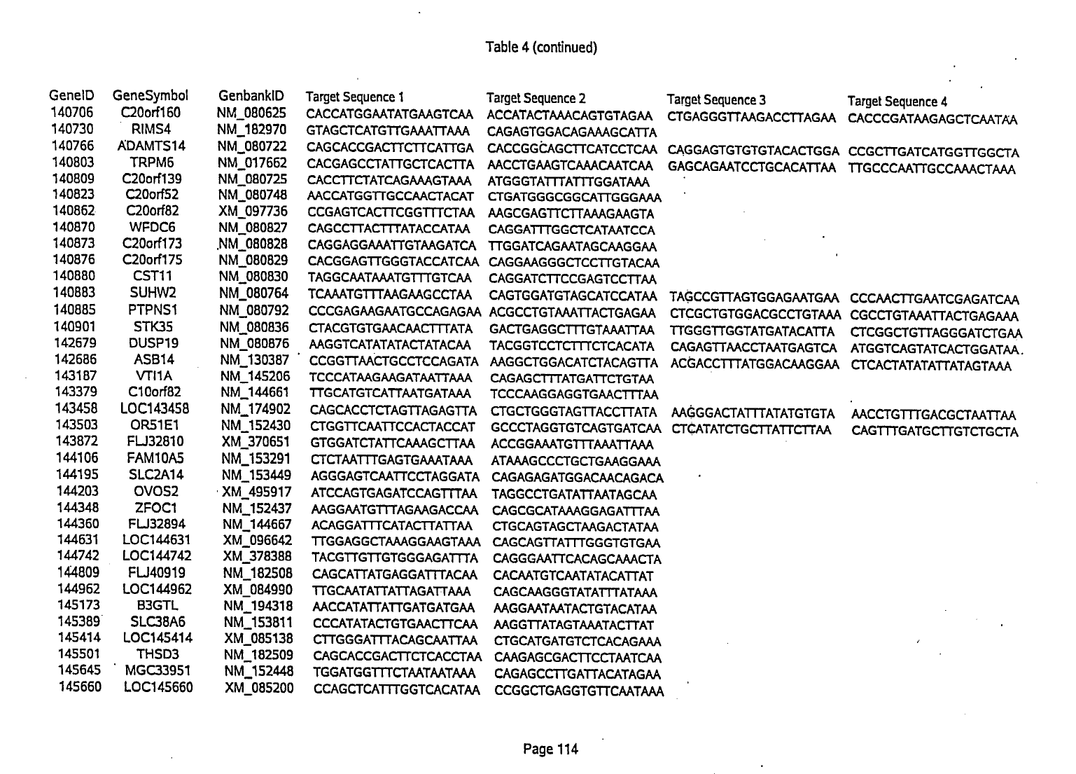

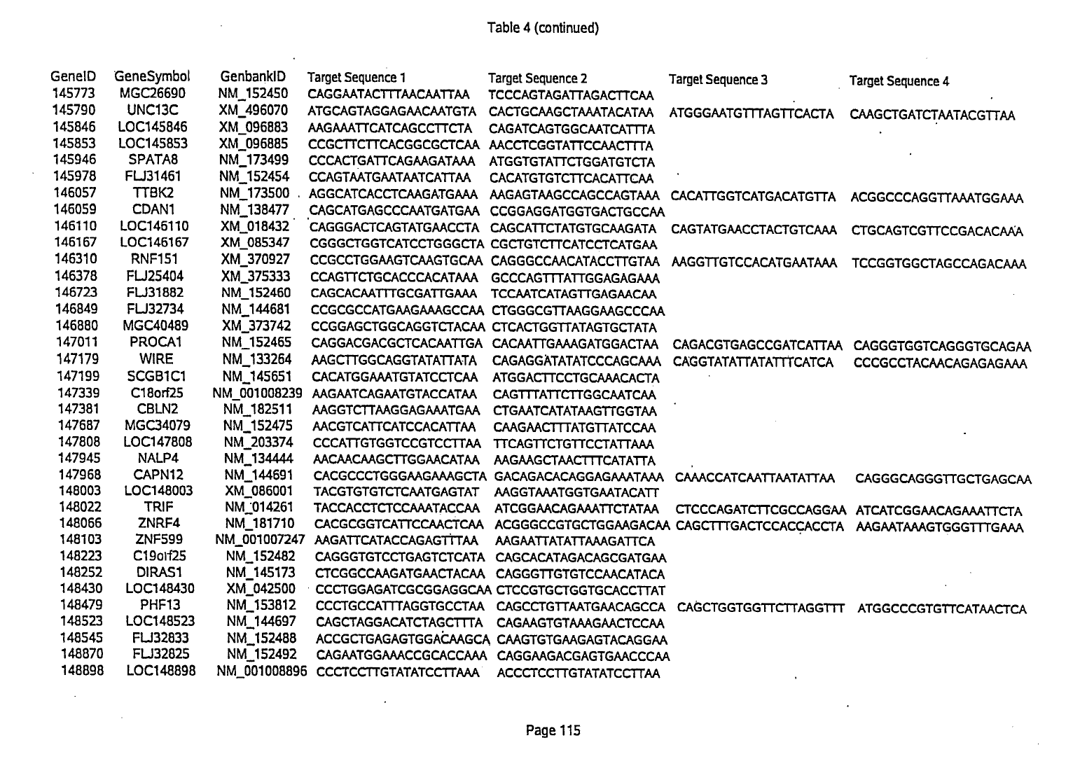

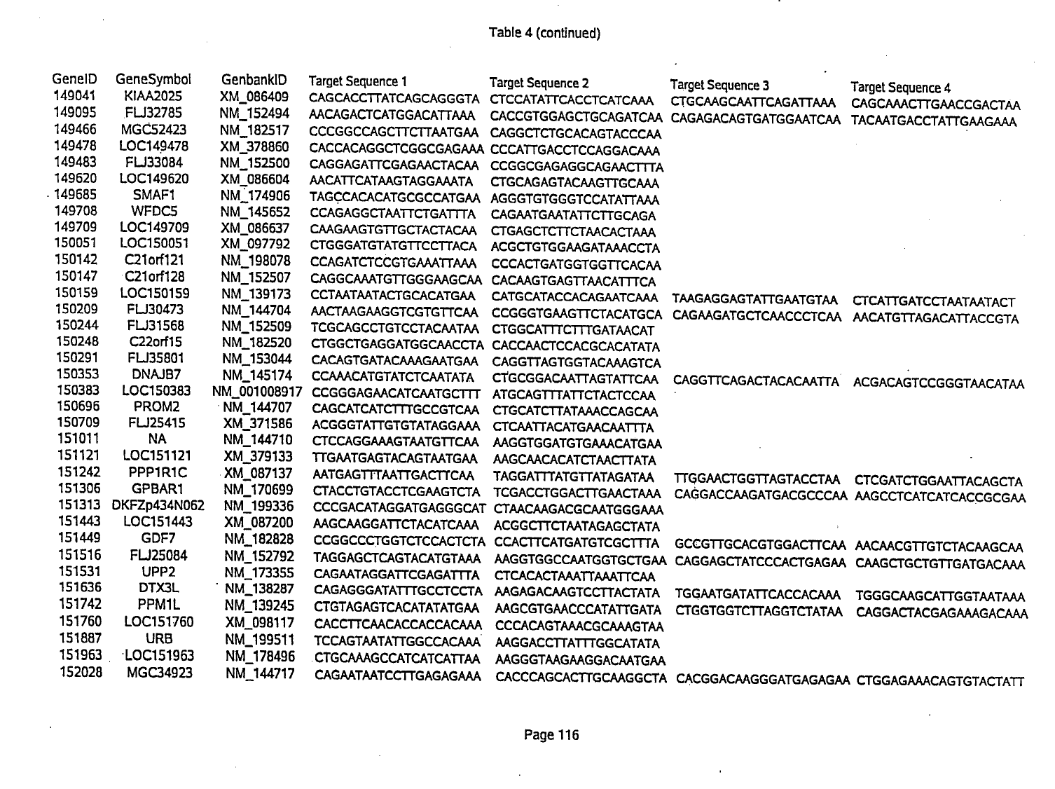

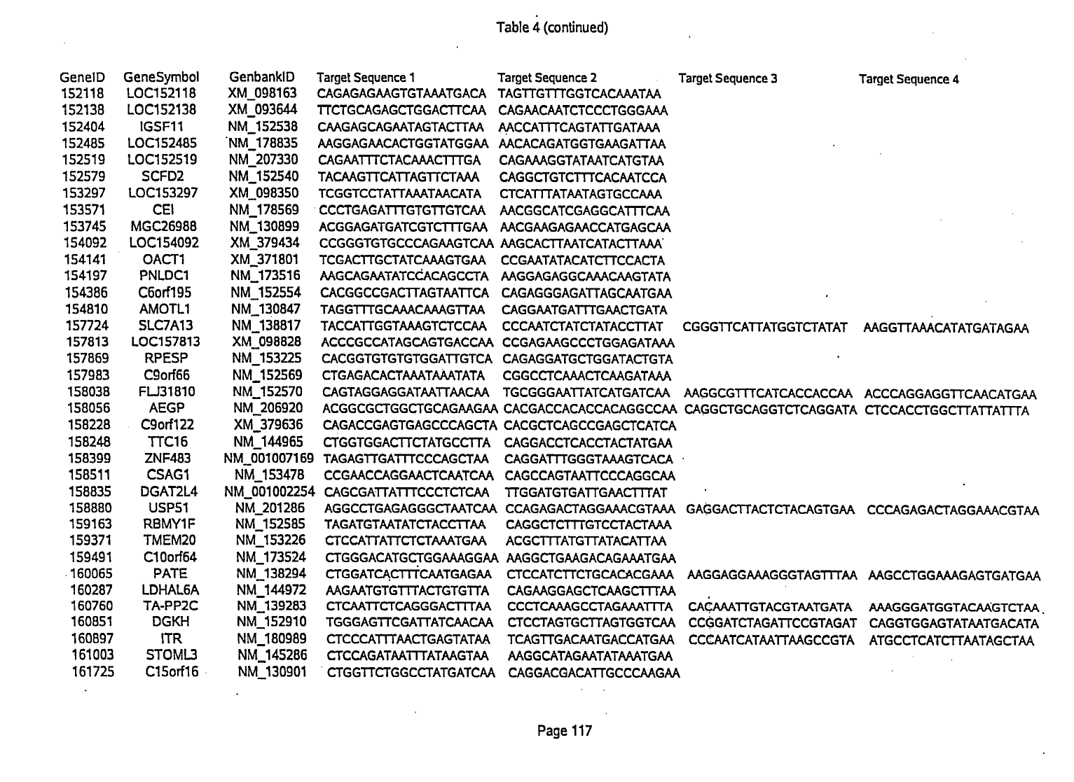

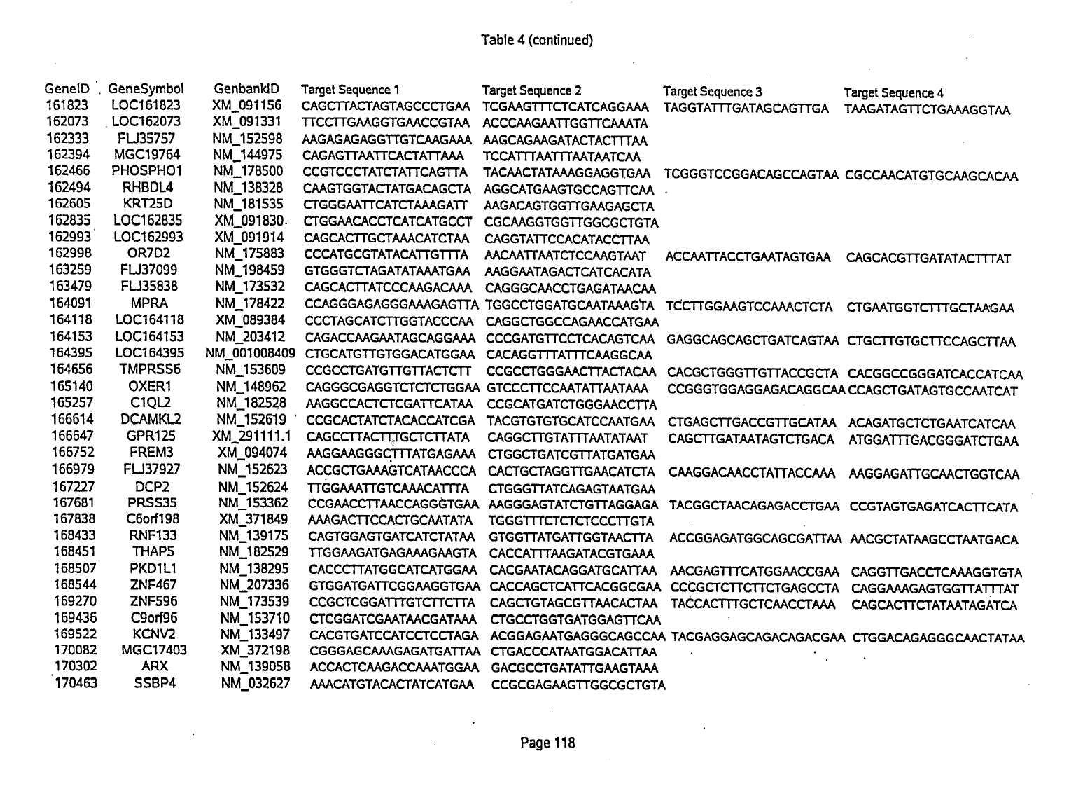

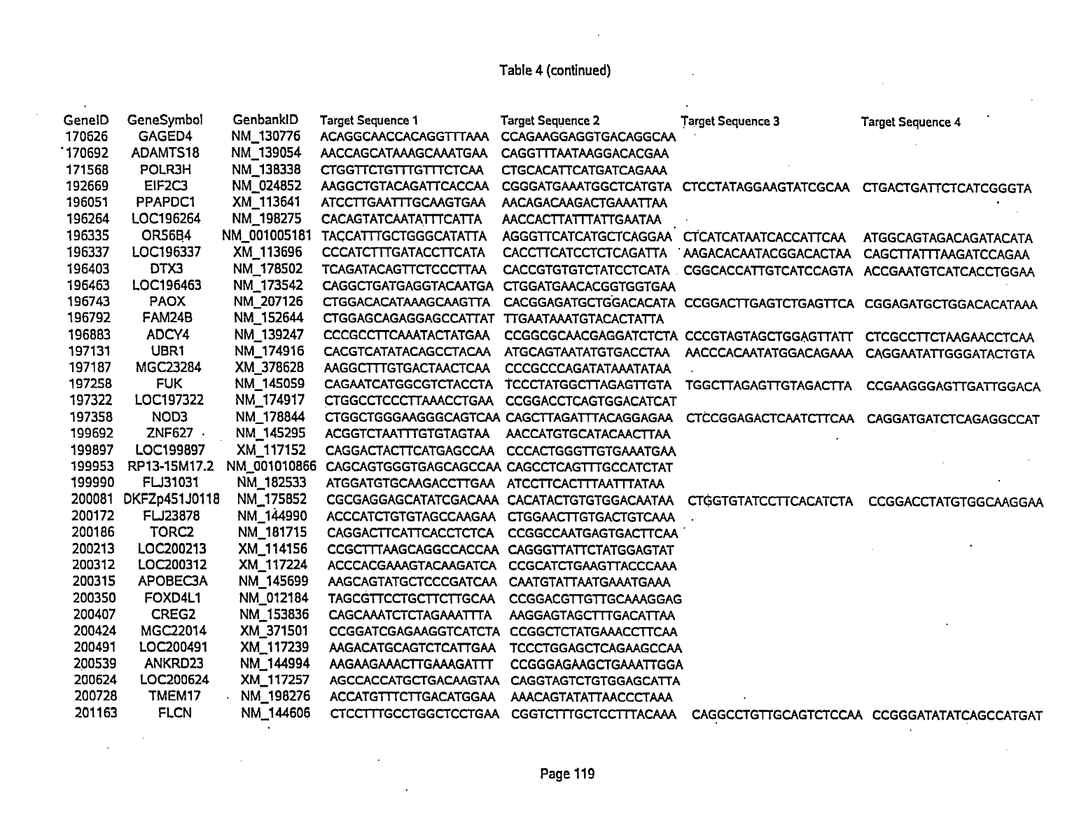

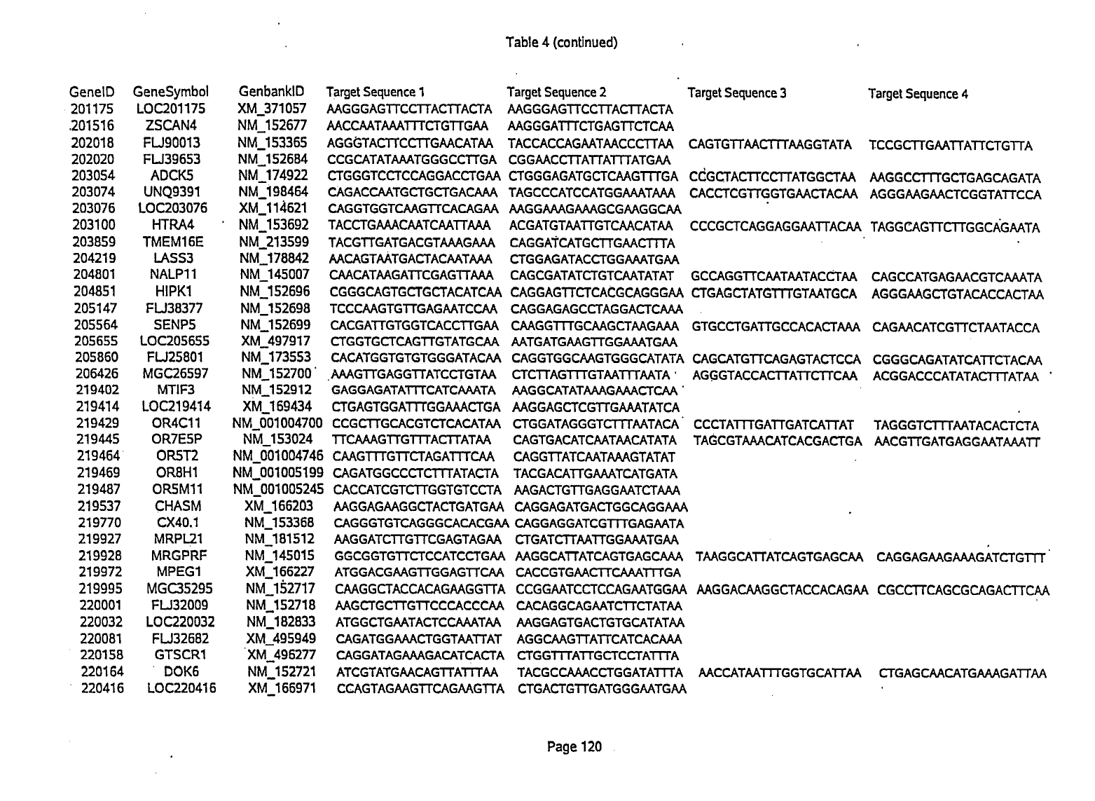

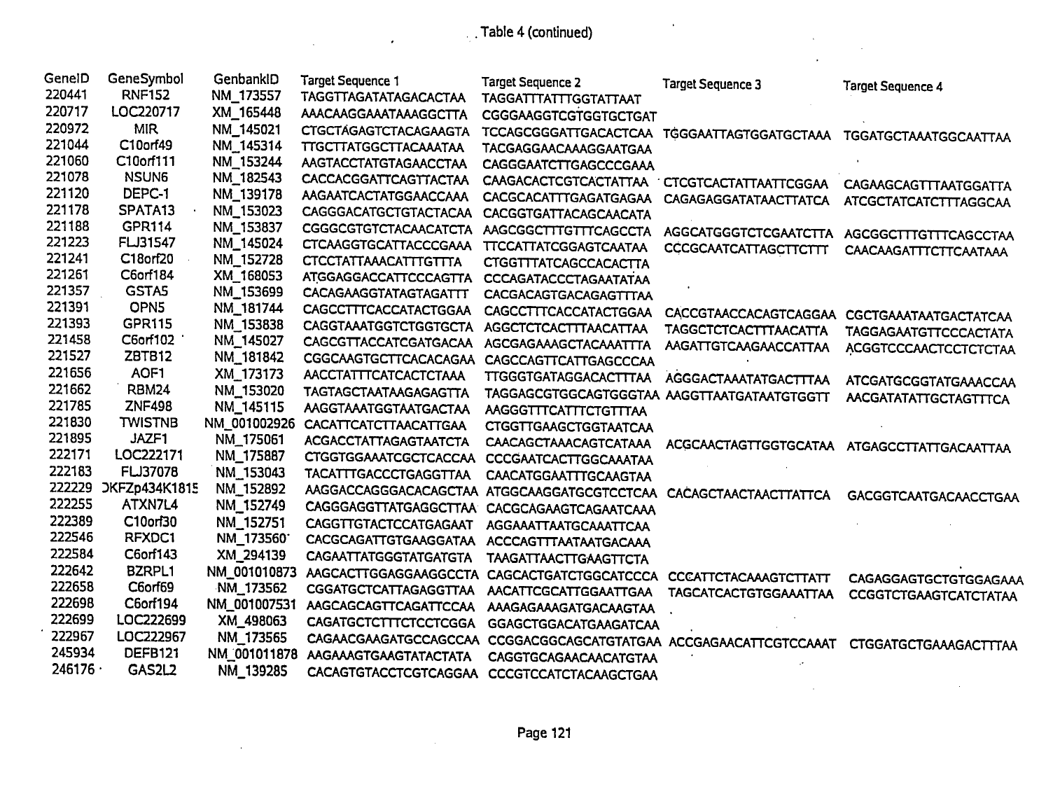

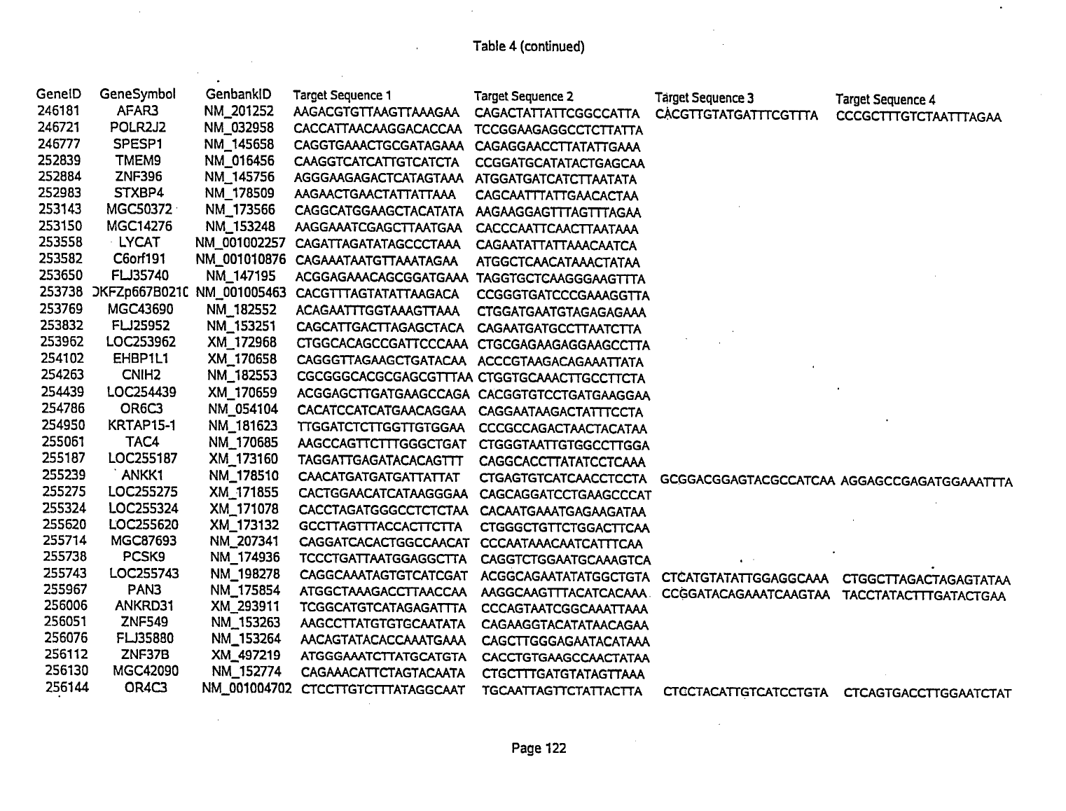

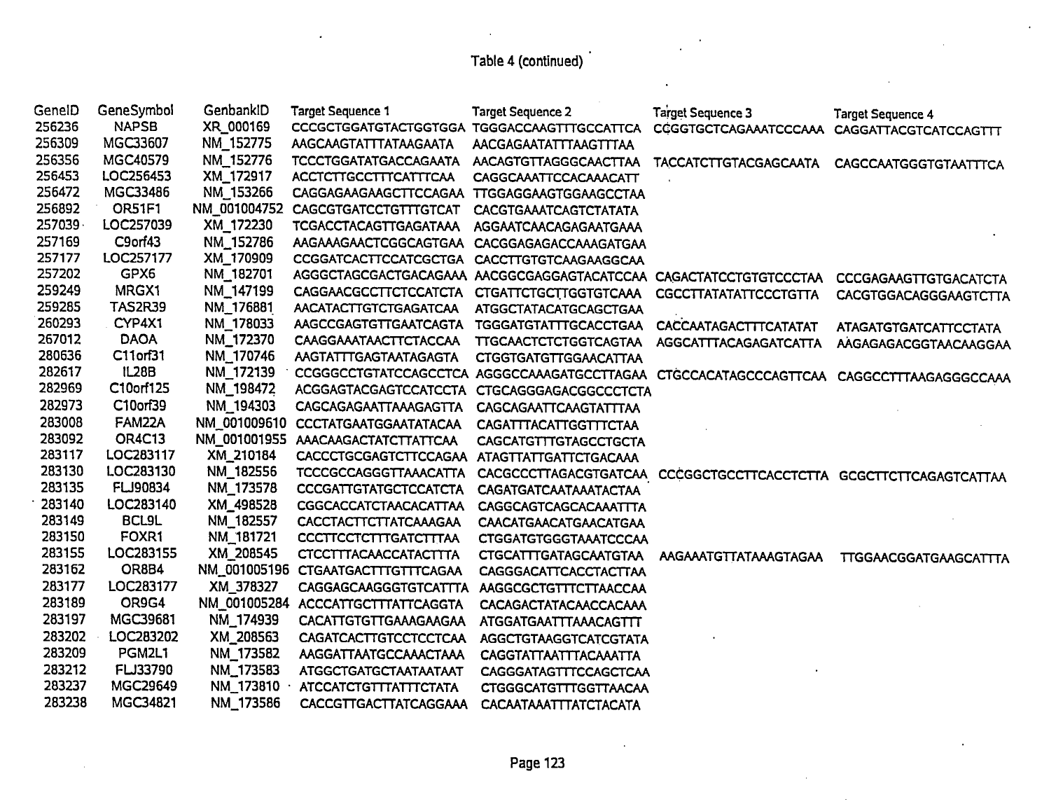

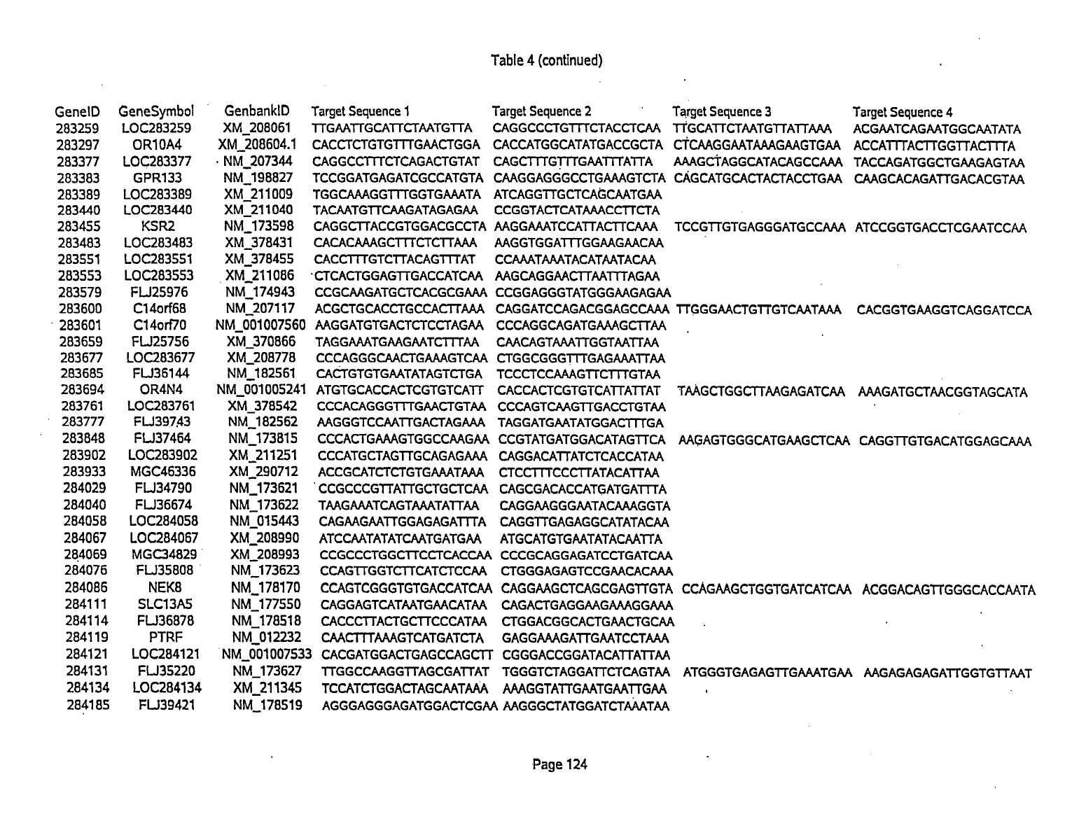

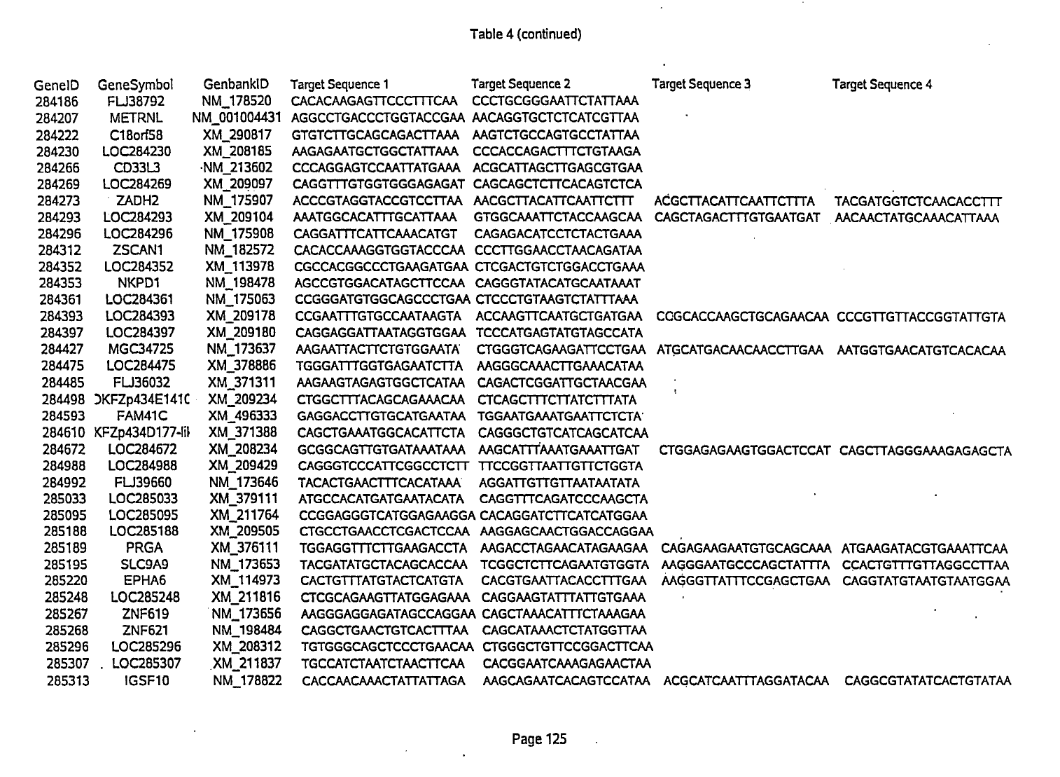

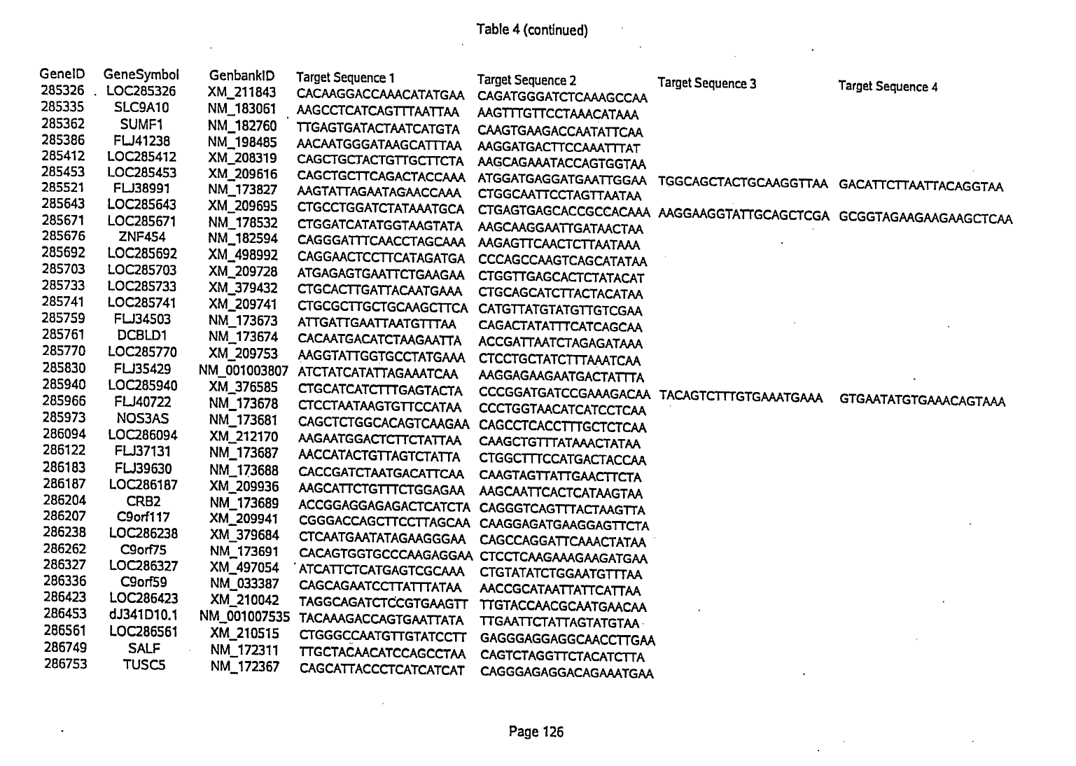

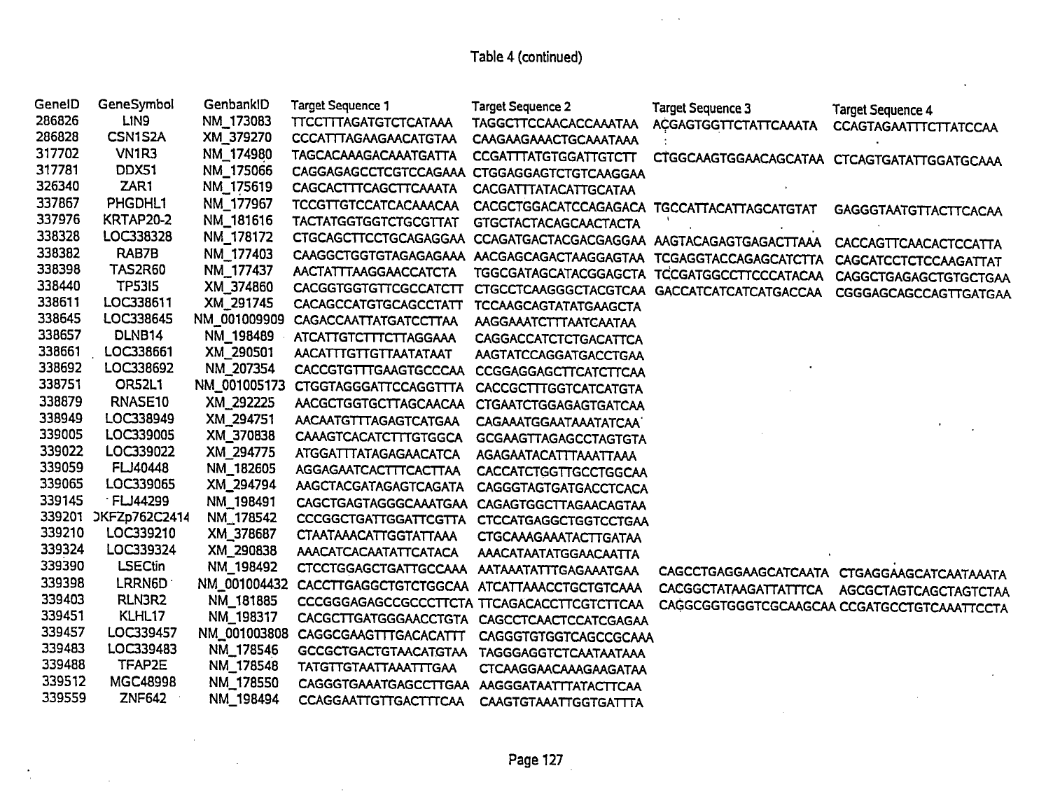

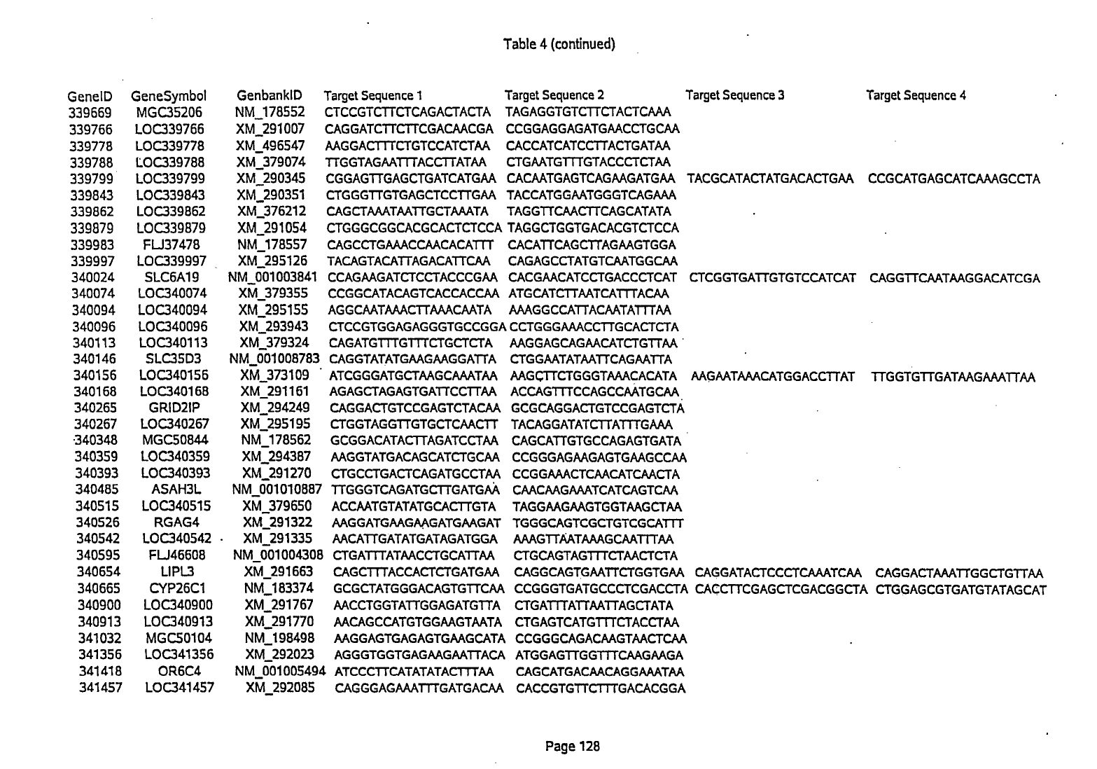

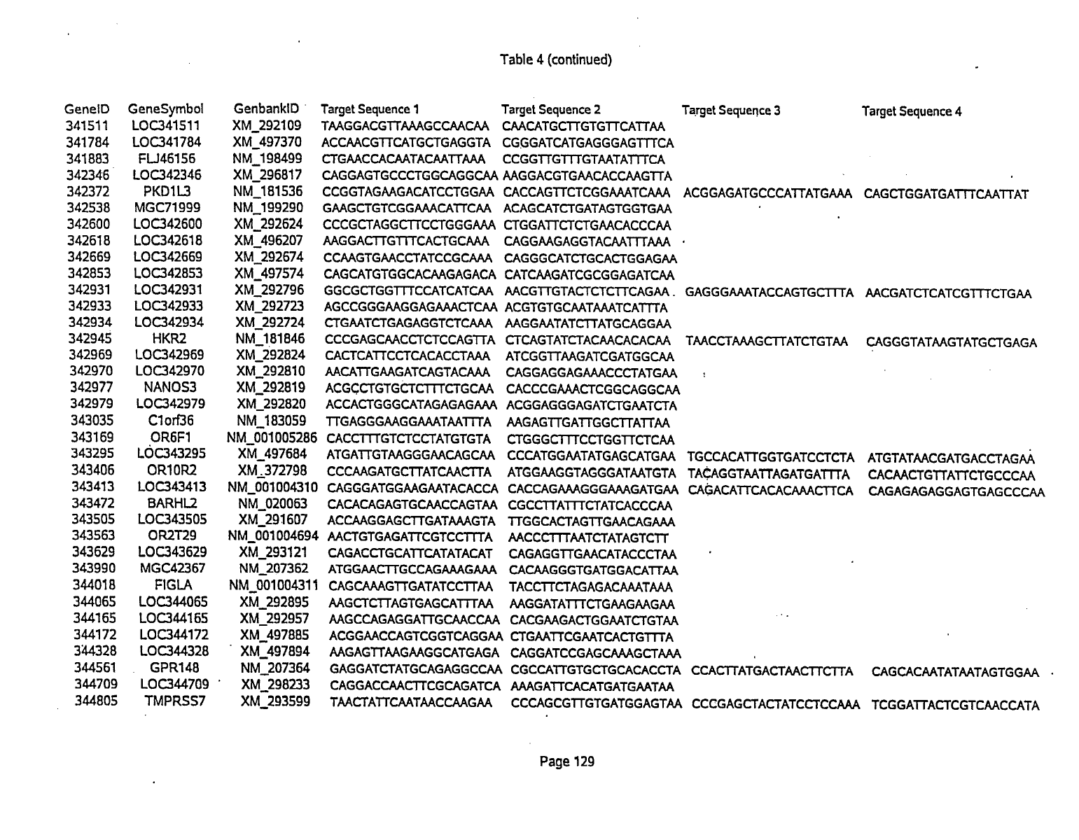

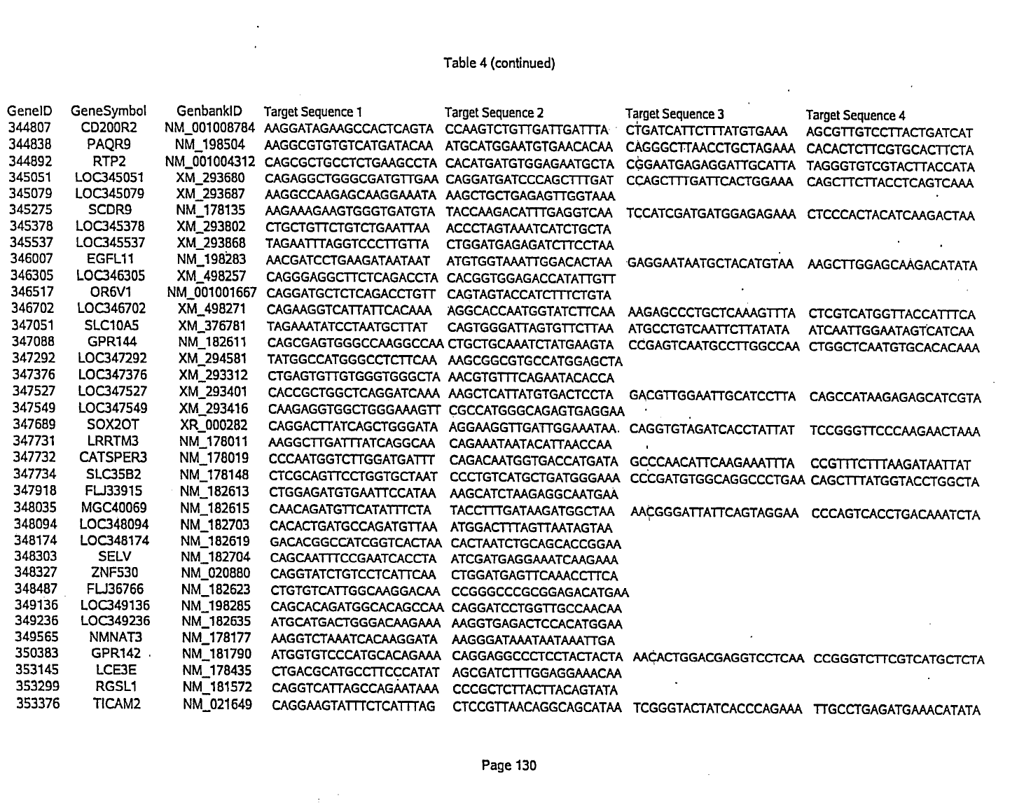

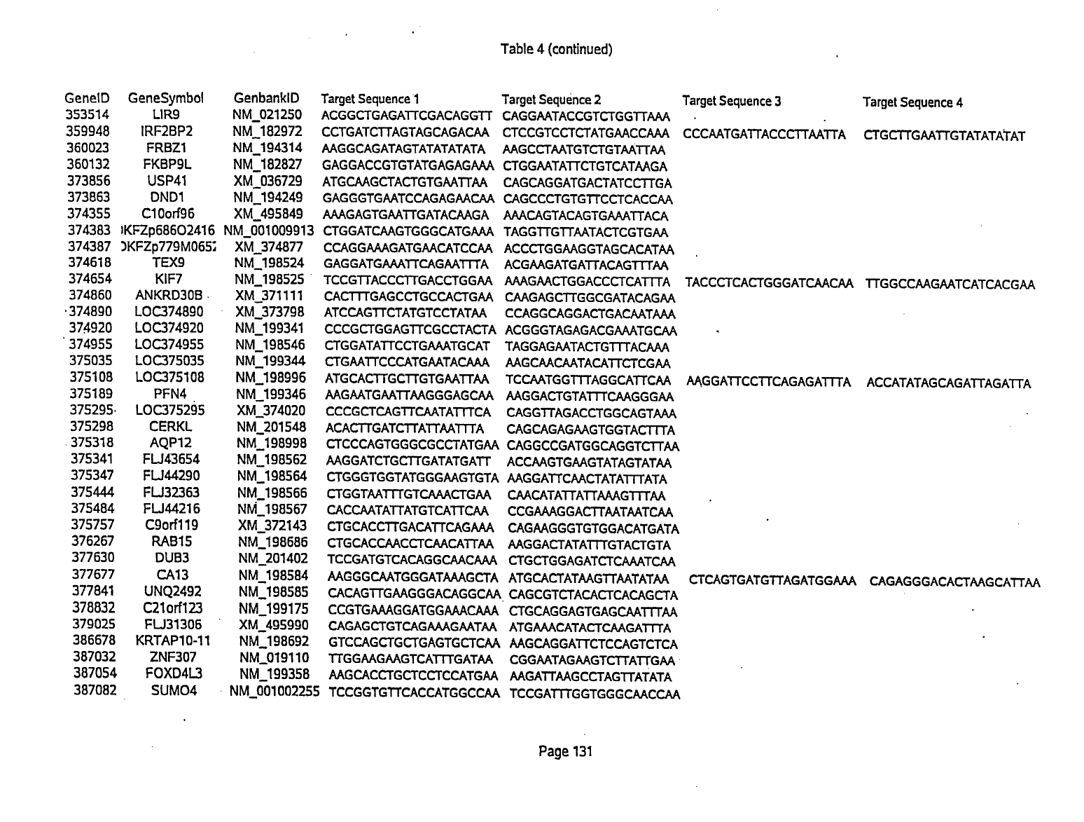

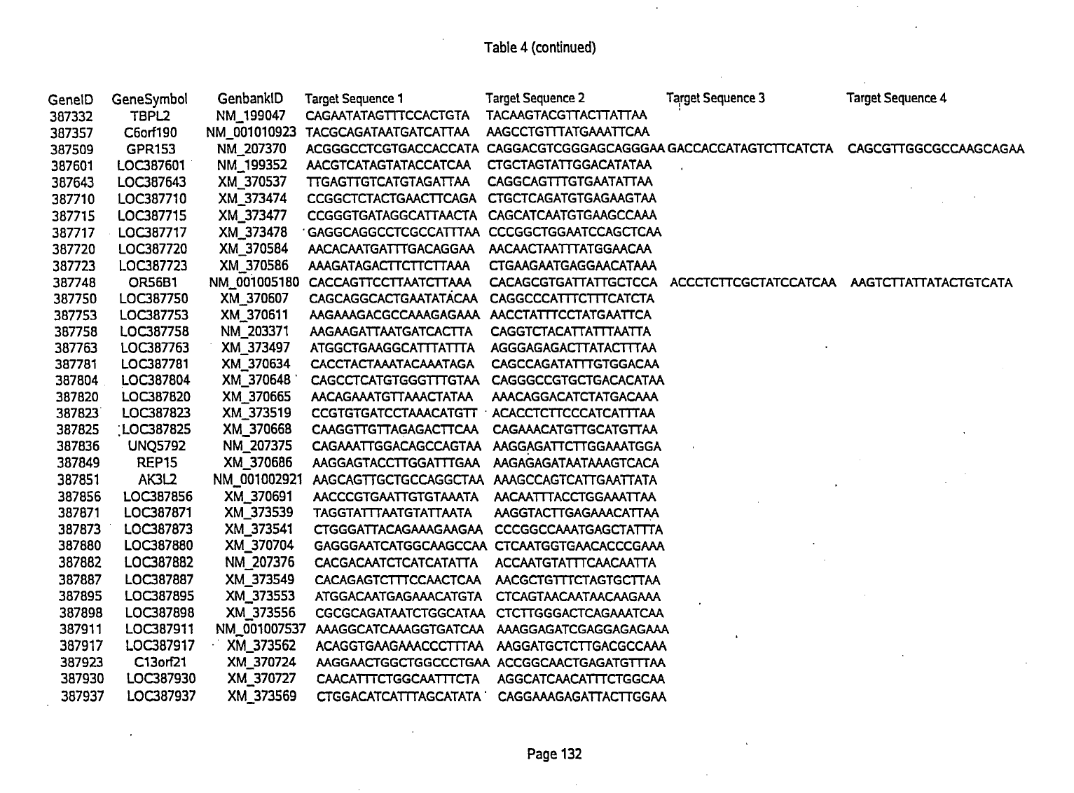

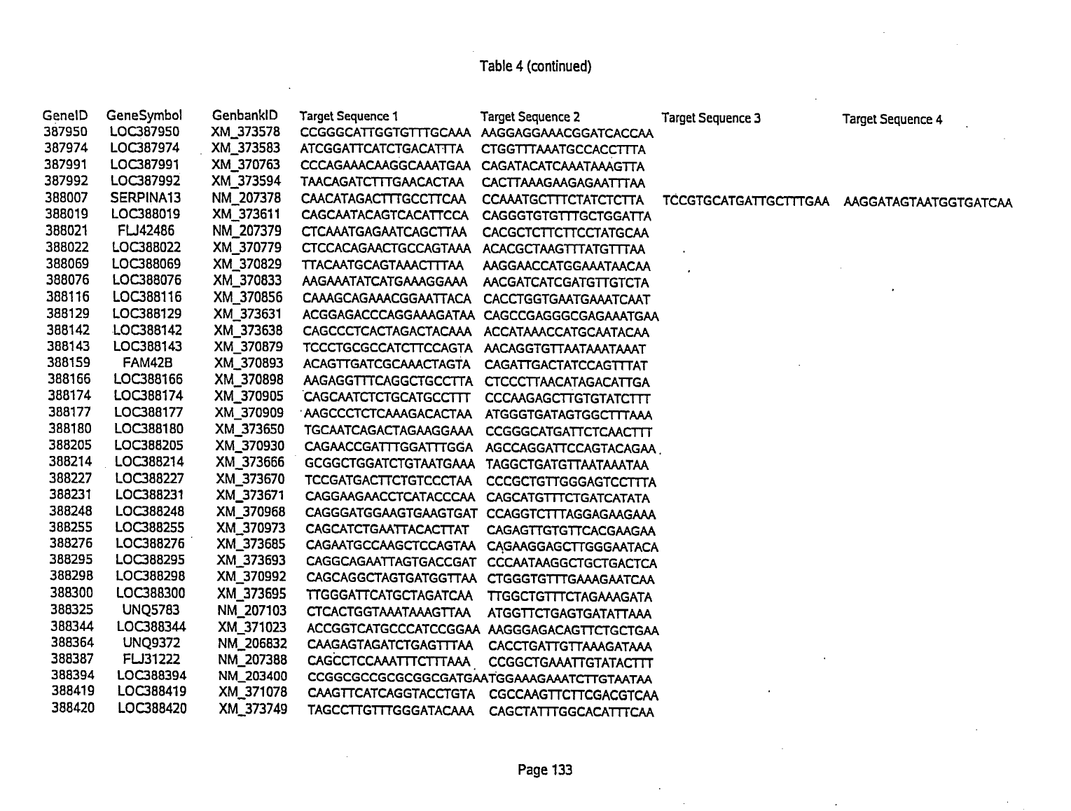

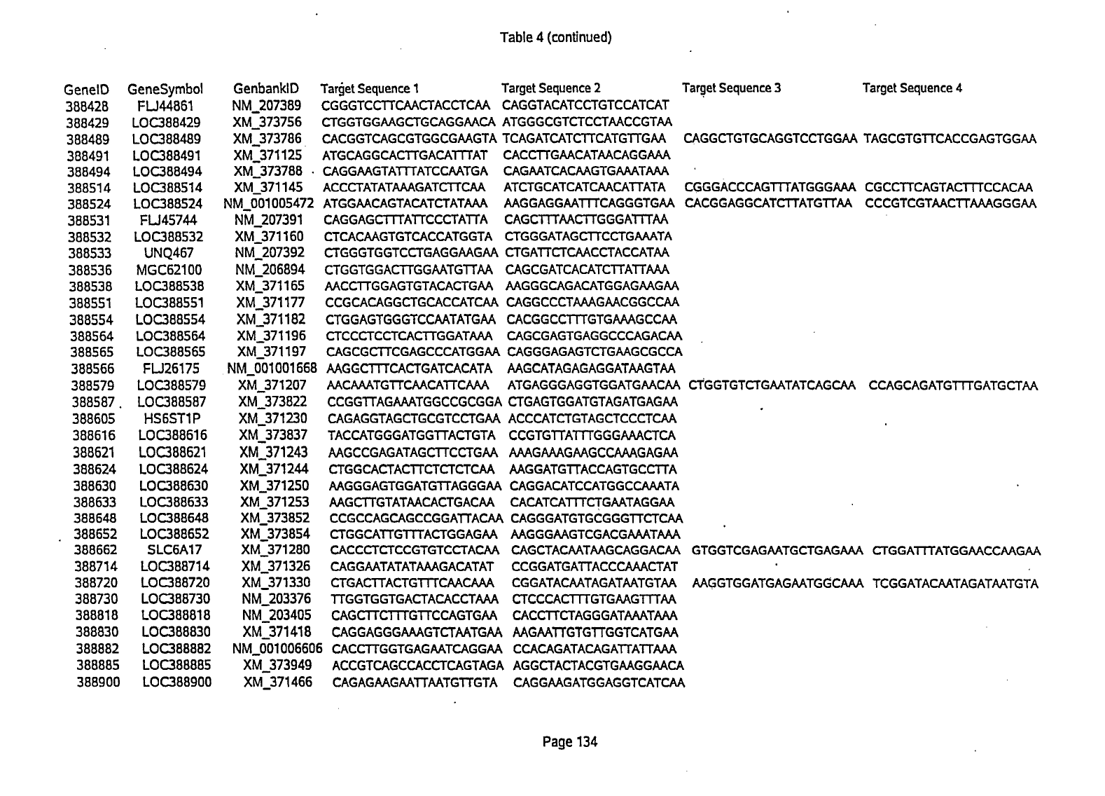

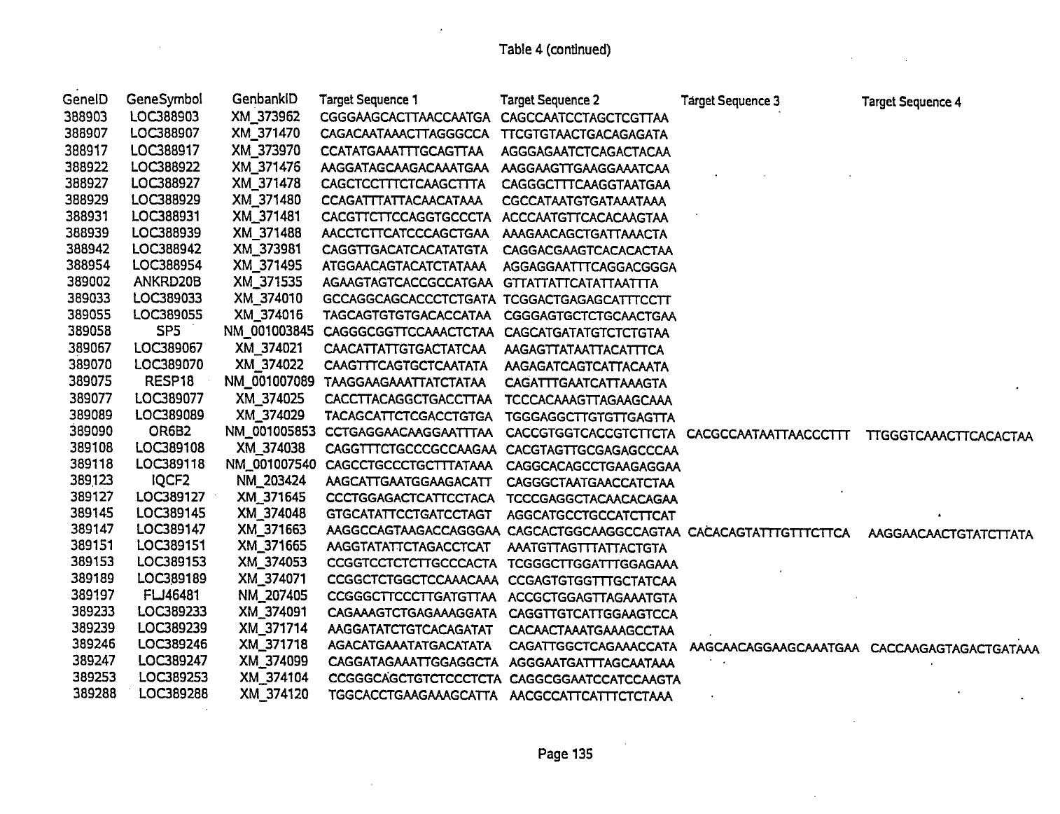

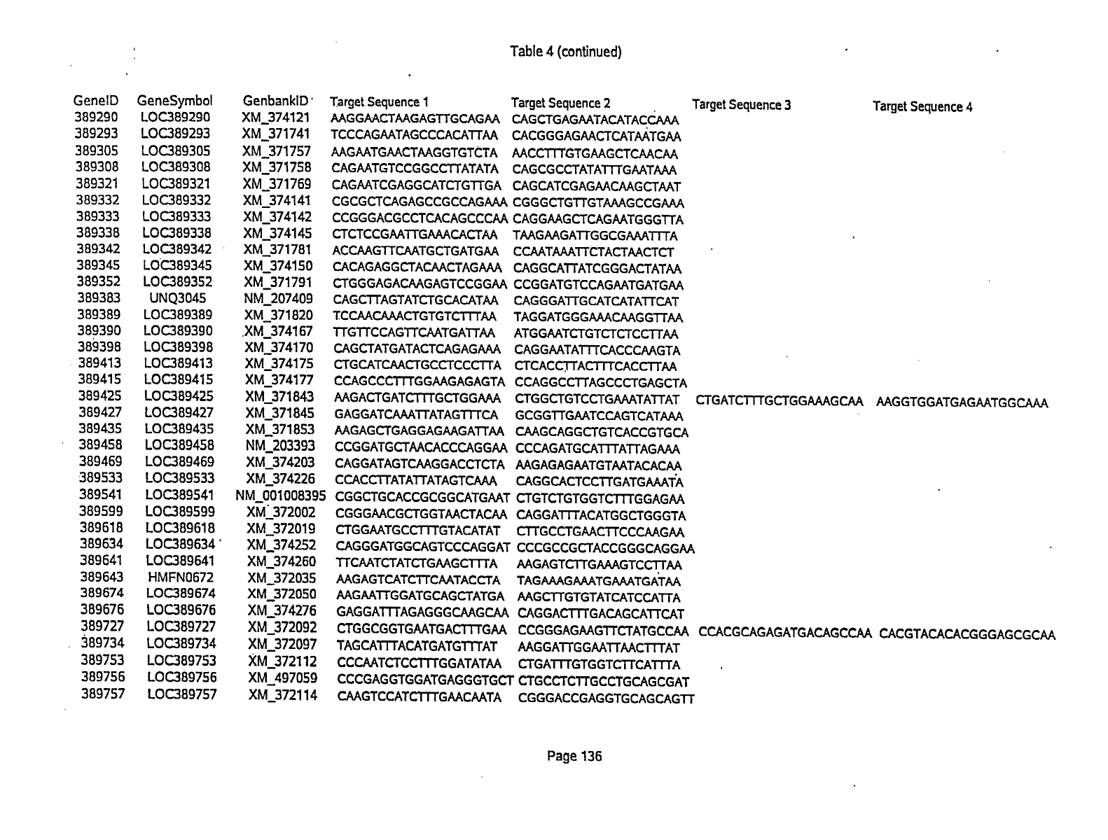

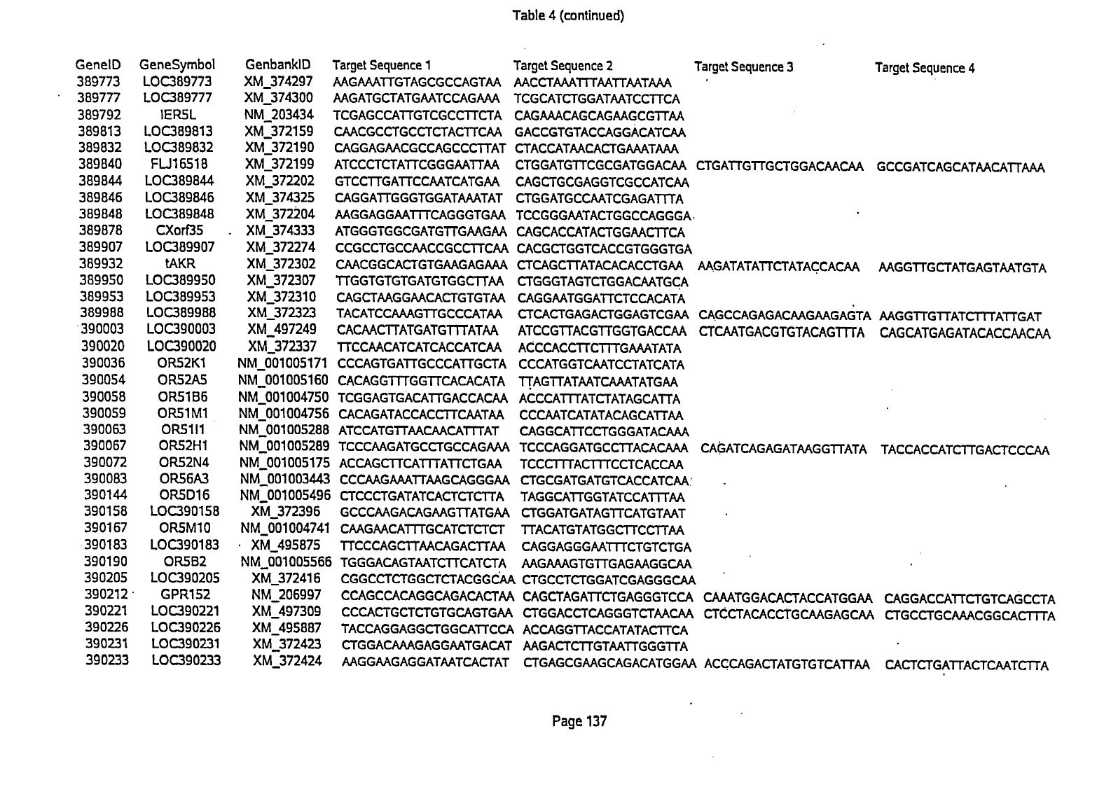

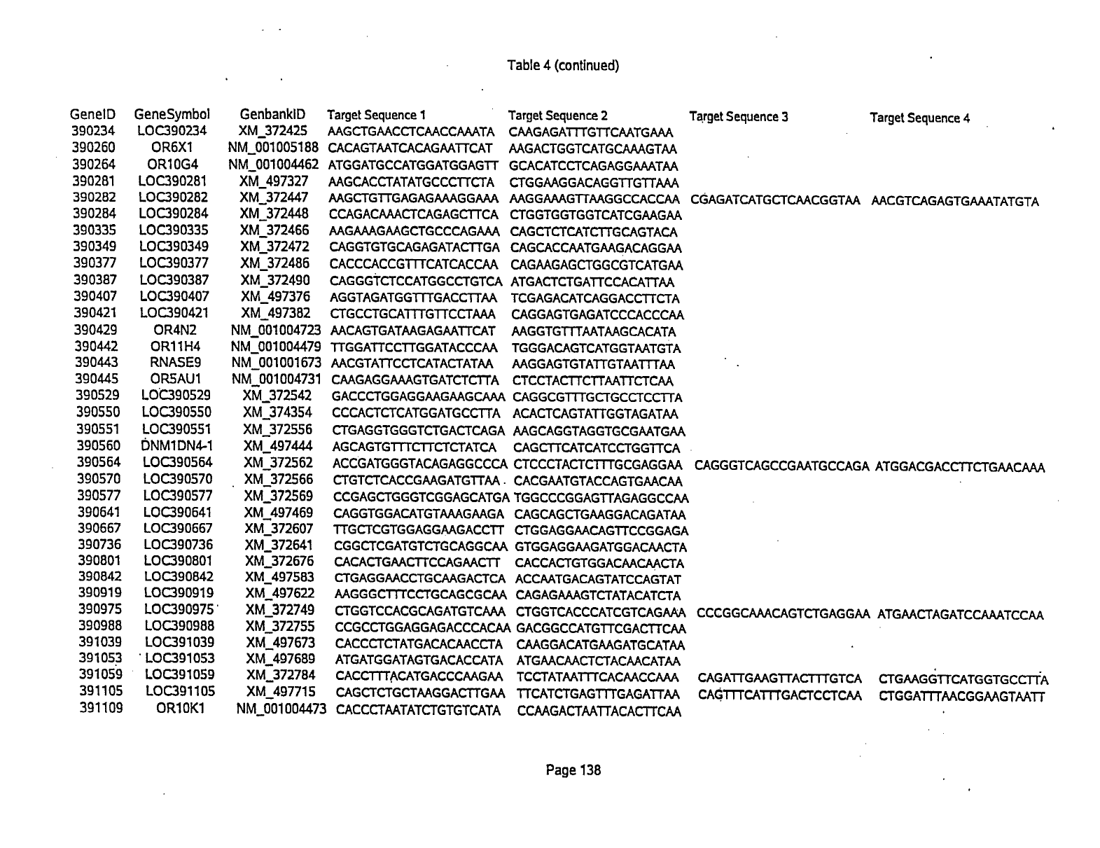

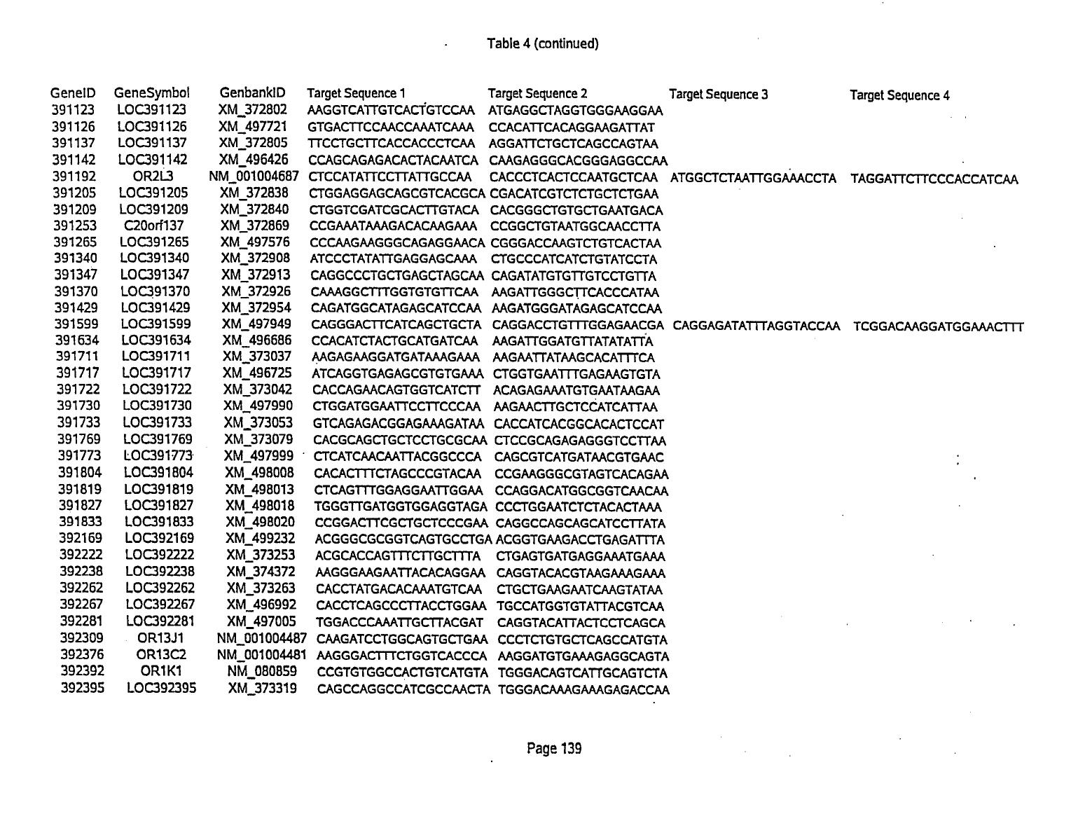

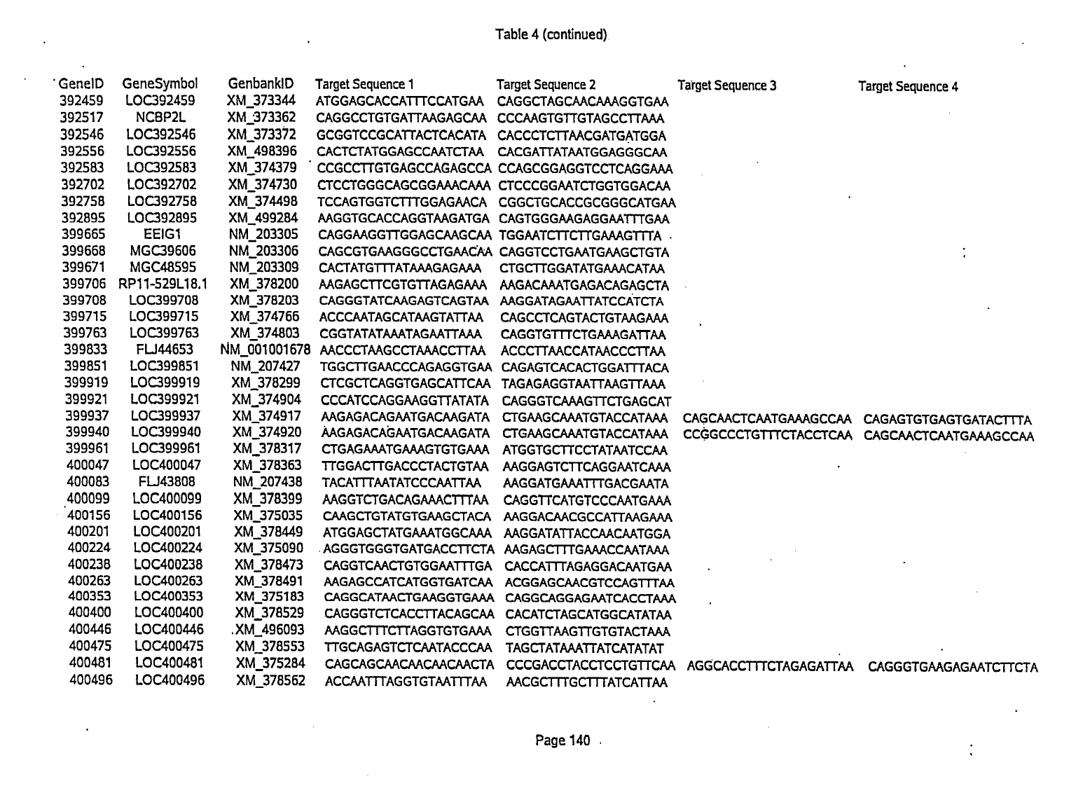

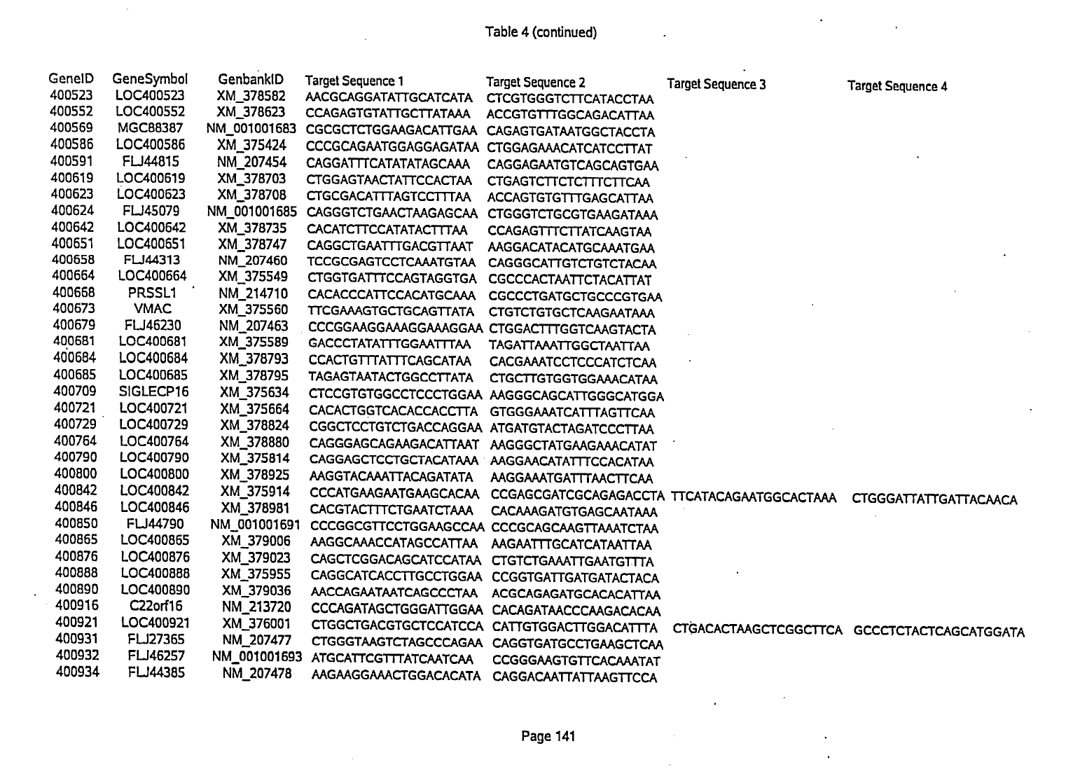

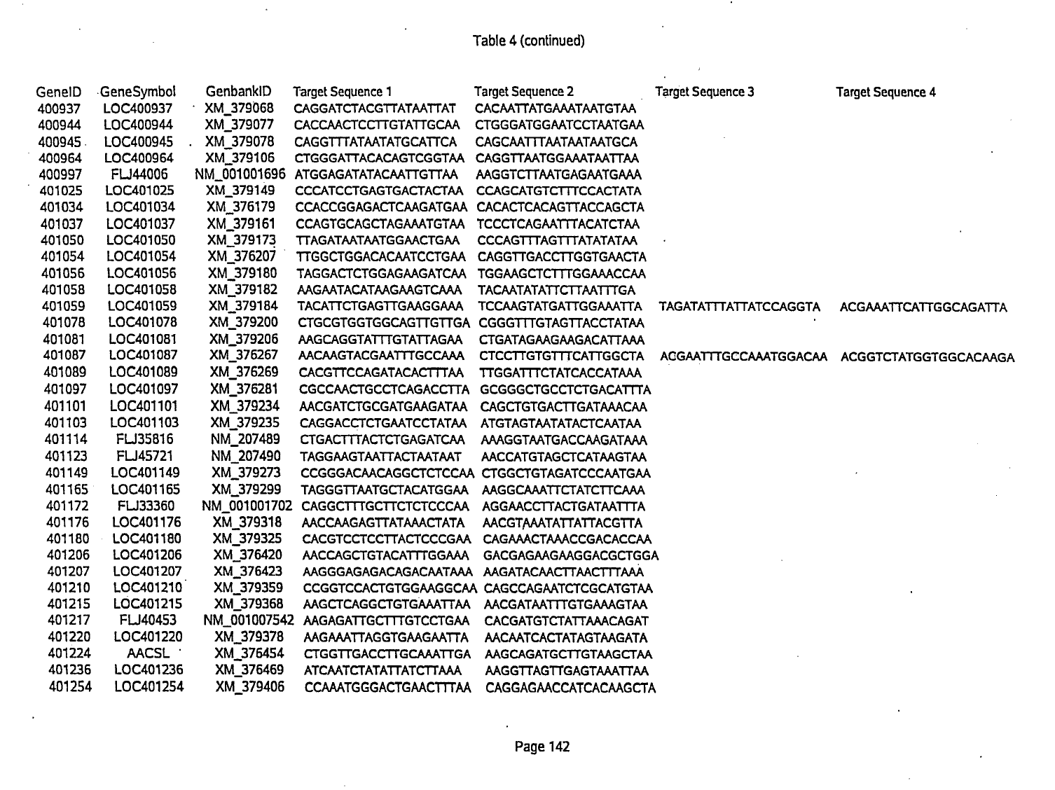

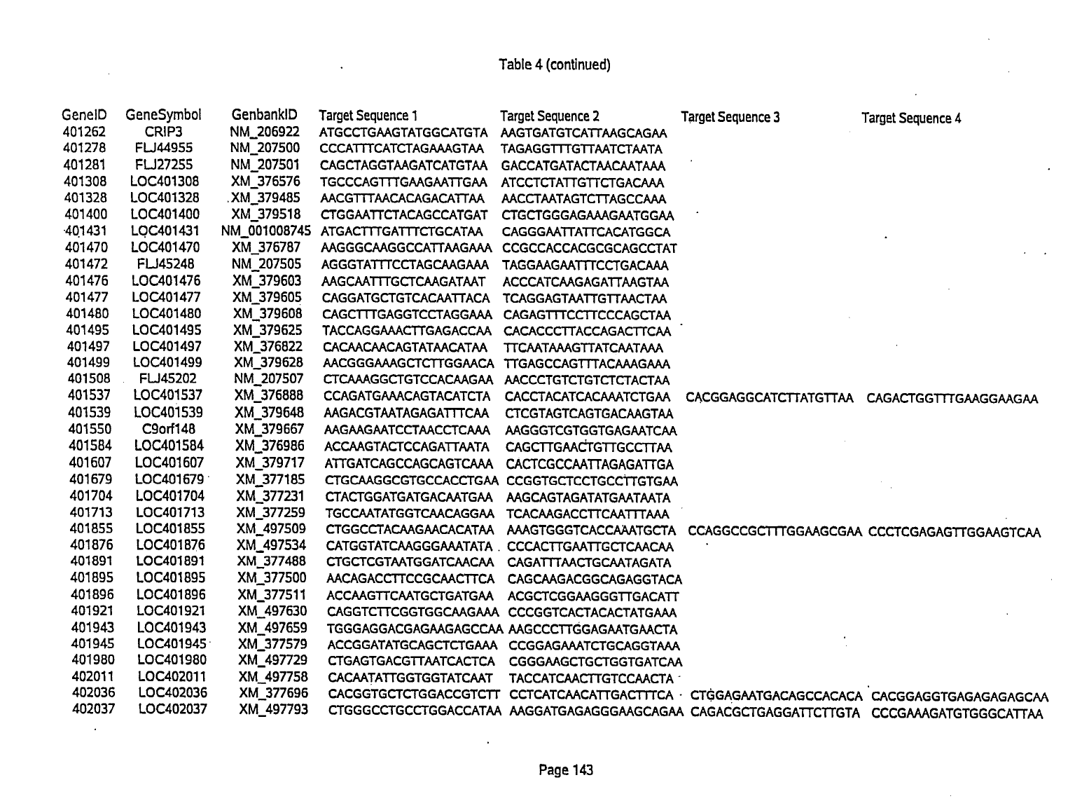

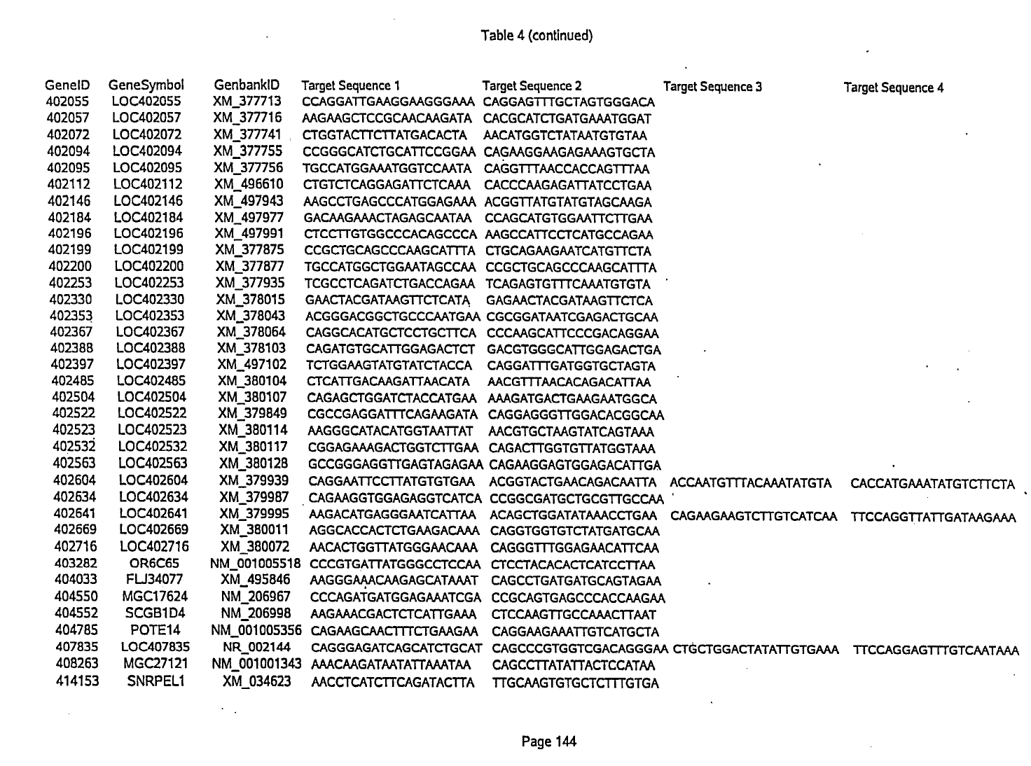

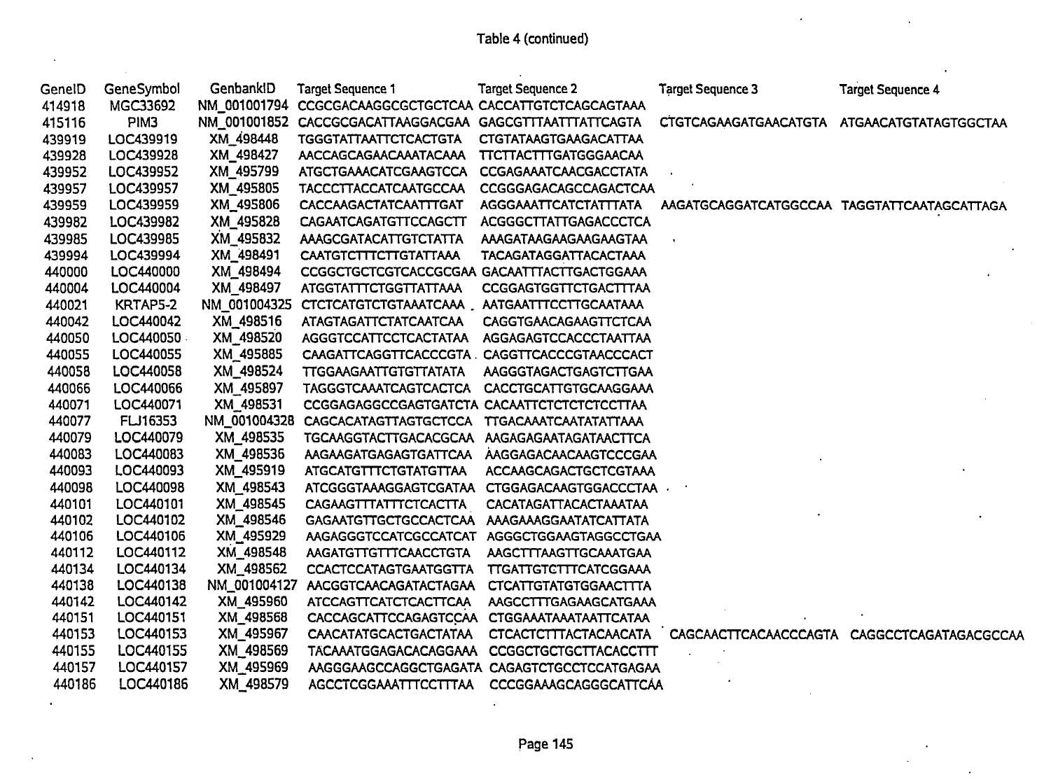

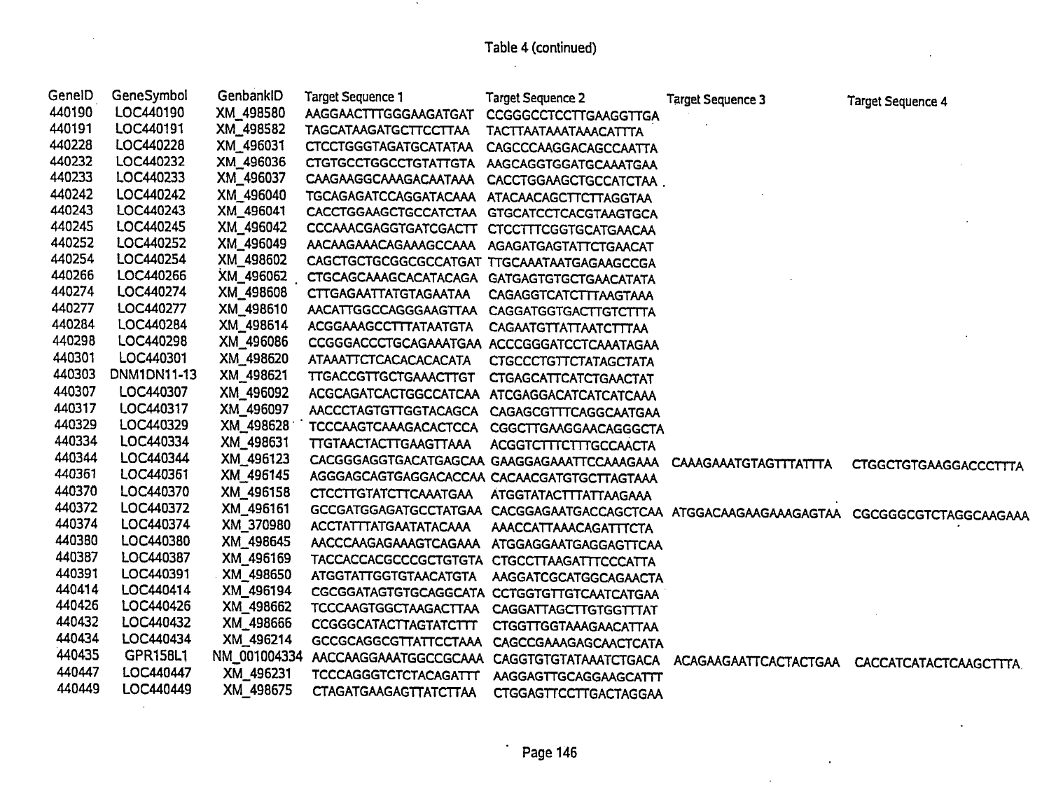

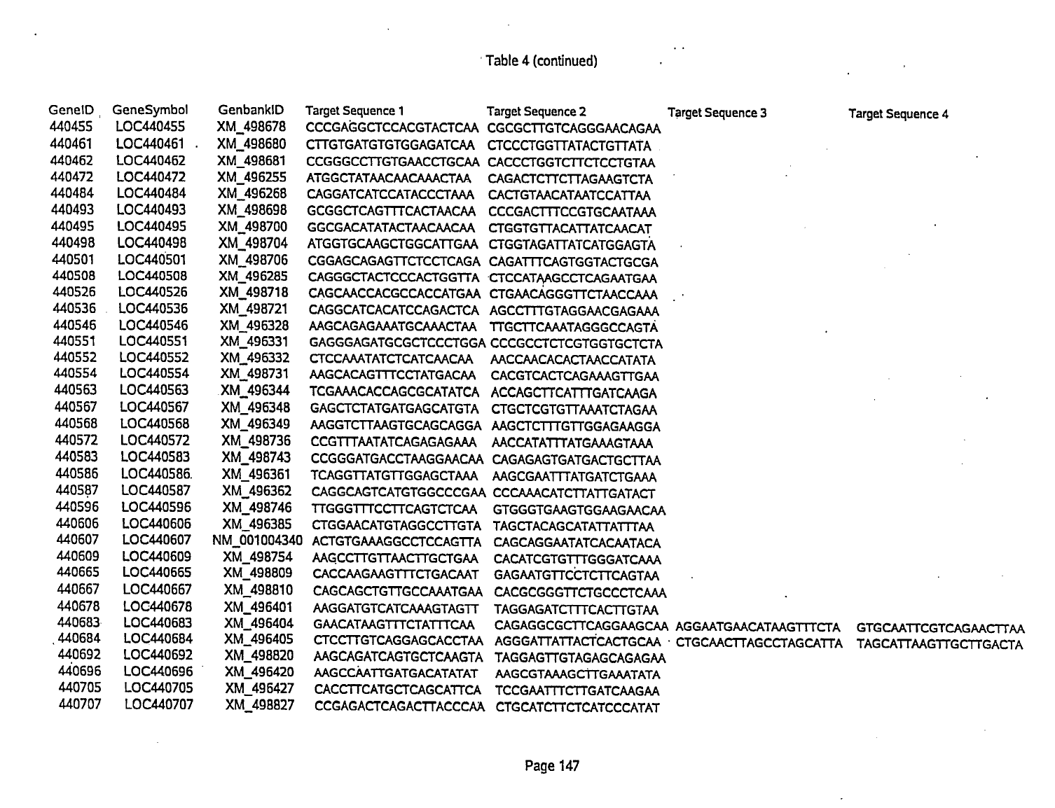

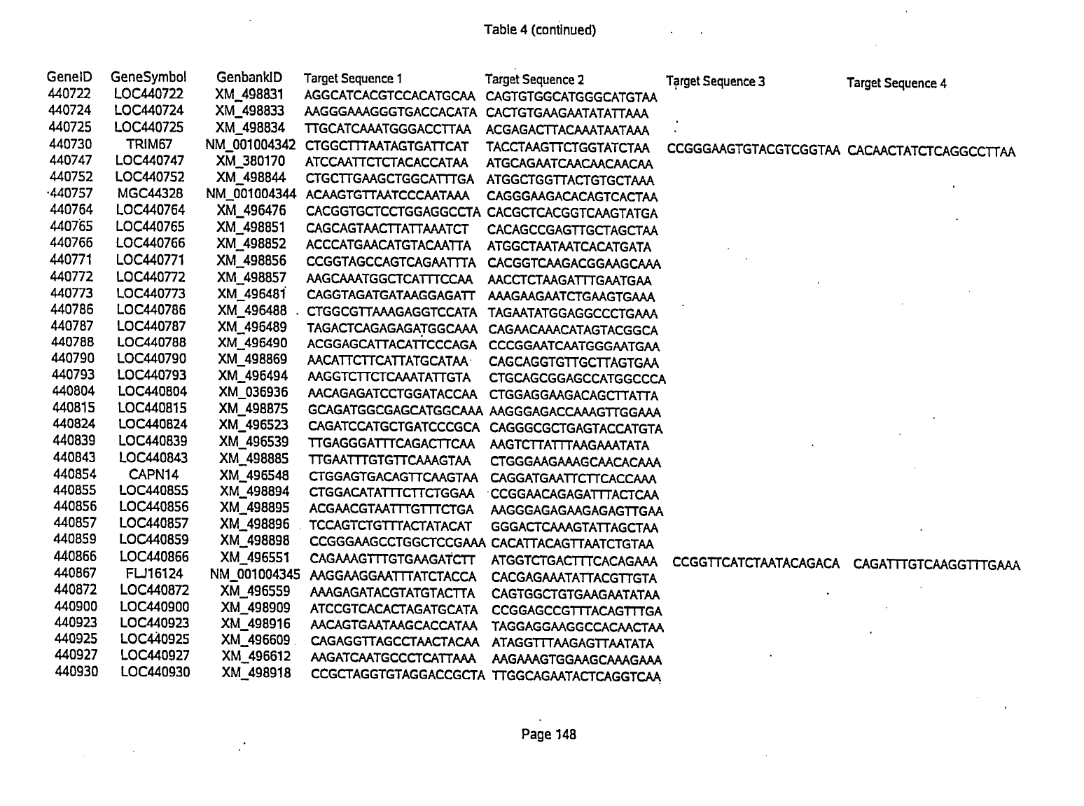

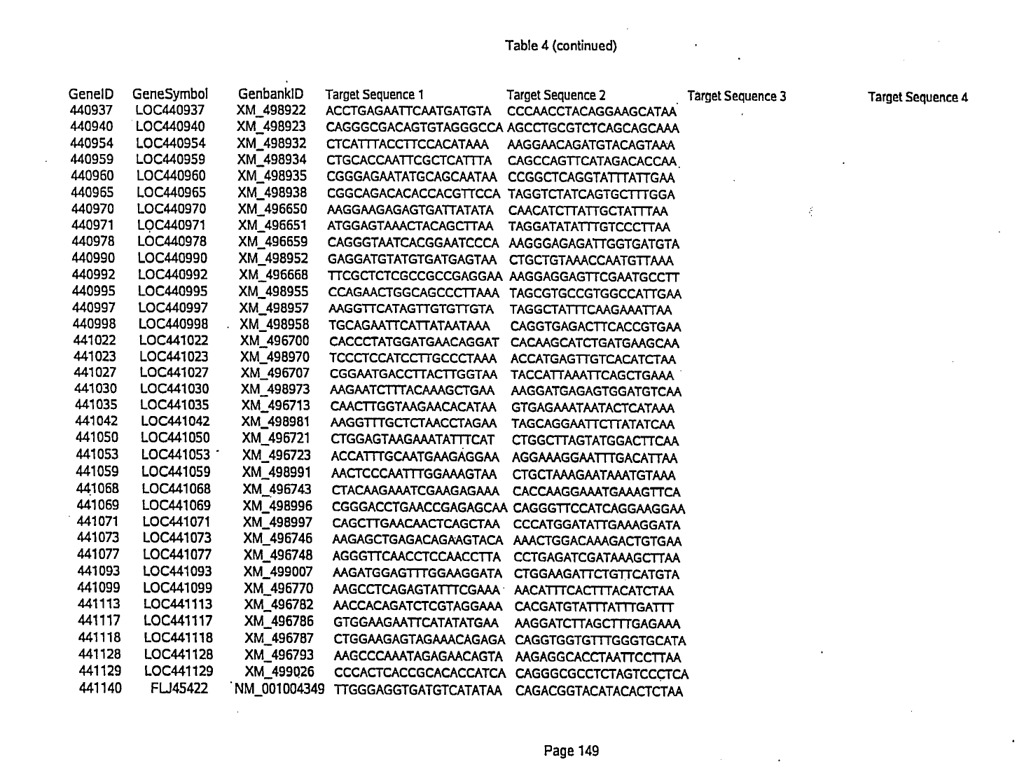

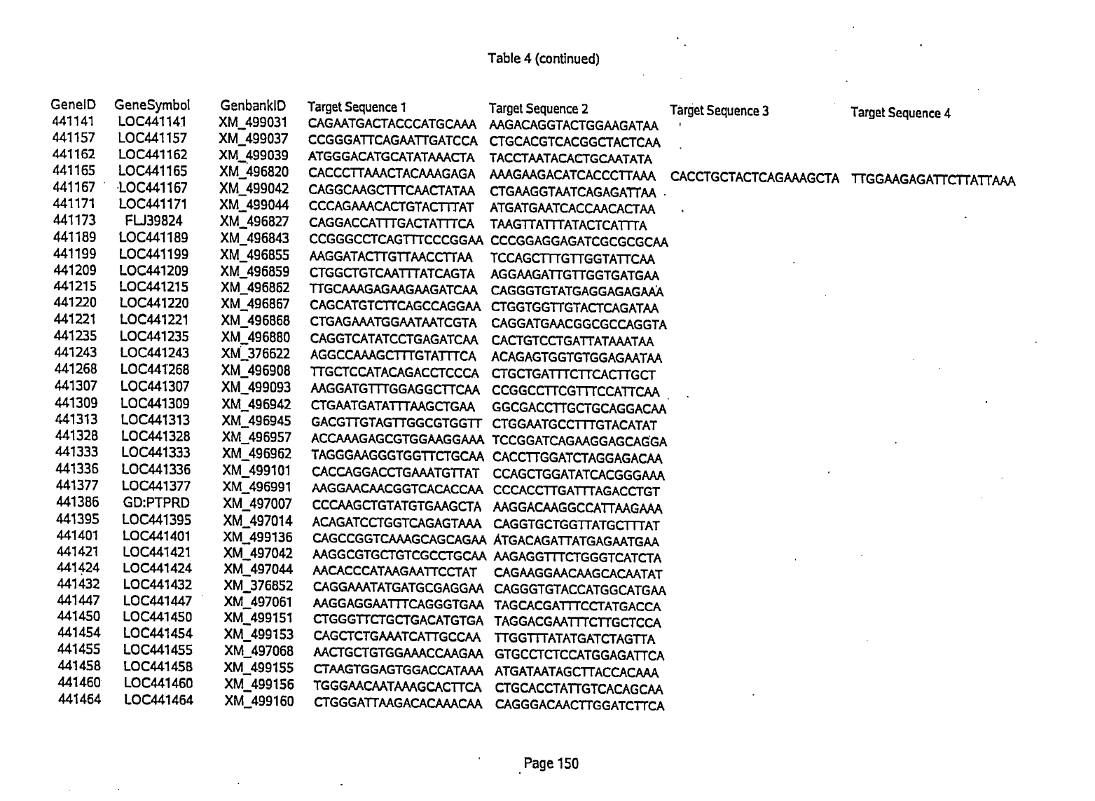

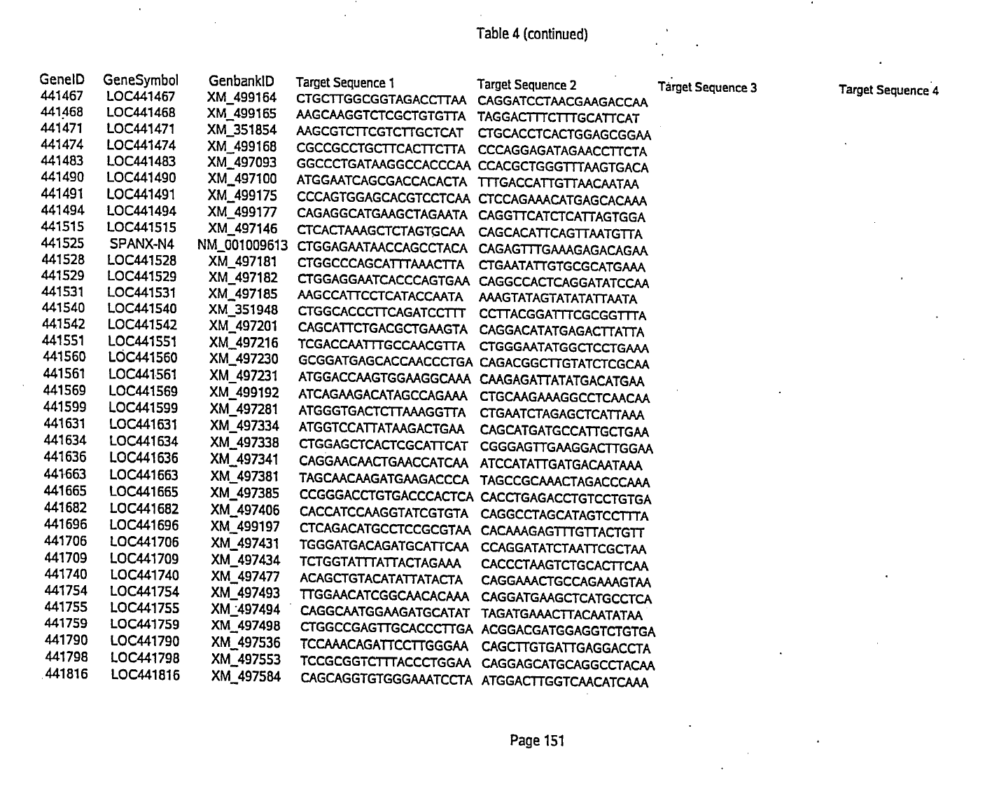

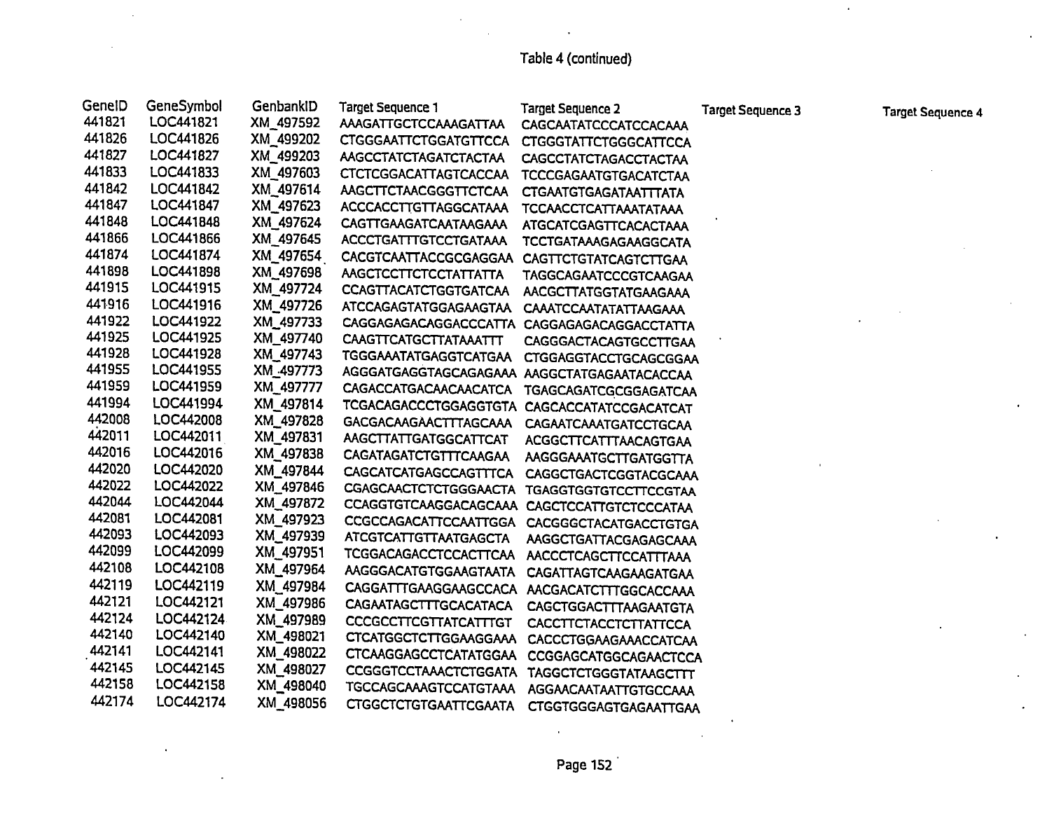

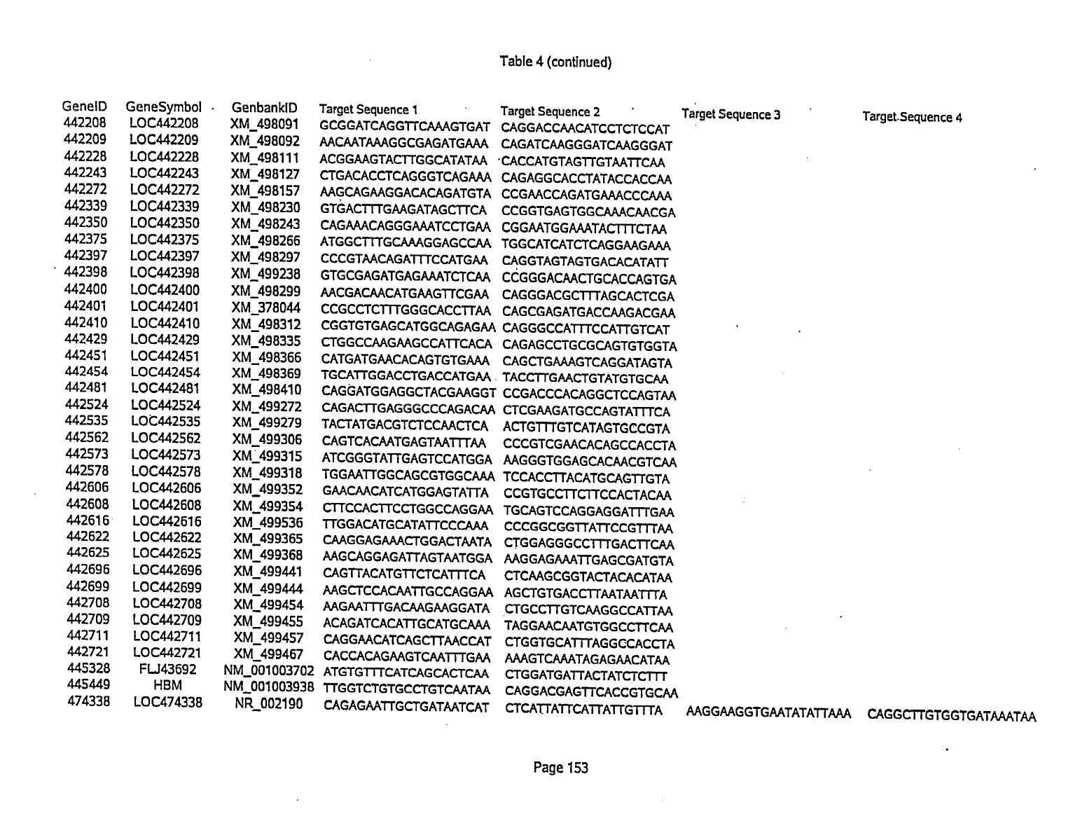

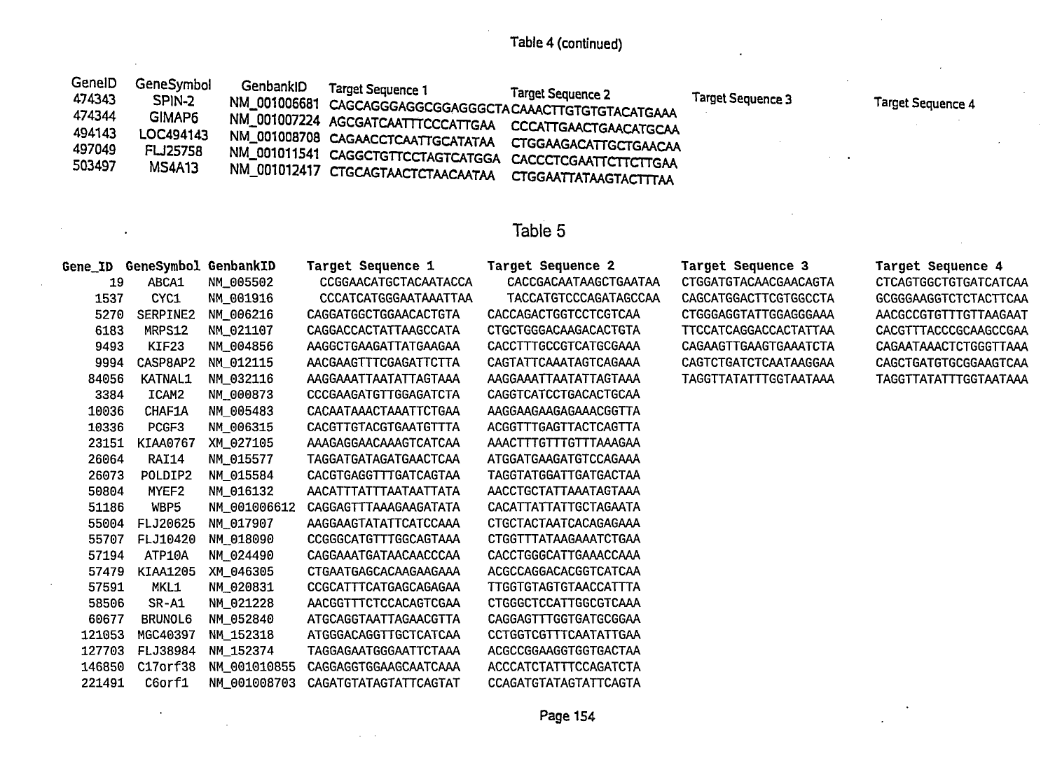

The cell based assay comprised the following steps (see also FIG. 5 which describes the experimental setup of the genome wide siRNA screen:
Day 1: Seeding of A549 cells (lung epithelial cells)+reverse transfection of siRNAs
Day 3: Infection with influenza A/WSN/33+transfection of 293T cells zq with indicator plasmid
Day 4: Infection of 293T cells with the supernatant of A549 cells+fixation of A549 cells with formaldehyde
Day 5: Luciferase Assay to quantify virus replication in 293T cells
Day x: Determination of infection rate by the automated microscope.

### REFERENCE LIST

1. T. D. Carroll et al., J. Immunol. 180, 2385 (2008).
2. K. Subbarao et al., Science 279, 393 (1998).
3. E. C. Claas et al., Lancet 351, 472 (1998).
4. C. Fraser et al., Science (2009).
5. T. Wolff et al., Biol. Chem. 389, 1299 (2008).
6. H. Kato et al., Nature 441, 101 (2006).
7. B. Opitz et al., Cell Microbiol. 9, 930 (2007).
8. J. Lindenmann, Virology 16, 203 (1962).
9. O. Haller, H. Arnheiter, I. Gresser, J. Lindenmann, J. Exp. Med. 49, 601 (1979).
10. T. M. Tumpey et al., J. Virol. 81, 10818 (2007).
11. O. Haller, P. Staeheli, G. Kochs, Biochimie 89, 812 (2007).
12. J. Pavlovic et al., J. Virol. 69, 4506 (1995).
13. B. G. Hale, R. E. Randall, J. Ortin, D. Jackson, J. Gen. Virol. 89, 359 (2008).
14. E. Gottwein, B. R. Cullen, Cell Host. Microbe 3, 375 (2008).
15. B. R. Cullen, Nature 457, 421 (2009).
16. C. L. Jopling, M. Yi, A. M. Lancaster, S. M. Lemon, P. Sarnow, Science 309, 1577 (2005).

1. A method for the preparation of an influenza virus, comprising the steps:
(a) providing a modified cell, a modified embryonated egg or/and a modified non-human organism capable of replicating an influenza virus, wherein the capability of influenza virus replication is increased compared with influenza virus replication in the absence of the modification,
(b) contacting the cell, the embryonated egg or/and the organism of (a) with an influenza virus,
(c) cultivating the cell, the embryonated egg or/and the non-human organism under conditions allowing the replication of the influenza virus, and
(d) isolating the influenza virus or/and at least one component thereof produced in step (c).

2. The method of claim 1, wherein step (a) includes contacting the cell, the embryonated egg or/and the non-human organism with at least one modulator capable of increasing the influenza virus replication in the cell or/and the organism, compared with influenza virus replication in the absence of the modulator.

3. The method of claim 1, wherein step (a) includes the production or/and provision of a recombinant cell, a recombinant embryonated egg or/and a recombinant non-human organism, wherein the expression or/and activity of at least one gene or/and gene product is modified so that the capability of the cell, the embryonated egg or/and the non-human organism of replicating an influenza virus is increased compared with influenza virus replication in the absence of the modification.

4. The method of claim 1, wherein the influenza virus is an influenza A virus or/and an influenza B virus, preferably a strain selected from H1N1, H3N2, H7N7, H5N1.

5. The method of claim 1, wherein modification of the cell, the embryonated egg or/and non-human organism includes inhibition of the expression or/and gene product activity of the MxA gene.

6. The method of claim 5, wherein the expression or/and gene product activity of the MxA gene is inhibited by at least one modulator selected from miR-141, miR-141*, miR-200c, miR-200c*, precursors thereof, derivatives thereof, antisense nucleic acids, siRNAs, shRNAs and influenza virus sequences.

7. The method of claim 5, wherein at least one of miR-141, miR-141*, miR-200c, miR-200c* and precursors thereof is over-expressed in the cell, in the embryonated egg or/and in the non-human organism.

8. The method of claim 1, wherein modification of the cell, of the embryonated egg or/and the non-human organism includes the inhibition of the expression or/and gene product activity of a gene, wherein the gene comprises
(a) a nucleotide sequence selected from the sequences of Table 1A and Table 5
(b) a fragment of the sequence of (a) having a length of at least 70%, at least 80%, at least 90%, at least 95%, at least 98%, at least 99% of the sequence of (a),
(c) a sequence which is at least 70%, preferably at least 80%, more preferably at least 90% identical to the sequence of (a) or/and (b), or/and
(d) a sequence complementary to a sequence of (a), (b) or/and (c).

9. The method of claim 1, wherein modification of the cell, of the embryonated egg or/and of the non-human organism includes the activation of the expression or/and gene product activity of a gene, wherein the gene comprises
(i) a nucleotide sequence selected from the sequences of Table 1B and Table 4,
(ii) a fragment of the sequence of (i) having a length of at least 70%, at least 80%, at least 90%, at least 95%, at least 98%, at least 99% of the sequence of (i),
(iii) a sequence which is at least 70%, preferably at least 80%, more preferably at least 90% identical to the sequence of (i) or/and (ii), or/and
(iv) a sequence complementary to a sequence of (i), (ii) or/and (iii).

10. The method according to claim 2, wherein the at least one modulator is selected from the group consisting of nucleic acids, nucleic acid analogues, peptides, polypeptides, antibodies, aptamers, spiegelmers, small molecules and decoy nucleic acids.

11. The method of claim 10, wherein the nucleic acid is selected from
(a) RNA, analogues and derivatives thereof,
(b) DNA, analogues and derivatives thereof, and
(c) combinations of (a) and (b).

12. The method according to claim 10, wherein the nucleic acid is
(i) an RNA molecule capable of RNA interference, such as sRNA or/and shRNA
(ii) a miRNA,
(iii) a precursor of the RNA molecule (i) or/and (ii),
(iv) a fragment of the RNA molecule (i), (ii) or/and (iii),
(v) a derivative of the RNA molecule of (i), (ii) (iii) or/and (iv), or/and
(vi) a DNA molecule encoding the RNA molecule of (i), (ii) (iii) or/and (iv).

13. The method according to claim 10, wherein the RNA molecule is a double-stranded RNA molecule, preferably a double-stranded sRNA molecule with or without a single-stranded overhang alone at one end or at both ends.

14. The method according to claim 10, wherein the RNA molecule comprises at least one nucleotide analogue or/and deoxyribonucleotide.

15. The method according to claim 10, wherein, the nucleic acid is selected from (a) aptamers, (b) DNA molecules encoding an aptamer, and (c) spiegelmers.

16. The method according to claim 10, wherein the nucleic acid is an antisense nucleic acid or and a DNA encoding the antisense nucleic acid.

17. The method according to claim 10, wherein the nucleic acid has a length of at least 15, preferably at least 17, more preferably at least 19, most preferably at least 21 nucleotides.

18. The method according to claim 10, wherein the nucleic acid has a length of at the maximum 29, preferably at the maximum 27, more preferably at the maximum 25, especially more preferably at the maximum 23, most preferably at the maximum 22 nucleotides.

19. The method according to claim 10, wherein the antibody is directed against a polypeptide comprising
(a) an amino acid sequence encoded by a nucleic acid or/and gene selected from Table 1A , Table 1B, Table 4, and Table 5,
(b) a fragment of the sequence of (a) having a length of at least 70%, at least 80%, at least 90%, at least 95%, at least 98%, at least 99% of the sequence of (a), or/and
(c) an amino acid sequence which is at least 70%, preferably at least 80%, more preferably at least 90% identical to the sequence of (a).

20. The method according to claim 10, wherein the small molecule is directed against a polypeptide comprising
(a) an amino acid sequence encoded by a nucleic acid or/and gene selected from Table 1A, Table 1B, Table 4, and Table 5,
(b) a fragment of the sequence of (a) having a length of at least 70%, at least 80%, at least 90%, at least 95%, at least 98%, at least 99% of the sequence of (a), or/and
(c) an amino acid sequence which is at least 70%, preferably at least 80%, more preferably at least 90% identical to the sequence of (a).

21. A recombinant cell produced in the method of claim 3.

22. A recombinant embryonated egg produced in the method of claim 3.

23. A recombinant non-human organism produced in the method of claim 3.

24. A pharmaceutical composition comprising an activator of the expression or/and gene product activity of the MxA gene, optionally together with a pharmaceutically acceptable carrier, adjuvant, diluent or/and additive.

25. The pharmaceutical composition of claim 21, wherein the activator of the expression or/and gene product activity is at least one inhibitor capable of inhibiting the activity of an miRNA selected from miR-141, miR-141*, miR-200c, miR-200c*, and precursors thereof.
